(19) United States
(12) Patent Application Publication
(10) Pub. No.: US 2024/0400975 A1
(43) Pub. Date: Dec. 5, 2024

(54) METHODS FOR IMPROVING PHAGE RESISTANCE IN CLOSTRIDIUM SPECIES

(71) Applicant: NORTH CAROLINA STATE UNIVERSITY, Raleigh, NC (US)

(72) Inventors: Rodolphe BARRANGOU, Raleigh, NC (US); Claudio Hidalgo CANTABRANA, Cary, NC (US); Matthew A. NETHERY, Raleigh, NC (US)

(21) Appl. No.: 18/691,098

(22) PCT Filed: Sep. 12, 2022
(86) PCT No.: PCT/US2022/076259

§ 371 Date: Mar. 12, 2024

Related U.S. Application Data
(60) Provisional application No. 63/243,395, filed on Sep. 13, 2021.

(51) Int. Cl. C12N 1/20; C12N 9/22; C12N 15/11; C12N 15/90

(52) U.S. Cl. CPC C12N 1/20; C12N 9/22; C12N 15/11; C12N 15/902

(57) ABSTRACT

This invention relates to methods for increasing phage resistance in Clostridium species through incorporation of novel recombinant Clustered Regularly Interspaced Short Palindromic Repeats (CRISPR) RNAs using endogenous CRISPR systems and/or recombinant nucleic acid constructs encoding clostridia Type I-B and Type I-C CASCADE complexes, and/or expression cassettes and vectors comprising the same.

### STATEMENT OF PRIORITY

This application claims the benefit, under 35 U.S.C. § 119 (e), of U.S. Provisional Application No. 63/243,395 filed on Sep. 13, 2021, the entire contents of which is incorporated by reference herein.

### STATEMENT REGARDING ELECTRONIC FILING OF A SEQUENCE LISTING

A Sequence Listing in XML text format, entitled 5051-990WO.xml, 459,739 bytes in size, generated on Sep. 9, 2022 and filed herewith, is hereby incorporated by reference into the specification for its disclosures.

### FIELD OF THE INVENTION

This invention relates to methods for increasing phage resistance in Clostridium species through incorporation of novel recombinant Clustered Regularly Interspaced Short Palindromic Repeats (CRISPR) nucleic acids using endogenous CRISPR systems and/or recombinant nucleic acid constructs encoding clostridia Type I-B and Type I-C CASCADE complexes, and/or expression cassettes and vectors comprising the same.

### BACKGROUND OF THE INVENTION

Clustered Regularly Interspaced Short Palindromic Repeats (CRISPR), in combination with CRISPR-associated genes (cas) constitute the CRISPR-Cas system, which confers adaptive immunity in many bacteria and most archaea. CRISPR-mediated immunization occurs through the integration of DNA from invasive genetic elements such as plasmids and phages that can be used to thwart future infections by invaders containing the same sequence.

CRISPR-Cas systems consist of CRISPR arrays of short DNA “repeats” interspaced by hypervariable “spacer” sequences and a set of flanking cas genes. The system acts by providing adaptive immunity against invasive genetic elements such as phage and plasmids through the sequence-specific targeting and interference of foreign nucleic acids (Barrangou et al. 2007. Science. 315:1709-1712; Brouns et al. 2008. Science 321:960-4; Horvath and Barrangou. 2010. Science. 327:167-70; Marraffini and Sontheimer. 2008. Science. 322:1843-1845; Bhaya et al. 2011. Annu. Rev. Genet. 45:273-297; Terns and Terns. 2011. Curr. Opin. Microbiol. 14:321-327; Westra et al. 2012. Annu. Rev. Genet. 46:311-339; Barrangou R. 2013. RNA. 4:267-278). Typically, invasive DNA sequences are acquired as novel “spacers” (Barrangou et al. 2007. Science. 315:1709-1712), each paired with a CRISPR repeat and inserted as a novel repeat-spacer unit in the CRISPR locus. The “spacers” are acquired by the Cas1 and Cas2 proteins that are universal to all CRISPR-Cas systems (Makarova et al. 2011. Nature Rev. Microbiol. 9:467-477; Yosef et al. 2012. Nucleic Acids Res. 40:5569-5576), with involvement by the Cas4 protein in some systems (Plagens et al. 2012. J. Bact. 194: 2491-2500; Zhang et al. 2012. PLoS One 7:e47232). The resulting repeat-spacer array is transcribed as a long pre-CRISPR RNA (pre-CRISPR, pre-crRNA) (Brouns et al. 2008. Science 321:960-4), which is processed into CRISPR RNAs (CRISPRs, crRNAs) that drive sequence-specific recognition of DNA or RNA. Specifically, crRNAs guide nucleases towards complementary targets for sequence-specific nucleic acid cleavage mediated by Cas endonucleases (Garneau et al. 2010. Nature. 468:67-71; Haurwitz et al. 2010. Science. 329:1355-1358; Sapranauskas et al. 2011. Nucleic Acid Res. 39:9275-9282; Jinek et al. 2012. Science. 337:816-821; Gasiunas et al. 2012. Proc. Natd. Acad. Sci. 109:E2579-E2586; Magadan et al. 2012. PLoS One. 7:e40913; Karvelis et al. 2013. RNA Biol. 10:841-851).

These widespread systems occur in nearly half of bacteria (about 46%) and the large majority of archaea (about 90%). CRISPR/Cas are subdivided into six main classes and types based on the cas gene content, organization and variation in the biochemical processes that drive crRNA biogenesis, and Cas protein complexes that mediate target recognition and cleavage (Makarova et al. 2011. Nature Rev. Microbiol. 9:467-477; Makarova et al. 2013. Nucleic Acid Res. 41:4360-4377; Makarova et al. 2015. Nature Rev. Microbiol. 13:722-736). In types I, III and IV, the specialized Cas endonucleases process the pre-crRNAs, which then assemble into a large multi-Cas protein complex capable of recognizing and cleaving nucleic acids complementary to the crRNA. A different process is involved in Type II CRISPR-Cas systems. Here, the pre-CRNAs are processed by a mechanism in which a trans-activating crRNA (tracrRNA) hybridizes to repeat regions of the crRNA. The hybridized crRNA-tracrRNA are cleaved by RNase III and following a second event that removes the 5′ end of each spacer, mature crRNAs are produced that remain associated with the both the tracrRNA and Cas9. The mature complex then locates a target dsDNA sequence (‘protospacer’ sequence) that is complementary to the spacer sequence in the complex and cuts both strands. Target recognition and cleavage by the complex in the Type II system not only requires a sequence that is complementary between the spacer sequence on the crRNA-tracrRNA complex and the target ‘protospacer’ sequence but also requires a protospacer adjacent motif (PAM) sequence located at the 3′ end of the protospacer sequence. The exact PAM sequence that is required can vary between different Type II systems.

Class 1 uses multiple Cas proteins in a cascade complex to degrade nucleic acids. Class 2 uses a single large Cas protein to degrade nucleic acids. The type I systems are the most prevalent in bacteria and in archaea (Makarova et al. 2011. Nature Rev. Microbiol. 9:467-477) and target DNA (Brouns et al. 2008. Science 321:960-4). A complex of 3-8 Cas proteins called the CRISPR associated complex for antiviral defense (Cascade) processes the pre-crRNAs (Brouns et al. 2008. Science 321:960-4), retaining the crRNA to recognize DNA sequences called “protospacers” that are complementary to the spacer portion of the crRNA. Aside from complementarity between the crRNA spacer and the protospacer, targeting requires a protospacer-adjacent motif (PAM) located at the 5′ end of the protospacer (Mojica et al. 2009. Microbiology 155:733-740; Sorek et al. 2013. Ann. Rev. Biochem. 82:237-266). For type I systems, the PAM is directly recognized by Cascade (Sashital et al. 2012. Mol. Cell 46:606-615; Westra et al. 2012. Mol. Cell 46:595-605). The exact PAM sequence that is required can vary between different type I systems. Once a protospacer is recognized, Cascade generally recruits the endonuclease Cas3, which cleaves and degrades the target DNA (Sinkunas et al. 2011. EMBO J. 30:1335-1342; Sinkunas et al. 2013. EMBO J. 32:385-394).

### SUMMARY OF THE INVENTION

A first aspect of the invention provides a method of enhancing resistance of a bacterial cell to one or more bacteriophage species or strains, the method comprising introducing into the bacterial cell a recombinant nucleic acid construct comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and each of the one or more spacer sequences is complementary to a target sequence (protospacer) in a target nucleic acid of a bacteriophage species or strain, wherein the target sequence is located immediately adjacent (3′) to a protospacer adjacent motif (PAM).

A second aspect of the invention provides a method of enhancing resistance of a bacterial cell to one or more bacteriophage species or strains, the method comprising introducing into the bacterial cell (a) at least one protein-RNA complex comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence, and each of the one or more spacer sequences is complementary to a target sequence (protospacer) in a target nucleic acid of a bacteriophage species or strain, wherein the target sequence is located immediately adjacent (3′) to a protospacer adjacent motif (PAM) and (b) at least one polypeptide of a Type I CRISPR-Cas system, a Type II CRISPR-Cas system, a Type III CRISPR-Cas system, a Type IV CRISPR-Cas system, a Type V CRISPR-Cas system or a Type VI CRISPR CRISPR-Cas system.

A third aspect of the invention provides a method of enhancing resistance of a bacterial cell to one or more bacteriophage species or strains, the method comprising introducing into the bacterial cell at least one protein-RNA complex comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence, and each of the one or more spacer sequences is complementary to a target sequence (protospacer) in a target nucleic acid of a bacteriophage species or strain, wherein the target sequence is located immediately adjacent (3′) to a protospacer adjacent motif (PAM) and (a) a Cas3 polypeptide and a Type I-B Cascade complex comprising a Cas6 polypeptide, a Cas8 polypeptide, a Cas7 polypeptide, and a Cas5 polypeptide; or (b) a Cas3 polypeptide and a Type I-C Cascade complex comprising a Cas5 polypeptide, a Cas8 polypeptide and a Cas7 polypeptide.

Further provided are cells and/or organisms produced by the methods of the invention and nucleic acid constructs for carrying out the methods. These and other aspects of the invention are set forth in more detail in the description of the invention below.

### BRIEF DESCRIPTION OF THE DRAWINGS

FIGS. 1A-1B provides a schematic representation of CRISPR-Cas loci architecture of Clostridium spp. 1141A1FAA Type I-B (FIG. 1A) and Erysipelatoclostridium ramosum Type I-B (FIG. 1B).

FIGS. 2A-2G provides a schematic of the Type I-C CRISPR-Cas loci architecture of Clostridium bolteae DSM15670 (BAA-613) (FIG. 2A), Clostridium bolteae WAL14578 (FIG. 2B), Clostridium clostridioforme WAL7855 (FIG. 2C), Clostridium clostridioforme 2149FAA (FIG. 2D), Clostridium clostridioforme YL32 (FIG. 2E), Clostridium clostridioforme NCTC11224 (FIG. 2F) and Clostridium scindens ATCC 35704 (FIG. 2G).

FIG. 3 shows the comparison of CRISPR-Cas system subtype I-C of select Clostridium species of interest to the canonical subtype I-C from Bacillus halodurans C-125.

FIG. 4 provides 16S phylogenetic tree of several Clostridium species and E. coli.

FIG. 5 shows PAM prediction data for Clostridium spp. 1141A1FAA.

FIG. 6 shows PAM prediction data for Erysipelatoclostridium ramosum.

FIG. 7 shows PAM prediction data for the Type I-C CRISPR-Cas system of Clostridium bolteae.

FIG. 8 shows PAM prediction data for the Type I-C CRISPR-Cas system of Clostridium clostidioforme.

FIG. 9 shows PAM prediction data for the Type I-C CRISPR-Cas system of Clostridium scindens.

FIG. 10 provides E. ramosum DSM 1402 type I-B mRNA-seq reads data.

FIG. 11 provides E. ramosum DSM 1402 type I-B smRNA-seq reads showing CRISPR array transcription and mature crRNA biogenesis.

FIG. 12 shows boundaries of mature crRNAs of E. ramosum DSM 1402 and the sequence of the mature crRNA of E. ramosum DSM 1402 at the bottom of the figure along with the hairpin structure of the mature crRNA.

FIG. 13 shows targeting using a CRISPR array having a spacer that is complementary to the target sequence having the PAM sequence of ATCG flanking the 5′ edge of the protospacer (upper panel) and a CRISPR array having a spacer that is complementary to the target sequence having the PAM sequence of ATCA flanking the 5′ edge of the protospacer (lower panel).

FIG. 14 shows round 1 testing 0.5 nM E. ramosum Type 1-B PAM ATCA or ATCG plasmid using a non-targeting plasmid as a negative control.

FIG. 15 shows round 2 testing 0.5 nM E. ramosum Type 1-B PAM ATCA or ATCG plasmid using a non-targeting plasmid as a negative control.

FIG. 16 shows round 2 testing 0.5 nM E. ramosum Type 1-B PAM ATCA or ATCG plasmid using a non-targeting plasmid as a negative control.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancement,” “improve” and “improvement” (and the like and grammatical variations thereof) describe an elevation of at least about 5%, 10%, 15%, 20%, 25%, 50%, 75%, 100%, 150%, 200%, 300%, 400%, 500%, 750%, 1000%, 2500%, 5000%, 10,000%, 20,000% or more as compared to a control (e.g., a CRISPR targeting a particular gene having, for example, more spacer sequences targeting different regions of that gene and therefore having increased repression of that gene as compared to a CRISPR targeting the same gene but having, for example, fewer spacer sequences targeting different regions of that gene).

As used herein, the terms “reduce,” “reduced,” “reducing,” “reduction,” “diminish,” “suppress,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount. As an example, a mutation in a Cas3 nuclease can reduce the nuclease activity of the Cas3 by at least about 90%, 95%, 97%, 98%, 99%, or 100% as compared to a control (e.g., wild-type Cas3).

The terms “complementary” or “complementarity,” as used herein, refer to the natural binding of polynucleotides under permissive salt and temperature conditions by base-pairing. For example, the sequence “A-G-T” binds to the complementary sequence “T-C-A.” Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement” as used herein can mean 100% complementarity with the comparator nucleotide sequence or it can mean less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

As used herein, the phrase “substantially complementary,” or “substantial complementarity” in the context of two nucleic acid molecules, nucleotide sequences or protein sequences, refers to two or more sequences or subsequences that are at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue complementary, when compared and aligned for maximum correspondence, as measured using one of the following sequence comparison algorithms or by visual inspection. In some embodiments, substantial complementarity can refer to two or more sequences or subsequences that have at least about 80%, at least about 85%, at least about 90%, at least about 95, 96, 96, 97, 98, or 99% complementarity (e.g., about 80% to about 90%, about 80% to about 95%, about 80% to about 96%, about 80% to about 97%, about 80% to about 98%, about 80% to about 99% or more, about 85% to about 90%, about 85% to about 95%, about 85% to about 96%, about 85% to about 97%, about 85% to about 98%, about 85% to about 99% or more, about 90% to about 95%, about 90% to about 96%, about 90% to about 97%, about 90% to about 98%, about 90% to about 99% or more, about 95% to about 97%, about 95% to about 98%, about 95% to about 99% or more). Two nucleotide sequences can be considered to be substantially complementary when the two sequences hybridize to each other under stringent conditions. In some representative embodiments, two nucleotide sequences considered to be substantially complementary hybridize to each other under highly stringent conditions.

As used herein, “contact,” contacting,” “contacted,” and grammatical variations thereof, refers to placing the components of a desired reaction together under conditions suitable for carrying out the desired reaction (e.g., integration, transformation, site-specific cleavage (nicking, cleaving), amplifying, site specific targeting of a polypeptide of interest and the like). The methods and conditions for carrying out such reactions are well known in the art (See, e.g., Gasiunas et al. (2012) Proc. Natl. Acad. Sci. 109:E2579-E2586; M. R. Green and J. Sambrook (2012) Molecular Cloning: A Laboratory Manual. 4th Ed., Cold Spring Harbor Laboratory Press, Cold Spring Harbor, NY).

As used herein, the term “commensal bacteria” refers to a bacterium that is naturally present in a microbiome, such as in the gut microbiome of a host (e.g., human gut microbiome), without causing harm to the host. In some cases, a commensal bacterium may confer a benefit to the host organism.

As used herein, Type I Clustered Regularly Interspaced Short Palindromic Repeats (CRISPR)-associated complex for antiviral defense (Cascade) refers to a complex of polypeptides involved in processing of pre-crRNAs and subsequent binding to the target DNA in Type I CRISPR-Cas systems (e.g., cas3 and Cas6, Cas8, Cas7 and Cas5 or Cas8, Cas7 and Cas5). Other CRISPR Cas systems may also be used with this invention for enhancing the resistance of a bacterium or a bacterial population (e.g., a Type II, Type III, Type IV, Type V or Type VI CRISPR-Cas system; e.g., a CRISPR and CRISPR-Cas polypeptides from these systems).

Type I CRISPR-Cas systems are well known in the art and include, for example, Archaeoglobus fulgidus comprises an exemplary Type I-A CRISPR-Cas system, Clostridium kluyveri DSM 555 comprises an exemplary Type I-B CRISPR-Cas system, Bacillus halodurans C-125 comprises an exemplary Type I-C CRISPR-Cas system, Cyanothece sp. PCC 802 comprises an exemplary Type I-D CRISPR-Cas system, Escherichia coli K-12 and Lactobacillus crispatus comprise exemplary Type I-E CRISPR-Cas systems, Geobacter sulfurreducens comprises an exemplary Type I-U CRISPR-Cas system and Yersinia pseudotuberculosis YPIII comprises an exemplary Type I-F CRISPR-Cas system.

As used herein, “Type I polypeptide” refers to any of a Cas3 polypeptide, Cas3′ polypeptide, a Cas3″ polypeptide and any one or more of the Type I Clustered Regularly Interspaced Short Palindromic Repeats (CRISPR)-associated complex for antiviral defense (“Cascade”) polypeptides. Thus, the term “Type I polypeptide” refers to the polypeptides that make up a Type I-A CRISPR-Cas system, a Type I-B CRISPR-Cas system, a Type I-C CRISPR-Cas system, a Type I-D CRISPR-Cas system, a Type I-E CRISPR-Cas system, and/or a Type I-F CRISPR-Cas system. Each Type-I CRISPR-Cas system comprises at least one Cas3 polypeptide. Cas3 polypeptides generally comprise both a helicase domain and an HD domain. However, in some Type I CRISPR-Cas systems, the helicase and HD domain are found in separate polypeptides, Cas3′ and Cas3″. In particular, Cas3′ encodes the helicase domain whereas Cas3″ encodes the HD domain. Consequently, because both domains are required for Cas3 function, Type I subtypes either encode Cas3 (I-C, I-D, I-E, I-F) or Cas3′ and Cas3″ (I-A, I-B).

As used herein, “Type I Cascade polypeptides” refers to a complex of polypeptides involved in processing of pre-crRNAs and subsequent binding to the target DNA in type I CRISPR-Cas systems. These polypeptides include, but are not limited to, the Cascade polypeptides of Type I subtypes I-A, I-B, I-C, 1-D, I-E and 1-F. Non-limiting examples of Type I-A polypeptides include Cas7 (Csa2), Cas8al (Csx13), Cas8a2 (Csx9), Cas5, Csa5, Cas6a, Cas3′ and/or a Cas3″. Non-limiting examples of Type I-B polypeptides include Cas6b, Cas8b (Cshl), Cas7 (Csh2) and/or Cas5. Non-limiting examples of Type-IC polypeptides include Cas5d, Cas8c (Csdl), and/or Cas7 (Csd2). Non-limiting examples of Type-ID polypeptides include CaslOd (Csc3), Csc2, Cscl, and/or Cas6d. Non-limiting examples of Type I-E polypeptides include Csel (CasA), Cse2 (CasB), Cas7 (CasC), Cas5 (CasD) and/or Cas6e (CasE). Non-limiting examples of Type I-F polypeptides include Cysl, Cys2, Cas7 (Cys3) and/or Cas6f (Csy4).

Exemplary Type I-B polypeptides useful with this invention include a Cas6 polypeptide (SEQ ID NO:122 or SEQ ID NO:140), a Cas8 polypeptide (SEQ ID NO:123 or SEQ ID NO:141), a Cas7 polypeptide (SEQ ID NO:124 or SEQ ID NO:142), a Cas5 polypeptide (SEQ ID NO:125 or SEQ ID NO:143), a Cas3 polypeptide (SEQ ID NO:126 or SEQ ID NO:144). Type I-B Cascade polypeptides that function in spacer acquisition include Cas1 (SEQ ID NO:127 or SEQ ID NO:145), Cas2 (SEQ ID NO:128 or SEQ ID NO:146) and Cas4 (SEQ ID NO:129 or SEQ ID NO:147). In some embodiments of this invention, a recombinant nucleic acid construct may comprise, consist essentially of, or consist of a recombinant nucleic acid encoding a subset of Type I-B Cascade polypeptides that function to process a CRISPR array and subsequently bind to a target DNA using the spacer of the processed CRISPR as a guide. A further Type I-B polypeptide useful with this invention includes a Cas3 nuclease.

In some embodiments of this invention, a recombinant nucleic acid construct may encode the Type I-B polypeptides comprising, consisting essentially of, or consisting of a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:122 or SEQ ID NO:140, a Cas8 polypeptide having at least 80% sequence identity to the amino acid sequence of SEQ ID NO:123 or SEQ ID NO:141, a Cas7 polypeptide having at least 80% sequence identity to the amino acid sequence of SEQ ID NO:124 or SEQ ID NO:142, and a Cas5 polypeptide having at least 80% sequence identity to the amino acid sequence of SEQ ID NO:125 or SEQ ID NO:143 and optionally encoding a Cas3 polypeptide having at least 80% sequence identity to the amino acid sequence of SEQ ID NO:126 or SEQ ID NO:144. Exemplary Type I-C polypeptides useful with this invention include a Cas5 polypeptide (SEQ ID NOs:2, 21, 37, 55, 73, 90, or 107), a Cas8 polypeptide (SEQ ID NOs:3, 22, 38, 56, 74, 91, or 108), a Cas7 polypeptide (SEQ ID NOs:4, 23, 39, 57, 75, 92, or 109), and/or a Cas3 polypeptide (SEQ ID NOs:1, 20, 36, 54, 72, 89, or 106). Type I-C Cascade polypeptides that function in spacer acquisition include Cas4 (SEQ ID NO:5, 24, 40, 57, 76, 93 or 110), Cas1 (SEQ ID NOs:6, 25, 41, 58, 77, 94 or 111), and/or Cas2 (SEQ ID NOs:7, 26, 42, 59, 78, 95 or 112). In some embodiments of this invention, a recombinant nucleic acid construct may comprise, consist essentially of, or consist of a recombinant nucleic acid encoding a subset of Type I-C Cascade polypeptides that function to process a CRISPR array and subsequently bind to a target DNA using the spacer of the processed CRISPR as a guide. A further Type I-C polypeptide useful with this invention includes a Cas3 nuclease.

In some embodiments of this invention, a recombinant nucleic acid construct may encode the Type I-B polypeptides comprising, consisting essentially of, or consisting of (1) a Cas5 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:2, a Cas8 polypeptide having at least 80% sequence identity to the amino acid sequence of SEQ ID NO:3, a Cas7 polypeptide having at least 80% sequence identity to the amino acid sequence of SEQ ID NO:4 and optionally, a Cas3 polypeptide having at least 80% sequence identity to the amino acid sequence of SEQ ID NO:1; (2) a Cas5 polypeptide having at least 80% sequence identity to the amino acid sequence of SEQ ID NO:21, a Cas8 polypeptide having at least 80% sequence identity to the amino acid sequence of SEQ ID NO:22, a Cas7 polypeptide having at least 80% sequence identity to the amino acid sequence of SEQ ID NO:23 and optionally, a Cas3 polypeptide having at least 80% sequence identity to the amino acid sequence of SEQ ID NO:20; (3) a Cas5 polypeptide having at least 80% sequence identity to the amino acid sequence of SEQ ID NO:37, a Cas8 polypeptide having at least 80% sequence identity to the amino acid sequence of SEQ ID NO:38, a Cas7 polypeptide having at least 80% sequence identity to the amino acid sequence of SEQ ID NO:39 and optionally, a Cas3 polypeptide having at least 80% sequence identity to the amino acid sequence of SEQ ID NO:36; (4) a Cas5 polypeptide having at least 80% sequence identity to the amino acid sequence of SEQ ID NO:55, a Cas8 polypeptide having at least 80% sequence identity to the amino acid sequence of SEQ ID NO:56, a Cas7 polypeptide having at least 80% sequence identity to the amino acid sequence of SEQ ID NO:57 and optionally, a Cas3 polypeptide having at least 80% sequence identity to the amino acid sequence of SEQ ID NO:54; (5) a Cas5 polypeptide having at least 80% sequence identity to the amino acid sequence of SEQ ID NO:73, a Cas8 polypeptide having at least 80% sequence identity to the amino acid sequence of SEQ ID NO:74, a Cas7 polypeptide having at least 80% sequence identity to the amino acid sequence of SEQ ID NO:75 and optionally, a Cas3 polypeptide having at least 80% sequence identity to the amino acid sequence of SEQ ID NO:72; (6) a Cas5 polypeptide having at least 80% sequence identity to the amino acid sequence of SEQ ID NO:90, a Cas8 polypeptide having at least 80% sequence identity to the amino acid sequence of SEQ ID NO:91, a Cas7 polypeptide having at least 80% sequence identity to the amino acid sequence of SEQ ID NO:92 and optionally, a Cas3 polypeptide having at least 80% sequence identity to the amino acid sequence of SEQ ID NO:89; or (7) a Cas5 polypeptide having at least 80% sequence identity to the amino acid sequence of SEQ ID NO:107, a Cas8 polypeptide having at least 80% sequence identity to the amino acid sequence of SEQ ID NO:108, a Cas7 polypeptide having at least 80% sequence identity to the amino acid sequence of SEQ ID NO:109 and optionally, a Cas3 polypeptide having at least 80% sequence identity to the amino acid sequence of SEQ ID NO:106.

In some embodiments of this invention, a recombinant nucleic acid construct may encode polypeptides from other Type I CRISPR-Cas systems or may encode polypeptides from any Type II CRISPR-Cas system, Type III CRISPR-Cas system, Type IV CRISPR-Cas system, Type V CRISPR-Cas system or Type VI CRISPR-Cas system that is now known or later discovered that can then be used in combination with a CRISPR comprising the repeat sequences from the corresponding Type I CRISPR-CAs system, Type II CRISPR-Cas system, Type III CRISPR-Cas system, Type IV CRISPR-Cas system, Type V CRISPR-Cas system or Type VI CRISPR-Cas system. In some embodiments, the polypeptide may be, for example, a Type I CRISPR-Cas nuclease, a Type II CRISPR-Cas nuclease, a Type III CRISPR-Cas nuclease, a Type IV CRISPR-Cas nuclease, Type V CRISPR-Cas nuclease and/or a Type VI CRISPR-Cas nuclease (e.g., a Cas9 nuclease, a Cas3 nuclease, Cas10 nuclease, a Csf4 nuclease, a Cas12 nuclease or a Cas13 nuclease). Non-limiting examples of additional CRISPR-Cas polypeptides include a Type I CRISPR associated complex for antiviral defense complex (Cascade complex) polypeptide, a Type III Csm complex polypeptide, a Type III Csr complex polypeptide, a Type IV polypeptide, Type V polypeptide and/or a Type VI polypeptide.

A “fragment” or “portion” of a nucleic acid will be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising a nucleotide sequence of contiguous nucleotides that are identical or almost identical (e.g., 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the invention may be, where appropriate, included in a larger polynucleotide of which it is a constituent. In some embodiments, a fragment of a polynucleotide can be a fragment that encodes a polypeptide that retains its function (e.g., encodes a fragment of a Type I-C Cascade polypeptide that is reduce in length as compared to the wild type polypeptide but which retains at least one function of a Type I-C Cascade protein or encodes a fragment of a Type I-B Cascade polypeptide that is reduce in length as compared to the wild type polypeptide but which retains at least one function of a Type I-B Cascade protein; e.g., processes CRISPR nucleic acids, bind DNA and/or form a complex). In some embodiments, a fragment of a polynucleotide can be a fragment of a native repeat sequence (e.g., a native repeat sequence from for example, Erysipelatoclostridium ramosum or Clostridium spp. 1141A1FAA, Clostridium scindens, Clostridium clostridioforme or Clostridium bolteae) that is shortened by about 1 nucleotide to about 7 nucleotides (e.g., 1, 2, 3, 4, 5, 6, or 7) or by about 1 nucleotide to about 8 nucleotides (e.g., 1, 2, 3, 4, 5, 6, 7 or 8) from the 3′ end of a native repeat sequence). In some embodiments, a fragment of a polynucleotide can be a fragment of a native repeat sequence that remains at the 3′ end of a spacer (e.g., from the 5′ end of the native repeat) when the native repeat sequence is shortened by 1 nucleotide to about 7 nucleotides or by 1 nucleotide to about 8 nucleotides from the 3′ end of a native repeat sequence (e.g., a portion of a repeat sequence having a length of about 11, 12, 13, 14, 15, 16, 17, 18, 19, 20 21, 22, 23, 24, 25, or 26 nucleotides).

As used herein, “chimeric” refers to a nucleic acid molecule or a polypeptide in which at least two components are derived from different sources (e.g., different organisms, different coding regions).

A “heterologous” or a “recombinant” nucleic acid is an exogenous nucleic acid not naturally associated with a host cell into which it is introduced, including non-naturally occurring multiple copies of a naturally occurring nucleic acid. In some embodiments, “heterologous” may include a nucleic acid that is endogenous to a host cell but is in a non-natural position relative to the wild type as a result of human intervention.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to said nucleotide sequence of the invention.

As used herein, hybridization, hybridize, hybridizing, and grammatical variations thereof, refer to the binding of two complementary nucleotide sequences or substantially complementary sequences in which some mismatched base pairs are present. The conditions for hybridization are well known in the art and vary based on the length of the nucleotide sequences and the degree of complementarity between the nucleotide sequences. In some embodiments, the conditions of hybridization can be high stringency, or they can be medium stringency or low stringency depending on the amount of complementarity and the length of the sequences to be hybridized. The conditions that constitute low, medium and high stringency for purposes of hybridization between nucleotide sequences are well known in the art (See, e.g., Gasiunas et al. (2012) Proc. Natl. Acad. Sci. 109:E2579-E2586; M. R. Green and J. Sambrook (2012) Molecular Cloning: A Laboratory Manual. 4th Ed., Cold Spring Harbor Laboratory Press, Cold Spring Harbor, NY).

A “native” or “wild type” nucleic acid, nucleotide sequence, polypeptide or amino acid sequence refers to a naturally occurring or endogenous nucleic acid, nucleotide sequence, polypeptide, or amino acid sequence. Thus, for example, a “wild type mRNA” is a mRNA that is naturally occurring in or endogenous to the organism. A “homologous” nucleic acid is a nucleic acid naturally associated with a host cell into which it is introduced.

As used herein, the terms “nucleic acid,” “nucleic acid molecule,” “nucleic acid construct,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The nucleic acid constructs of the present disclosure can be DNA or RNA. In some embodiments, the nucleic acid constructs of the present disclosure are DNA. Thus, although the nucleic acid constructs of this invention may be described and used in the form of DNA, depending on the intended use, they may also be described and used in the form of RNA.

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

A “synthetic” nucleic acid or nucleotide sequence, as used herein, refers to a nucleic acid or nucleotide sequence that is not found in nature but is constructed by human intervention, and as a consequence, it is not a product of nature.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide,” and “polynucleotide” are also used interchangeably herein to refer to a heteropolymer of nucleotides. Except as otherwise indicated, nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end. Thus, for example, an element in the 3′ region of a polynucleotide can be located anywhere from the first nucleotide located at the 3′ end of the polynucleotide to the nucleotide located halfway through the polynucleotide. An element that is described as being “at the 5′end” or “at the 3′end” of a polynucleotide (5′ to 3′) refers to an element located immediately adjacent to (upstream of) the first nucleotide at the 5′ end of the polynucleotide, or immediately adjacent to (downstream of) the last nucleotide located at the 3′ end of the polynucleotide, respectively.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, a “hairpin sequence” is a nucleotide sequence comprising hairpins. A hairpin (e.g., stem-loop, fold-back) refers to a nucleic acid molecule having a secondary structure that includes a region of nucleotides that form a single strand that are further flanked on either side by a double stranded-region. Such structures are well known in the art. As known in the art, the double stranded region can comprise some mismatches in base pairing or can be perfectly complementary. In some embodiments, a repeat sequence may comprise, consist essentially of, consist of a hairpin sequence that is located within the repeat nucleotide sequence (i.e., at least one nucleotide (e.g., one, two, three, four, five, six, seven, eight, nine, ten, or more) of the repeat nucleotide sequence is present on either side of the hairpin that is within the repeat nucleotide sequence).

A “CRISPR” as used herein comprises one or more repeat sequences and one or more spacer sequence(s), wherein each of the one or more spacer sequences is linked at least at its 5′-end to a repeat sequence or portion thereof. A “CRISPR” as used herein can include a CRISPR array, an unprocessed CRISPR, or a mature/processed CRISPR or a CRISPR that comprises one repeat, or a portion thereof, and a spacer (e.g., repeat-spacer). A “CRISPR” as used herein refers to a nucleic acid molecule that comprises at least two CRISPR repeat sequences, or a portion(s) thereof, and at least one spacer sequence, wherein one of the two repeat sequences, or a portion thereof, is linked to the 5′ end of the spacer sequence and the other of the two repeat sequences, or portion thereof, is linked to the 3′ end of the spacer sequence. In some embodiments, in a recombinant CRISPR of the invention, the combination of repeat nucleotide sequences and spacer sequences is synthetic and not found in nature. A CRISPR may be introduced into a cell or cell free system as RNA, or as DNA in an expression cassette or vector (e.g., plasmid, bacteriophage).

A CRISPR useful with the present invention may be, for example, a Type I CRISPR, Type II CRISPR, Type III CRISPR, Type IV CRISPR, Type V CRISPR or Type VI CRISPR. In some embodiments, the CRISPR may comprise at least one repeat sequence linked to the 5′ end and/or the 3′ end of the at least one spacer sequence. In some embodiments, the CRISPR may comprise a repeat sequence linked to the 5′ end and to the 3′ end of the at least one spacer sequence.

In some embodiments, the bacterial cell (e.g., a target bacterial cell) comprising the target nucleic acid (e.g., target DNA, target RNA) may comprise an endogenous CRISPR-Cas system that is operable with the CRISPR array in the composition, thereby editing/modifying the target nucleic acid in the genome of the bacterial cell. The endogenous CRISPR-Cas system of the bacterial cell may be a Type I CRISPR-Cas system, Type II CRISPR-Cas system, Type III CRISPR-Cas system, Type IV CRISPR-Cas system, Type V CRISPR system or Type VI CRISPR system.

A “repeat sequence” as used herein refers, for example, to any repeat sequence of a wild-type CRISPR locus or a repeat sequence of a synthetic CRISPR that are separated by “spacer sequences” (e.g., a repeat-spacer sequence or a repeat-spacer-repeat sequence of the invention). A repeat sequence useful with this invention can be any known or later identified repeat sequence of a CRISPR locus. Accordingly, in some embodiments, a repeat-spacer sequence or a repeat-spacer-repeat comprises a repeat that is substantially identical (e.g. at least about 70% identical (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a repeat from a wild type Type I CRISPR, a wild type Type II CRISPR, wild type Type III CRISPR, wild type Type IV CRISPR, wild type Type V CRISPR or wild type Type VI CRISPR. In embodiments, a repeat sequence useful with this invention can comprise a nucleotide sequence comprising a partial repeat that is a fragment or portion of consecutive nucleotides of a repeat sequence of a CRISPR locus or synthetic CRISPR array of any of a Type I crRNA, Type II crRNA, Type III crRNA, Type IV crRNA, Type V crRNA or Type VI crRNA.

As used herein, the term “spacer sequence” refers to a nucleotide sequence that is complementary to a targeted portion (i.e., “protospacer”) of a nucleic acid or a genome. The term “genome,” as used herein, refers to both chromosomal and non-chromosomal elements (i.e., extrachromosomal (e.g., mitochondrial, plasmid, a chloroplast, and/or extrachromosomal circular DNA (eccDNA))) of a target organism. The spacer sequence guides the CRISPR machinery to the targeted portion of the genome, wherein the targeted portion of the genome may be, for example, modified (e.g., a deletion, an insertion, a single base pair addition, a single base pair substitution, a single base pair removal, a stop codon insertion, and/or a conversion of one base pair to another base pair (base editing)). As another example, the spacer sequence may be used to guide the CRISPR machinery to the targeted portion of the genome, wherein the targeted portion of the genome may be cut and degraded, thereby killing the cell(s) comprising the target sequence.

A “target sequence” or “protospacer” (or “target DNA) refers to a region of a nucleic acid in a genome or in a cell free system that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR (crRNA) of this invention. In some embodiments, a target sequence may be about 10 to about 100 consecutive nucleotides in length (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides, or any value or range therein), about 25 to about 100 consecutive nucleotides in length (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides, or any value or range therein) or about 10 to about 60 consecutive nucleotides in length (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides, or any value or range therein), which region may be located immediately 3′ or 5′ to a PAM sequence, respectively, in the nucleic acid of the cell free system of in the genome of the organism. Type III CRISPR-Cas systems, for example, do not require a PAM.

In some embodiments, a protospacer adjacent motif (PAM) may comprise, consist essentially of, or consist of a nucleotide sequence of 5′-NAA-3′ and/or 5′-AAA-3′, 5′-NGG-3′, 5′-NGAAA-3′, 5′-NNG-3′, 5′-NGA-3′, 5′-NTAA-3′, 5′-NTG-3′, 5′-NNC-3′, 5′-NNAAC-3′, 5′-AGA-3′, 5′-NNNANNA-3′, 5′-NNANAA-3′, 5′-NNAAAA-3′, 5′-AAAA-3′, 5′-TTC-3′, 5′-TTT-3′, 5′-CTC-3′, 5′-TTTA-3′, 5′-ATC-3′, 5′-ATCN-3′ (N=T, A, C, or G, e.g., 5′-ATCA-3′, and/or 5′-ATCG-3′) that is located 5′ or 3′ of the protospacer or target sequence, the location dependent on the CRISPR-Cas system that is being introduced.

In some embodiments, a target sequence or protospacer useful with this invention may be, for example, located immediately adjacent to the 3′ end of a PAM (protospacer adjacent motif) (e.g., 5′-PAM-Protospacer-3′). In some embodiments, a PAM may comprise, consist essentially of, or consist of a sequence of 5′-TTT-3′, 5′-CTC-3′ or 5′-TTC-3′. A non-limiting example may be the following, 5′-3′, . . . ATGCTAATGGAGTTTACTACAAGTTAATCCGGCAAAGCTAAATGGCCGGCCC GT (SEQ ID NO:195).

Example target sequences useful with this invention includes any sequence in the genome of a bacteriophage, for example, a sequence that is conserved across a bacteriophage species, strain or population. Such target sequences include but are not limited to a tail protein, a portal protein, a capsid protein, a holin, a lysin, and/or a DNA packaging protein.

As used herein, the terms “target genome” or “targeted genome” refer to a genome of an organism of interest.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the phrase “substantially identical,” or “substantial identity” in the context of two nucleic acid molecules, nucleotide sequences or protein sequences, refers to two or more sequences or subsequences that have at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity can refer to two or more sequences or subsequences that have at least about 80%, at least about 85%, at least about 90%, at least about 95, 96, 96, 97, 98, or 99% sequence identity.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology—Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1×SSC, 0.1% SDS at 50° C. or in 7% sodium dodecyl sulfate (SDS), 0.5 M NaPO₄, 1 mM EDTA at 50° C. with washing in 0.5×SSC, 0.1% SDS at 50° C. Instill further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1×SSC, 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

Any polynucleotide and/or nucleic acid construct useful with this invention may be codon optimized for expression in any species of interest. Codon optimization is well known in the art and involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. As is understood in the art, codon optimization of a nucleotide sequence results in a nucleotide sequence having less than 100% sequence identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function (and in some embodiments, the same structure) as that encoded by the original nucleotide sequence. Thus, in some embodiments of the invention, polynucleotides and/or nucleic acid constructs useful with the invention may be codon optimized for expression in the particular organism/species of interest.

In some embodiments, the polynucleotides and polypeptides of the invention are “isolated.” An “isolated” polynucleotide sequence or an “isolated” polypeptide is a polynucleotide or polypeptide that, by human intervention, exists apart from its native environment and is therefore not a product of nature. An isolated polynucleotide or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated polynucleotide and/or the isolated polypeptide may be at least about 1%, 5%, 10%, 20%, 30%, 40%, 50%, 60%, 70%, 80%, 90%, 95%, or more pure.

In other embodiments, an isolated polynucleotide or polypeptide may exist in a non-natural environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the polynucleotides and their encoded polypeptides are “isolated” in that, through human intervention, they exist apart from their native environment and therefore are not products of nature, however, in some embodiments, they can be introduced into and exist in a recombinant host cell.

In some embodiments of the invention, a recombinant nucleic acid of the invention comprising/encoding a CRISPR and/or a Cascade complex and/or a Cas3 polypeptide (e.g., Type I-B or Type I-C polypeptides) may be operatively associated with a variety of promoters, terminators and other regulatory elements for expression in various organisms or cells. Thus, in some embodiments, at least one promoter and/or at least one terminator may be operably linked to a recombinant nucleic acid of the invention comprising/encoding a CRISPR and/or a Cascade complex and/or a Cas3 polypeptide. In some embodiments, when comprised in the same nucleic acid construct (e.g., expression cassette), the CRISPR and/or recombinant nucleic acid encoding a Cascade complex and/or a Cas3 polypeptide may be operably linked to separate (independent) promoters that may be the same promoter or a different promoter. In some embodiments, when comprised in the same nucleic acid construct, a CRISPR and a recombinant nucleic acid encoding a Cascade complex and/or a Cas3 polypeptide may be operably linked to a single promoter.

Any promoter useful with this invention can be used and includes, for example, promoters functional with the organism of interest. A promoter useful with this invention can include, but is not limited to, constitutive, inducible, developmentally regulated, tissue-specific/preferred-promoters, and the like, as described herein. A regulatory element as used herein can be endogenous or heterologous. In some embodiments, an endogenous regulatory element derived from the subject organism can be inserted into a genetic context in which it does not naturally occur (e.g., a different position in the genome than as found in nature), thereby producing a recombinant or non-native nucleic acid.

By “operably linked” or “operably associated” as used herein, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

Any promoter that initiates transcription of a recombinant nucleic acid construct of the invention, for example, in an organism/cell of interest may be used. A promoter useful with this invention can include, but is not limited to, a constitutive, inducible, developmentally regulated, tissue-specific/preferred-promoter, and the like, as described herein. A regulatory element as used herein can be endogenous or heterologous. In some embodiments, an endogenous regulatory element derived from the subject organism can be inserted into a genetic context in which it does not naturally occur (e.g., a different position in the genome than as found in nature (e.g., a different position in a chromosome or in a plasmid), thereby producing a recombinant or non-native nucleic acid.

Promoters can include, for example, constitutive, inducible, temporally regulated, developmentally regulated, chemically regulated, tissue-preferred and/or tissue-specific promoters for use in the preparation of recombinant nucleic acid molecules, i.e., “chimeric genes” or “chimeric polynucleotides.” These various types of promoters are known in the art. Thus, expression can be made constitutive, inducible, temporally regulated, developmentally regulated, chemically regulated, tissue-preferred and/or tissue-specific promoters using the recombinant nucleic acid constructs of the invention operatively linked to the appropriate promoter functional in an organism of interest. Expression may also be made reversible using the recombinant nucleic acid constructs of the invention operatively linked to, for example, an inducible promoter functional in an organism of interest. In some embodiments, promoters useful with the constructs of the invention may be any combination of heterologous/exogenous and/or endogenous promoters.

The choice of promoter will vary depending on the quantitative, temporal and spatial requirements for expression, and also depending on the host cell of interest. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

Promoters useful with this invention include promoters functional in a Clostridium spp.

In additional embodiments, a promoter useful with bacteria can include, but is not limited to, L-arabinose inducible (araBAD, P_{BAD}) promoter, any lac promoter, L-rhamnose inducible (rhaP_{BAD}) promoter, T7 RNA polymerase promoter, trc promoter, tac promoter, lambda phage promoter (p, p_{L}-9G-50), anhydrotetracycline-inducible (tetA) promoter, trp, lpp, phoA, recA, proU, cst-1, cadA, nar, lpp-lac, cspA, T7-lac operator, T3-lac operator, T4 gene 32, T5-lac operator, nprM-lac operator, Vhb, Protein A, corynebacterial-Escherichia coli like promoters, thr, hom, diphtheria toxin promoter, sig A, sig B, nusG, SoxS, katb, α-amylase (Pamy), Ptms, P43 (comprised of two overlapping RNA polymerase a factor recognition sites, σA, σB), Ptms, P43, rplK-rplA, ferredoxin promoter, and/or xylose promoter. (See, K. Terpe Appl. Microbiol, Biotechnol. 72:211-222 (2006); Hannig et al. Trends in Biotechnology 16:54-60 (1998); and Srivastava Protein Expr Purif 40:221-229 (2005)).

Translation elongation factor promoters may be used with the invention. Translation elongation factor promoters may include but are not limited to elongation factor Tu promoter (Tuf) (e.g., Ventura et al., Appl. Environ. Microbiol. 69:6908-6922 (2003)), elongation factor P (Pefp) (e.g., Tauer et al., Microbial Cell Factories, 13:150 (2014), rRNA promoters including but not limited to a P3, a P6 a P15 promoter (e.g., Djordjevic et al., Canadian Journal Microbiology, 43:61-69 (1997); Russell and Klaenhammer, Appl. Environ. Microbiol. 67:1253-1261 (2001)) and/or a P11 promoter. In some embodiments, a promoter may be a synthetic promoter derived from a natural promoter (e.g., Rud et al., Microbiology, 152:1011-1019 (2006). In some embodiments, a sakacin promoter may be used with the recombinant nucleic acid constructs of the invention (e.g., Mathiesen et al., J. Appl. Microbial., 96:819-827 (2004).

In some embodiments of the invention, inducible promoters can be used. Thus, for example, chemical-regulated promoters can be used to modulate the expression of a gene in an organism through the application of an exogenous chemical regulator. Regulation of the expression of nucleotide sequences of the invention via promoters that are chemically regulated enables the nucleic acids and/or the polypeptides of the invention to be synthesized only when, for example, a crop of plants are treated with the inducing chemicals. Depending upon the objective, the promoter may be a chemical-inducible promoter, where application of a chemical induces gene expression, or a chemical-repressible promoter, where application of the chemical represses gene expression. In some aspects, a promoter can also include a light-inducible promoter, where application of specific wavelengths of light induces gene expression (Levskaya et al. 2005. Nature 438:441-442). In other aspects, a promoter can include a light-repressible promoter, where application of specific wavelengths of light repress gene expression (Ye et al. 2011. Science 332:1565-1568).

Chemically inducible promoters useful with plants are known in the art and include, but are not limited to, steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

In some embodiments, a promoter useful with the recombinant nucleic acid constructs of the invention may be a promoter from any bacterial species. In some embodiments, for example, a promoter from a Clostridium spp. may be operably linked to a recombinant nucleic acid construct of the invention (e.g., a CRISPR, Cas3, and/or a Cascade complex). In some embodiments, an endogenous promoter from Clostridium bolteae, Clostridium clostridioforme, Clostridium scindens, Erysipelatoclostridium ramosum or Clostridium spp. 1141A1FAA may be operably linked to a recombinant nucleic acid construct of the invention. In some embodiments, a heterologous/exogenous promoter may be used.

In some embodiments, a promoter may be operably linked to a recombinant nucleic acid construct of the invention for expression in a Clostridium spp. cell (e.g., C. bolteae, C. scindens, C. clostridioforme, Clostridium scindens, Erysipelatoclostridium ramosum or Clostridium spp. 1141A1FAA)).

In some embodiments, a promoter or leader sequence useful with the invention may be any Clostridium promoter/leader sequence. (e.g., Clostridium spp. CRISPR leader sequences). In some embodiments, a promoter or leader sequence) useful with the invention includes, but is not limited to, any one of the nucleotide sequences of SEQ ID NOs:122-133 or SEQ ID NOs:177-185.

In some embodiments of this invention, one or more terminators may be operably linked to a polynucleotide encoding a Cascade complex and/or a CRISPR of the invention. In some embodiments, a terminator sequence may be operably linked to the 3′ end of a terminal repeat in a CRISPR.

In some embodiments, when comprised in the same nucleic acid construct (e.g., expression cassette), each of a CRISPR and a recombinant nucleic acid encoding a Cascade complex and/or a Cas3 may be operably linked to separate (independent) terminators (that may be the same terminator or a different terminator) or to a single terminator. In some embodiments, only the CRISPR may be operably linked to a terminator. Thus, in some embodiments, a terminator sequence may be operably linked to the 3′ end of a CRISPR (e.g., linked to the 3′ end of the repeat sequence located at the 3′ end of the CRISPR).

Any terminator that is useful for defining the end of a transcriptional unit (such as the end of a CRISPR or a Cascade complex) and initiating the process of releasing the newly synthesized RNA from the transcription machinery may be used with this invention (e.g., an terminator that is functional with a polynucleotide comprising a CRISPR and/or a polynucleotide encoding a Cascade complex of the invention may be utilized (e.g., that can define the end of a transcriptional unit (such as the end of a CRISPR, Cas3 or Cascade complex) and initiate the process of releasing the newly synthesized RNA from the transcription machinery).

A non-limiting example of a terminator useful with this invention may be a Rho-independent terminator sequence. In some embodiments, a Rho-independent terminator sequence from L. crispatus may be the nucleotide sequence of (5′-3′) AAAAAAAAACCCCGCCCCTGACAGGGCGGGGTTTTTTTT (SEQ ID NO:190). Further non-limiting examples of useful terminator sequences (5′-3′) include: AAAAGATCCCGGATTCTGTATGATGCAGAGTCCGGGATTTTT (SEQ ID NO:186); GGAACCCCTGGCCAATATGGTCAGGGGTTCT (SEQ ID NO:187); ATGAATTGCAGAAATGCATTTCAGATATTTTTGAACCTTGAAAAC (SEQ ID NO:188); CCCCTATTTTTGTGCAATATGTAGAAAAATA (SEQ ID NO:189); CAAAAAAAGCATGAGAATTAATTTTCTCATGCTTTTTTG (SEQ ID NO:191); AAAAAAGATGCACTTCTTCACAGGAGCGCATCTTTTTT (SEQ ID NO:192); CAAAAAGAGCGGCTATAGGCCGCTTTTTTTGC (SEQ ID NO:193); and/or GTAAAAATGGCTTGCGTGTTGCAAGCCATTTTTTTAC (SEQ ID NO:194).

In some embodiments, a recombinant nucleic acid construct of the invention may be an “expression cassette” or may be comprised within an expression cassette. As used herein, “expression cassette” means a recombinant nucleic acid construct comprising a polynucleotide of interest (e.g., a Cascade complex; Cas3) and/or a CRISPR of the invention, wherein said polynucleotide of interest and/or a CRISPR is operably associated with at least one control sequence (e.g., a promoter). Thus, some aspects of the invention provide expression cassettes designed to express the polynucleotides of the invention (e.g., the Cascade complexes, Cas3) and/or CRISPR of the invention.

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression.

An expression cassette may also optionally include a transcriptional and/or translational termination region (i.e., termination region) that is functional in the selected host cell. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked polynucleotide of interest, may be native to the host cell, or may be derived from another source (i.e., foreign or heterologous to the promoter, to the polynucleotide of interest, to the host, or any combination thereof).

An expression cassette (e.g., recombinant nucleic acid construct(s) of the invention) may also include a nucleotide sequence for a selectable marker, which can be used to select a transformed host cell (e.g., force a cell to acquire and keep an introduced nucleic acid (e.g., expression cassette, vector (e.g., plasmid) comprising the recombinant nucleic acid constructs of the invention)). As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Of course, many examples of suitable selectable markers are known in the art and can be used in the expression cassettes described herein. In some embodiments, a selectable marker useful with this invention includes polynucleotide encoding a polypeptide conferring resistance to an antibiotic. Non-limiting examples of antibiotics useful with this invention include tetracycline, chloramphenicol, and/or erythromycin. Thus, in some embodiments, a polynucleotide encoding a gene for resistance to an antibiotic may be introduced into the organism, thereby conferring resistance to the antibiotic to that organism.

In addition to expression cassettes, the nucleic acid construct and nucleotide sequences described herein may be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered or introduced. Vectors for use in transformation of host organisms are well known in the art. Non-limiting examples of general classes of vectors include but are not limited to a viral vector, a plasmid vector, a phage vector, a phagemid vector, a cosmid vector, a fosmid vector, a bacteriophage, an artificial chromosome, transposon, retrovirus or an Agrobacterium binary vector in double or single stranded linear or circular form which may or may not be self-transmissible or mobilizable. A vector as defined herein can transform a prokaryotic or eukaryotic host either by integration into the cellular genome or exist extrachromosomally (e.g., autonomous replicating plasmid with an origin of replication). Additionally, included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). A nucleic acid construct in the vector may be under the control of, and operably linked to, an appropriate promoter or other regulatory elements for transcription in a host cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell. Accordingly, the recombinant nucleic acid constructs of this invention and/or expression cassettes comprising the recombinant nucleic acid constructs of this invention may be comprised in vectors as described herein and as known in the art. In some embodiments, the constructs of the invention may be delivered in combination with polypeptides (e.g., Cascade complex polypeptides, Cas3) as ribonucleoprotein particles (RNPs). Thus, for example, a Cascade complex (or one or more polypeptides comprised in said Cascade complex) can be introduced as a DNA expression plasmid, e.g., in vitro transcripts, or as a recombinant protein bound to the RNA portion in a ribonucleoprotein particle (RNP) (e.g., protein-RNA complex), whereas the sgRNA can be delivered either expressed as a DNA plasmid or as an in vitro transcript. Any type of CRISPR system described herein may be delivered as polypeptides, including as an RNP.

Accordingly, in some embodiments, the invention provides a recombinant nucleic acid construct comprising a Clustered Regularly interspaced Short Palindromic Repeats (CRISPR) comprising one or more repeat sequence(s) (e.g., 1, 2, 3, 4, 5, 6, 7, 8, 9, 10, 11, 12, 13, 14, 15 or more) and one or more spacer sequence(s) (e.g., 1, 2, 3, 4, 5, 6, 7, 8, 9, 10, 11, 12, 13, 14, 15 or more), wherein each spacer sequence and each repeat sequence have a 5′ end and a 3′ end and each spacer sequence is linked at its 5′ end and at its 3′ end to a repeat sequence, and the spacer sequence is complementary to a target sequence (protospacer) in a target DNA of a target organism that is located immediately adjacent (3′) to a protospacer adjacent motif (PAM). In some embodiments, a CRISPR of the present invention comprises a minimum of two repeats, flanking a spacer, to be expressed as a premature CRISPR (e.g., pre-CRISPR) that will be processed internally in the cell to constitute the final mature CRISPR. A CRISPR array (crRNA, crDNA) useful with this invention may be an array from any Type I CRISPR-Cas system, Type II CRISPR-Cas system, Type III CRISPR-Cas system, Type IV CRISPR-Cas system, Type V CRISPR-Cas system or a Type VI CRISPR-Cas system.

A repeat sequence useful with this invention can be any known or later identified repeat sequence of a CRISPR locus. Accordingly, in some embodiments, a repeat-spacer sequence or a repeat-spacer-repeat comprises a repeat that is substantially identical (e.g. at least about 70% identical (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a repeat from a wild-type Type II CRISPR array. In some embodiments, a repeat sequence is 100% identical to a repeat from a wild type Type I CRISPR, a wild type Type II CRISPR, wild type Type III CRISPR, wild type Type IV CRISPR, wild type Type V CRISPR or wild type Type VI CRISPR. In embodiments, a repeat sequence useful with this invention can comprise a nucleotide sequence comprising a partial repeat that is a fragment or portion of consecutive nucleotides of a repeat sequence of a CRISPR locus or synthetic CRISPR of any of a Type I crRNA, Type II crRNA, Type III crRNA, Type IV crRNA, Type V crRNA or Type VI crRNA.

In some embodiments, a repeat sequence as used herein may comprise any known repeat sequence of a wild-type Clostridium CRISPR Type I-B locus (e.g., Clostridium spp. 1141A1FAA). In some embodiments, a repeat sequence useful with the invention may include a synthetic repeat sequence having a different nucleotide sequence than those known in the art for Clostridium but sharing similar structure to that of wild-type Clostridium repeat sequences of a hairpin structure with a loop region. Thus, in some embodiments, a repeat sequence may be identical to (i.e., having 100% identity) or substantially identical (e.g., having 80% to 99% identity (e.g., about 80, 81, 82, 83, 84, 85, 86, 87, 88, 89, 90, 91, 92, 93, 94, 95, 96, 97, 98, 99% identity)) to a repeat sequence from a wild-type Clostridium CRISPR Type I-B locus.

In some embodiments, a repeat sequence as used herein may comprise any known repeat sequence of a wild-type Erysipelatoclostridium CRISPR Type I-B locus (e.g., E. ramosum). In some embodiments, a repeat sequence useful with the invention may include a synthetic repeat sequence having a different nucleotide sequence than those known in the art for Erysipelatoclostridium but sharing similar structure to that of wild-type Erysipelatoclostridium repeat sequences of a hairpin structure with a loop region. Thus, in some embodiments, a repeat sequence may be identical to (i.e., having 100% identity) or substantially identical (e.g., having 80% to 99% identity (e.g., about 80, 81, 82, 83, 84, 85, 86, 87, 88, 89, 90, 91, 92, 93, 94, 95, 96, 97, 98, 99% identity)) to a repeat sequence from a wild-type Erysipelatoclostridium CRISPR Type I-B locus.

In some embodiments, a repeat sequence as used herein may comprise any known repeat sequence of a wild-type Clostridium CRISPR Type I-C locus (e.g., C. bolteae, C. scindens, C. clostridioforme). In some embodiments, a repeat sequence useful with the invention may include a synthetic repeat sequence having a different nucleotide sequence than those known in the art for Clostridium but sharing similar structure to that of wild-type Clostridium repeat sequences of a hairpin structure with a loop region. Thus, in some embodiments, a repeat sequence may be identical to (i.e., having 100% sequence identity) or substantially identical (e.g., having 80% to 99% sequence identity (e.g., 80, 81, 82, 83, 84, 85, 86, 87, 88, 89, 90, 91, 92, 93, 94, 95, 96, 97, 98, 99% sequence identity)) to a repeat sequence from a wild-type Clostridium CRISPR Type I-C locus.

The length of a CRISPR repeat sequence useful with this invention may be the full length of a Type I-C Clostridium (e.g., C. bolteae, C. scindens, C. clostridioforme) repeat sequence (i.e., about 32 nucleotides or 33 nucleotides) (see, e.g., SEQ ID NOs:15-19, 34, 35, 50-53, 68-71, 86-88, 103-105, 120, or 121). In some embodiments, a repeat sequence may comprise a portion of a wild type Clostridium repeat nucleotide sequence, the portion being reduced in length by as much as 7 to 8 nucleotides (e.g., 1, 2, 3, 4, 5, 6, 7, or 8 nucleotides) from the 3′ end as compared to a wild type Clostridium repeat (e.g., comprising about 24 to 25 or 25 to 26 or more contiguous nucleotides from the 5′ end of a wild type Clostridium CRISPR Type I-C locus repeat sequence; e.g., about 24, 25, 26, 27, 28, 29, 30, 31 or 32 contiguous nucleotides from the 5′ end, or any range or value therein). In some embodiments, a repeat sequence useful with this invention may comprise, consist essentially of or consist of at least 24 consecutive nucleotides (e.g., about 24, 25, 26, 27, 28, 29, 30, 31, 32 or 33 consecutive nucleotides) having at least 80% sequence identity (e.g., 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the nucleotide sequences of SEQ ID NOs:15-19, any one of the nucleotide sequences of SEQ ID NOs: 34-35, any one of the nucleotide sequences of SEQ ID NOs:50-53, any one of the nucleotide sequences of SEQ ID NOs: 68-71, any one of the nucleotide sequences of SEQ ID NOs:86-88, any one of the nucleotide sequences of SEQ ID NOs: 103-105, or any one of the nucleotide sequences of SEQ ID NOs: 120-121, optionally about 24, 25, 26, 27, 28 to about 29, 30, 31 or 32 consecutive nucleotides, about 25, 26, 27, 28 to about 29, 30, 31, 32 or 33, or about 30 to 33 consecutive nucleotides of the repeat sequences.

Thus, in some embodiments, a repeat sequence may comprise, consist essentially of, or consist of any of the nucleotide sequences of (or a portion thereof):

SEQ ID NO: 15
GTCGTTCCCTGCAATGGGAACGTGGATTGAAAT
SEQ ID NO: 16
GCGTTGTTCCCATGCGGGAACTTGGATTGAAAT
SEQ ID NO: 17
GTCTCTCCCTGTATAGGGAGAGTGGATTGAAAT
SEQ ID NO: 18
GTCTTTCCCTGCATAGGGAGAGTGGATTGAAAT
SEQ ID NO: 19
GTCTCCACCTGTGTGGTGGAGTGGATTGAAAG
SEQ ID NO: 34
GTCTCCACCCTCGTGGTGGAGTGGATTGAAAT
SEQ ID NO: 35
GTCGAGGCCCGCGAGGGCCTTGTGGATTGAAAT
SEQ ID NO: 50
GTCTCCGTCCTCGCGGGCGGAGTGGGTTGAAAT
SEQ ID NO: 51
GTCTCCGTCCTCGCGGGGGGAGTGGCTTTTCCT
SEQ ID NO: 52
GTCGAGGCTCGCGAGAGCCTTGTGGATTGAAAT
SEQ ID NO: 53
GTCGAGGCTCGCGAGAGCCTTGCAGACCAAAAG
SEQ ID NO: 68
GTCGAGGCTCGCGAGAGCCTTGTGGATTGAAAT
SEQ ID NO: 69
GTCGAGGCTCGCGAGAGCCTTGCAGACCAAAAG
SEQ ID NO: 70
GTCTCCGTCCTCGCGGGCGGAGTGGGTTGAAAT
SEQ ID NO: 71
GTCTCCGTCCTCGCGGGCGGAGTGGCTTTTCCT
SEQ ID NO: 86
GTCGAGGCCCGCGAGGGCCTTGTGGATTGAAAT
SEQ ID NO: 87
GTCTCCACCCTCGCGGTGGAGTGGATTGAAAT
SEQ ID NO: 88
ATCTCCACCCTCGCGGTGGAGTGGATTGAAAT
SEQ ID NO: 103
GTCTCCACCCTCGCGGTGGAGTGGATTGAAAT
SEQ ID NO: 104
GTCGAGGCCCGCGAGGGCCTTGTGGATTGAAAT
SEQ ID NO: 105
GTCGAGGCCCGCGAGAGCCTTGTGGATTGAAAT
SEQ ID NO: 120
GTCTCCACCCTCGTGGTGGAGTGGATTGAAAT
SEQ ID NO: 121
GTCGAGGCCCGCGAGGGCCTTGTGGATTGAAAT

The length of a CRISPR repeat sequence useful with this invention may be the full length of a Type I-B Clostridium spp. or Erysipelatoclostridium spp. (e.g., E. ramosum or C. spp. 1141A1FAA) repeat sequence (i.e., about 25 to 32 nucleotides) (see, e.g., SEQ ID NOs:138, 139 or 156-164). In some embodiments, a repeat sequence may comprise a portion of a wild type Clostridium or Erysipelatoclostridium repeat nucleotide sequence, the portion being reduced in length by as much as 7 to 8 nucleotides (e.g., 1, 2, 3, 4, 5, 6, 7, or 8 nucleotides) from the 3′ end as compared to a wild type Clostridium or Erysipelatoclostridium repeat (e.g., comprising about 19 to 31 contiguous nucleotides from the 5′ end of a wild type Clostridium or Erysipelatoclostridium CRISPR Type I-B locus repeat sequence; e.g., about 19, 20, 20, 21, 22, 23, 24, 25, 26, 27, 28, 29, 30 or 31 contiguous nucleotides from the 5′ end, or any range or value therein). In some embodiments, a repeat sequence useful with this invention may comprise, consist essentially of or consist of at least 19 consecutive nucleotides (e.g., about 19, 20, 21, 22, 23, 24, 25, 26, 27, 28, 29, 30, 31, 32 or 33 consecutive nucleotides) having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the nucleotide sequences of SEQ ID NOs:138, 139 or 156-164, optionally about 19, 20, 21, 22, 23, 24, or 25 consecutive nucleotides to about 26, 27, 28 29, 30, or 31 consecutive nucleotides, about 21, 22, 23, 24, or 25 consecutive nucleotides to about 26, 27, 28, 29, 30, 31, 32 or 33, or about 27 to about 32 consecutive nucleotides of the repeat sequences.

Thus, in some embodiments, a repeat sequence may comprise, consist essentially of, or consist of any of the nucleotide sequences of: GTTTAATAACAACATAAGATGTATTGAAAT SEQ ID NO:138, GTTTAATAACAACGAGATATTAAAGTGAAA SEQ ID NO:139, GTTTCAATCCACGCACCCGTGCAGGGTGCGAC SEQ ID NO:156, GTTTCAATTCCAACATGGTACGATTAAAGC SEQ ID NO:157, GTTTCAATTCCAATATGGCACGATTAAAGC SEQ ID NO:158, TGCTTTATTACAATGTGGTAAGAGTAAAGC SEQ ID NO:159, CTTTCAATTCCAACTTGGTACGATTAAAAC SEQ ID NO:160, AACGCAATTCCAACTTGGTACGATTAAAAC SEQ ID NO:161, GTTTCAATTCCAACTTGGTACGATTAAAAC SEQ ID NO:162, GTTGCAATTCCAATATGGTAAGATTAAAGC SEQ ID NO:163, and/or TCAATTCCAATATGGTACGATTAAAAC SEQ ID NO:164, or any combination thereof.

The bolded nucleotides indicate the nucleotides that differ from the consensus sequence.

In some embodiments, when two or more (e.g., 2, 3, 4, 5, 6, 7, 8, 9, 10, 11, 12, 13, 14, 15 or more) repeat sequences are present in a CRISPR may comprise the same repeat sequence, may comprise different repeat sequences, or any combination thereof. In some embodiments, each of the two or more repeat sequences in a single CRISPR may comprise, consist essentially of, or consist of the same repeat sequence.

A CRISPR useful with the invention may comprise one spacer sequence or more than one spacer sequence, wherein each spacer sequence is flanked at least at its 5′ end by a repeat sequence. In some embodiments, a CRISPR may comprise, 5′-3′, a repeat-spacer, wherein the repeat is a full-length repeat sequence or a portion thereof. In some embodiments, a CRISPR useful with this invention may comprise a spacer sequence linked at its 5′ end and its 3′ end to a repeat sequence (e.g., a repeat-spacer-repeat), wherein the repeat is a full-length repeat sequence or a portion thereof. When more than one spacer sequence is present in a CRISPR of the invention, each spacer sequence is separated from the next spacer sequence by a repeat sequence. Thus, each spacer sequence is linked at the 3′ end and at the 5′ end to a repeat sequence. The repeat sequence that is linked to each end of the one or more spacers may be the same repeat sequence or it may be a different repeat sequence or any combination thereof.

A spacer sequence (e.g., one or more spacer sequences) for use with a CRISPR of the invention may be about 10 nucleotides to about 60 nucleotides in length (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length, and any value or range therein. In some embodiments, the spacer may comprise a length of about 20 nucleotides to about 40 nucleotides in length (e.g., about 20, 21, 22, 23, 24, 25, 26, 27, 28, 29, 30, 31, 32, 33, 34, 35, 36, 37, 38, 39, or 40 nucleotides in length, and any value or range therein), about 30 nucleotides to about 40 nucleotides (e.g., about 30, 31, 32, 33, 34, 35, 36, 37, 38, 39, 40 nt), about 32 nucleotides to about 40 nucleotides (e.g., about 32, 33, 34, 35, 36, 37, 38, 39, 40 nt), or about 34, 35, 36 or 37 nucleotides. In some embodiments, the spacer may comprise a length of about 20, 22, 31, 33, 34, or 38 nucleotides in length. In some embodiments, a spacer sequence may comprise, consist essentially of, or consist of a length of about 34 nucleotides in length.

A spacer sequence (e.g., one or more spacer sequences) for use with a Type I-C CRISPR system of the present invention may be about 20 nucleotides to about 40 nucleotides in length (e.g., about 20, 21, 22, 23, 24, 25, 26, 27, 28, 29, 30, 31, 32, 33, 34, 35, 36, 37, 38, 39, or 40 nucleotides in length, and any value or range therein). In some embodiments, a spacer sequence may be a length of about 30 nucleotides to about 40 nucleotides (e.g., about 30, 31, 32, 33, 34, 35, 36, 37, 38, 39, or 40 nucleotides in length, and any value or range therein), or about 20, 22, 31, 33, 34, or 38 nucleotides in length. In some embodiments, a spacer sequence may comprise, consist essentially of, or consist of a length of about 34 nucleotides in length.

In some embodiments, a spacer sequence (e.g., one or more spacer sequences) for use with a Type I-B CRISPR system of the present invention may be about 25 nucleotides to about 60 nucleotides in length (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length, and any value or range therein). In some embodiments, a spacer sequence may be a length of about 25 nucleotides to about 45 nucleotides (e.g., about 25, 26, 27, 28, 29, 30, 31, 32, 33, 34, 35, 36, 37, 38, 39, 40, 41, 42, 43, 44, or 45 nucleotides in length, and any value or range therein), about 30 nucleotides to about 40 nucleotides (e.g., about 30, 31, 32, 33, 34, 35, 36, 37, 38, 39, or 40 nucleotides in length, and any value or range therein) or about 32 nucleotides to about 40 nucleotides (e.g., about 30, 31, 32, 33, 34, 35, 36, 37, 38, 39, or 40 nucleotides in length, and any value or range therein). In some embodiments, a spacer sequence may comprise, consist essentially of, or consist of a length of about 34, 35, 36 or 37 nucleotides in length. In some embodiments, a spacer sequence may comprise, consist essentially of, or consist of a length of about 34 nucleotides in length. In some embodiments, a spacer sequence may comprise, consist essentially of, or consist of a length of about 35 nucleotides in length. In some embodiments, a spacer sequence may comprise, consist essentially of, or consist of a length of about 36 nucleotides in length. In some embodiments, a spacer sequence may comprise, consist essentially of, or consist of a length of about 37 nucleotides in length.

A spacer sequence may be fully complementary to a target sequence (e.g., 100% complementary to a target sequence across its full length). In some embodiments, a spacer sequence may be substantially complementary (e.g., at least about 80% complementary (e.g., about 80%, 81%, 82%, 83%, 84%, 85%, 86%, 87%, 88%, 89%, 90%, 91%, 92%, 93%, 94%, 95%, 96%, 97%, 98%, 98.5%, 99%, 99.5%, or more complementary)) to a target sequence from a target genome. Thus, in some embodiments, a spacer sequence may have one, two, three, four, five or more mismatches that may be contiguous or noncontiguous as compared to a target sequence from a target genome. In some embodiments, a spacer sequence may be about 80% to 100% (e.g., about 80%, 81%, 82%, 83%, 84%, 85%, 86%, 87%, 88%, 89%, 90%, 91%, 92%, 93%, 94%, 95%, 96%, 97%, 98%, 99%, or 100)) complementary to a target sequence from a target genome. In some embodiments, a spacer sequence may be about 85% to 100% (e.g., about 85%, 86%, 87%, 88%, 89%, 90%, 91%, 92%, 93%, 94%, 95%, 96%, 97%, 98%, 99%, or 100%)) complementary to a target sequence from a target genome. In some embodiments, a spacer sequence may be about 90% to 100% (e.g., about 90%, 91%, 92%, 93%, 94%, 95%, 96%, 97%, 98%, 99%, or 100%)) or about 95% to 100% (e.g., about 95%, 96%, 97%, 98%, 98.5%, 99%, 99.5% or 100%) complementary to a target sequence from a target genome.

In some embodiments, the 5′ region of a spacer sequence may be fully complementary to a target sequence while the 3′ region of the spacer sequence may be substantially complementary to the target sequence. Accordingly, in some embodiments, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) may be about 100% complementary to a target sequence, while the remainder of the spacer sequence may be about 80% or more complementary to the target sequence.

In some embodiments, at least the first eight contiguous nucleotides at the 5′ end of a spacer sequence of the invention are fully complementary to the portion of the target sequence adjacent to the PAM (termed a “seed sequence”). Thus, in some embodiments, the seed sequence may comprise the first 8 nucleotides of the 5′ end of each of one or more spacer sequence(s), which first 8 nucleotides are fully complementary (100%) to the target sequence, and the remaining portion of the one or more spacer sequence(s) (3′ to the seed sequence) may be at least about 80% complementarity (e.g., about 80, 81, 82, 83, 84, 85, 86, 87, 88, 89, 90, 91, 92, 93, 94, 95, 96, 97, 98, 99, or 100% complementarity) to the target sequence. Thus, for example, a spacer sequence having a length of 20 nucleotides may comprise a seed sequence of eight contiguous nucleotides located at the 5′ end of the spacer sequence, which is 100% complementary to the target sequence, while the remaining 12 nucleotides may be about 80% to about 100% complimentary to the target sequence (e.g., 0 to 2 non-complementary nucleotides out of the remaining 12 nucleotides in the spacer sequence). As another example, a spacer sequence having a length of 34 nucleotides may comprise a seed sequence of eight nucleotides from the 5′ end, which is 100% complementary to the target sequence, while the remaining 26 nucleotides may be at least about 80% (e.g., 0 to 5 non-complementary nucleotides out of the remaining 26 nucleotides in the spacer sequence) or a spacer sequence having a length of 38 nucleotides may comprise a seed sequence of eight nucleotides from the 5′ end, which is 100% complementary to the target sequence, while the remaining 30 nucleotides may be at least about 80% (e.g., 0 to 6 non-complementary nucleotides out of the remaining 30 nucleotides in the spacer sequence).

A CRISPR useful with the invention comprising more than one spacer sequence may be designed to target one or more than one target sequence (protospacer). Thus, in some embodiments, when a recombinant nucleic acid construct of the invention comprises a CRISPR that comprises at least two spacer sequences, the at least two spacer sequences may be complementary to two or more different target sequences. In some embodiments, when a recombinant nucleic acid construct of the invention comprises a CRISPR that comprises at least two spacer sequences, the at least two spacer sequences may be complementary to the same target sequence. In some embodiments, a CRISPR comprising at least two spacer sequences, the at least two spacer sequences may be complementary to different portions of one gene.

### Type I-C CRISPR System Polypeptides and Polynucleotides

In some embodiments, a recombinant nucleic acid construct of the invention may encode a Type I-C CRISPR associated complex for antiviral defense complex (Cascade complex) comprising: a Cas5 polypeptide, a Cas8 polypeptide, and a Cas7 polypeptide. In some embodiments, a recombinant nucleic acid construct of the invention may further comprise a Cas3 polypeptide of a Type I-C CRISPR-Cas system.

In some embodiments, a Cas5 polypeptide comprises any one of the amino acid sequences of SEQ ID NOs:2, 21, 37, 55, 73, 90, or 107 or a polypeptide sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the amino acid sequences of SEQ ID NOs:2, 21, 37, 55, 73, 90, or 107. In some embodiments, a Cas8 polypeptide comprises any one of the amino acid sequences of SEQ ID NOs:3, 22, 38, 56, 74, 91, or 108 or a polypeptide sequence having at least 80% sequence identity to any one of the amino acid sequences of SEQ ID NOs:3, 22, 38, 56, 74, 91, or 108. In some embodiments, a Cas7 polypeptide comprises any one of the amino acid sequences of SEQ ID NOs:4, 23, 39, 57, 75, 92, or 109 or a polypeptide sequence having at least 80% sequence identity to any one of the amino acid sequences of SEQ ID NOs:4, 23, 39, 57, 75, 92, or 109. In some embodiments, a Cas3 polypeptide comprises any one of the amino acid sequences of SEQ ID NOs:1, 20, 36, 54, 72, 89, or 106 or a polypeptide sequence having at least 80% sequence identity to any one of the amino acid sequences of 1, 20, 36, 54, 72, 89, or 106.

In some embodiments, a Cas5 polypeptide is encoded by any one of the nucleotide sequences of SEQ ID NOs:9, 28, 44, 62, 80, 97, or 114 or a nucleotide sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of 9, 28, 44, 62, 80, 97, or 114. In some embodiments, a Cas8 polypeptide is encoded by any one of the nucleotide sequences of SEQ ID NOs:10, 29, 45, 63, 81, 98, or 115 or a nucleotide sequence having at least 80% sequence identity to any one of 10, 29, 45, 63, 81, 98, or 115. In some embodiments, a Cas8 polypeptide is encoded by any one of the nucleotide sequences of SEQ ID NOs:11, 30, 46, 64, 82, 99, or 116 or a nucleotide sequence having at least 80% sequence identity to any one of 11, 30, 46, 64, 82, 99, or 116. In some embodiments, a Cas3 polypeptide is encoded by any one of the nucleotide sequences of SEQ ID NOs:8, 27, 43, 61, 79, 96, or 113 or a nucleotide sequence having at least 80% sequence identity to any one of 8, 27, 43, 61, 79, 96, or 113.

Accordingly, in some embodiments, the present invention provides recombinant nucleic acid molecules encoding one or more polypeptides of a Type I-C Cascade complex, the one or more polypeptides of a Type I-C Cascade complex comprising a Cas5 polypeptide comprising an amino acid sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO:2 or encoded by a nucleotide sequence having at least 80% sequence identity to SEQ ID NO:9, a Cas8 polypeptide comprising an amino acid sequence having at least 80% sequence identity to SEQ ID NO:3 or encoded by a nucleotide sequence having at least 80% sequence identity to SEQ ID NO:10, and a Cas7 polypeptide comprising an amino acid sequence having at least 80% sequence identity to SEQ ID NO:4 or encoded by a nucleotide sequence having at least 80% sequence identity to SEQ ID NO:11, optionally, wherein when used in combination with a CRISPR, the CRISPR comprises any combination of one or more repeat sequences, or portion thereof, having at least 80% sequence identity to a nucleotide sequence of SEQ ID NOs:15-19; or a Cas5 polypeptide comprising an amino acid sequence having at least 80% sequence identity to SEQ ID NO:2 or encoded by a nucleotide sequence having at least 80% sequence identity to SEQ ID NO:9, a Cas8 polypeptide comprising an amino acid sequence having at least 80% sequence identity to SEQ ID NO:3 or encoded by a nucleotide sequence having at least 80% sequence identity to SEQ ID NO:10, and a Cas7 polypeptide comprising an amino acid sequence having at least 80% sequence identity to SEQ ID NO:4 or encoded by a nucleotide sequence having at least 80% sequence identity to SEQ ID NO:11; and a Cas3 polypeptide comprising an amino acid sequence having at least 80% sequence identity to SEQ ID NO:1 or encoded by a nucleotide sequence having at least 80% sequence identity to SEQ ID NO:8, optionally, wherein when used in combination with a CRISPR, the CRISPR comprises any combination of one or more repeat sequences, or portion thereof, having at least 80% sequence identity to the nucleotide sequence of SEQ ID NOs:15-19.

In some embodiments, the present invention provides recombinant nucleic acid molecules encoding one or more polypeptides of a Type I-C Cascade complex, the one or more polypeptides of a Type I-C Cascade complex comprising a Cas5 polypeptide comprising an amino acid sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO:21 or encoded by a nucleotide sequence having at least 80% sequence identity to SEQ ID NO:28, a Cas8 polypeptide comprising an amino acid sequence having at least 80% sequence identity to SEQ ID NO:22 or encoded by a nucleotide sequence having at least 80% sequence identity to SEQ ID NO:29, and a Cas7 polypeptide comprising an amino acid sequence having at least 80% sequence identity to SEQ ID NO:23 or encoded by a nucleotide sequence having at least 80% sequence identity to SEQ ID NO:30, optionally, wherein when used in combination with a CRISPR, the CRISPR comprises any combination of one or more repeat sequences, or portion thereof, having at least 80% sequence identity to the nucleotide sequence of SEQ ID NO:34 or SEQ ID NO:35, or optionally SEQ ID NOs:86-88, 103-105, 120, or 121; or a Cas5 polypeptide comprising an amino acid sequence having at least 80% sequence identity to SEQ ID NO:21 or encoded by a nucleotide sequence having at least 80% sequence identity to SEQ ID NO:28, a Cas8 polypeptide comprising an amino acid sequence having at least 80% sequence identity to SEQ ID NO:22 or encoded by a nucleotide sequence having at least 80% sequence identity to SEQ ID NO:29, and a Cas7 polypeptide comprising an amino acid sequence having at least 80% sequence identity to SEQ ID NO:23 or encoded by a nucleotide sequence having at least 80% sequence identity to SEQ ID NO:30; and a Cas3 polypeptide comprising an amino acid sequence having at least 80% sequence identity to SEQ ID NO:20 or encoded by a nucleotide sequence having at least 80% sequence identity to SEQ ID NO:27, optionally, wherein when used in combination with a CRISPR, the CRISPR comprises any combination of one or more repeat sequences, or portion thereof, having at least 80% sequence identity to the nucleotide sequence of SEQ ID NO:34 or SEQ ID NO:35, or optionally SEQ ID NOs: 86-88, 103-105, 120, or 121.

In some embodiments, the present invention provides recombinant nucleic acid molecules encoding one or more polypeptides of a Type I-C Cascade complex, the one or more polypeptides of a Type I-C Cascade complex comprising a Cas5 polypeptide comprising an amino acid sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO:37 or encoded by a nucleotide sequence having at least 80% sequence identity to SEQ ID NO:44, a Cas8 polypeptide comprising an amino acid sequence having at least 80% sequence identity to SEQ ID NO:38 or encoded by a nucleotide sequence having at least 80% sequence identity to SEQ ID NO:45, and a Cas7 polypeptide comprising an amino acid sequence having at least 80% sequence identity to SEQ ID NO:39 or encoded by a nucleotide sequence having at least 80% sequence identity to SEQ ID NO:46, optionally, wherein when used in combination with a CRISPR, the CRISPR comprises any combination of one or more repeat sequences, or portion thereof, having at least 80% sequence identity to the nucleotide sequence of SEQ ID NOs:50-53, or optionally SEQ ID NOs: 68-71; or a Cas5 polypeptide comprising an amino acid sequence having at least 80% sequence identity to SEQ ID NO:37 or encoded by the nucleotide sequence of SEQ ID NO:44, a Cas8 polypeptide comprising an amino acid sequence having at least 80% sequence identity to SEQ ID NO:38 or encoded by the nucleotide sequence of SEQ ID NO:45, and a Cas7 polypeptide comprising an amino acid sequence having at least 80% sequence identity to SEQ ID NO:39 or encoded by the nucleotide sequence of SEQ ID NO:46; and a Cas3 polypeptide comprising an amino acid sequence having at least 80% sequence identity to SEQ ID NO:36 or encoded by a nucleotide sequence having at least 80% sequence identity to SEQ ID NO:43, optionally, wherein when used in combination with a CRISPR, the CRISPR comprises any combination of one or more repeat sequences, or portion thereof, having at least 80% sequence identity to the nucleotide sequence of SEQ ID NOs: 50-53, or optionally SEQ ID NOs: 68-71.

In some embodiments, the present invention provides recombinant nucleic acid molecules encoding one or more polypeptides of a Type I-C Cascade complex, the one or more polypeptides of a Type I-C Cascade complex comprising a Cas5 polypeptide comprising an amino acid sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO:55 or encoded by a nucleotide sequence having at least 80% sequence identity to SEQ ID NO:62, a Cas8 polypeptide comprising an amino acid sequence having at least 80% sequence identity to SEQ ID NO:56 or encoded by a nucleotide sequence having at least 80% sequence identity to SEQ ID NO:63, and a Cas7 polypeptide comprising an amino acid sequence having at least 80% sequence identity to SEQ ID NO:57 or encoded by a nucleotide sequence having at least 80% sequence identity to SEQ ID NO:64, optionally, wherein when used in combination with a CRISPR, the CRISPR comprises any combination of one or more repeat sequences, or portion thereof, having at least 80% sequence identity to the nucleotide sequence of SEQ ID NOs:68-71, or optionally SEQ ID NOs: 50-53; a Cas5 polypeptide comprising an amino acid sequence having at least 80% sequence identity to SEQ ID NO:55 or encoded by a nucleotide sequence having at least 80% sequence identity to SEQ ID NO:62, a Cas8 polypeptide comprising an amino acid sequence having at least 80% sequence identity to SEQ ID NO:56 or encoded by a nucleotide sequence having at least 80% sequence identity to SEQ ID NO:63, and a Cas7 polypeptide comprising an amino acid sequence having at least 80% sequence identity to SEQ ID NO:57 or encoded by a nucleotide sequence having at least 80% sequence identity to SEQ ID NO:64; and a Cas3 polypeptide comprising an amino acid sequence having at least 80% sequence identity to SEQ ID NO:54 or encoded by a nucleotide sequence having at least 80% sequence identity to SEQ ID NO:61, optionally, wherein when used in combination with a CRISPR, the CRISPR comprises any combination of one or more repeat sequences, or portion thereof, having at least 80% sequence identity to the nucleotide sequence of SEQ ID NOs:68-71, or optionally SEQ ID NOs:50-53.

In some embodiments, the present invention provides recombinant nucleic acid molecules encoding one or more polypeptides of a Type I-C Cascade complex, the one or more polypeptides of a Type I-C Cascade complex comprising a Cas5 polypeptide comprising an amino acid sequence having at least 80% sequence identity to SEQ ID NO:73 (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) or encoded by a nucleotide sequence having at least 80% sequence identity to SEQ ID NO:80, a Cas8 polypeptide comprising an amino acid sequence having at least 80% sequence identity to SEQ ID NO:74 or encoded by a nucleotide sequence having at least 80% sequence identity to SEQ ID NO:81, and a Cas7 polypeptide comprising an amino acid sequence having at least 80% sequence identity to SEQ ID NO:75 or encoded by a nucleotide sequence having at least 80% sequence identity to SEQ ID NO:82, optionally, wherein when used in combination with a CRISPR, the CRISPR comprises any combination of one or more repeat sequences, or portion thereof, having at least 80% sequence identity to the nucleotide sequence of SEQ ID NOs:86-88, or optionally SEQ ID NOs: 34, 35, 103-105, 120, or 121; a Cas5 polypeptide comprising an amino acid sequence having at least 80% sequence identity to SEQ ID NO:73 or encoded by a nucleotide sequence having at least 80% sequence identity to SEQ ID NO:80, a Cas8 polypeptide comprising an amino acid sequence having at least 80% sequence identity to SEQ ID NO:74 or encoded by a nucleotide sequence having at least 80% sequence identity to SEQ ID NO:81, and a Cas7 polypeptide comprising an amino acid sequence having at least 80% sequence identity to SEQ ID NO:75 or encoded by a nucleotide sequence having at least 80% sequence identity to SEQ ID NO:82; and a Cas3 polypeptide comprising an amino acid sequence having at least 80% sequence identity to SEQ ID NO:72 or encoded by a nucleotide sequence having at least 80% sequence identity to SEQ ID NO:79, optionally, wherein when used in combination with a CRISPR, the CRISPR comprises any combination of one or more repeat sequences, or portion thereof, having at least 80% sequence identity to the nucleotide sequence of SEQ ID NOs:86-88, or optionally SEQ ID NOs:34, 35, 103-105, 120, or 121.

In some embodiments, the present invention provides recombinant nucleic acid molecules encoding one or more polypeptides of a Type I-C Cascade complex, the one or more polypeptides of a Type I-C Cascade complex comprising a Cas5 polypeptide comprising an amino acid sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO:90 or encoded by a nucleotide sequence having at least 80% sequence identity to SEQ ID NO:97, a Cas8 polypeptide comprising an amino acid sequence having at least 80% sequence identity to SEQ ID NO:91 or encoded by a nucleotide sequence having at least 80% sequence identity to SEQ ID NO:98, and a Cas7 polypeptide comprising an amino acid sequence having at least 80% sequence identity to SEQ ID NO:92 or encoded by a nucleotide sequence having at least 80% sequence identity to SEQ ID NO:99, optionally, wherein when used in combination with a CRISPR, the CRISPR comprises any combination of one or more repeat sequences, or portion thereof, having at least 80% sequence identity to the nucleotide sequence of SEQ ID NOs: 103-105, or optionally SEQ ID NOs: 34, 35, 86-88, 120, or 121; a Cas5 polypeptide comprising an amino acid sequence having at least 80% sequence identity to SEQ ID NO:90 or encoded by a nucleotide sequence having at least 80% sequence identity to SEQ ID NO:97, a Cas8 polypeptide comprising an amino acid sequence having at least 80% sequence identity to SEQ ID NO:91 or encoded by a nucleotide sequence having at least 80% sequence identity to SEQ ID NO:98, and a Cas7 polypeptide comprising an amino acid sequence having at least 80% sequence identity to SEQ ID NO:92 or encoded by the nucleotide sequence of SEQ ID NO:99; and a Cas3 polypeptide comprising an amino acid sequence having at least 80% sequence identity to SEQ ID NO:89 or encoded by a nucleotide sequence having at least 80% sequence identity to SEQ ID NO:96, optionally, wherein when used in combination with a CRISPR, the CRISPR comprises any combination of one or more repeat sequences, or portion thereof, having at least 80% sequence identity to the nucleotide sequence of SEQ ID NOs: 103-105, or optionally SEQ ID NOs: 34, 35, 86-88, 120, or 121.

In some embodiments, the present invention provides recombinant nucleic acid molecules encoding one or more polypeptides of a Type I-C Cascade complex, the one or more polypeptides of a Type I-C Cascade complex comprising a Cas5 polypeptide comprising an amino acid sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO:107 or encoded by a nucleotide sequence having at least 80% sequence identity to SEQ ID NO:114, a Cas8 polypeptide comprising an amino acid sequence having at least 80% sequence identity to SEQ ID NO:108 or encoded by a nucleotide sequence having at least 80% sequence identity to SEQ ID NO:115, and a Cas7 polypeptide comprising an amino acid sequence having at least 80% sequence identity to SEQ ID NO:109 or encoded by a nucleotide sequence having at least 80% sequence identity to SEQ ID NO:116, optionally, wherein when used in combination with a CRISPR, the CRISPR comprises any combination of one or more repeat sequences, or portion thereof, having at least 80% sequence identity to the nucleotide sequence of SEQ ID NO:120 or SEQ ID NO:121, or optionally SEQ ID NOs:34, 35, 86-88, or 103-105; a Cas5 polypeptide comprising an amino acid sequence having at least 80% sequence identity to SEQ ID NO:107 or encoded by a nucleotide sequence having at least 80% sequence identity to SEQ ID NO:114, a Cas8 polypeptide comprising an amino acid sequence having at least 80% sequence identity to SEQ ID NO:108 or encoded by a nucleotide sequence having at least 80% sequence identity to SEQ ID NO:115, and a Cas7 polypeptide comprising an amino acid sequence having at least 80% sequence identity to SEQ ID NO:109 or encoded by a nucleotide sequence having at least 80% sequence identity to SEQ ID NO:116; and a Cas3 polypeptide comprising an amino acid sequence having at least 80% sequence identity to SEQ ID NO:106 or encoded by a nucleotide sequence having at least 80% sequence identity to SEQ ID NO:113, optionally, wherein when used in combination with a CRISPR, the CRISPR comprises any combination of one or more repeat sequences, or portion thereof, having at least 80% sequence identity to the nucleotide sequence of SEQ ID NO:120 or SEQ ID NO:121, or optionally SEQ ID NOs:34, 35, 86-88, or 103-105.

### Type I-B CRISPR System Polypeptides and Polynucleotides

In some embodiments, a recombinant nucleic acid construct of the invention may encode a Type I-B CRISPR associated complex for antiviral defense complex (Cascade complex) comprising: (1) a Cas6 polypeptide, a Cas8 polypeptide, a Cas7 polypeptide, and a Cas5 polypeptide. In some embodiments, a recombinant nucleic acid construct of the invention may further comprise a Cas3 polypeptide of a Type I-B CRISPR-Cas system.

In some embodiments, a Cas6 polypeptide comprises the amino acid sequence of SEQ ID NO:122 or SEQ ID NO:140 (or a polypeptide sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity to the amino acid sequence of SEQ ID NO:122 or SEQ ID NO:140). In some embodiments, a Cas8 polypeptide comprises the amino acid sequence of SEQ ID NO:123 or SEQ ID NO:141 (or a polypeptide sequence having at least 80% sequence identity to the amino acid sequence of SEQ ID NO:123 or SEQ ID NO:141). In some embodiments, a Cas7 polypeptide comprises the amino acid sequence of SEQ ID NO:124 or SEQ ID NO:142 (or a polypeptide sequence having at least 80% sequence identity to any one of the amino acid sequence of SEQ ID NO:124 or SEQ ID NO:142). In some embodiments, a Cas5 polypeptide comprises a sequence having at least 80% sequence identity to the amino acid sequence of SEQ ID NO:125 or SEQ ID NO:143 (or a polypeptide sequence having at least 80% sequence identity to any one of the amino acid sequence of SEQ ID NO:125 or SEQ ID NO:143). In some embodiments, a Cas3 polypeptide comprises the amino acid sequence of SEQ ID NO:5 or SEQ ID 23 (or a polypeptide sequence having at least 80% sequence identity to the amino acid sequence of SEQ ID NO:5 or SEQ ID NO:23).

In some embodiments, a Cas6 polypeptide is encoded by a nucleotide sequence of SEQ ID NO:130 or SEQ ID NO:148 or a nucleotide sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO:130 or SEQ ID NO:148. In some embodiments, a Cas8 polypeptide is encoded by the nucleotide sequence of SEQ ID NO:131 or SEQ ID NO:149 or a nucleotide sequence having at least 80% sequence identity to SEQ ID NO:131 or SEQ ID NO:149. In some embodiments, a Cas7 polypeptide is encoded by the nucleotide sequence of SEQ ID NO:132 or SEQ ID NO:150 or a nucleotide sequence having at least 80% sequence identity to SEQ ID NO:132 or SEQ ID NO:150. In some embodiments, a Cas5 polypeptide is encoded by the nucleotide sequence of SEQ ID NO:133 or SEQ ID NO:151 or a nucleotide sequence having at least 80% sequence identity to SEQ ID NO:133 or SEQ ID NO:151. In some embodiments, a Cas3 polypeptide is encoded by the nucleotide sequence of SEQ ID NO:134 or SEQ ID NO:152 or a nucleotide sequence having at least 80% sequence identity to SEQ ID NO:134 or SEQ ID NO:152.

Accordingly, in some embodiments, the present invention provides recombinant nucleic acid molecules encoding one or more polypeptides of a Type I-B Cascade complex, the one or more polypeptides of a Type I-B Cascade complex comprising a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:122 or encoded by a nucleotide sequence having at least 80% sequence identity to SEQ ID NO:140, a Cas8 polypeptide having at least 80% sequence identity to the amino acid sequence of SEQ ID NO:123 or encoded by a nucleotide sequence having at least 80% sequence identity to SEQ ID NO:141, a Cas7 polypeptide having at least 80% sequence identity to the amino acid sequence of SEQ ID NO:124 or encoded by a nucleotide sequence having at least 80% sequence identity to SEQ ID NO:142, and a Cas5 polypeptide having at least 80% sequence identity to the amino acid sequence of SEQ ID NO:125 or encoded by a nucleotide sequence having at least 80% sequence identity to SEQ ID NO:143, optionally, wherein when used in combination with a CRISPR, the CRISPR comprises one or more repeat sequences, or portion thereof, having at least 80% sequence identity to a nucleotide sequence of SEQ ID NO:138 or SEQ ID NO:139, or any combination thereof.

In some embodiments, the one or more polypeptides of a Type I-B Cascade complex comprising a Cas6 polypeptide having at least 80% sequence identity to SEQ ID NO:122 or encoded by a nucleotide sequence having at least 80% sequence identity to SEQ ID NO:140, a Cas8 polypeptide having at least 80% sequence identity to the amino acid sequence of SEQ ID NO:123 or encoded by a nucleotide sequence having at least 80% sequence identity to SEQ ID NO:141, a Cas7 polypeptide having at least 80% sequence identity to the amino acid sequence of SEQ ID NO:124 or encoded by a nucleotide sequence having at least 80% sequence identity to SEQ ID NO:142, and a Cas5 polypeptide having at least 80% sequence identity to the amino acid sequence of SEQ ID NO:125 or encoded by a nucleotide sequence having at least 80% sequence identity to SEQ ID NO:143; and a Cas3 polypeptide having at least 80% sequence identity to the amino acid sequence of SEQ ID NO:126 or encoded by a nucleotide sequence having at least 80% sequence identity to SEQ ID NO:144, optionally, wherein when used in combination with a CRISPR, the CRISPR comprises one or more repeat sequences, or portion thereof, having at least 80% sequence identity to a nucleotide sequence of SEQ ID NO:138 or SEQ ID NO:139, or any combination thereof.

In some embodiments, the present invention provides recombinant nucleic acid molecules encoding one or more polypeptides of a Type I-B Cascade complex, the one or more polypeptides of a Type I-B Cascade complex comprising a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to SEQ ID NO:140 or encoded by the nucleotide sequence of SEQ ID NO:148, a Cas8 polypeptide having at least 80% sequence identity to SEQ ID NO:141 or encoded by a nucleotide sequence having at least 80% sequence identity to SEQ ID NO:149, a Cas7 polypeptide having at least 80% sequence identity to SEQ ID NO:142 or encoded by a nucleotide sequence having at least 80% sequence identity to SEQ ID NO:150, and a Cas5 polypeptide having at least 80% sequence identity to SEQ ID NO:143 or encoded by a nucleotide sequence having at least 80% sequence identity to SEQ ID NO:151, optionally wherein when used in combination with a CRISPR, the CRISPR comprises one or more repeat sequences, or portion thereof, having at least 80% sequence identity to the nucleotide sequence of SEQ ID NOs:156-164 or any combination thereof.

In some embodiments, recombinant nucleic acid molecules encoding one or more polypeptides of a Type I-B Cascade complex are provided wherein the one or more polypeptides of a Type I-B Cascade complex comprise a Cas6 polypeptide having at least 80% sequence identity to SEQ ID NO:140 or encoded by the nucleotide sequence of SEQ ID NO:148, a Cas8 polypeptide having at least 80% sequence identity to SEQ ID NO:141 or encoded by a nucleotide sequence having at least 80% sequence identity to SEQ ID NO:149, a Cas7 polypeptide having at least 80% sequence identity to SEQ ID NO:142 or encoded by a nucleotide sequence having at least 80% sequence identity to SEQ ID NO:150, and a Cas5 polypeptide having at least 80% sequence identity to SEQ ID NO:143 or encoded by a nucleotide sequence having at least 80% sequence identity to SEQ ID NO:151; and a Cas3 polypeptide having at least 80% sequence identity to SEQ ID NO:144 or encoded by a nucleotide sequence having at least 80% sequence identity to SEQ ID NO:152, optionally wherein when used in combination with a CRISPR, the CRISPR comprises one or more repeat sequences, or portion thereof, having at least 80% sequence identity to the nucleotide sequence of SEQ ID NOs:156-164 or any combination thereof.

### Protein-RNA Complexes

In some embodiments, the invention provides a CRISPR and the polypeptides of a Type I-C Cascade complex and a Cas3 in a protein-RNA complex (ribonucleoprotein, RNP). Thus, is some embodiments a protein-RNA complex is provided that comprises (a) a Cas3 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the amino acid sequences of SEQ ID NOs:1, 20, 36, 54, 72, 89, or 106, and a Type I-C CRISPR associated complex for antiviral defense complex (Cascade complex) comprising a Cas5 polypeptide having at least 80% sequence identity to any one of the amino acid sequences of SEQ ID NOs:2, 21, 37, 55, 73, 90, or 107, a Cas8 polypeptide having at least 80% sequence identity to any one of the amino acid sequences of SEQ ID NOs:3, 22, 38, 56, 74, 91, or 108, and a Cas7 polypeptide having at least 80% sequence identity to any one of the amino acid sequences of SEQ ID NOs:4, 23, 39, 57, 75, 92, or 109; and
(b) a Clustered Regularly Interspaced Short Palindromic Repeats (CRISPR) comprising one or more (e.g., 1, 2, 3, 4, 5, 6, 7, 8, 9, 10 or more) repeat sequence(s) and one or more (e.g., 1, 2, 3, 4, 5, 6, 7, 8, 9, 10 or more) spacer sequence(s), wherein each spacer sequence is linked at its 5′ end and at its 3′ end to a repeat sequence, and the spacer sequence is complementary to a target sequence (protospacer) in a target DNA of a target organism, wherein the target DNA is located immediately adjacent (3′) to a protospacer adjacent motif (PAM).

In contrast to the recombinant nucleic acid constructs and protein-RNA constructs of the present invention, a wild type Type I-C Cascade complex of C. scindens, C. clostridioforme or C. bolteae further comprises Cas4, Cas1 and Cas2 (see, e.g., polypeptide sequences of SEQ ID NOs:5, 6 and 7, respectively; SEQ ID NOs:24, 25 and 26, respectively; SEQ ID NOs:40, 41 and 42, respectively; SEQ ID NOs:57, 58 and 59, respectively; SEQ ID NOs:76, 77 and 78, respectively; SEQ ID NOs:93, 94 and 95, respectively; SEQ ID NOs:110, 111 and 112, respectively; or the nucleotide sequences of SEQ ID NOs:12, 13, and 14, respectively; SEQ ID NOs:31, 32 and 33, respectively; SEQ ID NOs:47, 48 and 49, respectively; SEQ ID NOs:65, 66 and 67, respectively; SEQ ID NOs:83, 84 and 85, respectively; SEQ ID NOs:100, 101 and 102 respectively; SEQ ID NOs:117, 118 and 119, respectively), which are responsible for spacer acquisition in wild type CRISPR-Cas systems.

In some embodiments a protein-RNA complex is provided comprising: (a) a Type I-B CRISPR associated complex for antiviral defense complex (Cascade complex) comprising (i) a Cas6 polypeptide having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:122, a Cas8 polypeptide having at least 80% sequence identity to the amino acid sequence of SEQ ID NO:123, a Cas7 polypeptide having at least 80% sequence identity to the amino acid sequence of SEQ ID NO:124, and a Cas5 polypeptide having at least 80% sequence identity to the amino acid sequence of SEQ ID NO:125; and a Cas3 polypeptide having at least 80% sequence identity to the amino acid sequence of SEQ ID NO:126; or (ii) a Cas6 polypeptide having at least 80% sequence identity to the amino acid sequence of SEQ ID NO:140, a Cas8 polypeptide having at least 80% sequence identity to the amino acid sequence of SEQ ID NO:141, a Cas7 polypeptide having at least 80% sequence identity to the amino acid sequence of SEQ ID NO:142, and a Cas5 polypeptide having at least 80% sequence identity to the amino acid sequence of SEQ ID NO:143; and a Cas3 polypeptide having at least 80% sequence identity to the amino acid sequence of SEQ ID NO:144; and (b) a Clustered Regularly Interspaced Short Palindromic Repeats (CRISPR) comprising one or more (e.g., 1, 2, 3, 4, 5, 6, 7, 8, 9, 10, 11, 12, 13, 14, 15 or more) repeat sequence(s) and one or more (e.g., 1, 2, 3, 4, 5, 6, 7, 8, 9, 10, 11, 12, 13, 14, 15 or more) spacer sequence(s), wherein each spacer sequence is linked at its 5′ end and at its 3′ end to a repeat sequence, and the spacer sequence is complementary to a target sequence (protospacer) in a nucleic acid of a target organism, wherein the target sequence is located immediately adjacent (3′) to a protospacer adjacent motif (PAM).

In contrast to the recombinant nucleic acid constructs and protein-RNA constructs of the present invention, a wild type Type I-B Cascade complex of Erysipelatoclostridium ramosum further comprises Cas4, Cas1 and Cas2 (see, polypeptide sequences of SEQ ID NOs:127, 128 and 129, respectively; nucleotide sequences of SEQ ID NOs:135, 136 and 137), which are responsible for spacer acquisition in wild type CRISPR-Cas systems. Similarly, a wild type Type I-B Cascade complex of Clostridium spp. 1141A1FAA further comprises Cas4, Cas1 and Cas2 (see, polypeptide sequences of SEQ ID NOs:145, 146 and 147, respectively; nucleotide sequences of SEQ ID NOs:153, 154, and 155), which are responsible for spacer acquisition in wild type CRISPR-Cas systems.

In some embodiments, the recombinant nucleic acid constructs of the invention may be comprised in a vector (e.g., a plasmid, a phagemid, a transposon, a bacteriophage, and/or a retrovirus. Thus, in some embodiments, the invention further provides phagemid, plasmid, bacteriophage, transposon, and/or retroviral vectors comprising the recombinant nucleic acid constructs of the invention.

Plasmids useful with the invention may be dependent on the target organism, that is, dependent on where the plasmid is to replicate. Non-limiting examples of plasmids that express in Lactobacillus include pNZ and derivatives, pGK12 and derivatives, pTRK687 and derivatives, pTRK563 and derivatives, pTRKH2 and derivatives, pIL252, and/or pIL253. Additional, non-limiting plasmids of interest include pORI-based plasmids or other derivatives and homologs.

Accordingly, the present invention provides one vector or more than one vector encoding a recombinant nucleic acid of the invention. In some embodiments, vector may comprise, consist essentially of or consist or a recombinant nucleic acid encoding a Type I-C CRISPR associated complex for antiviral defense complex (Cascade complex) comprising a Cas5 polypeptide, a Cas8 polypeptide, and a Cas7 polypeptide; or comprising a Cas5 polypeptide, a Cas8 polypeptide, and a Cas7 polypeptide; and a Cas3 polypeptide, wherein the Cas5 polypeptide comprises an amino acid sequence of SEQ ID NOs:2, 21, 37, 55, 73, 90, or 107, the Cas8 polypeptide comprises an amino acid sequence of SEQ ID NOs:3, 22, 38, 56, 74, 91, or 108, the Cas7 polypeptide comprises an amino acid sequence of SEQ ID NOs:4, 23, 39, 57, 75, 92, or 109, and when present, the Cas3 polypeptide comprises an amino acid sequence of SEQ ID NOs:1, 20, 36, 54, 72, 89, or 106, as described herein.

In some embodiments, vector may comprise, consist essentially of or consist or a recombinant nucleic acid encoding a Cas3 polypeptide and a Type I-B CRISPR associated complex for antiviral defense complex (Cascade complex) comprising a Cas6 polypeptide, a Cas8 polypeptide, a Cas7 polypeptide, and a Cas5 polypeptide, wherein the Cas6 polypeptide comprises the amino acid sequence of SEQ ID NO:122, the Cas8 polypeptide comprises the amino acid sequence of SEQ ID NO:123, the Cas7 polypeptide comprises the amino acid sequence of SEQ ID NO:124, the Cas5 polypeptide comprises the amino acid sequence of SEQ ID NO:125, and the Cas3 polypeptide comprises the amino acid sequence of SEQ ID NO:126; or the Cas6 polypeptide comprises the amino acid sequence of SEQ ID NO:140, the Cas8 polypeptide comprises the amino acid sequence of SEQ ID NO:141, the Cas7 polypeptide comprising the amino acid sequence of SEQ ID NO:142, the Cas5 polypeptide comprising the amino acid sequence of SEQ ID NO:143 and the Cas3 polypeptide comprises the amino acid sequence of SEQ ID NO:145.

### Enhancing Bacteriophage Resistance in Bacteria

The compositions (e.g., recombinant nucleic acid constructs) of the present invention may be used, for example, in methods for enhancing the resistance of bacterial cells to one or more bacteriophage species or strains.

Accordingly, the recombinant nucleic acid constructs of the invention may be introduced into a cell of an organism, or where relevant, the constructs may be contacted with a target nucleic acid in a cell free system. In some embodiments, the recombinant nucleic acid constructs of the invention may be stably or transiently introduced into a cell of a bacterium of interest for the purpose of modifying the genome and/or for altering expression in the cell or for modifying the target nucleic acid or its expression in the cell free system.

In some embodiments, when a bacterial cell for which enhanced resistance to one or more bacteriophage species or strains may be desired comprises an endogenous CRISPR-Cas system that is compatible with the recombinant CRISPR nucleic acids of the invention (e.g., a Type I-C CRISPR Cas system; e.g., a Type I-C CRISPR Cas system of C. scindens, C. clostridioformes, C. bolteae; e.g., a Type I-B CRISPR Cas system; e.g., a Type I-B CRISPR Cas system of E. ramosum or Clostridium spp. 1141A1FAA)), the endogenous CRISPR-Cas system of a cell (e. g., endogenous Cascade complex and Cas3) may be co-opted for use with the recombinant CRISPR nucleic acids of the invention (e.g., the recombinant nucleic acid constructs comprising a CRISPR) for the purpose of modifying the genome and/or for altering expression in the cell.

Accordingly, in some embodiments, a method of enhancing resistance of a bacterial cell to one or more bacteriophage species or strains is provided, the method comprising introducing into the bacterial cell a recombinant nucleic acid construct comprising a non-natural Type I-B Clustered Regularly Interspaced Short Palindromic Repeats (CRISPR) or a Type I-C CRISPR comprising one or more repeat sequences and one or more spacer sequence(s), wherein each of the one or more spacer sequences is linked at least at its 5′-end to a repeat sequence or portion thereof, and each of the one or more spacer sequences is complementary to a target sequence (protospacer) in a target nucleic acid of a bacteriophage species or strain, wherein the target sequence is located immediately adjacent (3′) to a protospacer adjacent motif (PAM). The bacterial cell may be any bacterial cell or bacterial population for which an increased resistance to a bacteriophage species or strain is desirable. Such bacteria include, for example, commensal, probiotic or other commercially important bacteria or strain of bacteria including, but not limited to, bacteria from genus Lactobacillus or Bifidobacterium. In some embodiments, bacteria useful with the invention include, but are not limited to, those in the species of L. acidophilus, L. casei, L. paracasei, L. crispatus, L. gasseri, L. plantarum, L. rhamnosus, L. salivarius, L. fermentum, L. reuteri, L. johnsonii, B. longum, B. lactis, B. infantis, or any combination thereof.

A bacterial cell for use with this invention may be a single cell or a cell within a population of bacterial cells of the same species or strain or may be a cell within a population comprising a mixture of two or more bacterial species or strains. In some embodiments, the methods of this invention (e.g., enhancing resistance to one or more bacteriophage species or strains) may be carried out on a portion of a population of bacterial cells. As used herein, “a portion of the population of cells” means at least one cell of a population of two or more cells (e.g., 2, 3, 4, 5, 6, 7, 8, 9, 10, or more cells, e.g., 10², 10³, 10⁴, 10⁵, 10⁶, 10⁷, 10⁸, 10⁹, 10¹⁰ or more cells). In some embodiments, the bacterial cell is a cell of a commensal bacterial species or strain, optionally the bacterial cell is a cell of a commensal Clostridium spp. or strain. In some embodiments, the bacterial cell may be a Clostridium spp., a Erysipelatoclostridium spp., a Lactococcus spp., a Streptococcus spp., a Klebsiella spp., a Propionibacterium spp., a Cutibacterium spp., a Lactobacillus spp., a Pseudomonas spp., a Faecalibacterium spp., a Akkermansia spp., a Bifidobacterium spp., a Roseburia spp., an E. coli spp., or a Clostridiodis spp. In some embodiments, the bacterial cell may be a Clostrium spp. cell, a Clostridium scindens cell, a Clostridium clostridioforme cell, a Clostridium bolteae cell, or a Erysipelatoclostridium ramosum cell.

In some embodiments, when a bacterial cell for which enhanced resistance to one or more bacteriophage species or strains may be desired does not comprise an endogenous CRISPR-Cas system that is functional and/or compatible with the recombinant CRISPR nucleic acids of the invention (e.g., a Type I-C CRISPR Cas system; e.g., a Type I-C CRISPR Cas system of C. scindens, C. clostridioformes, C. bolteae; e.g., a Type I-B CRISPR Cas system; e.g., a Type I-B CRISPR Cas system of E. ramosum or Clostridium spp. 1141A1FAA)), a recombinant nucleic acid construct comprising a Cas3 polynucleotide and a Type I-B Cascade complex and/or a recombinant nucleic acid construct comprising a Cas3 polynucleotide and a Type I-C Cascade complex may also be introduced together with a CRISPR or CRISPR array of the invention.

Thus, in some embodiments, a method of enhancing resistance of a bacterial cell to one or more bacteriophage species or strains is provided, the method comprising introducing into the bacterial cell (a) a recombinant nucleic acid construct comprising a non-natural Type I-C Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and each of the one or more spacer sequences is complementary to a target sequence (protospacer) in a target nucleic acid of a bacteriophage species or strain, wherein the target sequence is located immediately adjacent (3′) to a protospacer adjacent motif (PAM); and (b) one or more recombinant nucleic acid constructs encoding Cas3 polypeptide and a Type I-C Cascade complex, wherein the Type I-C Cascade complex comprises a Cas5 polypeptide, a Cas8 polypeptide, and a Cas7 polypeptide. In some embodiments, the Cas3 polypeptide comprises a sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the amino acid sequences of SEQ ID NOs:1, 20, 36, 54, 72, 89, or 106, the Cas5 polypeptide comprises a sequence having at least 80% sequence identity to any one of the amino acid sequences of SEQ ID NOs:2, 21, 37, 55, 73, 90, or 107, the Cas8 polypeptide comprises a sequence having at least 80% sequence identity to any one of the amino acid sequences of SEQ ID NOs:3, 22, 38, 56, 74, 91, or 108, and the Cas7 polypeptide comprises a sequence having at least 80% sequence identity to any one of the amino acid sequences of SEQ ID NOs:4, 23, 39, 57, 75, 92, or 109, thereby enhancing resistance of a bacterial cell to one or more bacteriophage species or strains.

In some embodiments, a method of enhancing resistance of a bacterial cell to one or more bacteriophage species or strains is provided, the method comprising introducing into the bacterial cell (a) a recombinant nucleic acid construct comprising a non-natural Type I-B Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and each of the one or more spacer sequences is complementary to a target sequence (protospacer) in a target nucleic acid of a bacteriophage species or strain, wherein the target sequence is located immediately adjacent (3′) to a protospacer adjacent motif (PAM); and (b) one or more recombinant nucleic acid constructs encoding Cas3 polypeptide and a Type I-B Cascade complex, wherein the Type I-B Cascade complex comprises a Cas6 polypeptide, a Cas8 polypeptide, a Cas7 polypeptide, and a Cas5 polypeptide. In some embodiments, the Cas3 polypeptide comprises a sequence having at least 80% sequence identity to the amino acid sequence of SEQ ID NO:126 or SEQ ID NO:144, the Cas6 polypeptide comprises a sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:122 or SEQ ID NO:140, the Cas8 polypeptide comprises a sequence having at least 80% sequence identity to the amino acid sequence of SEQ ID NO:123 or SEQ ID NO:141, the Cas7 polypeptide comprises a sequence having at least 80% sequence identity to the amino acid sequence of SEQ ID NO:124 or SEQ ID NO:142, and the Cas5 polypeptide comprises a sequence having at least 80% sequence identity to the amino acid sequence of SEQ ID NO:125 or SEQ ID NO:142.

A CRISPR of the invention may also be introduced into a cell (or cell free environment) in the form of a protein-RNA complex (RNP). Accordingly, in some embodiments, the present invention provides a method of enhancing resistance of a bacterial cell to one or more bacteriophage species or strains, the method comprising introducing into the bacterial cell (a) at least one protein-RNA complex comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence, and each of the one or more spacer sequences is complementary to a target sequence (protospacer) in a target nucleic acid of a bacteriophage species or strain, wherein the target sequence is located immediately adjacent (3′) to a protospacer adjacent motif (PAM), (b) a Cas3 polypeptide, and (c) a Type I-B Cascade complex comprising a Cas6 polypeptide, a Cas8 polypeptide, a Cas7 polypeptide, and a Cas5 polypeptide, wherein the Cas3 polypeptide comprises a sequence having at least 80% sequence identity to the amino acid sequence of SEQ ID NO:126 or SEQ ID NO:144, the Cas6 polypeptide comprises a sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:122 or SEQ ID NO:140, the Cas8 polypeptide comprises a sequence having at least 80% sequence identity to the amino acid sequence of SEQ ID NO:123 or SEQ ID NO:141, the Cas7 polypeptide comprises a sequence having at least 80% sequence identity to the amino acid sequence of SEQ ID NO:124 or SEQ ID NO:142, and the Cas5 polypeptide comprises a sequence having at least 80% sequence identity to the amino acid sequence of SEQ ID NO:125 or SEQ ID NO:142.

In some embodiments, a method of enhancing resistance of a bacterial cell to one or more bacteriophage species or strains is provided, the method comprising introducing into the bacterial cell (a) at least one protein-RNA complex comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence, and each of the one or more spacer sequences is complementary to a target sequence (protospacer) in a target nucleic acid of a bacteriophage species or strain, wherein the target sequence is located immediately adjacent (3′) to a protospacer adjacent motif (PAM), (b) a Cas3 polypeptide, and (c) a Type I-C Cascade complex comprising a Cas5 polypeptide, a Cas8 polypeptide and a Cas7 polypeptide, wherein the Cas3 polypeptide comprises a sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the amino acid sequences of SEQ ID NOs:1, 20, 36, 54, 72, 89, or 106, the Cas5 polypeptide of the Type I-C Cascade complex comprises a sequence having at least 80% sequence identity to any one of the amino acid sequences of SEQ ID NOs:2, 21, 37, 55, 73, 90, or 107, the Cas8 polypeptide of the Type I-C Cascade complex comprises a sequence having at least 80% sequence identity to any one of the amino acid sequences of SEQ ID NOs:3, 22, 38, 56, 74, 91, or 108, and the Cas7 polypeptide of the Type I-C Cascade complex comprises a sequence having at least 80% sequence identity to any one of the amino acid sequences of SEQ ID NOs:4, 23, 39, 57, 75, 92, or 109.

A CRISPR useful with this invention is as defined herein and may be an unprocessed or a processed (e.g., mature) CRISPR or a CRISPR that is non-natural (e.g., repeat-spacer). A processed CRISPR comprises a spacer linked at its 5′ end and its 3′ end to a repeat sequence, wherein the repeat sequence is a portion of a full-length repeat sequence. An unprocessed CRISPR comprises at least one spacer linked at both its 5′ end and its 3′ end to a full-length repeat sequence. A “non-natural CRISPR,” as used herein, refers also to a CRISPR comprising a spacer (e.g., a non-native spacer) that has not previously been acquired by the target bacterial cell (e.g., the bacterial cell for which enhanced resistance to one or more bacteriophage species or strains is desired).

In some embodiments, a bacteriophage for which increased resistance is desired may be a new bacteriophage species or strain that has not been previously recognized or targeted by an endogenous CRISPR system of the bacterial cell (e.g., the CRISPR system of the bacterial cell does not comprise a spacer sequence having complementary to a nucleic acid of the new bacteriophage species or strain, e.g., no naturally occurring/endogenous spacer sequence having complementarity (e.g., having less than 100% (e.g., about 99, 98, 97, 96, 95, 94, 93, 92, 91, 90, 89, 88, 87, 86, 85, 84, 83, 82, 81, 80, 79, 78, 77, 76, 75, 74, 73, 72, 71, 70% or less complementarity) to a nucleic acid of the new bacteriophage species or strain is present in a CRISPR array of the bacterium). Thus, the methods of the present invention modify the resistance of the bacterium to include resistance to at least one bacteriophage species or strain to which the bacterium has not previously been resistant or to which the bacterium has had poor resistance.

In some embodiments, a bacteriophage for which increased or enhanced resistance is desired may be a bacteriophage species or strain that is targeted or recognized by an endogenous CRISPR system of the target bacterial cell (e.g., the endogenous CRISPR system of the bacterial cell comprises at least one spacer that has complementarity to a nucleic acid sequence from the genome of the at least one bacteriophage species or strain); however, the at least one non-natural spacer sequence (introduced in a CRISPR of the invention) is complementary to a different nucleic acid sequence than a spacer sequence of the endogenous CRIPSR system, thereby increasing resistance to the at least one bacteriophage. In some embodiments the “different” nucleic acid sequence may comprise about 95% or less complementarity (e.g., about 95, 94, 93, 92, 91, 90, 89, 88, 87, 86, 85, 84, 83, 82, 81, 80, 79, 78, 77, 76, 75, 74, 73, 72, 71, 70% or less complementarity) to a spacer that has been naturally acquired by a bacterium's endogenous CRISPR-Cas system. Thus, the methods of the present invention modify the resistance of the bacterium to include an increase or enhancement of resistance to at least one bacteriophage to which the bacterium may have had poor or no resistance. Accordingly, enhanced resistance to a bacteriophage that may have been previously targeted or recognized by the endogenous CRISPR-Cas system of the bacterial cell (e.g., the endogenous CRISPR-Cas system may comprise a spacer having complementarity to a sequence of the target bacteriophage) may be improved by the introduction of the CRISPR comprising at least one non-natural spacer sequence that is complementary to a different nucleic acid sequence than a spacer sequence of the endogenous CRISPR-Cas system of the bacterium, thereby increasing/improving resistance to the at least one bacteriophage. In some embodiments, the spacer in the endogenous CRISPR system may not be effective for targeting the bacteriophage species or strain, while the newly introduced spacer in a CRISPR of the invention may be sufficiently different to provide effective killing of the bacteriophage species or strain, thereby providing not only an increase in resistance, but in some instances, resistance to a bacteriophage species or strain not previously observed for this bacteria.

Accordingly, in some embodiments, the bacterial cell (e.g., a target bacterial cell) comprising the target nucleic acid (e.g., target DNA/RNA) may comprise an endogenous CRISPR-Cas system that is operable with a CRISPR as described herein, thereby increasing the resistance of the bacterial cell to a bacteriophage. The endogenous CRISPR-Cas system of the bacterial cell may be a Type I CRISPR-Cas system, Type II CRISPR-Cas system, Type III CRISPR-Cas system, Type IV CRISPR-Cas system, Type V CRISPR-Cas system or Type VI CRISPR-Cas system.

Notably, though it is possible to screen for natural vaccination events during which novel spacers are acquired in the adaptation stage of CRISPR-Cas system-encoded adaptive immunity in bacteria, this is a relatively rare phenomenon which has only been documented in very few model systems in laboratory conditions. Indeed, novel spacer acquisition from invasive genetic elements has been estimated to occur in approximately one in 10 million cells (Barrangou et al., Science 315(5819):1709-12 (2007); McGinn and Marraffini, Mol Cell 64(3):616-623 (2016); Heler et al., Nature 519(7542):199-202 (2015); Hynes et al., Nature Comm 5:4399 (2014); McGinn and Marraffini Nature Rev Microbiol 17(1):7-12 (2019)). Consequently, it is possible but highly unlikely to design novel spacers that would match what would be rarely obtained in nature, especially since the typical phage contains hundreds of potential protospacers that can be acquired by active CRISPR-Cas systems (Paez-Espino et al., Nature Comm 4:1430 (2013)). Furthermore, it is known that because natural naive adaptation is rare, most acquisition studies rely on engineered systems in which the acquisition machinery is engineered, de-repressed and/or induced, or needs to be “primed” (Jackson et al., Cell Host Microbe 25(2):250-260 (2019); Jackson et al., Science 356(6333) (2017); Staals et al., Nature Comm 7:12853 (2016); Nussenzweig et al., Cell Host Microbe 26(4):515-526 (2019)). From a method standpoint, an inventor may screen for such rare natural events strategically, or design and integrate using engineering and molecular biology novel spacers into a CRISPR array. The engineering through synthetic biology approach overcomes the shortcoming inherent to the rarity of natural acquisition events, however, the outcomes rarely overlap.

In some embodiments, a target region may be about 10 to about 40 consecutive nucleotides in length located immediately adjacent to a PAM sequence (PAM sequence located immediately 3′ of the target region) in the genome of the organism (e.g., Type I CRISPR-Cas systems and Type II CRISPR-Cas systems). In the some embodiments, e.g., Type I systems, the PAM is on the alternate side of the protospacer (the 5′ end). There is no known PAM for Type III systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

In some embodiments, a target nucleic acid (e.g., target DNA/RNA) is located adjacent to a protospacer adjacent motif (PAM). In some embodiments, the CRISPR array is a Type I CRISPR array and the target nucleic acid (protospacer) is located adjacent to the 3′ end of a PAM (PAM is 5′ to the protospacer). In some embodiments, the CRISPR array is a Type II CRISPR array and the target nucleic acid (e.g., target DNA/RNA) is located adjacent to the 5′ end of the PAM (PAM is 3′ to the protospacer). In some embodiments, the CRISPR array is a Type V CRISPR array, and the target nucleic acid (e.g., target DNA/RNA) is typically located adjacent to the 5′ end of the PAM (PAM is 3′ to the protospacer) similar to Type II. In some embodiments, a target nucleic acid useful with a Type V CRISPR-Cas system may be located adjacent to the 3′ end of the PAM (PAM is 5′ to the protospacer), similar to Type I. In some embodiments, the CRISPR array is a Type III CRISPR array or a Type VI CRISPR array and the target nucleic acid (e.g., target DNA/RNA) is not adjacent to a PAM but instead may be located within the target RNA. In the context of a “protospacer” and a “spacer,” “adjacent” can mean immediately adjacent to (5′ or 3′ depending on the system) or it can mean one to seven nucleotides between the PAM and the protospacer. For example, in a Type I system, a PAM is typically immediately upstream of the protospacer. In a Type II system, a PAM is typically one to two nucleotides downstream of the protospacer but can be from about one to seven nucleotides downstream of the protospacer (e.g., 1, 2, 3, 4, 5, 6, 7 nucleotides, e.g., 1 to 2, 1 to 3, 1 to 4, 1 to 5, 3 to 5, 3 to 7, 4 to 7, 5 to 7 nucleotides downstream of the protospacer). For example, a PAM for a Type II-C CRISPR system may be 5 to 7 nucleotides downstream of the protospacer. In a Type V system, similar to a Type II system, a PAM is typically one to two nucleotides downstream of the protospacer.

In some embodiments, a protospacer adjacent motif (PAM) may comprise, consist essentially of, or consist of a nucleotide sequence of 5′-NAA-3′ and/or 5′-AAA-3′, 5′-NGG-3′, 5′-NGAAA-3′, 5′-NNG-3′, 5′-NGA-3′, 5′-NTAA-3′, 5′-NTG-3′, 5′-NNC-3′, 5′-NNAAC-3′, 5′-AGA-3′, 5′-NNNANNA-3′, 5′-NNANAA-3′, 5′-NNAAAA-3′, 5′-AAAA-3′, 5′-TTC-3′, 5′-TTT-3′, 5′-CTC-3′, 5′-TTTA-3′, 5′-ATC-3′, 5′-ATCN-3′ (N=T, A, C, or G, e.g., 5′-ATCA-3′, and/or 5′-ATCG-3′).

In some embodiments, PAM sequences useful with a Type I-C CRISPR-Cas system of this invention are located immediately adjacent to and 5′ of the target sequence (protospacer) and may include, but are not limited to, the nucleotide sequence of 5′-TTC-3′, the nucleotide sequence of 5′-TTT-3′ and/or the nucleotide sequence of 5′-CTC-3′. In some embodiments, PAM sequences useful with a Type I-B CRISPR-Cas system of this invention are located immediately adjacent to and 5′ of the target sequence (protospacer) and may include, but are not limited to, the nucleotide sequence of 5′-TTTA-3′ and/or the nucleotide sequence of 5′-ATC-3′ and/or 5′-ATCN-3′ (e.g., 5′-ATCA-3′, and/or 5′-ATCG-3′).

CRISPR nucleic acids useful with the methods of the invention may comprise one or more or two or more repeat sequences and one or more spacer sequence(s), wherein each spacer sequence and each repeat sequence have a 5′ end and a 3′ end and each spacer sequence is linked at its 5′ end, and optionally at its 3′ end, to a repeat sequence or to portion of a repeat sequence, and the spacer sequence is complementary to a target sequence (protospacer) in a target DNA of a target organism that is located immediately adjacent (3′) to a protospacer adjacent motif (PAM). In some embodiments, a CRISPR of the present invention comprising at least one spacer sequence and at least two repeat sequences (or portion thereof) flanking the spacer, may be expressed as a premature CRISPR that will be processed internally in the cell to constitute the final mature CRISPR. In some embodiments, a CRISPR of the present invention may comprise a processed CRISPR comprising a portion of a repeat sequence at the 5′ end and 3′ end of the spacer sequence. In some embodiments, a CRISPR of the present invention may comprise a non-native CRISPR comprising a spacer linked at its 5′ end to a repeat sequence or a portion of a repeat sequence.

An at least one additional component of a CRISPR-Cas system useful with the methods of the invention for killing a bacterial cell include, but are not limited to, a nucleic acid encoding a CRISPR-Cas nuclease, a trans-encoded CRISPR (tracr) nucleic acid, and/or a nucleic acid encoding an additional CRISPR-Cas polypeptide. When the at least one additional component is a tracr nucleic acid, the tracr nucleic acid and CRISPR array may be introduced as a single guide. A CRISPR-Cas nuclease useful with the methods of the invention may be, for example, a Type I CRISPR-Cas nuclease, a Type II CRISPR-Cas nuclease, a Type III CRISPR-Cas nuclease, a Type IV CRISPR-Cas nuclease, a Type V CRISPR-Cas nuclease and/or a Type VI CRISPR-Cas nuclease. In some embodiments, the Type II CRISPR-Cas nuclease is a Cas9 nuclease, the Type I nuclease is a Cas3 nuclease, the Type III nuclease is a Cas10 nuclease, the Type IV nuclease is a Csf4 nuclease, the Type V nuclease is a Cas12 nuclease and/or the Type VI nuclease is a Cas13 nuclease. Non-limiting examples of an additional CRISPR-Cas polypeptide may be a Type I CRISPR associated complex for antiviral defense complex (Cascade complex) polypeptide, a Type III Csm complex polypeptide, a Type III Csr complex polypeptide, a Type IV polypeptide, a Type V polypeptide and/or a Type VI polypeptide.

In some embodiments, a repeat sequence (i.e., CRISPR repeat sequence) as used herein may comprise any known repeat sequence of a wild-type Clostridium CRISPR Type I-C locus (e.g., C. bolteae, C. scindens, C. clostridioforme)). In some embodiments, a repeat sequence useful with the invention may include a synthetic repeat sequence having a different nucleotide sequence than those known in the art for Clostridium but sharing similar structure to that of wild-type Clostridium repeat sequences of a hairpin structure with a loop region. Thus, in some embodiments, a repeat sequence may be identical to (i.e., having 100% sequence identity) or substantially identical (e.g., having 80% to 99% sequence identity (e.g., 80, 81, 82, 83, 84, 85, 86, 87, 88, 89, 90, 91, 92, 93, 94, 95, 96, 97, 98, 99% sequence identity)) to a repeat sequence from a wild-type Clostridium CRISPR Type I-C locus.

The length of a Type I-C CRISPR repeat sequence useful with the recombinant nucleic acid constructs and methods of the invention may be the full length of a Clostridium (e.g., C. bolteae, C. scindens, C. clostridioforme) repeat sequence (i.e., about 32 nucleotides or 33 nucleotides) (see, e.g., SEQ ID NOs:15-19, 34, 35, 50-53, 68-71, 86-88, 103-105, 120, or 121). In some embodiments, a repeat sequence may comprise a portion of a wild type Clostridium repeat nucleotide sequence, the portion being reduced in length by as much as 7 to 8 contiguous nucleotides (e.g., 1, 2, 3, 4, 5, 6, 7, or 8 nucleotides) from the 3′ end as compared to a wild type Clostridium repeat (e.g., comprising about 24 to 25 or 25 to 26 or more contiguous nucleotides from the 5′ end of a wild type Clostridium CRISPR Type I-C locus repeat sequence; e.g., about 24, 25, 26, 27, 28, 29, 30, 31 or 32 contiguous nucleotides from the 5′ end, or any range or value therein or). In some embodiments, the portion of a repeat sequence that is reduced in length by about 7 to 8 contiguous nucleotides may be linked to the 3′ end of a spacer sequence. In some embodiments, a portion of a repeat sequence may comprise a length of about 7 to 8 nucleotides (e.g., 1, 2, 3, 4, 5, 6, 7, or 8 nucleotides), wherein the about 7 to 8 nucleotides are from the 3′ end of a wild type Clostridium repeat (e.g., the last 7-8 contiguous nucleotides from the 3′ end of a wild type Clostridium CRISPR Type I-C locus repeat sequence). In some embodiments, the portion of a repeat sequence having a length of about 7 to 8 contiguous nucleotides from a wild type Clostridium repeat sequence may be linked to the 5′ end of a spacer sequence.

In some embodiments, a repeat sequence or portion thereof useful with this invention may comprise, consist essentially of or consist of at least 24 consecutive nucleotides (e.g., about 24, 25, 26, 27, 28, 29, 30, 31, 32 or 33 consecutive nucleotides) having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the nucleotide sequences of SEQ ID NOs:15-19, any one of the nucleotide sequences of SEQ ID NOs: 34-35, any one of the nucleotide sequences of SEQ ID NOs:50-53, any one of the nucleotide sequences of SEQ ID NOs: 68-71, any one of the nucleotide sequences of SEQ ID NOs:86-88, any one of the nucleotide sequences of SEQ ID NOs: 103-105, or any one of the nucleotide sequences of SEQ ID NOs: 120-121, optionally about 24, 25, 26, 27, 28 to about 29, 30, 31 or 32 consecutive nucleotides, about 25, 26, 27, 28 to about 29, 30, 31, 32 or 33, or about 30 to 33 consecutive nucleotides of the repeat sequences. In some embodiments, a repeat sequence or portion thereof useful with this invention may comprise, consist essentially of or consist of about 7 or 8 consecutive nucleotides having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the nucleotide sequences of SEQ ID NOs:15-19, any one of the nucleotide sequences of SEQ ID NOs: 34-35, any one of the nucleotide sequences of SEQ ID NOs:50-53, any one of the nucleotide sequences of SEQ ID NOs: 68-71, any one of the nucleotide sequences of SEQ ID NOs:86-88, any one of the nucleotide sequences of SEQ ID NOs: 103-105, or any one of the nucleotide sequences of SEQ ID NOs: 120-121.

Thus, in some embodiments, a Type I-C CRISPR repeat sequence useful with this invention may comprise, consist essentially of, or consist of any of the nucleotide sequences of (or a portion thereof)

SEQ ID NO: 15
GTCGTTCCCTGCAATGGGAACGTGGATTGAAAT
SEQ ID NO: 16
GCGTTGTTCCCATGCGGGAACTTGGATTGAAAT
SEQ ID NO: 17
GTCTCTCCCTGTATAGGGAGAGTGGATTGAAAT
SEQ ID NO: 18
GTCTTTCCCTGCATAGGGAGAGTGGATTGAAAT
SEQ ID NO: 19
GTCTCCACCTGTGTGGTGGAGTGGATTGAAAG
SEQ ID NO: 34
GTCTCCACCCTCGTGGTGGAGTGGATTGAAAT
SEQ ID NO: 35
GTCGAGGCCCGCGAGGGCCTTGTGGATTGAAAT
SEQ ID NO: 50
GTCTCCGTCCTCGCGGGCGGAGTGGGTTGAAAT
SEQ ID NO: 51
GTCTCCGTCCTCGCGGGGGGAGTGGCTTTTCCT
SEQ ID NO: 52
GTCGAGGCTCGCGAGAGCCTTGTGGATTGAAAT
SEQ ID NO: 53
GTCGAGGCTCGCGAGAGCCTTGCAGACCAAAAG
SEQ ID NO: 68
GTCGAGGCTCGCGAGAGCCTTGTGGATTGAAAT
SEQ ID NO: 69
GTCGAGGCTCGCGAGAGCCTTGCAGACCAAAAG
SEQ ID NO: 70
GTCTCCGTCCTCGCGGGCGGAGTGGGTTGAAAT
SEQ ID NO: 71
GTCTCCGTCCTCGCGGGCGGAGTGGCTTTTCCT
SEQ ID NO: 86
GTCGAGGCCCGCGAGGGCCTTGTGGATTGAAAT
SEQ ID NO: 87
GTCTCCACCCTCGCGGTGGAGTGGATTGAAAT
SEQ ID NO: 88
ATCTCCACCCTCGCGGTGGAGTGGATTGAAAT
SEQ ID NO: 103
GTCTCCACCCTCGCGGTGGAGTGGATTGAAAT
SEQ ID NO: 104
GTCGAGGCCCGCGAGGGCCTTGTGGATTGAAAT
SEQ ID NO: 105
GTCGAGGCCCGCGAGAGCCTTGTGGATTGAAAT
SEQ ID NO: 120
GTCTCCACCCTCGTGGTGGAGTGGATTGAAAT
SEQ ID NO: 121
GTCGAGGCCCGCGAGGGCCTTGTGGATTGAAAT

The bolded nucleotides indicate the nucleotides that differ from the consensus sequence.

In some embodiments, Type I-C CRISPR one or more repeat sequences useful with this invention may comprise, consist essentially of, or consist of a portion of contiguous nucleotides as described herein of any of the nucleotide sequences of SEQ ID NOs:15-19, 34, 35, 50-53, 68-71, 86-88, 103-105, 120, or 121, or any combination thereof.

In some embodiments, a Type I-B CRISPR repeat sequence (i.e., CRISPR repeat sequence) as used herein may comprise any known repeat sequence of a wild-type Erysipelatoclostridium CRISPR Type I-B locus (e.g., E. ramosum). In some embodiments, a repeat sequence useful with the invention may include a synthetic repeat sequence having a different nucleotide sequence than those known in the art for Erysipelatoclostridium but sharing similar structure to that of wild-type Erysipelatoclostridium repeat sequences of a hairpin structure with a loop region. Thus, in some embodiments, a repeat sequence may be identical to (i.e., having 100% identity) or substantially identical (e.g., having 80% to 99% identity (e.g., about 80, 81, 82, 83, 84, 85, 86, 87, 88, 89, 90, 91, 92, 93, 94, 95, 96, 97, 98, 99% identity)) to a repeat sequence from a wild-type Erysipelatoclostridium CRISPR Type I-B locus.

The length of a Type I-B CRISPR repeat sequence useful with this invention may be the full length of a Clostridium spp. or Erysipelatoclostridium spp. (e.g., E. ramosum or C. spp. 1141A1FAA) repeat sequence (i.e., about 25 to 32 nucleotides) (see, e.g., SEQ ID NOs:138, 139 or 156-164). In some embodiments, a repeat sequence may comprise a portion of a wild type Clostridium or Erysipelatoclostridium repeat nucleotide sequence, the portion being reduced in length by as much as 7 to 8 contiguous nucleotides (e.g., 1, 2, 3, 4, 5, 6, 7, or 8 nucleotides) from the 3′ end as compared to a wild type Clostridium or Erysipelatoclostridium repeat (e.g., comprising about 19 to 31 contiguous nucleotides from the 5′ end of a wild type Clostridium or Erysipelatoclostridium CRISPR Type I-B locus repeat sequence; e.g., about 19, 20, 20, 21, 22, 23, 24, 25, 26, 27, 28, 29, 30 or 31 contiguous nucleotides from the 5′ end, or any range or value therein). In some embodiments, a repeat sequence useful with this invention may comprise, consist essentially of or consist of at least 19 consecutive nucleotides (e.g., about 19, 20, 21, 22, 23, 24, 25, 26, 27, 28, 29, 30, 31, 32 or 33 consecutive nucleotides) having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the nucleotide sequences of SEQ ID NOs:NOs:138, 139 or 156-164, optionally about 19, 20, 21, 22, 23, 24, or 25 consecutive nucleotides to about 26, 27, 28 29, 30, or 31 consecutive nucleotides, about 21, 22, 23, 24, or 25 consecutive nucleotides to about 26, 27, 28, 29, 30, 31, 32 or 33, or about 27 to about 32 consecutive nucleotides of the repeat sequences. In some embodiments, the portion of a repeat sequence that is reduced in length by about 7 to 8 contiguous nucleotides may be linked to the 3′ end of a spacer sequence. In some embodiments, a portion of a repeat sequence may comprise a length of about 7 to 8 nucleotides (e.g., 1, 2, 3, 4, 5, 6, 7, or 8 nucleotides), wherein the about 7 to 8 nucleotides are from the 3′ end of a wild type Clostridium repeat (e.g., the last 7-8 contiguous nucleotides from the 3′ end of a wild type Clostridium CRISPR Type I-C locus repeat sequence). In some embodiments, the portion of a repeat sequence having a length of about 7 to 8 contiguous nucleotides from a wild type Clostridium repeat sequence may be linked to the 5′ end of a spacer sequence.

Accordingly, in some embodiments, a Type I-C CRISPR repeat sequence may comprise, consist essentially of, or consist of any of the nucleotide sequences of: GTTTAATAACAACATAAGATGTATTGAAAT SEQ ID NO:138, GTTTAATAACAACGAGATATTAAAGTGAAA SEQ ID NO:139, GTTTCAATCCACGCACCCGTGCAGGGTGCGAC SEQ ID NO:156, GTTTCAATTCCAACATGGTACGATTAAAGC SEQ ID NO:157, GTTTCAATTCCAATATGGCACGATTAAAGC SEQ ID NO:158, TGCTTTATTACAATGTGGTAAGAGTAAAGC SEQ ID NO:159, CTTTCAATTCCAACTTGGTACGATTAAAAC SEQ ID NO:160, AACGCAATTCCAACTTGGTACGATTAAAAC SEQ ID NO:161, GTTTCAATTCCAACTTGGTACGATTAAAAC SEQ ID NO:162, GTTGCAATTCCAATATGGTAAGATTAAAGC SEQ ID NO:163, and/or TCAATTCCAATATGGTACGATTAAAAC SEQ ID NO:164, or any combination thereof.

The bolded nucleotides indicate the nucleotides that differ from the consensus sequence.

In some embodiments, Type I-B CRISPR one or more repeat sequences useful with this invention may comprise, consist essentially of, or consist of a portion of contiguous nucleotides as described herein of any of the nucleotide sequences of SEQ ID NOs:138, 139 or 156-164, or any combination thereof.

In some embodiments, when two or more repeat sequences are present in a CRISPR, they may comprise the same repeat sequence, may comprise different repeat sequences, or any combination thereof. In some embodiments, each of the two or more repeat sequences in a CRISPR may comprise, consist essentially of, or consist of the same repeat sequence.

A CRISPR useful with the methods of the invention may comprise one spacer sequence or more than one spacer sequence, wherein each spacer sequence is flanked by at least one repeat sequence (e.g., a repeat-spacer (non-natural) or a repeat-spacer-repeat), wherein the at least one repeat may be a full-length repeat sequence, or a portion thereof as described herein. In some embodiments, a CRISPR useful with this invention may comprise a spacer sequence linked at its 5′ end and its 3′ end to a repeat sequence (e.g., a repeat-spacer-repeat), wherein the repeat is a full-length repeat sequence or a portion thereof. When more than one spacer sequence is present in a CRISPR of the invention, each spacer sequence is separated from the next spacer sequence by a repeat sequence. Thus, each spacer sequence is linked at the 3′ end and at the 5′ end to a repeat sequence. The repeat sequence that is linked to each end of the one or more spacers may be the same repeat sequence or it may be a different repeat sequence or any combination thereof.

In some embodiments, the one or more spacer sequences of a Type I-C CRISPR of the present invention may be about 20 nucleotides to about 40 nucleotides in length (e.g., about 20, 21, 22, 23, 24, 25, 26, 27, 28, 29, 30, 31, 32, 33, 34, 35, 36, 37, 38, 39, or 40 nucleotides in length, and any value or range therein). In some embodiments, a spacer sequence may be a length of about 30 nucleotides to about 40 nucleotides (e.g., about 30, 31, 32, 33, 34, 35, 36, 37, 38, 39, or 40 nucleotides in length, and any value or range therein), or about 20, 22, 31, 33, 34, or 38 nucleotides in length. In some embodiments, a spacer sequence may comprise, consist essentially of, or consist of a length of about 34 nucleotides in length.

In some embodiments, the one or more spacer sequences of a Type I-B CRISPR of the present invention may be about 25 nucleotides to about 60 nucleotides in length (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length, and any value or range therein). In some embodiments, a spacer sequence may be a length of about 25 nucleotides to about 45 nucleotides (e.g., about 25, 26, 27, 28, 29, 30, 31, 32, 33, 34, 35, 36, 37, 38, 39, 40, 41, 42, 43, 44, or 45 nucleotides in length, and any value or range therein), about 30 nucleotides to about 40 nucleotides (e.g., about 30, 31, 32, 33, 34, 35, 36, 37, 38, 39, or 40 nucleotides in length, and any value or range therein) or about 32 nucleotides to about 40 nucleotides (e.g., about 30, 31, 32, 33, 34, 35, 36, 37, 38, 39, or 40 nucleotides in length, and any value or range therein). In some embodiments, a spacer sequence may comprise, consist essentially of, or consist of a length of about 35, 36 or 37 nucleotides in length. In some embodiments, a spacer sequence may comprise, consist essentially of, or consist of a length of about 35 nucleotides in length. In some embodiments, a spacer sequence may comprise, consist essentially of, or consist of a length of about 37 nucleotides in length.

In some embodiments, a spacer sequence useful with the methods of this invention may be fully complementary to a target sequence (e.g., 100% complementary to a target sequence across its full length). In some embodiments, a spacer sequence may be substantially complementary (e.g., at least about 80% complementary (e.g., about 80%, 81%, 82%, 83%, 84%, 85%, 86%, 87%, 88%, 89%, 90%, 91%, 92%, 93%, 94%, 95%, 96%, 97%, 98%, 98.5%, 99%, 99.5%, or more complementary)) to a target sequence from a target genome. Thus, in some embodiments, a spacer sequence may have one, two, three, four, five or more mismatches that may be contiguous or noncontiguous as compared to a target sequence from a target genome. In some embodiments, a spacer sequence may be about 80% to 100% (e.g., about 80%, 81%, 82%, 83%, 84%, 85%, 86%, 87%, 88%, 89%, 90%, 91%, 92%, 93%, 94%, 95%, 96%, 97%, 98%, 99%, or 100)) complementary to a target sequence from a target genome. In some embodiments, a spacer sequence may be about 85% to 100% (e.g., about 85%, 86%, 87%, 88%, 89%, 90%, 91%, 92%, 93%, 94%, 95%, 96%, 97%, 98%, 99%, or 100%)) complementary to a target sequence from a target genome. In some embodiments, a spacer sequence may be about 90% to 100% (e.g., about 90%, 91%, 92%, 93%, 94%, 95%, 96%, 97%, 98%, 99%, or 100%)) or about 95% to 100% (e.g., about 95%, 96%, 97%, 98%, 98.5%, 99%, 99.5% or 100%) complementary to a target sequence from a target genome.

In some embodiments, the 5′ region of a spacer sequence may be fully complementary to a target sequence while the 3′ region of the spacer sequence may be substantially complementary to the target sequence. Accordingly, in some embodiments, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) may be about 100% complementary to a target sequence, while the remainder of the spacer sequence may be about 80% or more complementary to the target sequence.

In some embodiments, at least the first eight contiguous nucleotides at the 5′ end of a spacer sequence of the invention are fully complementary to the portion of the target sequence adjacent to the PAM (termed a “seed sequence”). Thus, in some embodiments, the seed sequence may comprise the first 6-8 nucleotides (e.g., 6, 7, 8) of the 5′ end of each of one or more spacer sequence(s), which first 6-8 nucleotides are fully complementary (100%) to the target sequence, and the remaining portion of the one or more spacer sequence(s) (3′ to the seed sequence) may be at least about 80% complementarity (e.g., about 80, 81, 82, 83, 84, 85, 86, 87, 88, 89, 90, 91, 92, 93, 94, 95, 96, 97, 98, 99, or 100% complementarity) to the target sequence.

Thus, for example, a spacer sequence having a length of 20 nucleotides may comprise a seed sequence of eight contiguous nucleotides located at the 5′ end of the spacer sequence, which is 100% complementary to the target sequence, while the remaining 12 nucleotides may be about 80% to about 100% complimentary to the target sequence (e.g., 0 to 2 non-complementary nucleotides out of the remaining 12 nucleotides in the spacer sequence). As another example, a spacer sequence having a length of 33 nucleotides may comprise a seed sequence of six nucleotides from the 5′ end, which is 100% complementary to the target sequence, while the remaining 27 nucleotides may be at least about 80% (e.g., 0 to 5 non-complementary nucleotides out of the remaining 27 nucleotides in the spacer sequence) or a spacer sequence having a length of 32 nucleotides may comprise a seed sequence of eight nucleotides from the 5′ end, which is 100% complementary to the target sequence, while the remaining 24 nucleotides may be at least about 80% (e.g., 0 to 4 non-complementary nucleotides out of the remaining 24 nucleotides in the spacer sequence).

In an additional example, a spacer sequence having a length of 28 nucleotides may comprise a seed sequence of eight contiguous nucleotides located at the 5′ end of the spacer sequence, which is 100% complementary to the target sequence, while the remaining 20 nucleotides may be about 80% to about 100% complimentary to the target sequence (e.g., 0 to 4 non-complementary nucleotides out of the remaining 20 in the spacer sequence or 0 to 2 non-complementary nucleotides out of the remaining 12 nucleotides in the spacer sequence). As another example, a spacer sequence having a length of 33 nucleotides may comprise a seed sequence of eight nucleotides from the 5′ end, which is 100% complementary to the target sequence, while the remaining 25 nucleotides may be at least about 80% (e.g., 0 to 5 non-complementary nucleotides out of the remaining 25 nucleotides in the spacer sequence) or a spacer sequence having a length of 37 nucleotides may comprise a seed sequence of eight nucleotides from the 5′ end, which is 100% complementary to the target sequence, while the remaining 29 nucleotides may be at least about 80% (e.g., 0 to 5 non-complementary nucleotides out of the remaining 29 nucleotides in the spacer sequence). In a further example, a spacer sequence having a length of 34 nucleotides may comprise a seed sequence of eight nucleotides from the 5′ end, which is 100% complementary to the target sequence, while the remaining 26 nucleotides may be at least about 80% (e.g., 0 to 5 non-complementary nucleotides out of the remaining 26 nucleotides in the spacer sequence) or a spacer sequence having a length of 38 nucleotides may comprise a seed sequence of eight nucleotides from the 5′ end, which is 100% complementary to the target sequence, while the remaining 30 nucleotides may be at least about 80% (e.g., 0 to 6 non-complementary nucleotides out of the remaining 30 nucleotides in the spacer sequence).

A CRISPR of the invention comprising more than one spacer sequence may target one or more than one target sequence (protospacer) of a bacteriophage. Thus, in some embodiments, when a recombinant nucleic acid construct of the invention comprises a CRISPR that comprises at least two spacer sequences, the at least two spacer sequences may be complementary to two or more different target sequences in a bacteriophage or in two or more bacteriophage species or strains. In some embodiments, when a recombinant nucleic acid construct of the invention comprises a CRISPR that comprises at least two spacer sequences, the at least two spacer sequences may be complementary to the same target sequence. In some embodiments, a CRISPR comprising at least two spacer sequences, the at least two spacer sequences may be complementary to different portions of one gene or the at least two spacer sequences may target entirely different genes or any combination thereof.

In some embodiments, more than one CRISPR may be introduced into a bacterial cell using various combinations of the constructs as described herein. In some embodiments, a recombinant nucleic acid construct comprising one CRISPR or a recombinant nucleic acid construct comprising more than one CRISPR may be introduced into a bacterial cell. In some embodiments, more than one recombinant nucleic acid construct each comprising one CRISPR or more than one CRISPR may be introduced into a bacterial cell.

In some embodiments, a recombinant nucleic acid construct comprising a CRISPR, a recombinant nucleic acid construct encoding a Cas3 and a recombinant nucleic acid construct encoding a Cascade complex may be introduced into a bacterium simultaneously, separately and/or sequentially. Thus, in some embodiments, a recombinant nucleic acid construct comprising a CRISPR and/or the recombinant nucleic acid construct encoding a Cas3 and/or the recombinant nucleic acid construct encoding a Cascade complex may be comprised in a single vector and/or expression cassette or may be comprised in two or three separate vectors and/or expression cassettes, optionally wherein the vector may be, for example, a recombinant plasmid, bacteriophage, transposon, or phagemid. In some embodiments, the recombinant nucleic acid construct comprising Cas3 and the recombinant nucleic acid construct encoding a Cascade complex are comprised in the same vector and therefore, introduced together. In some embodiments, the recombinant nucleic acid construct comprising a CRISPR and the recombinant nucleic acid construct encoding Cas3 and a Cascade complex are comprised in the same vector and therefore, introduced together.

When introduced into a cell of a bacterial cell, a recombinant nucleic acid construct comprising a CRISPR, a recombinant nucleic acid construct encoding a Cas3, and a recombinant nucleic acid construct encoding a Cascade complex may be introduced into the bacterial cell simultaneously, separately and/or sequentially, in any order. In some embodiments, a recombinant nucleic acid construct comprising a CRISPR, a recombinant nucleic acid construct encoding a Cas3, and a recombinant nucleic acid construct encoding a Cascade complex may be introduced simultaneously on the same or on separate expression cassettes and/or vectors. In some embodiments, the recombinant nucleic acid construct comprising a CRISPR and the recombinant nucleic acid construct encoding a Cascade complex are introduced simultaneously on a single expression cassette and/or vector and the Cas3 is introduced simultaneously on a separate or in the same expression cassette. In some embodiments, when co-opting an endogenous CRISPR-Cas Type I-C system or an endogenous CRISPR-Cas Type I-B system of a bacterium only a recombinant nucleic acid construct comprising a CRISPR of the invention is introduced.

In some embodiments, when a recombinant nucleic acid construct comprising a CRISPR and a recombinant nucleic acid construct encoding a Cascade complex polypeptide and a Cas3 polypeptide are introduced into a cell, they may be comprised in a single expression cassette and/or vector in any order. In some embodiments, when a recombinant nucleic acid construct comprising a CRISPR and a recombinant nucleic acid construct encoding a Cascade complex are introduced into a cell, they may be comprised in two or three separate vectors and/or expression cassettes in any order. When more than one expression cassette and/or vector is used to introduce the constructs of the invention, each may encode different selection agents/markers (e.g., may encode nucleic acids conferring resistance to different antibiotics) so that the transformed cell maintains each expression cassette/vector that is introduced.

Non-limiting examples of vectors useful with this invention include plasmids, bacteriophage, transposons, or phagemids.

TABLE 1
Combinations of Type I-C Cascade polypeptides and nucleotide sequences, repeat
sequences and PAM sequences of the invention.

| | | | SEQ ID NOs |
|---|---|---|---|
| | SEQ ID NOS | SEQ ID NOS | PAM |
| Organism | Cascade proteins/polynucleotides | Repeat sequences | sequences |
| C. scindens | SEQ ID NO: 1/SEQ ID NO: 8 (Cas3); | SEQ ID NOs: 15, | 5′-TTT-3′ |
| ATCC35704 | SEQ ID NO: 2/SEQ ID NO: 9 (Cas5), | 16, 17, 18, 19 | |
| | SEQ ID NO: 3/SEQ ID NO: 10 (Cas8), | | |
| | SEQ ID NO: 4/SEQ ID NO: 11 (cas7); | | |
| C. clostridioforme | SEQ ID NO: 20/SEQ ID NO: 27 (Cas3); | SEQ ID NO: 34, | 5′-TTC-3′ |
| WAL7855 | SEQ ID NO: 21/SEQ ID NO: 28 (Cas5), | SEQ ID NO: 35, | |
| | SEQ ID NO: 22/SEQ ID NO: 29 (Cas8), | optionally 86, 87, | |
| | SEQ ID NO: 23/SEQ ID NO: 30 (cas7), | 88, 103, 104, 105, | |
| | | 120, 121 | |
| C. bolteae | SEQ ID NO: 36/SEQ ID NO: 43 (Cas3), | SEQ ID NOs: 50, | 5′-TTC-3′ |
| DSM15670 | SEQ ID NO: 37/SEQ ID NO: 44 (Cas5), | 51, 52, 53, | 5′-CTC-3′ |
| | SEQ ID NO: 38/SEQ ID NO: 45 (Cas8), | optionally SEQ ID | |
| | SEQ ID NO: 39/SEQ ID NO: 46 (cas7) | NOs: 68, 69, 70, 71 | |
| C. bolteae | SEQ ID NO: 54/SEQ ID NO: 61 (Cas3), | SEQ ID NOs: 68, | 5′-TTC-3′ |
| WAL14578 | SEQ ID NO: 55/SEQ ID NO: 62 (Cas5), | 69, 70, 71, | 5′-CTC-3′ |
| | SEQ ID NO: 56/SEQ ID NO: 63 (Cas8), | optionally SEQ ID | |
| | SEQ ID NO: 57/SEQ ID NO: 64 (cas7) | NOs: 50, 51, 52, 53 | |
| C. clostridioforme | SEQ ID NO: 72/SEQ ID NO: 79 (Cas3), | SEQ ID NOS: 86, | 5′-TTC-3′ |
| NCTC11224 | SEQ ID NO: 73/SEQ ID NO: 80 (Cas5), | 87, 88, optionally | |
| | SEQ ID NO: 74/SEQ ID NO: 81 (Cas8), | 34, 35, 103, 104, | |
| | SEQ ID NO: 75/SEQ ID NO: 82 (cas7) | 105, 120, 121 | |
| C. clostridioforme | SEQ ID NO: 89/SEQ ID NO: 96 (Cas3) | SEQ ID NOs: 103, | 5′-TTC-3′ |
| YL32 | SEQ ID NO: 90/SEQ ID NO: 97 (Cas5), | 104, 105, optionally | |
| | SEQ ID NO: 91/SEQ ID NO: 98 (Cas8), | 34, 35, 86, 87, 88, | |
| | SEQ ID NO: 92/SEQ ID NO: 99 (cas7) | 120, 121 | |
| C. clostridioforme | SEQ ID NO: 106/SEQ ID NO: 113 (Cas3), | SEQ ID NO: 120, | 5′-TTC-3′ |
| 2149FAA | SEQ ID NO: 107/SEQ ID NO: 114 (Cas5), | SEQ ID NO: 121, | |
| | SEQ ID NO: 108/SEQ ID NO: 115 (Cas8), | optionally 34, 35, | |
| | SEQ ID NO: 109/SEQ ID NO: 116 (cas7) | 86, 87, 88, 103, | |
| | | 104 | |

TABLE 2
Example combinations of Type I-B Cascade polypeptides and nucleotide
sequences, repeat sequences and PAM sequences of the invention.

| | Cascade proteins/polynucleotides | Repeat | |
|---|---|---|---|
| Organism | SEQ ID NOS | SEQ ID NOS | PAM |
| E. ramosum | SEQ ID NO: 122/SEQ ID NO130 (Cas6) | SEQ ID | 5′-ATC-3′ |
| DSM1402 | SEQ ID NO: 123/SEQ ID NO: 131 (Cas8) | NO: 138 | 5′-ATCN*-3′ |
| | SEQ ID NO: 1243/SEQ ID NO: 132 (Cas7) | SEQ ID | 5′-ATCA-3′, |
| | SEQ ID NO: 125/SEQ ID NO: 133 (cas5) | NO: 139 | 5′-ATCG-3′ |
| | SEQ ID NO: 126/SEQ ID NO: 134 (Cas3) | | |
| Clostridium | SEQ ID NO: 140/SEQ ID NO: 148 (Cas6) | SEQ ID | 5′-TTTA-3′ |
| spp. | SEQ ID NO: 141/SEQ ID NO: 149 (Cas8) | NOs: 156, 157, | |
| 1141A1FAA | SEQ ID NO: 142/SEQ ID NO: 150 (Cas7) | 158, 159, 160, | |
| | SEQ ID NO: 143/SEQ ID NO: 151 (Cas5) | 161, 162, 163, | |
| | SEQ ID NO: 144/SEQ ID NO: 152 (Cas3) | 164 | |

*N = A, T, C, or G

As described herein, the constructs of the invention may optionally comprise regulatory elements, including, but not limited to, promoters and terminators. Promoters useful with the methods of the invention are as described herein, and include, but are not limited to the nucleotide sequences of SEQ ID NOs:165-176 (Type I-C) or SEQ ID NOs:177-185 (Type I-B), and any combination thereof. In some embodiments, when more than one construct is introduced, promoters useful with the constructs may be any combination of heterologous and/or endogenous promoters.

Thus, in some embodiments, a recombinant nucleic acid construct comprising a CRISPR, a recombinant nucleic acid construct encoding a Cascade complex and a recombinant nucleic acid construct encoding a Cas3 polypeptide may be operably linked to a single promoter, in any order or in any combination thereof, or they may each be operably linked to independent (e.g., separate) promoters. In some embodiments, when a recombinant nucleic acid construct comprising a CRISPR, a recombinant nucleic acid construct encoding a Cascade complex and a recombinant nucleic acid construct encoding a Cas3 polypeptide are present in the same expression cassette and/or vector, they may be operably linked to the same promoter. In some embodiments, when a recombinant nucleic acid construct comprising a CRISPR, a recombinant nucleic acid construct encoding a Cascade complex and a recombinant nucleic acid construct encoding a Cas3 polypeptide are present in the same expression cassette or vector, the recombinant nucleic acid construct encoding a Cascade complex, the recombinant nucleic acid construct encoding a Cas3 polypeptide, and the recombinant nucleic acid construct encoding a CRISPR may be operably linked to separate promoters that may be the same or different. In some embodiments, the Cascade complex and the Cas3 polypeptide are encoded on the same recombinant nucleic acid construct and the CRISPR is comprised in the same or a separate recombinant nucleic acid construct. Promoters useful with the methods of the invention are as described herein, and include, but are not limited to the nucleotide sequences of SEQ ID NOs:165-176 (Type I-C) or SEQ ID NOs:177-185 (Type I-B), in any combination.

In some embodiments, a recombinant nucleic acid construct comprising a CRISPR may be operably linked to a terminator and a recombinant nucleic acid construct encoding a Cascade complex and/or a Cas3 polypeptide may be optionally operably linked to a terminator. In some embodiments, a recombinant nucleic acid construct comprising a CRISPR, a recombinant nucleic acid construct encoding a Cascade complex and/or a Cas3 polypeptide may each be operably linked to a single terminator, in any order or in any combination thereof, or they may each be operably linked to independent (e.g., separate) terminators. In some embodiments, when a recombinant nucleic acid construct comprising a CRISPR and a recombinant nucleic acid construct encoding a Cascade complex and/or a Cas3 polypeptide are present in the same expression cassette or vector, they may be operably linked to the same terminator. In some embodiments, when a recombinant nucleic acid construct comprising a CRISPR and a recombinant nucleic acid construct encoding a Cascade complex and/or a Cas3 polypeptide are present in the same expression cassette and/or vector, only the recombinant nucleic acid construct encoding a CRISPR is operably linked to a terminator sequence. Terminator sequences useful with the methods of the invention are as described herein. In some embodiments, a terminator sequence useful with the invention may include, but is not limited to, the nucleotide sequence of any one of SEQ ID NOs:186-194, and/or any combination thereof.

Notably, the recombinant nucleic acid constructs, protein-RNA complexes, and their methods of use as described herein are advantageous over other known CRISPR systems in that their activity (as measured by repression reaching up to 98%) is quite high. In addition, the PAM (TTTA, ATC, or ATCN) which are quite distinct from and complementary to known systems that are GC rich (for example, the TTTA PAM enables targeting of AAAT complementary sequences on the other strand, with noteworthy AT bias highly distinct from and complementary to GC-rich PAMs previously reported). Another advantage is the long spacer (e.g., about 34-37 nt) which provides expanded opportunities for specificity. The present invention further provides sequence and structural diversity from other known Type I systems (see, e.g., the widely used E. coli system), with different CRISPR repeat sequences and longer 5′ handle and 3′ hairpins, which provides opportunities for concurrent use of two (or more) orthogonal systems that provide multiplexed opportunities to perform multiple reactions that are different all at the same time (e.g., up-regulation, and down-regulation and/or genome editing).

“Introducing,” “introduce,” “introduced” (and grammatical variations thereof) in the context of a polynucleotide of interest and a cell of an organism means presenting the polynucleotide of interest to the host organism or cell of said organism (e.g., host cell) in such a manner that the nucleotide sequence gains access to the interior of a cell and includes such terms as transformation,” “transfection,” and/or “transduction.” Transformation may be electrical (electroporation and electrotransformation), or chemical (with a chemical compound, and/or though modification of the pH and/or temperature in the growth environment. Where more than one nucleotide sequence is to be introduced these nucleotide sequences can be assembled as part of a single polynucleotide or nucleic acid construct, or as separate polynucleotide or nucleic acid constructs, and can be located on the same or different expression constructs or transformation vectors. Accordingly, these polynucleotides can be introduced into cells in a single transformation event, in separate transformation events, or, for example, they can be incorporated into an organism by conventional breeding or growth protocols. Thus, in some aspects of the present invention one or more recombinant nucleic acid constructs of this invention may be introduced into a host bacterium.

“Introducing,” “introduce,” “introduced” (and grammatical variations thereof) in the context of a protein-RNA complex of the invention and a cell of an organism means presenting the polynucleotide of interest to the host organism or cell of said organism and includes such terms as transformation,” “transfection,” and/or “transduction.” Thus, in some embodiments, the terms “transformation,” “transfection,” and “transduction” as used herein may also refer to the introduction of a protein-RNA complex of the invention into a cell.

The terms “transformation,” “transfection,” and “transduction” as used herein refer to the introduction of a heterologous nucleic acid into a cell. Such introduction into a cell may be stable or transient. Thus, in some embodiments, a host cell or host organism is stably transformed with a nucleic acid construct of the invention. In other embodiments, a host cell or host organism is transiently transformed with a recombinant nucleic acid construct of the invention.

As used herein, the term “stably introduced” means that an introduced polynucleotide is stably incorporated into the genome of the cell, and thus the cell is stably transformed with the polynucleotide. When a nucleic acid construct is stably transformed and therefore integrated into a cell, the integrated nucleic acid construct is capable of being inherited by the progeny thereof, more particularly, by the progeny of multiple successive generations. In some embodiments, the term “stably introduced” means that an introduced protein-RNA complex of the invention is stably maintained in the cell into which it is introduced.

“Transient transformation” in the context of a polynucleotide or a protein-RNA complex means that a polynucleotide or the protein-RNA complex is introduced into the cell and does not integrate into the genome of the cell or is not otherwise maintained by the cell.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant, a mammal, an insect, an archaea, a bacterium, and the like). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, the nucleotide sequences, constructs, expression cassettes may be expressed transiently and/or they may be stably incorporated into the genome of the host organism. In some embodiments, when transient transformation is desired, the loss of the plasmids and the recombinant nucleic acids comprised therein may achieved by removal of selective pressure for plasmid maintenance. In some embodiments, a recombinant nucleic acid construct is maintained in a bacterial cell as an extrachromosomal element (e.g., a plasmid). In some embodiments, a recombinant nucleic acid construct is incorporated into the chromosome of the bacterial cell. When incorporated into the genome or into an extrachromosomal element a recombinant/non-natural CRISPR may be incorporated into an existing endogenous CRISPR array (e.g., in a repeat-spacer format) or may be incorporated as a newly independent non-endogenous CRISPR in the chromosome (e.g., in a repeat-spacer-repeat format), wherein the spacer is expressed from (the chromosome or the extrachromosomal element), thereby conferring resistance to the one or more bacteriophage species or strains comprising in its genome the nucleic acid sequence to which the spacer sequence is complementary. In some embodiments, when a non-natural CRISPR of the invention is incorporated into an endogenous array of the host bacterium the non-natural CRISPR is incorporated into the endogenous array in the 5′ portion of endogenous CRISPR array, the near the 5′ end of the endogenous CRISPR array and/or adjacent to and 5′ of the 5′ end of the first repeat of the endogenous CRISPR array. In some embodiments, when a non-natural CRISPR of the invention is incorporated into an endogenous CRISPR array of the host bacterium and the 5′ end of the endogenous CRISPR array is operably linked to a leader sequence, the non-natural CRISPR is incorporated into the endogenous CRISPR array at a site located between the leader sequence and the first repeat at the 5′ end of the endogenous CRISPR array (e.g., adjacent to the 3′-end of the leader sequence and to the 5′ of the endogenous CRISPR array).

A recombinant nucleic acid construct of the invention or a protein-RNA complex of the invention may be introduced into a cell by any method known to those of skill in the art. Exemplary methods of transformation or transfection include biological methods using viruses and bacteria (e.g., Agrobacterium), physicochemical methods such as electroporation, floral dip methods, particle or ballistic bombardment, microinjection, whiskers technology, pollen tube transformation, calcium-phosphate-mediated transformation, nanoparticle-mediated transformation, polymer-mediated transformation including cyclodextrin-mediated and polyethylene glycol-mediated transformation, sonication, infiltration, as well as any other electrical, chemical, physical (mechanical) and/or biological mechanism that results in the introduction of nucleic acid into a cell, including any combination thereof.

In some embodiments of the invention, transformation of a cell comprises nuclear transformation. In other embodiments, transformation of a cell comprises plastid transformation (e.g., chloroplast transformation). In still further embodiments, the recombinant nucleic acid construct of the invention can be introduced into a cell via conventional breeding techniques.

Procedures for transforming prokaryotic organisms are well known and routine in the art and are described throughout the literature (See, for example, Jiang et al. 2013. Nat. Biotechnol. 31:233-239; Ran et al. Nature Protocols 8:2281-2308 (2013)) A nucleotide sequence therefore can be introduced into a host organism or its cell in any number of ways that are well known in the art. The methods of the invention do not depend on a particular method for introducing one or more nucleotide sequences into the organism, only that they gain access to the interior of at least one cell of the organism. Where more than one polynucleotide is to be introduced, they can be assembled as part of a single nucleic acid construct, or as separate nucleic acid constructs, and can be located on the same or different nucleic acid constructs. Accordingly, the polynucleotides can be introduced into the cell of interest in a single transformation event, or in separate transformation events.

Spacer sequences are used to guide the recombinant nucleic acid constructs of the invention or the co-opted endogenous CRISPR-Cas machinery of the target organism (e.g., Cascade complex) to the target sequences and are as described herein. Target sequences useful for enhancing the resistance of a bacterial cell or a bacterial population to one or more bacteriophage species or strains may be any nucleic acid sequence (e.g., genomic sequence (e.g., an essential, a non-essential, expendable, non-expendable genomic sequence)) that is located immediately adjacent to the 3′ end of a PAM sequence (e.g., 5′-TTT-3′, 5′-TTC-3′ and/or 5′-CTC-3′; or 5′-TTTA-3′, 5′-ATC-3′, and/or 5′-ATCN-3′ (N=T, A, C, or G, e.g., 5′-ATCA-3′, and/or 5′-ATCG-3′). In some embodiments, the target sequences may be conserved among the one or more bacteriophage or within a bacteriophage population. In some embodiments, the target sequence may be an essential and/or non-expendable genomic sequence that is located immediately adjacent (3′) to a PAM as defined herein (e.g., 5′-TTT-3′, 5′-TTC-3′ and/or 5′-CTC-3′; or 5′-TTTA-3′, 5′-ATC-3′, and/or 5′-ATCN-3′ (N=T, A, C, or G, e.g., 5′-ATCA-3′, and/or 5′-ATCG-3′)) and that is conserved among the one or more bacteriophage within a population of bacteriophage. In some embodiments of the invention, the PAM may comprise, consist essentially of, or consist of a sequence of 5′-TTT-3′, 5′-TTC-3′ and/or 5′-CTC-3′ or 5′-TTTA-3′, 5′-ATC-3′, and/or 5′-ATCN-3′ (e.g., 5′-ATCA-3′, and/or 5′-ATCG-3′) located immediately adjacent to and 5′ of the protospacer).

In some embodiments, targeting of a nucleic acid sequence in a bacteriophage may result in the death of the bacteriophage.

Accordingly, in some embodiments, a recombinant nucleic acid construct of the invention may target any region of the bacteriophage genome including, but not limited to, coding regions, non-coding regions, intragenic regions, and intergenic regions. In some embodiments, a recombinant nucleic acid construct of the invention may target, for example, a conserved coding region, a conserved non-coding region, a conserved intragenic region, and/or a conserved intergenic region. In some embodiments, a target sequence may target a gene encoding a tail protein, a portal protein, a capsid protein, a holin, a lysin, and/or a DNA packaging protein.

In some embodiments, a target sequence may be located in a gene, which can be in the upper (sense, coding) strand or in the bottom (antisense, non-coding) strand. In some embodiments, a target sequence may be located in an intragenic region of a gene, optionally located in the upper (sense, coding) strand or in the bottom (antisense, non-coding) strand. In some embodiments, a gene that is targeted by constructs of this invention may encode a promoter. In some embodiments, a target sequence may be located in an intergenic region, optionally in the upper (plus) strand or in the bottom (minus) strand.

In some embodiments, a target sequence may be a highly conserved gene, which may carry out essential biological functions and be part of the core genome (e.g., genes encoding tail, capsid, holin, terminase, portal proteins).

A target bacterium useful with his invention may be a probiotic bacterium. In some embodiments, the target bacterium may be any Clostridium spp. now known or later identified, optionally a commensal Clostridium spp. In some embodiments, a target bacterium may be a Clostridium spp., a Erysipelatoclostridium spp., a Lactococcus spp., a Streptococcus spp., a Klebsiella spp., a Propionibacterium spp., a Cutibacterium spp., a Lactobacillus spp., a Pseudomonas spp., a Faecalibacterium spp., a Akkermansia spp., a Bifidobacterium spp., a Roseburia spp., an E. coli spp., or a Clostridiodes spp. In some embodiments, the target bacterium may be Clostridium spp. 1141A1FAA. In some embodiments, the target bacterium may be Erysipelatoclostridium ramosum. In some embodiments, the target bacterium may be Clostridium bolteae (e.g., C. bolteae DSM15670 (BAA-613), C. bolteae WAL14578), Clostridium clostridioforme (e.g., C. clostridioforme WAL7855, C. clostridioforme 2149FAA, C. clostridioforme YL32, C. clostridioforme NCTC11224), Clostridium scindens (e.g., C. scindens ATCC 35704, C. scindens VE20205).

In some embodiments, the invention further comprises recombinant bacterial cells produced by the methods of the invention, comprising the recombinant nucleic acid constructs of the invention, and/or the recombinant plasmid and/or bacteriophage comprising the recombinant nucleic acid constructs of the invention, and/or the genome modifications and/or modifications in expression generated by the methods of the invention.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1. CRISPR-Cas System Identification and Characterization in Clostridium bolteae, Clostridium Clostridioforme, Clostridium Scindens

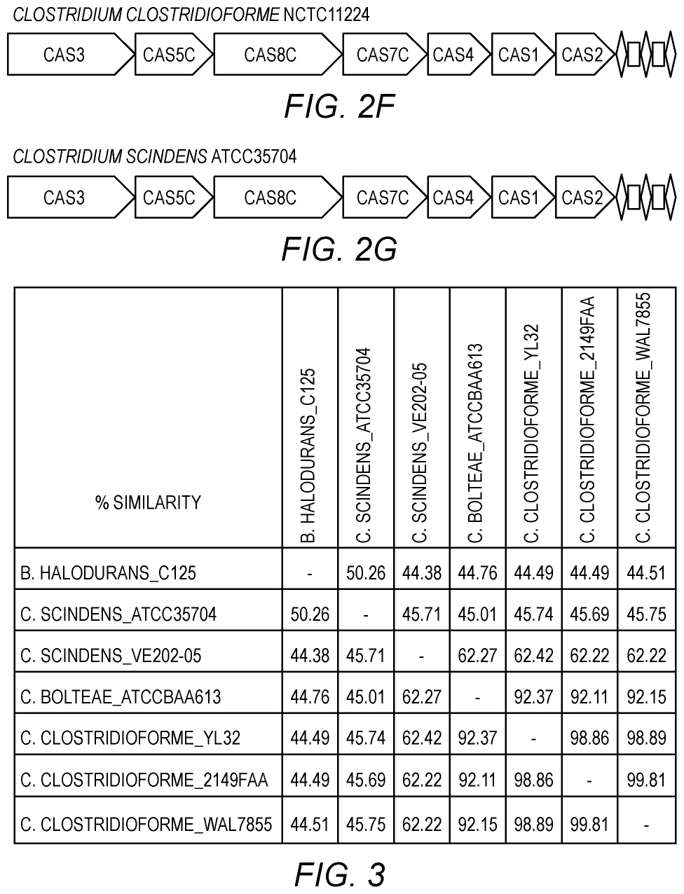

FIGS. 2A-2G show the results of the characterization of the Cascade complex for each of the Clostridium species analyzed. Each were shown to comprise a Cas3, Cas5, a Cas8, and a Cas7 in addition to the spacer acquisition polypeptides Cas4, Cas1 and Cas2 (Clostridium bolteae BAA-613 (Clostridium bolteae DSM15670 (BAA-613) (FIG. 2A), Clostridium bolteae WAL14578 (FIG. 2B), Clostridium clostridioforme WAL7855 (FIG. 2C), Clostridium clostridioforme 2149FAA (FIG. 2D), Clostridium clostridioforme YL32 (FIG. 2E), Clostridium clostridioforme NCTC11224 (FIG. 2F) and Clostridium scindens ATCC 35704 (FIG. 2G)).

The Type I-C Cascade polynucleotides of Clostridium scindens ATCC 35704, Clostridium scindens VE202-05, (Clostridium bolteae ATCCBAA613, Clostridium clostridioforme YL32, Clostridium clostridioforme 2149FAA and Clostridium clostridioforme WAL 7855 were compared to those from the canonical subtype I-C from Bacillus halodurans C-125. Both MUSCLE and ClustalW algorithm were used for the nucleotide sequence alignment. The results are provided in FIG. 3 and show that nucleotide sequence similarity of the Cas genes of the each of the Clostridium species analyzed is <50% compared to the canonical B. halodurans C125 CRISPR subtype I-C. This comparison further demonstrates the distinctiveness of these newly characterized CRISPR-Cas systems. The analysis also shows diversity of the Cascade polypeptides even within species. (see, e.g., Clostridium scindens ATCC 35704, and Clostridium scindens VE202-05).

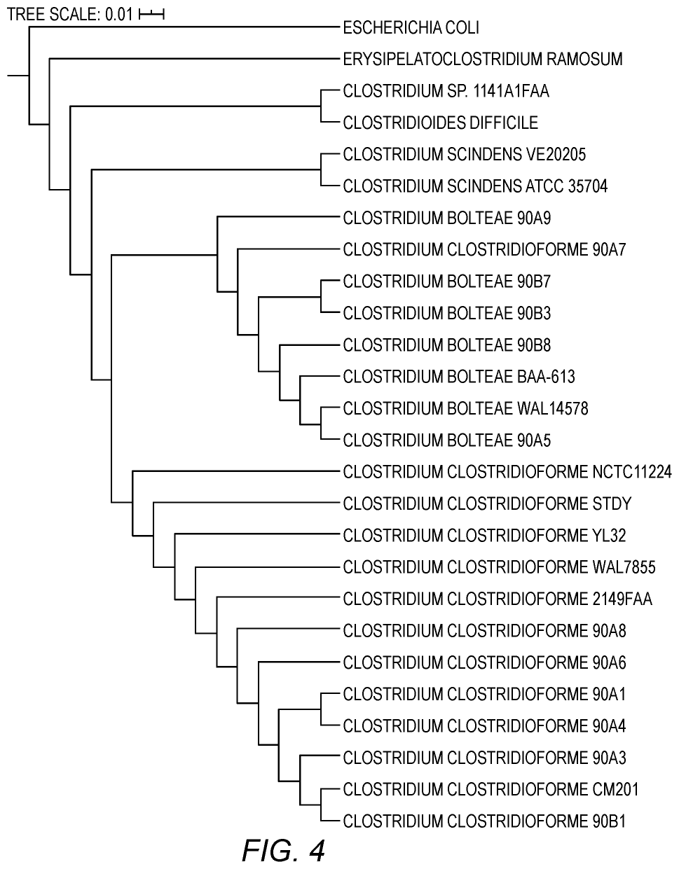

A phylogenomic analysis comparing the different Clostridium species of this invention and including E. coli, C. difficile and Erysipelatoclostridium ramosum is provided in FIG. 4 shows the phylogenetic distance among this species.

### Example 2. PAM Prediction for the Bacteria of Example 1

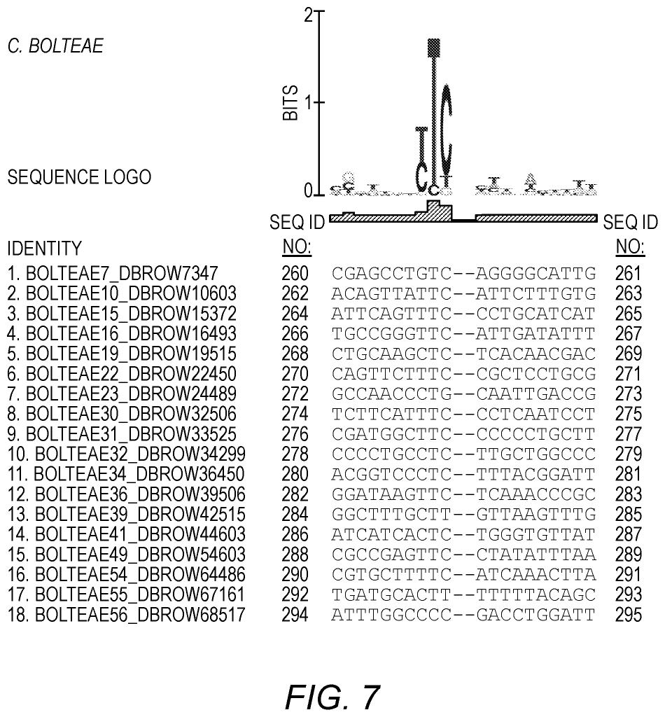

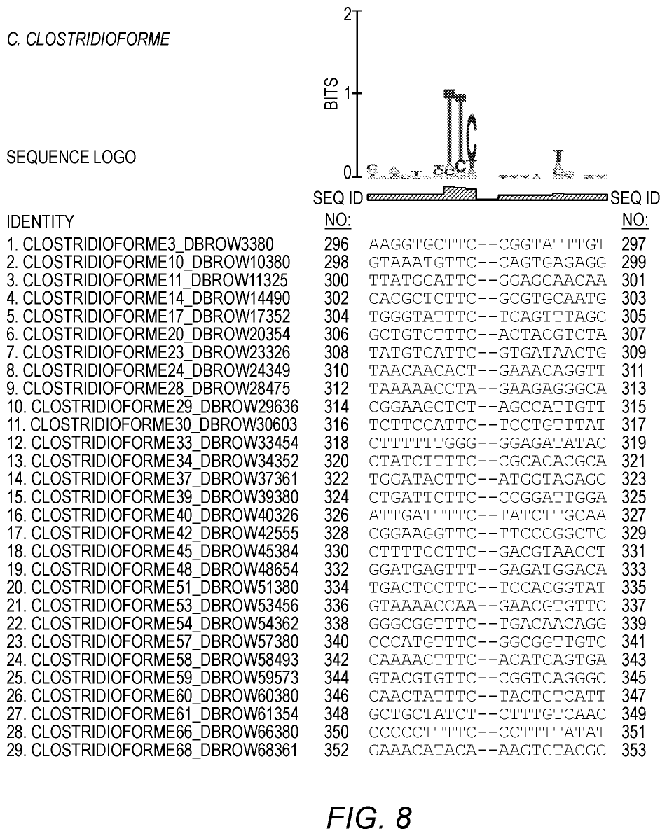

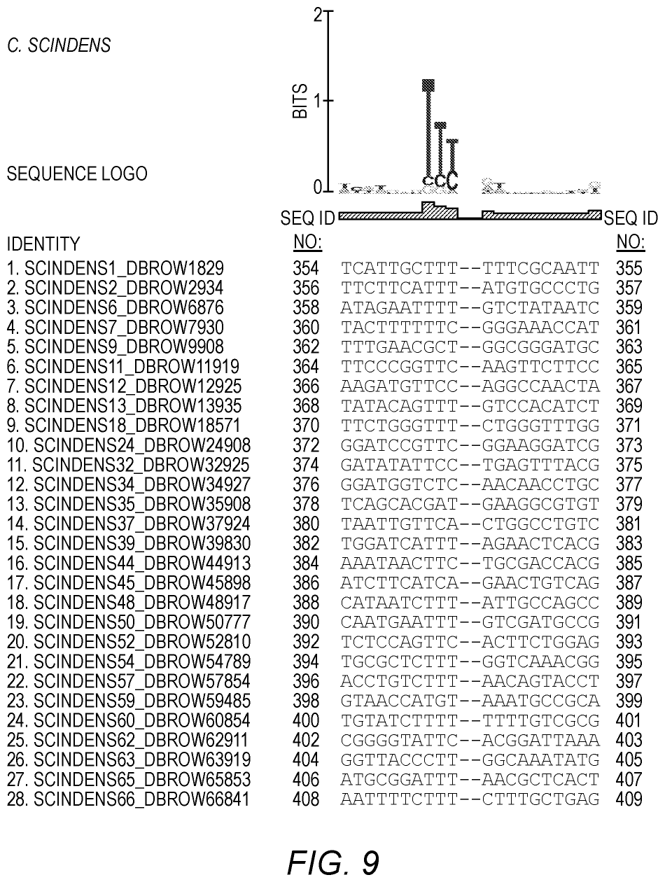

The CRISPR spacers were extracted from the CRISPR array of each of the strains described in Example 1, and a blastn was performed against different NCBI databases. The spacer-protospacer positive matches obtained were used to extract 10 nt of the adjacent (upstream and downstream) regions of the protospacer to elucidate the PAM sequence. The PAM sequences for the CRISPR-Cas system Type I-C from Clostridium bolteae (FIG. 7), Clostridium clostridioforme (FIG. 8) and Clostridium scindens (FIG. 9) were predicted.

### Example 3. CRISPR-Cas System Identification and Characterization in

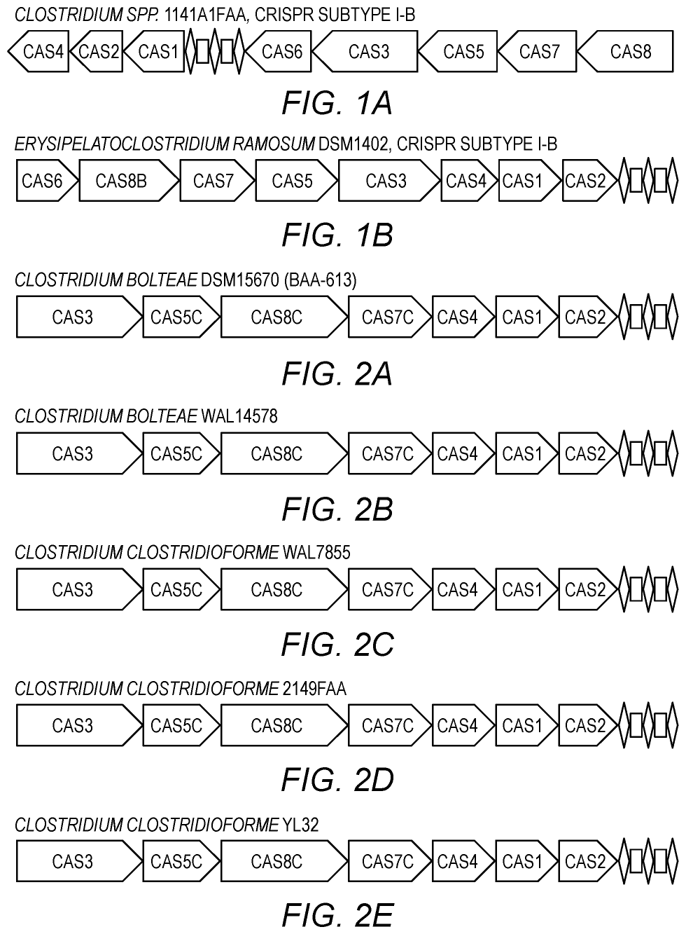

Erysipelatoclostridium ramosum and Clostridium spp. 1141A1FAA FIGS. 1A and 1B show the results of the characterization of the E. ramosum and C. spp. 1141A1FAA Cascade complex in which E. ramosum is shown to comprise a Cas3, Cas6, a Cas8, Cas7 and a Cas5 in addition to the spacer acquisition polypeptides Cas1, Cas2, and Cas4 (FIG. 1). C. spp. 1141A1FAA is shown to comprise a Cas3, Cas6, a Cas8, Cas7 and a Cas5 in addition to the spacer acquisition polypeptides Cas1, Cas2, and Cas4 (FIG. 1A). The CRISPR-Cas system subtype I-B of E. ramosum DSM1402 (Cas6-Cas8-Cas7-Cas5b-Cas3-Cas4-Cas1-Cas2) was compared with the canonical subtype I-B from Clostridium kluyveri DSM555 using MUSCLE algorithm for the nucleotide sequence alignment. The comparison showed that the nucleotide sequences of the CRISPR-Cas system subtype I-B of E. ramosum share less than 56% sequence identity with the nucleotide sequences from the canonical subtype I-B from Clostridium kluyveri DSM555. In addition, the E. ramosum Cas3 amino acid sequence was found to share less than 40% sequence identity with the Cas3 amino acid sequence of the Clostridium kluyveri DSM555 canonical subtype I-B system. This further demonstrates the distinctiveness of this newly characterized CRISPR-Cas system.

A phylogenomic analysis comparing the different Clostridium species of this invention and including E. coli, C. difficile and Erysipelatoclostridium ramosum is provided in FIG. 4 shows the phylogenetic distance among this species.

### Example 4. PAM Prediction of the Bacteria of Example 3

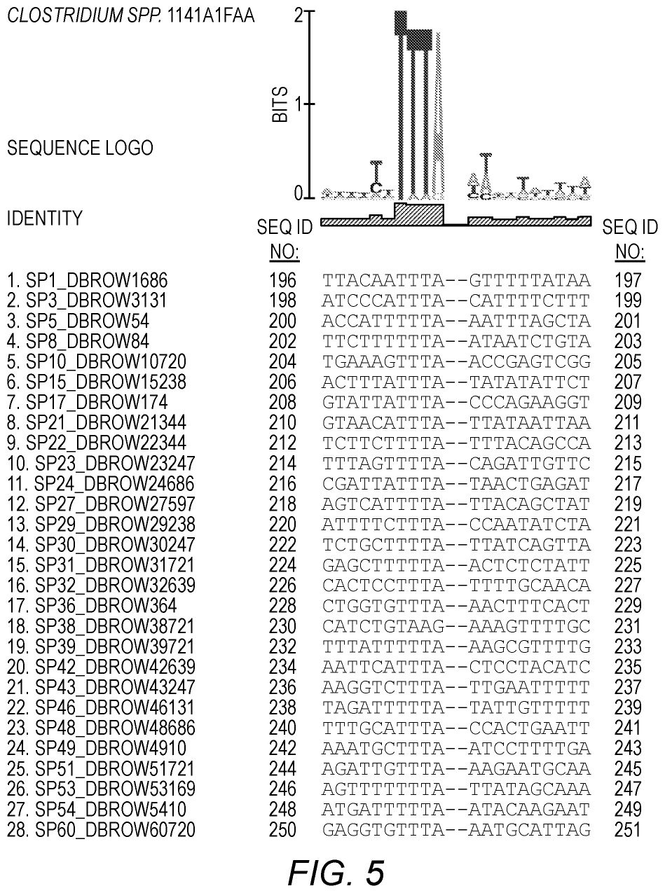

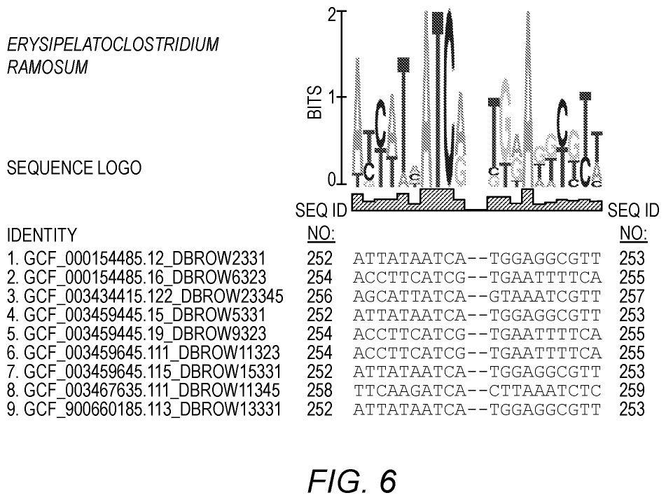

The CRISPR spacers were extracted from the CRISPR array of each of the strains described in Example 2, and a blastn was performed against different NCBI databases. The spacer-protospacer positive matches obtained were used to extract 10 nt of the adjacent (upstream and downstream) regions of the protospacer to elucidate the PAM sequence. The PAM sequences for the CRISPR-Cas system Type I-B locus from C. spp 1141A1FAA and E. ramosum were predicted as shown in FIGS. 5 and 6, respectively.

### Example 5. Bacterial Strains and Growth Conditions

The bacterial strains described in this invention are generally grown in broth or agar media, at anaerobic conditions and 37° C. for 2-5 days. The media to be used is species dependent and in some cases even strain dependent. The media used can include but not limited to Brain Heart Infusion (BHI) with or without 0.05-0.5% (w/v) L-cysteine, Reinforced Clostridial Medium (RCM) with or without 0.05-0.5% (w/v) as examples.

### Example 6. Validating the Functionality of the E. ramosum Type I CRISPR-Cas System

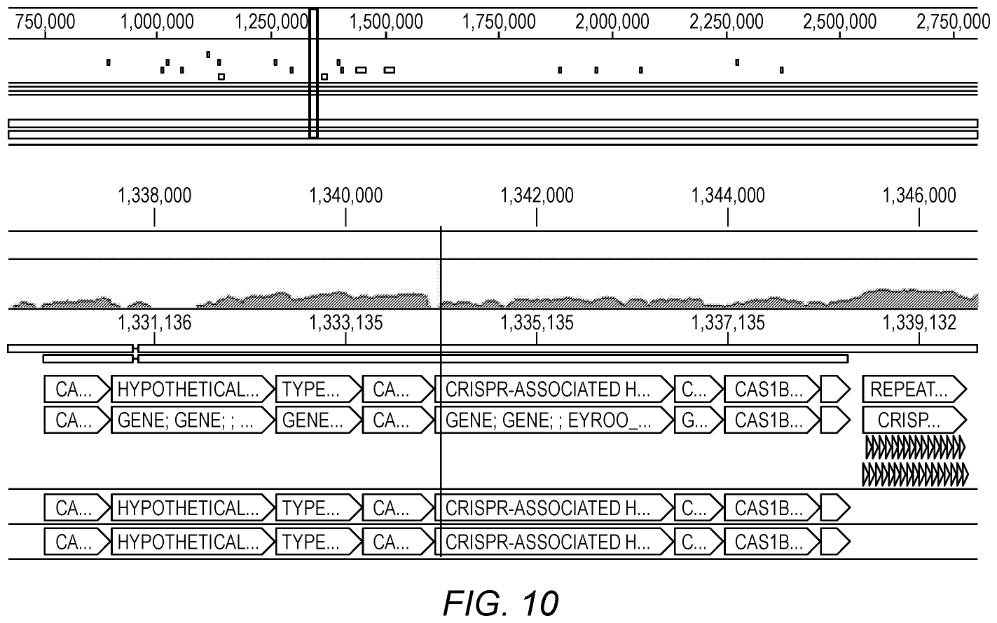

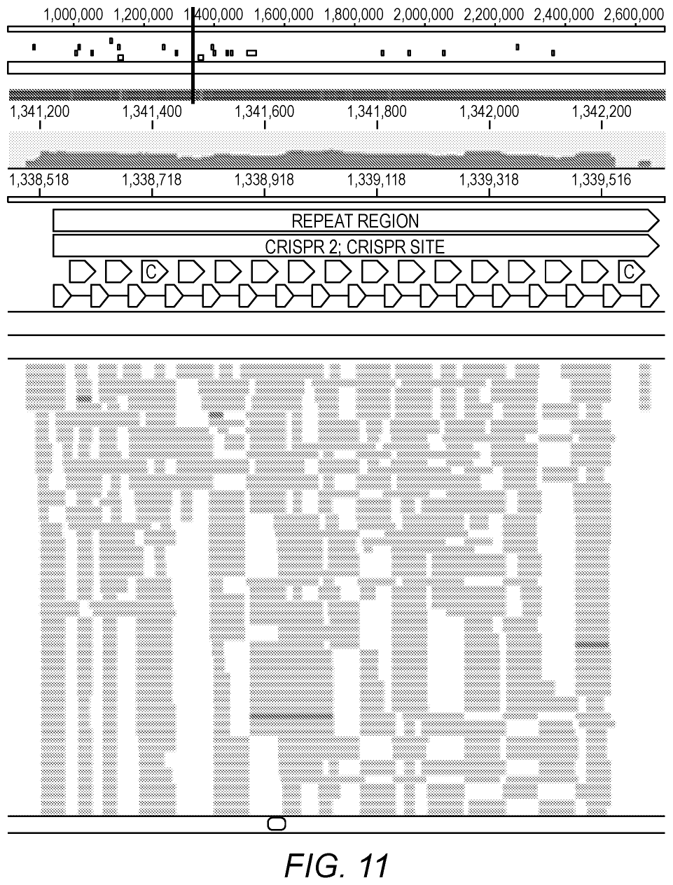

In order for CRISPR-Cas systems to be functional, it is necessary to have transcription of the cas genes to form the Cascade complex and transcription of the CRISPR array to generate mature CRISPR RNAs (crRNAs) that can guide the Cas machinery to the complementary sequence. We determined cas and CRISPR array transcriptional profiles in the native host to show activity of the endogenous E. ramosum type I CRISPR-Cas system, revealing cas transcription and the boundaries and sequence of the corresponding mature crRNA (See, FIG. 10 and FIG. 11). Sequencing was performed by UIUC using Illumina paired ends, and data was assembled, mapped and analyzed in Geneious Prime using the Geneious mapper.

### Example 7. The Mature E. ramosum crRNA

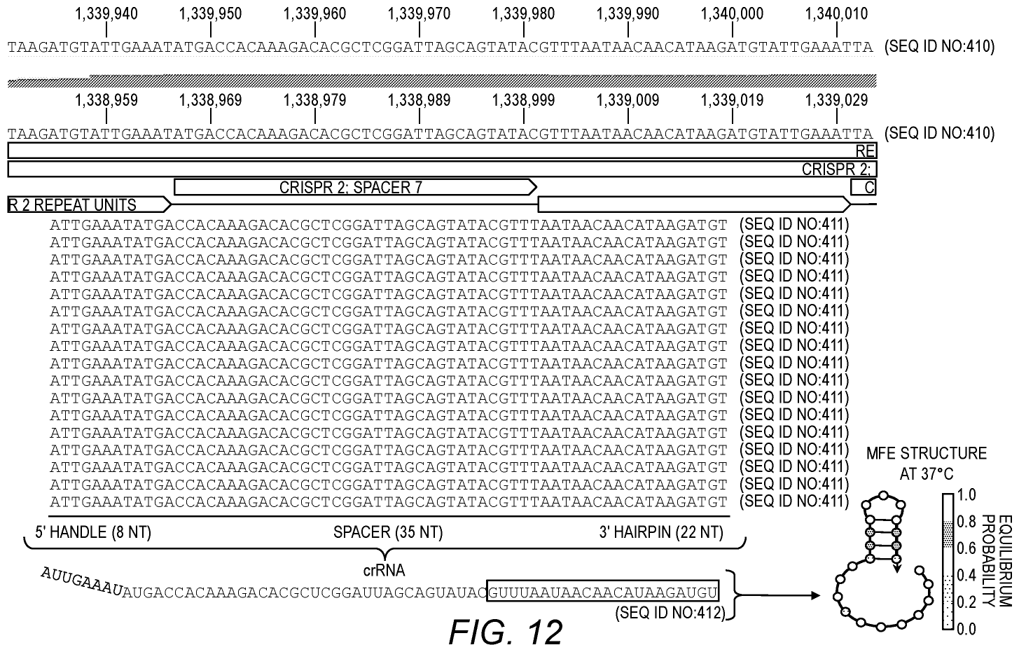

The composition, structure and boundaries of the mature E. ramosum crRNA was determined. The mature E. ramosum crRNA comprised of a full CRISPR spacer (can range between 34 nt and 37 nt) flanked by two sections of the CRISPR repeat, the 5′ handle (comprised of the 8 nt of the 3′ portion of the CRISPR repeat) and the 3′ hairpin (comprised of the 22 nt of the 5′ portion of the CRISPR repeat, which carries the palindrome within the CRISPR, and reveals processing at the base of the hairpin to generate the mature crRNA from the pre-crRNA full transcript) (see, FIG. 12). The hairpin structure shown in FIG. 12 was visualized in NUPACK. For CRISPR locus #2, spacer length can vary between about 34 to 37 nucleotides.

### Example 8. PAM Selection and deGFP Protospacer Sequence Determination

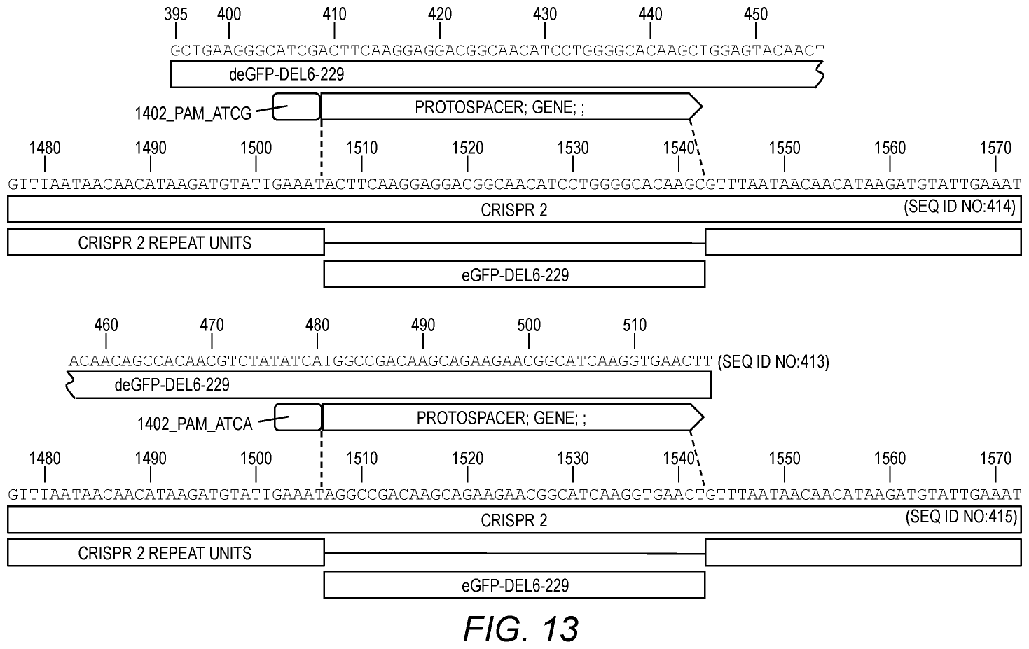

FIG. 13 provides a schematic of an example targeting CRISPR array with a spacer that is complementary to the sequence as shown in FIG. 13, enabling the genesis of a mature crRNA with a 36 nt targeting spacer flanked by the E. ramosum CRISPR repeat sections. The upper panel of FIG. 13 shows the target with a ATCG PAM flanking the 5′ edge of the protospacer. The lower panel of FIG. 13 shows the target with a ATCA PAM flanking the 5′ edge of the protospacer. A PAM sequence logo is shown in FIG. 6.

### Example 9. Transcriptional Control in Cell-Free Transcription-Translation (TXTL)

A transcription (TX)-translation (TL) platform, TXTL, was used with a mastermix, which consists of a cell-free extract enabling in vitro analysis of CRISPR effectors (Daicel Arbor Biosciences). This system is based on RNA polymerase sigma factor 70 (σ⁷⁰) for recognition of promoters on synthetic plasmids engineered to provide Cas proteins, the corresponding guide crRNAs and the target sequences. Reactions were carried out in small volumes (5 μl) in scalable formats (96-well plates) with fluorescence outputs that show Cas protein activity (e.g., binding to the target sequence blocking transcription and preventing GFP fluorescence). In this example, we provided the E. ramosum Cas machinery on a plasmid in combination with a CRISPR array comprising a spacer targeting the promoter sequence of the GFP gene. Transcription was prevented by Cascade binding to the complementary sequence and preventing transcription, showing Cascade programmability by the engineered CRISPR array.

A GFP fluorescence assay was used to show targeting by the E. ramosum Cascade-crRNA complex. The targeting was revealed by lowering of GFP transcription due to binding to the target sequence (complementary to the CRISPR spacer) and percent repression was calculated in the following manner

$$1 - \frac{targeting endpoint}{non - targeting endpoint} * 100$$

### Example 10. TXTL Genetic Circuit/Reaction

The E. ramosum Cascade set of cas genes (see, FIG. 1B, FIG. 10) is provided in combination with a CRISPR array comprising two repeats flanking a targeting spacer for a TXTL genetic circuit/reaction.

The TXTL experimental set up was as follows:
1. TXTL master mix—positive control
   myTXTL Sigma 70 Master Mix (75 uL)—contains Sigma 70 Master Mix and pTXTL-T70a(2)-deGFP HP control plasmid
   p70a-deGFP—2 nM
   P70a-T7RNAP—1 nM
   IPTG—1 nM
   H2O
2. Sample prep
   Targeting plasmid: E_ramosum_1402_PAM_ATCG (0.5 nM concentration) or E_ramosum_1402_PAM_ATCA (0.5 nM concentration)
   Non-targeting plasmid—negative control: E_ramosum_1402 NT (0.5 nM concentration)
   Each of targeting plasmid and non-targeting plasmid contain TXTL master mix & H20
3. Blank—positive control—used to subtract out background: myTXTL Sigma 70 Master Mix only
4. Reaction
   Plate reader—BMG Labtech FLUOstar Omega
   deGFP RFU measured every 10 mins for 16 hrs, 97 cycles total
   29° C. reaction temperature

The E. coli cell-free transcription-translation (TXTL) system was used in vitro to test the functionality of the type I-B CRISPR-Cas system derived from Erysipelatoclostridium ramosum DSM 1402. Expressed in the targeting plasmid, are the multi-effector CRISPR nucleases, cas proteins cas6875b, that form the active CRISPR machinery (cascade—CRISPR associated complex for antiviral defense) needed for targeted gene repression (deGFP) in the TXTL reaction. In addition, the deGFP gRNA is expressed in the targeting plasmid which binds complementary to the protospacer region next to the PAM ATCG or ATCA sequence in the p70a-deGFP plasmid.

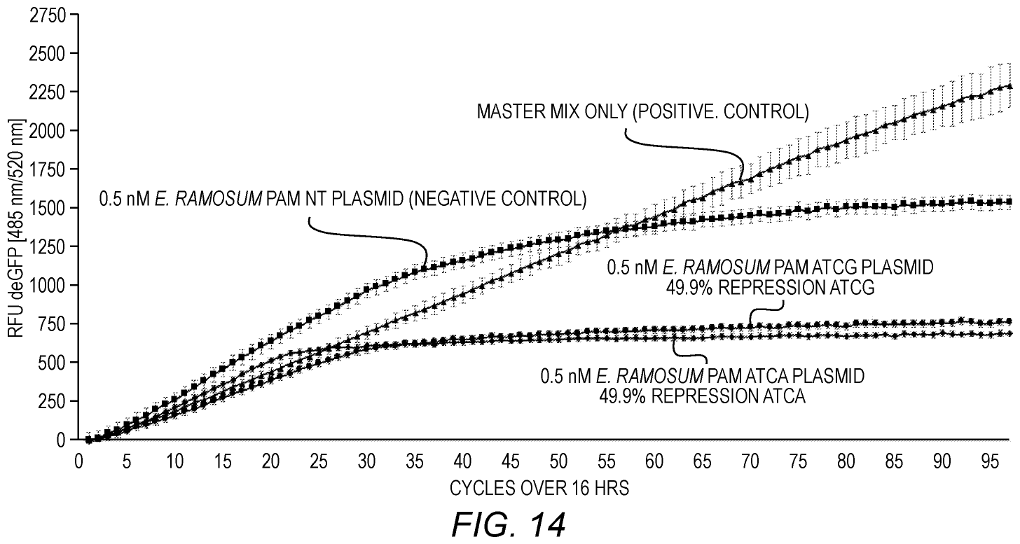

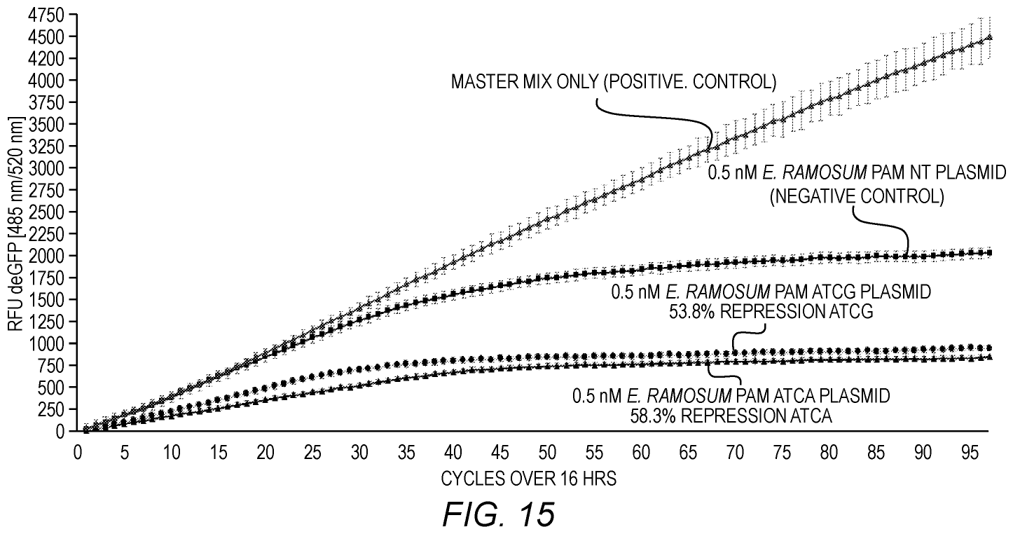

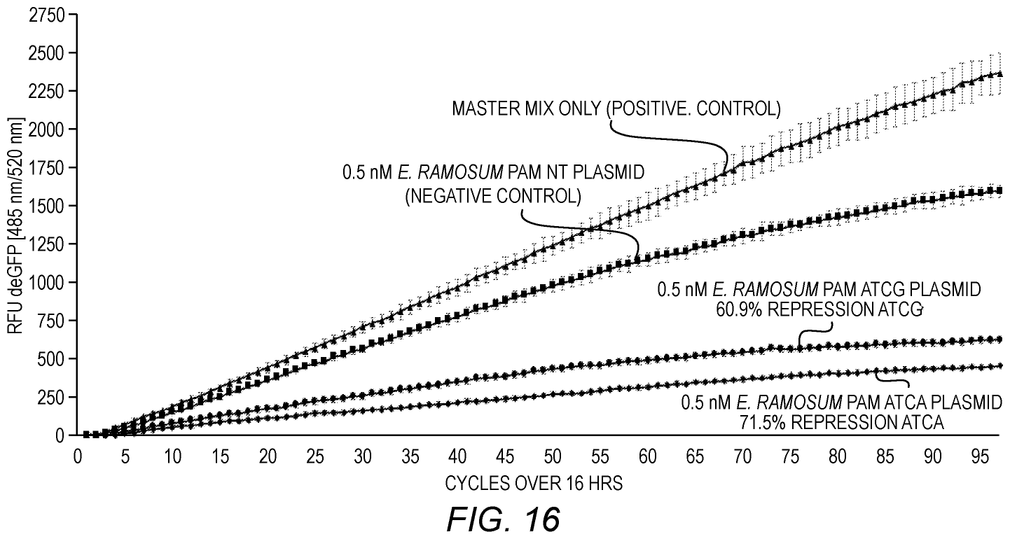

The results of the TXTL experiments are shown in FIGS. 14-16. FIG. 14 provides the results of round 1 testing of 0.5 nM E. ramosum PAM ATCA or ATCG plasmid using a non-targeting plasmid as a neg. control. FIG. 15 provides the results of round 2 testing of 0.5 nM E. ramosum PAM ATCA or ATCG plasmid using a non-targeting plasmid as a neg. control. FIG. 16 provides the results of round three testing of 0.5 nM E. ramosum PAM ATCA or ATCG plasmid using a non-targeting plasmid as a neg. control.

Using the in vitro TXTL system, 49.9%, 53.8%, and 60.9% deGFP repression was achieved at a 0.5 nM concentration using the PAM ATCG targeting plasmid. A 54.9%, 58.3%, and 71.5% deGFP repression was achieved at a 0.5 nM concentration using the PAM ATCA targeting plasmid. (see, FIGS. 14-16). The TXTL reaction confirmed system activity and efficient repression using the endogenous type I-B CRISPR cascade from E. ramosum DSM 1402 when provided with a guide RNA that matched a target (deGFP) positioned next to the predicted PAM (ATCG or ATCA).

The foregoing is illustrative of the present invention and is not to be construed as limiting thereof. The invention is defined by the following claims, with equivalents of the claims to be included therein.

That which is claimed is:

1. A method of enhancing resistance of a bacterial cell to one or more bacteriophage species or strains, the method comprising introducing into the bacterial cell a recombinant nucleic acid construct comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence or portion thereof, and each of the one or more spacer sequences is complementary to a target sequence (protospacer) in a target nucleic acid of a bacteriophage species or strain, wherein the target sequence is located immediately adjacent (3′) to a protospacer adjacent motif (PAM).

2. The method of claim 1, wherein the one or more spacer sequences are non-natural spacer sequences (i.e., not a previously acquired spacer sequence) to the bacterial cell.

3. The method of claim 1 or claim 2, wherein each of the one or more spacer sequences is linked at its 3′-end to a repeat sequence.

4. The method of any one of claims 1-3, wherein the spacer sequence is at least 80% complementary to the target sequence.

5. The method of any one of claims 1-4, wherein the target sequence (protospacer) is conserved in the genome of the bacteriophage species or strain.

6. The method of any one of claims 1-5, wherein the target sequence (protospacer) is a portion of a gene encoding a tail protein, a portal protein, a capsid protein, a holin, a lysin, and/or a DNA packaging protein.

7. The method of any one of claims 1-6, wherein the one or more spacer sequence(s) each have a length of about 20 nucleotides to about 40 nucleotides, optionally about 30 nucleotides to about 40 nucleotides (e.g., about 30, 31, 32, 33, 34, 35, 36, 37, 38 nucleotides) in length, or about 20, 22, 31, 33, 34, or 38 nucleotides in length, optionally about 34 nucleotides in length.

8. The method of any one of claims 1-7, wherein the one or more spacer sequence(s) each have a length of about 25 nucleotides to about 60 nucleotides, optionally about 30 nucleotides to about 40 nucleotides, about 32 nucleotides to about 40 nucleotides, or about 34, 35, 36 or 37 nucleotides.

9. The method of any one of claims 1-8, wherein at least two of the one or more spacer sequence(s) comprise nucleotide sequences that are complementary to different target sequences.

10. The method of any one of claim 9, wherein the different target sequences are from the same bacteriophage species or strain or from different bacteriophage species or strains.

11. The method of any one of claims 1-10, wherein the one or more spacer sequence(s) each comprise a 5′ region and a 3′ region, wherein the 5′ region comprises a seed sequence and the 3′ region comprises a remaining portion of the one or more spacer sequence(s).

12. The method of claim 11, wherein the seed sequence comprises the first 8 nucleotides of the 5′ end of each of the one or more spacer sequence(s), and is fully complementary (100%) to the target sequence, and the remaining portion of the one or more spacer sequence(s) is at least about 80% complementary (e.g., about 80, 81, 82, 83, 84, 85, 86, 87, 88, 89, 90, 91, 92, 93, 94, 95, 96, 97, 98, 99, or 100% complementarity) to the target sequence.

13. The method of any one of claims 1-12, wherein the CRISPR is a Type I, Type II, Type III, Type IV, Type V or Type VI CRISPR and the one or more repeat sequences of the CRISPR is from a Type I, Type II, Type III, Type IV, Type V or Type VI repeat sequence, respectively.

14. The method of claim 13, wherein the one or more repeat sequences are a full-length Type I, Type II, Type III, Type IV, Type V or Type VI CRISPR repeat sequence or a portion thereof.

15. The method of claim 14, wherein the portion of the full-length Type I, Type II, Type III, Type IV, Type V or Type VI CRISPR repeat sequence comprises about 20 consecutive nucleotides to about 40 consecutive nucleotides of the full-length Type I, Type II, Type III, Type IV, Type V or Type VI repeat sequence.

16. The method of any one of claims 1-7 or 9-12, wherein the one or more repeat sequences comprise at least 24 consecutive nucleotides (e.g., about 24, 25, 26, 27, 28, 29, 30, 31, 32 or 33 consecutive nucleotides) having at least 80% sequence identity to any one of the nucleotide sequences of SEQ ID NOs:15-19, any one of the nucleotide sequences of SEQ ID NOs: 34-35, any one of the nucleotide sequences of SEQ ID NOs:50-53, any one of the nucleotide sequences of SEQ ID NOs: 68-71, any one of the nucleotide sequences of SEQ ID NOs:86-88, any one of the nucleotide sequences of SEQ ID NOs: 103-105, or any one of the nucleotide sequences of SEQ ID NOs: 120-121, optionally about 25 to 33 consecutive nucleotides or about 30 to 33 consecutive nucleotides of the repeat sequence; or

17. The method of any one of claims 1-6 or 8-12, wherein the one or more repeat sequences comprise at least 19 consecutive nucleotides (e.g., about 19, 20, 21, 22, 23, 24, 25, 26, 27, 28, 29, 30, 31, or 32 consecutive nucleotides) having at least 80% sequence identity to any one of the nucleotide sequences of SEQ ID NOs:156-164 or any one of the nucleotide sequences of SEQ ID NO:138 or SEQ ID NO:139, optionally about 27 to 32 consecutive nucleotides or about 30 to 32 consecutive nucleotides

18. The method of any one of claims 1-7, 8-12, or 16 or, wherein the PAM comprises a nucleotide sequence of 5′-TTC-3′, 5′-CTC-3′ or 5′-TTT-3′ that is immediately adjacent to and 5′ of the target sequence (protospacer)

19. The method of any one of claims 1-6, 8-12 or 17, wherein the PAM comprises a nucleotide sequence of 5′-TTTA-3′, 5′-ATC-3′, and/or 5′-ATCN-3′ (e.g., 5′-ATCA-3′, and/or 5′-ATCG-3′) that is immediately adjacent to and 5′ of the target sequence (protospacer).

20. The method of any one of claims 1-19, wherein the CRISPR is operably linked to a promoter or leader sequence.

21. The method of claim 20, wherein the promoter is endogenous to the repeat sequences (e.g., for the Type I-C Cascade complex, the promoter is endogenous to the repeat sequences of Clostridium scindens (e.g., C. scindens ATCC35704), Clostridium clostridioforme (e.g., C. clostridioforme WAL7855, C. clostridioforme NCTC11224, C. clostridioforme YL32, C. clostridioforme 2149FAA) or Clostridium bolteae (e.g., C. bolteae DSM15670 (BAA-613), C. bolteae WAL14578) and for the Type I-B Cascade complex the promoter is endogenous to the repeat sequences of Erysipelatoclostridium ramosum or Clostridium spp. 1141A1FAA).

22. The method of claim 20 or claim 21, wherein the promoter is endogenous to the bacterial cell.

23. The method of claim 19, wherein the promoter is heterologous to the repeat sequences.

24. The method of claim 20-23, wherein the promoter comprises the nucleotide sequence of any of SEQ ID NOs:165-176 or 177-185.

25. The method of any one of claims 1-24, further comprising a terminator sequence operably linked to the CRISPR.

26. The method of claim 25, wherein the terminator sequence is a Rho-independent terminator sequence, a Clostridium scindens terminator sequence, a Clostridium clostridioforme terminator sequence, a Clostridium bolteae terminator sequence, a Erysipelatoclostridium ramosum terminator sequence, or a Clostridium spp. 1141A1FAA terminator sequence.

27. The method of claim 25 or claim 26, wherein the terminator comprises the nucleotide sequence of any of SEQ ID NOs:186-194.

28. The method of any one of claims 1-27, wherein the recombinant nucleic acid construct is comprised in a vector.

29. The method of claim 28, wherein the vector is a plasmid, a phagemid, a transposon, or a bacteriophage.

30. The method of any one of claims 1-29, wherein the recombinant nucleic acid construct is maintained in the bacterial cell as an extrachromosomal element (e.g., a plasmid).

31. The method of any one of claims 1-29, wherein the recombinant nucleic acid construct is incorporated into the chromosome of the bacterial cell.

32. The method of claim 31, wherein the recombinant nucleic acid construct is incorporated into an endogenous CRISPR array in the chromosome of the bacterial cell.

33. The method of any one of claims 1-32, wherein the bacterial cell is from a Clostridium spp., a Erysipelatoclostridium spp., a Lactococcus spp., a Streptococcus spp., a Klebsiella spp., a Propionibacterium spp., a Cutibacterium spp., a Lactobacillus spp., a Pseudomonas spp., a Faecalibacterium spp., a Akkermansia spp., a Bifidobacterium spp., a Roseburia spp., an E. coli spp., or a Clostridiodis spp.

34. The method of any one of claims 1-33, wherein the bacterial cell is a Clostridium spp. cell, Clostridium scindens cell, a Clostridium clostridioforme cell, a Clostridium bolteae cell, or a Erysipelatoclostridium ramosum cell.

35. The method of any one of claims 1 to 34, wherein the method further comprises introducing a recombinant nucleic acid encoding Cas3 polypeptide and a Type I-C Cascade complex and/or a Cas3 polypeptide and a Type I-B Cascade complex.

36. The method of claim 35, wherein the Type I-C Cascade complex comprises a Cas5 polypeptide, a Cas8 polypeptide, and a Cas7 polypeptide.

37. The method of claim 35, wherein the Cas3 polypeptide comprises a sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the amino acid sequences of SEQ ID NOs:1, 20, 36, 54, 72, 89, or 106, the Cas5 polypeptide comprises a sequence having at least 80% sequence identity to any one of the amino acid sequences of SEQ ID NOs:2, 21, 37, 55, 73, 90, or 107, the Cas8 polypeptide comprises a sequence having at least 80% sequence identity to any one of the amino acid sequences of SEQ ID NOs:3, 22, 38, 56, 74, 91, or 108, and the Cas7 polypeptide comprises a sequence having at least 80% sequence identity to any one of the amino acid sequences of SEQ ID NOs:4, 23, 39, 57, 75, 92, or 109.

38. The method of claim 35, wherein the Type I-B Cascade complex comprises a Cas6 polypeptide, a Cas8 polypeptide, a Cas7 polypeptide, and a Cas5 polypeptide.

39. The method of claim 38, wherein the Cas6 polypeptide comprises a sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:122 or SEQ ID NO:140, the Cas8 polypeptide comprises a sequence having at least 80% sequence identity to the amino acid sequence of SEQ ID NO:123 or SEQ ID NO:141, the Cas7 polypeptide comprises a sequence having at least 80% sequence identity to the amino acid sequence of SEQ ID NO:124 or SEQ ID NO:142, the Cas5 polypeptide comprises a sequence having at least 80% sequence identity to the amino acid sequence of SEQ ID NO:125 or SEQ ID NO:142, and the Cas3 polypeptide comprises a sequence having at least 80% sequence identity to the amino acid sequence of SEQ ID NO:126 or SEQ ID NO:144.

40. The method of any one of claims 1-39, wherein the bacterial cell is a commensal bacterial cell, optionally a Lactobacillus spp., or a Bifidiobacterium spp. or a Clostridium spp.

41. A method of enhancing resistance of a bacterial cell to one or more bacteriophage species or strains, the method comprising introducing into the bacterial cell
(a) at least one protein-RNA complex comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence, and each of the one or more spacer sequences is complementary to a target sequence (protospacer) in a target nucleic acid of a bacteriophage species or strain, wherein the target sequence is located immediately adjacent (3′) to a protospacer adjacent motif (PAM) and
(b) at least one polypeptide of a Type I CRISPR-Cas system, a Type II CRISPR-Cas system, a Type III CRISPR-Cas system, a Type IV CRISPR-Cas system, a Type V CRISPR-Cas system or a Type VI CRISPR CRISPR-Cas system.

42. A method of enhancing resistance of a bacterial cell to one or more bacteriophage species or strains, the method comprising introducing into the bacterial cell at least one protein-RNA complex comprising a Clustered Regularly Interspaced Short Palindromic Repeats (CRISPR) comprising one or more repeat sequences and one or more spacer sequence(s), wherein each of the one or more spacer sequences is linked at least at its 5′-end to a repeat sequence, and each of the one or more spacer sequences is complementary to a target sequence (protospacer) in a target nucleic acid of a bacteriophage species or strain, wherein the target sequence is located immediately adjacent (3′) to a protospacer adjacent motif (PAM) and
(a) a Cas3 polypeptide and a Type I-B Cascade complex comprising a Cas6 polypeptide, a Cas8 polypeptide, a Cas7 polypeptide, and a Cas5 polypeptide; or
(b) a Cas3 polypeptide and a Type I-C Cascade complex comprising a Cas5 polypeptide, a Cas8 polypeptide and a Cas7 polypeptide.

43. The method of claim 42, wherein the Cas6 polypeptide of the Type I-B Cascade complex comprises a sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to the amino acid sequence of SEQ ID NO:122 or SEQ ID NO:140, the Cas8 polypeptide of the Type I-B Cascade complex comprises a sequence having at least 80% sequence identity to the amino acid sequence of SEQ ID NO:123 or SEQ ID NO:141, the Cas7 polypeptide of the Type I-B Cascade complex comprises a sequence having at least 80% sequence identity to the amino acid sequence of SEQ ID NO:124 or SEQ ID NO:142, the Cas5 polypeptide of the Type I-B Cascade complex comprises a sequence having at least 80% sequence identity to the amino acid sequence of SEQ ID NO:125 or SEQ ID NO:142, and the Cas3 polypeptide comprises a sequence having at least 80% sequence identity to the amino acid sequence of SEQ ID NO:126 or SEQ ID NO:144.

44. The method of claim 42, wherein the Cas3 polypeptide comprises a sequence having at least 80% sequence identity (e.g., about 80, 81, 82, 83, 84, 85, 86, 87, 88, 89, 90, 91, 92, 93, 94, 95, 96, 97, 98, 99, 100%; or at least about 85%, 90%, 95%, 96%, 97%, 98%, 99% sequence identity) to any one of the amino acid sequences of SEQ ID NOs:1, 20, 36, 54, 72, 89, or 106, the Cas5 polypeptide of the Type I-C Cascade complex comprises a sequence having at least 80% sequence identity to any one of the amino acid sequences of SEQ ID NOs:2, 21, 37, 55, 73, 90, or 107, the Cas8 polypeptide of the Type I-C Cascade complex comprises a sequence having at least 80% sequence identity to any one of the amino acid sequences of SEQ ID NOs:3, 22, 38, 56, 74, 91, or 108, and the Cas7 polypeptide of the Type I-C Cascade complex comprises a sequence having at least 80% sequence identity to any one of the amino acid sequences of SEQ ID NOs:4, 23, 39, 57, 75, 92, or 109.

45. The method of any one of claims 1-44, wherein at least one of the one or more bacteriophage is a new bacteriophage species or strain not a previously targeted by an endogenous CRISPR system of the bacterial cell, thereby conferring resistance to the new bacteriophage species or strain.

46. The method of any one of claims 2-45, wherein at least one of the one or more bacteriophage is a bacteriophage species or strain that is previously targeted by an endogenous CRISPR system of the bacterial cell, and the at least one non-natural spacer sequence is complementary to a different nucleic acid sequence than a spacer sequence of the endogenous CRIPSR system, thereby increasing resistance to the at least one bacteriophage species or strain.

47. The method of any one of claims 2-46 wherein at least one of the one or more bacteriophage is a bacteriophage species or strain to which an endogenous CRISPR system of the bacterial cell comprises a spacer that is not effective for killing the bacteriophage species or strain, and the one or more spacer sequences of the introduced CRISPR are complementary to a different nucleic acid sequence than the spacer sequence of the endogenous CRISPR system, thereby conferring resistance to the bacteriophage species or strain.


SEQUENCE LISTING (415 sequences)

SEQ ID NO: 1 - protein, 807 aa, Clostridium scindens
MNDPYIAHFYKDSNNAIHIQSVSEHNLNVAYLAMSNSPLKCLSSVAWVSGALHDAGKYREGVQNYLRRAMNEEQVRRGEEDHSTGGGFLIDKMFPATNLSVLIQMAVFCHHGLHDCFSFEKSTIYIKERLSKKENFQNVEERYYQYLKKEDLEKRGLIAKQEVAGIIQEILGFIRIKNLSKNMYGNRDFFLGMYERTIMSLLVDADRTDTACFMEQRELPAPKSDEMMQDIWGKCITYLEQYLKSMKSESRLDEYRRDISRKCMEAGEREERLYRLTVPTGAGKTLSSLRFGLYHARKYHKRHIIYVAPYQSIIDQNAEEIRRAIHNDEIVLEHHCNVIHDDERERIRYEILTENWDSPIIVTTAVQFLNTLFASGIGNIRRMYSILNSVIIFDEVQALPVKITKLFNLSVNFLTAFGKSTVVLCSATQPLFDELDENRLLPPLSMAGNPKEYAEVFKRTKIIDATKGAGSGFAAEELQEFIWDKAETLKQVLVVVNTKKCAESLYKEIKKKCKGSDYILFHLSTNMCAEHRRDVLKRLEENLENEKKVICISTQLIEAGVNLSFKCVIRSLAGLDNIIQAAGRCNRHGEAGLGYVYVVGISSELENVERLAEIRKMQESMTEVLNQFRKNPEIFDHSLSSEESIRYYYQIYLRKRMPEMEYLVSVNGVDVTLLDLLSTSKTMWESMSESTKKDSKNLLLKQAFKTAGDVFEVIPEDGKVDVVVRYDEKAEKQIAILEDEYATLAEHKKALRRLQPYTVGISEQFRQKLGNAITSVCGGTVYVLSQNYYSRETGVSEEPLGMEFINF

SEQ ID NO: 2 - protein, 246 aa, Clostridium scindens
MKYRNTVEFEVYGDYALFSDPVTRVGGEKCSYHVPTYEALKGILQSVYWKPTLIWIIDAVRIMKPIQTETKAIRPISYNGGNDLSYYTYLKDVSYQVRAHFEWNRNRPELESDRNENKHHNIARRMISRGGRRDIFLGTRECQGYVVPCEFEEGEGYYDSQEGDLSYGLMYHGITYADEAVLPEDEGKMTVRFWHPVMKKGIIEFTRPEECAEKRHIREMAVKPFGEGNFTGIREFDGEEEDIELV

SEQ ID NO: 3 - protein, 659 aa, Clostridium scindens
MSWFSELYDLYEKNEEKIGRVEYKTRQGKKGPEQVPLVLLPIFHTTVAAQITVDIDSEGNILGASRVPDEDKMTIIPVTETSNSRTSDPKAHPFCDNLKYLAADYARYVKNGKKDFSKNHKLYIEGLKSWHLSEYTHPKVDALYAYLSKGTLMADLVEEGVLITDEKGVLLEKAKIQIVSQADAFVRFRIIEKADLSQDILNDPSGRYFSECWLDGTLQKSYIKYHRSRLTQTDFCYLTGENVPVSYLQPKKIRNEGDGAKLISSNDNKNYTFRGRFSDKTEAFAIGYEVSQKVHNALKWIIRKQGYSWGGLCIVIWESNLKPIPDWGADTDKIEEACQEAQESEYEGWGEEEEQEYETGAAAAARFKAAMRGYGENLKTDSKVMILAFDAATTGRLAMVENKEFAASRYVDNIKYWHDSCRWLQSKYKNGHNFRYFGMAGVKDIAEALYGTEQKGIISLGGNTRMYAEVCKRLLPCISERRRVPRDIVNAAVQKASSPVSYDNRYNWEKVLSIACALVKKQKMEVEKEEWKVSLNEESRNRDYLYGRLLAMADRIEYRTFDRDEDGKRVTNAKRYMNAFAQHPYQTWKVLEERIQPYLQKLDIKERNSYNKTLDEIYELFDEKEFTNNDRLEGLYLLGYHSQSYELKYRPKKAEEEKE

SEQ ID NO: 4 - protein, 284 aa, Clostridium scindens
MTTLKGKIDFALLVSATNANPNGDPLNGNRPRENYEGYGEISDVCIKRKIRNRFQDMGQKIFVQSDDRSDDGYTSLKKRAEGCEELNNEIKKKKADREHFAQIACQEWLDVRTFGQVFAFKGEEVSVGIRGPVSIHQAVSLSPVDVVSMQITKSVNSEAGKDSKASDTMGTKHRVGFGLYLVKGSVNVQLAEKTGFSQEDAELLKEALRTLFENDASSARPEGSMEVCRLYWWQHEEKTPSVPTAQIHRSIKIEALEDRPKSFKDYSIKLEELNCVKPEVYEFV

SEQ ID NO: 5 - protein, 220 aa, Clostridium scindens
MSLYKEEDYLQLSGIQHFAFCRRQWALAYIELQWDENVRTVEGKLLHEKAHDPTNAEKRGDLIISRGMPVYSRELGISGECDVVEFHRSQDGITLFGRDGLYEVIPIEYKRGKPKENDVDILQLMAQALCLEEMLCCQISFGYLYYGETRHRKIVEFTEEIRHKTKDMFLEMHQYYERRYTPRVKRSKACNACSLKDICLPALGIAKSATDYINSRLSEE

SEQ ID NO: 6 - protein, 342 aa, Clostridium scindens
KKLMNTLYVTSANRYLSLDGENVVILEEQKEIGRVPLHNLEGIVTFGYTGASPRLMGTCAQKNISLTFMTEHGRFLARVCGEVRGNVTLRRQQYRIADNTEKRIEVARNCIVGKVYNSRWILERAIRDYPLRLDVEKIKEKSSFLASRLKEIRTCESPDKLLGLEGESASVYFSVFDELILQQKETFYFHGRNRRPPLDNVNAMLSFAYSLLAGICTSALETVGLDPYVGFFHTDRPGRASLALDIMEELRSVMADRFVLTLINKRIVDSSGFLQKENGAVIMDDDTRKKFLAAWQDKKKEVITHPFLGEKMEWGMVPHVQAMLLARYIRGDLEEYPPFLWK

SEQ ID NO: 7 - protein, 96 aa, Clostridium scindens
MLVLITYDVNTETEAGKRRLRKVAKQCVNYGQRVQNSVFECNLDAAKLRQVKAILEDIIDKEVDSLRFYNLGNKYKDKIEHIGAKKSFDVTAPLIF

SEQ ID NO: 8 - DNA, 2424 bp, Clostridium scindens
  source 1..2424: mol_type = genomic DNA
atgaacgatccatacattgcacatttttataaagacagtaataatgctatacacatacaatccgtttccgagcacaatcttaatgtggcttatcttgccatgagtaattccccattgaaatgtctatcctctgtcgcctgggttagtggtgcgcttcatgacgctggaaaatatagagaaggtgtccaaaattatctcagaagagcgatgaatgaagaacaagtgcgtcgcggggaagaagaccactcgacaggcggcggttttctgatagataagatgtttcccgccacaaatctatctgtgctgatacagatggcagtattttgccatcatggtctgcatgactgtttttcattcgaaaaatccactatttacattaaagagcgattgtccaaaaaagagaatttccaaaatgtagaggaacgttattatcaatatttaaaaaaggaagatttggagaagagaggcctcatagcaaaacaggaagtagcaggcattatccaggagatattagggtttattcgtattaagaatttgagcaaaaatatgtatggaaacagagatttttttctgggtatgtatgaaaggacaattatgtcgctgctggtagatgcagataggactgatacggcatgctttatggagcagagagaattgcctgctcctaaaagtgatgaaatgatgcaagatatttggggaaaatgtattacatatctggaacagtatttaaagtccatgaaatcagaatcaaggctggatgaatatcgcagggatatctcgcggaaatgtatggaggccggagaaagagaagaaagactttaccgtctgacagtgcccaccggagcgggtaagacgcttagtagtctgcgttttgggctgtatcatgccaggaaatatcataagagacatatcatatatgtagcgccctatcagagtataatagaccagaatgcggaagaaatacgcagggcgattcataatgatgagattgttctagagcaccattgtaatgtgattcatgatgatgaaagagagagaatccgatatgaaattctgacagagaattgggattcgccgattatagtcactacagcggtacaatttttgaacacgttgtttgcctctggaattggaaatatcaggcgtatgtatagcattctgaatagtgtgattatatttgatgaagttcaagccttgccagtaaaaattaccaaactgtttaatctttctgtgaactttctgacggcatttgggaagtcgacagtggtcttatgttcagcgacacaaccgttatttgatgagctggatgaaaataggctgttgccgccactatctatggcaggcaatcccaaagaatatgctgaggtatttaaaagaacgaaaattatagatgctacgaaaggagcaggaagcggatttgcggcagaagaattacaagagtttatctgggacaaggcggagacgcttaaacaagttcttgtcgttgtgaatacaaagaaatgtgcagagagtctttataaagagattaagaaaaaatgtaagggatcggattatatattatttcatctgagcacgaatatgtgcgcggaacacaggagggatgtactaaagagactggaagagaatttagaaaatgagaagaaggtgatatgtatcagcactcagctgattgaggcgggagtgaatctatccttcaagtgcgtcatacgttccctggcaggccttgacaatatcattcaggcggcaggccgatgcaacaggcatggggaagcgggtttggggtacgtctatgttgtgggaataagcagcgagttagaaaatgtagaaagacttgcggagattcgaaaaatgcaggagagcatgacagaagtgctaaatcaattcaggaagaatccggaaatatttgatcatagtctgtcgtccgaggagtcaatccggtactattaccagatatatctaaggaagagaatgccggagatggaatatcttgtatcggtgaatggagtggatgttacactgcttgacttgctatccactagcaagacgatgtgggaaagcatgtcagagtcaaccaaaaaggatagtaagaatttgctgttaaagcaggcgtttaaaacggcgggagatgtattcgaagtgatacccgaagatggaaaggtagatgtcgtagttagatatgatgaaaaagcagagaagcagatagccattcttgaggacgaatatgctacgctggcagagcataagaaagcgcttcgaaggcttcagccatatacggtggggatatccgagcaatttcgtcagaagttaggaaatgcaattacatcggtatgtggaggaaccgtctatgtcctgagtcagaactattatagtagagaaactggtgtttcagaagaaccgctgggaatggaatttataaacttttag

SEQ ID NO: 9 - DNA, 741 bp, Clostridium scindens
  source 1..741: mol_type = genomic DNA
gtgaagtatcgcaatacagtagaatttgaagtgtatggagactacgcgttgttttcggatcccgtgacccgcgtaggaggggagaaatgttcttatcatgtgcccacgtatgaagcgcttaaagggattttgcaaagcgtgtactggaagcctacgttgatttggattatagatgcggtaaggattatgaagccaatccagacggagacaaaggctatccgtcccattagttataatgggggaaatgatttatcctattatacttatttgaaggatgtcagttatcaagtgcgggcacattttgaatggaacagaaacaggccggaactagaatcggaccgtaatgaaaataagcatcataatattgccaggcgaatgataagccgaggcggaagaagggatattttcctgggcacaagagaatgtcagggatatgtggtaccatgcgagtttgaagaaggggaaggatattatgacagccaggaaggcgatttgtcctatgggcttatgtatcatggcattacttatgcggatgaggcagttcttccggaagatgagggaaagatgacggtgaggttctggcatccggtcatgaaaaagggaatcatcgaatttacccgtcctgaggagtgtgcggaaaaaagacacattagggaaatggcagtaaagccatttggagagggcaattttacaggcattcgagaatttgacggggaggaggaagacattgagttggtttag

SEQ ID NO: 10 - DNA, 1980 bp, Clostridium scindens
  source 1..1980: mol_type = genomic DNA
ttgagttggtttagcgaattgtatgatttatatgagaaaaatgaagagaaaattggaagagtagaatataagacccgccaggggaaaaagggaccagagcaggttccgctggttcttttgccaatatttcatacgacggtagcagcacagattacggttgacattgacagtgaaggaaatattctcggagcatcaagagtgccggatgaagataagatgacaatcatccccgtgacggagacttccaacagcaggacatcggatccaaaggcgcatcctttctgtgacaacctaaaatacctggctgcagattacgcaagatatgtaaagaatggaaaaaaagacttttccaagaatcacaaactctacatagaaggtctaaagtcctggcatctgtctgagtatacacatccaaaagtagatgcgctttatgcttatttaagcaaggggacgctcatggctgatttggttgaagaaggagtcttgattacagatgaaaaaggagttcttttagaaaaggcaaagatacagattgtatcacaggcagatgcgtttgtaagatttcgtatcatagaaaaagcagatttaagccaggatattttaaacgatccttccgggcgatatttttcagaatgttggctggatgggaccttgcagaaaagttatatcaagtatcaccgttccagattaacgcagacagatttctgctatcttactggcgagaatgttccggtttcttatcttcagccaaagaaaatcagaaatgaaggagacggggcgaaacttatttcttcaaatgataataaaaattatacatttagaggaagattctcggataagacggaagcatttgccataggctatgaggtctcccaaaaagtacataatgcgcttaaatggatcatacgcaaacaaggatatagttggggtgggctttgcatcgtgatttgggaatctaatctgaagccgattccggattggggcgctgatacagataagattgaggaagcctgtcaggaggcgcaggaaagcgaatatgaaggatggggagaggaggaagaacaggaatatgagacgggagccgcggctgcggcaaggttcaaagcagcgatgcgcggatatggagagaatctgaagacggactcgaaagttatgattcttgcctttgatgctgccactactggaaggcttgctatggtggaaaataaagagtttgccgccagcaggtatgtagataatatcaaatactggcatgacagctgcaggtggctgcagtcaaaatataagaacggacataactttcgttatttcggaatggcaggcgtgaaagatattgcggaagccctgtacgggacggagcagaaaggaattatttcccttggaggcaatacaagaatgtatgctgaagtatgcaagaggcttttgccctgtatatctgagaggagacgcgtgccgagagatatagtaaatgccgcggtacaaaaagcgtcatccccagtttcgtatgacaatcggtataattgggaaaaagtgctttctatagcatgcgctctggtgaaaaagcagaaaatggaagtggaaaaggaggagtggaaagtgtcgctgaatgaggaaagcaggaatagagactatctgtacggacgacttttagcaatggctgacaggattgaataccgtacatttgacagagatgaagacgggaaaagagtgacgaatgcaaaaagatatatgaatgcatttgcacagcatccataccagacatggaaggtattagaggagcgtatacagccctatttgcaaaaattggatattaaggagcggaattcttataataagactcttgatgaaatctatgaattgttcgatgaaaaagaatttactaataatgacaggctggaaggattatatttactgggctatcacagccagtcttatgaactaaaatataggccgaaaaaagcggaggaggaaaaagaatga

SEQ ID NO: 11 - DNA, 855 bp, Clostridium scindens
  source 1..855: mol_type = genomic DNA
atgacaactttaaaaggaaaaattgattttgcattgcttgtttcagcaacaaatgctaatccgaatggagatcctttgaatggaaatcgtccaagagagaattatgaaggctatggggaaatctcagatgtctgtattaaaagaaagatccgtaatcgtttccaggatatgggacagaaaatatttgtccaatcggatgaccgttccgacgatggttacacaagcctgaagaaacgggcagaaggatgtgaagaattaaataatgaaattaagaagaaaaaagccgatagggaacattttgcccagattgcatgtcaggaatggctggatgtgaggacgttcggacaagtattcgcgttcaaaggagaggaagtttctgtgggaatacgaggacctgtttcaatccatcaggcagtcagtttgtcaccagtggatgttgtcagcatgcagatcacgaagagcgtcaacagcgaggcaggaaaggacagtaaggcttcagataccatgggaaccaagcatagagtagggtttggactatacctggtaaaaggaagcgtgaatgtccaattggcggagaaaaccggattttctcaagaagatgctgaacttttaaaggaggcgctgaggaccctgtttgaaaacgacgcatcatccgcaaggccggaaggaagtatggaagtctgccgactctactggtggcagcatgaggaaaagactccttcagtacctactgcccaaattcatagaagtataaaaattgaggcattggaagacaggcctaaatcatttaaagattactctatcaagttggaagaactgaactgtgtaaaaccggaggtatatgagtttgtataa

SEQ ID NO: 12 - DNA, 663 bp, Clostridium scindens
  source 1..663: mol_type = genomic DNA
atgagtttgtataaagaagaggattacctgcaattgtcgggtatccagcattttgcattctgccgccgccaatgggcgcttgcctacatagaactccaatgggatgaaaatgtgaggactgttgaaggaaaacttttacatgagaaggcacatgatcctacaaacgcggagaagagaggagatctgattatcagcagaggcatgccagtatattccagagaattggggatcagcggtgaatgtgacgttgttgaattccaccgctctcaggacggaataaccttatttggacgggatggcctgtatgaagtaattcccatcgaatacaagcggggaaagcccaaggaaaatgatgtggatattcttcagcttatggcgcaggcattatgcttggaagaaatgctttgctgccagatttcctttggatacttatactatggagagacaagacatagaaaaatagtagaatttacagaagaaattcggcacaagacaaaagacatgtttctggaaatgcatcagtattatgaacgaagatatactccaagggtaaaacgttccaaggcatgtaatgcgtgttctctaaaggatatctgtctgccggctttaggaatagcaaagtcggcaacagattatattaatagcaggctatcggaggaatag

SEQ ID NO: 13 - DNA, 1032 bp, Clostridium scindens
  source 1..1032: mol_type = genomic DNA
ttgaaaaaattaatgaatacgttatatgtgacctctgcgaatcggtatctttctttagatggagagaatgttgttattctggaagaacagaaggaaatcgggcgcgtgcccctacataacctggagggaatcgttacatttggatatacaggagcaagcccgagacttatgggaacttgcgcgcagaagaatattagccttacttttatgactgagcacggacgtttccttgcacgggtatgtggagaagtcaggggtaatgtcacactgcgcaggcagcaatatcgtatcgccgataatacggaaaaaaggatagaagtggcgcgaaattgtattgttggaaaagtctataattcccggtggattttagagcgggcaatccgagattatccgctgcggctggatgtagaaaagataaaagaaaaatcgtctttccttgcttcccgcttaaaagaaatccgtacgtgtgaaagcccggacaaacttctagggcttgagggagagtccgcctccgtttatttctctgtctttgatgaattgatcttgcagcaaaaggaaactttttattttcatggacgaaatagaaggcctcctttggataatgtaaatgcaatgctttcattcgcctattcgcttttggcaggtatatgtacgtctgccttggaaactgttggccttgatccgtatgttggcttctttcatacggatagaccaggccgcgcatcattggcgctggatatcatggaagaactaagaagtgtaatggctgaccgtttcgtactaacgctgattaataaaaggattgttgattccagcggattccttcagaaagaaaatggcgctgtcattatggacgatgatacgagaaagaaatttctcgctgcatggcaggataagaagaaggaagtcattacacatcctttcctgggtgaaaagatggagtgggggatggtaccgcatgttcaggcaatgcttctggccagatatattcgtggagatctggaagagtatccgccattcttatggaagtag

SEQ ID NO: 14 - DNA, 291 bp, Clostridium scindens
  source 1..291: mol_type = genomic DNA
atgctggtgttaataacttatgatgtgaatacagagacagaggcaggaaagagacgtctaaggaaagtagcaaagcaatgcgttaattatggacagcgggttcaaaattccgtgttcgagtgcaatctggacgccgcaaaacttcgccaggtaaaggctatcttggaagacataattgataaagaggttgatagtttaagattttataatctaggtaataagtataaagacaagattgaacatataggagcaaagaaaagttttgatgtgactgcccctcttatattctag

SEQ ID NO: 15 - DNA, 33 bp, Clostridium scindens
  source 1..33: mol_type = genomic DNA
gtcgttccctgcaatgggaacgtggattgaaat

SEQ ID NO: 16 - DNA, 33 bp, Clostridium scindens
  source 1..33: mol_type = genomic DNA
gcgttgttcccatgcgggaacttggattgaaat

SEQ ID NO: 17 - DNA, 33 bp, Clostridium scindens
  source 1..33: mol_type = genomic DNA
gtctctccctgtatagggagagtggattgaaat

SEQ ID NO: 18 - DNA, 33 bp, Clostridium scindens
  source 1..33: mol_type = genomic DNA
gtctttccctgcatagggagagtggattgaaat

SEQ ID NO: 19 - DNA, 32 bp, Clostridium scindens
  source 1..32: mol_type = genomic DNA
gtctccacctgtgtggtggagtggattgaaag

SEQ ID NO: 20 - protein, 725 aa, Clostridium clostridioforme
MQYYAHISDDKARRQTVKEHLTGTARRSELFAEAFRCGAWGYGCGILHDIGKYSQGFRKRLEGGSITDHATAGAQEMMGLYARTNPLAAYCAAYCISGHHSGLLDGGRNGDTAGEATLQGRLKKQLEDYSPYKDEVEMPDFPGLPFRQIGKGGFSLSFFIRMLFSCLVDGDYLDTEQFMLGHDAGRGDHDCMDVLLRRLESHVESWLSNDDLISVNGRRTAILKACLQAGRRKQGLYQLTVPTGGGKTVSSLGFALRHAVEHGLDRIIYVIPYTSIIEQNAQVFKEILGKKNVLENHCNVTYEDNESARELKLEQLAAENWDKPVVVTTNVQFFESLFASRTSACRKLHNIANSVIIFDEAQMLPVKYLMPCIRAVSELICNYHCTAVICTATQPSLQPFFPAKIQELEAAELCPDVKGQYDFFKRTRIHLAGRISQEHLVKELEGRHQVLCILNSRKRVQRIYESIEDRGTYHLSTLMYPKHRKELLKEIRGRLSSGEPCRLISTSLVEAGVDFDFPAVYRELAGIDSVIQAAGRCNREGKRDPEECITQVFTLEEEEDIHIPQELKLPISVAGQIVEKYEDISSPEAIGEYFTRLYRYKGEGLDAKDVVEQFEQGSHSFMFPFASVASEFRLIESNTRTILIDTEPQAAQIAMQIRRGEHNRELIRQAGQYCVNVFEHDFEALNGAGRLEQIDQEFYVLRNKEQYTRERGLVIDVSRGDAVMF

SEQ ID NO: 21 - protein, 219 aa, Clostridium clostridioforme
MSRGVRVRMWGDYALFSRPEMKVERCSYDVMTPSAARGMLEAIYWHPGMRWVIDKIYVRKPIQFTSIRRNEVKSKVLAGNVLSAINGGGKPLYISSREEIVQRAAILLRDVEYVVEAHFEMTPKAVPGDNEGKFKDIIMRRLRKGECYHRPYFGCREFPAQFALYEEDEIDTAYAGTERDLGYMLFDFDYSDTEDIKPMFFRAVMKDGVLDVRNCEVVR

SEQ ID NO: 22 - protein, 591 aa, Clostridium clostridioforme
MILQALVEYYEALERKGKITSPGWCRAKVAYGLDISEQGELKSVIPLKQERQKGKKTVTVPQNLTVPQMVSRSSGVSANFLCDHSGYMLGIDNKGKPERSRECFECAKKKHKDILEHISSPAARAVCRFFDRWAPDQAAENPCLIPELEEIISGSNLVFMMDGEYVHEDPDIRQCWEEYSHQSGTGPVGVCLVTGRREEIARIHGTIKGVQGAQSSGAALVSFNAPAFESYGKEQSFNAPVGTYAAYAYTTALNYLLGDRTHGTTIGDTAVVYWSEEGDERYQNIFACVSEPTMENQEIVDGVFKNLAEGKAVAVQDVQDSLDMDRKFYILGLAPNAARLAVRFFYQDSFGNILKHIKEHYDRMEIVRPAADAVEYLGMWRMLQETVNKKSRDKKPVPSMSGAVYRAIISGSRYPASLYQAVLGRIRAEQDDGDSRIYKITRGRAAIIKAYLLKNGNEKEEITMALNEDSNNTAYILGREFAVLEAIQEDVNPGINATIKDKYFNSACATPAAIFPILFKLKNSHIKKMNNGAKEIYYEKILGDLQGRLTVAEGQKAACPRRLTLEEQGMFILGYYHQTQKRYEKKIKEEA

SEQ ID NO: 23 - protein, 296 aa, Clostridium clostridioforme
MAEVIKNRYEFVVLFDVENGNPNGDPDAGNLPRIDPESGYGIVTDVCLKRKIRNYVETLKEDEPGYKIYIREDVPLNRSDNTAYIELKTDEKGIKELKKKDPQVDQKIRDFMCRNFFDIRAFGAVMTTFVKAALNCGQVRGPVQLGFARSIDPIVSQEVTITRVAITTEKDAENKSTEMGRKNIVPYALYRAEGYISANLARKSTGFSEEDLELLWDAIINMFEIDHSAARGKMAVRRLIVFKHSKELGDCPAYKLFDAVEVKKNEEVEYPRKYGDYTVAVHRDQIPETVEVSEKI

SEQ ID NO: 24 - protein, 220 aa, Clostridium clostridioforme
MEYREDDFLMVSGIQHFTFCRRQWALIHIEQQWKENEHTIEGELLHKKAHDPYLAERRGDVMISRALPVYSRSMGVSGECDIVEFHRAEDGIGLHGHRGLFRVFPVEYKRGSPKESEADILQLVAQAMCLEEMLSARIEAGALYYGEIHRRNVVEITDELRKRVREIFQEMHELYDKGYTPKVRWSKSCNACSMKDICMPKLGKASPVRDYIRGKIEEDV

SEQ ID NO: 25 - protein, 343 aa, Clostridium clostridioforme
MRKLLNTLYVTSPDGYLSLDGENVVIYDNDTELGRIPLHNLEGIVSFGYRGMSPALMGGCAERDISLCVLSPQGRFLARVTGRVRGNVLLRRKQYQVSMNQDASLMIAKNCILGKVYNARWVLERAVRDHGLQIDTEKVKDAAGFLKQSLRRIEESRDMAQLRGYEGEDASIYFGVFDQLILQQKKDFYFKGRNRRPPLDNVNAMLSFVYTLLANTVASALETVGLDPYVGFMHTDRPGRLSLALDLMEELRPVLADRFVLTLINRKMVNKKDFSRKEDGAVIMGDEARKLLLSEWQNKKKEVITHPYLNEKVEWGMIPFVQAMLLSRYLRGDIDEYPPFFWK

SEQ ID NO: 26 - protein, 96 aa, Clostridium clostridioforme
MLVLITYDVNTETAAGRARLRKVAKQCVNYGTRVQNSVFECILDNAQLIKLKAILTDIIDEQTDSLRFYNLGNKHTTKVNHVGMNKGINVEEPLIF

SEQ ID NO: 27 - DNA, 2178 bp, Clostridium clostridioforme
  source 1..2178: mol_type = genomic DNA
atgcaatactacgcgcacatcagcgacgacaaggcgcgaaggcagacagtgaaggagcatctgacagggacggccaggcgttctgaactgttcgcagaggctttccggtgcggagcatgggggtatggatgcggaattctgcacgacattgggaaatattcccagggattcagaaagcgtctggaaggcggttccattacagaccatgccacggcgggggcccaggagatgatggggctgtatgccaggaccaacccgctggctgcttattgcgccgcttactgtatcagcggccaccattcgggacttctggacggaggaaggaacggcgacacggcaggggaggccacgcttcagggaaggcttaagaagcaattagaggattacagtccatacaaggatgaagttgagatgccggattttcccgggctgccgttcagacagataggcaagggaggattcagcctgtcatttttcatcagaatgctgttttcatgccttgtggacggagattatctggatacggagcaatttatgctgggacacgatgcgggaaggggagaccatgattgtatggacgttcttctcaggcgtctggaaagccatgtggaatcctggctttccaatgatgatttgatatctgtaaatggaagaaggacggctatactgaaggcatgtctgcaggcaggcagaaggaagcaggggctgtaccagctcacggtacctaccggcggaggcaagaccgtatcctcccttgggtttgccctgcgccacgcagtggaacatggtcttgaccggattatttatgtgatcccgtatacgagtatcatcgaacagaacgcccaggtatttaaggaaattctgggaaagaaaaatgttctggaaaaccattgtaatgtgacgtatgaggacaatgagagcgcccgggagttaaaattggaacagttggccgccgagaattgggataagccggtggtggtcactacaaatgtccaattttttgaatcactctttgcctccaggacttcggcatgccgcaaactgcataacattgccaacagtgtgatcatatttgatgaggcacaaatgctgccggtgaaatatctgatgccatgtattcgggctgtcagcgagcttatatgtaattatcactgcacggctgttatctgtacggctacccagccgtccctgcagcccttctttcctgcaaagatacaggaactggaggcggcggaactatgtccggatgtaaaaggacaatatgattttttcaaaagaacaaggatacacctggcaggacggatatcccaggagcatctggtgaaggagctggaaggccggcatcaggtcctttgtatcctgaacagcagaaagcgtgtacagcgtatctacgagtccattgaggaccggggaacgtatcatttatccacattgatgtatccaaagcacaggaaggagcttttgaaggagattcgcggccgtctgtcatcaggagaaccgtgccggcttatttccaccagcctggtggaagcaggcgtggattttgattttccggccgtatacagggagctggccggaattgacagcgtgatccaggctgccggaaggtgcaacagggagggaaagagggatccggaagaatgcattacccaggtatttactttggaggaagaggaggatatccatataccacaggaattaaaactgcctatttcagttgcggggcagattgtggaaaagtatgaagatatatcatcgccggaggccattggagaatatttcaccaggctgtaccggtataaaggggagggattggacgcaaaggatgtggtggagcagtttgagcagggaagccattccttcatgtttccatttgcatcggtggcttccgagttccgcctgattgagagcaataccaggaccattttaattgatacagagccgcaagcagcgcagattgccatgcaaatccggcggggagagcataacagggagcttatcaggcaggcgggacagtactgtgtaaatgtttttgaacatgattttgaagcgttaaacggcgcgggccgccttgaacagatagaccaggaattttatgttctgaggaataaggagcagtatacaagggagagaggacttgtgattgacgtaagccgcggcgatgccgtcatgttttga

SEQ ID NO: 28 - DNA, 660 bp, Clostridium clostridioforme
  source 1..660: mol_type = genomic DNA
atgagccgaggagtacgggtccggatgtggggggattatgcccttttcagccgccctgagatgaaggtggaacggtgcagctatgatgtgatgactccctccgccgcaaggggtatgctggaggcgatctactggcatccggggatgcggtgggtgattgacaaaatatatgtgagaaaacccattcagtttaccagcatccggagaaatgaggtaaagagcaaggtattggcaggaaatgtattgtctgctataaacgggggaggaaaaccgttgtatataagcagcagggaggaaatcgtacagagagcggccattcttctgcgggatgtggaatatgtggttgaggctcactttgagatgacccccaaggcagtcccaggggataatgagggtaagtttaaggacattattatgcgccggctccgcaagggtgagtgctatcacagaccgtattttggatgccgggaatttcctgcccagtttgccctttatgaagaagatgagatagacactgcatatgccggaacggagagggatttggggtatatgctgtttgactttgattactcagatactgaggacattaaacccatgtttttcagggccgtcatgaaggatggtgttctggatgtaaggaattgtgaggtggtgcgatga

SEQ ID NO: 29 - DNA, 1776 bp, Clostridium clostridioforme
  source 1..1776: mol_type = genomic DNA
atgattctccaggcgcttgttgaatattatgaagcccttgaacggaagggaaagataaccagtccgggctggtgcagggcaaaggtagcgtacgggctggatatttcagagcagggggagcttaagagcgtcattccccttaagcaggagcggcagaagggtaaaaagacagtcacggtaccgcagaaccttacggtaccccagatggtttcccgttcttccggtgtatccgccaatttcctgtgcgaccattccggttatatgctgggaattgataataaggggaaaccggagcggtccagggaatgttttgagtgcgccaaaaagaagcataaggacattttagagcatatcagcagccccgcggccagggcggtgtgccggttttttgaccgctgggcgccggatcaggcagcggaaaacccatgcctgataccggaactggaggagattatatctggttctaatctggtgttcatgatggatggggaatatgtccatgaggacccggatataaggcagtgctgggaagaatacagccatcagtcaggcacaggacctgtgggcgtctgcctggtgacagggcgcagggaggaaatagccaggattcacggcaccataaaaggagtccagggggcccagtccagcggtgctgcgctggtttcctttaatgcacctgcctttgaatcatacggcaaggaacagagctttaacgcaccggtgggaacatatgcggcctatgcctataccacggcgctcaattatctccttggggaccggactcatgggacaaccatcggtgacacggcagtggtttactggtccgaggaaggcgacgaacgataccagaatatatttgcgtgcgtatcggagccaacgatggagaaccaggaaatagtggatggggtattcaagaacctggcagagggaaaagcggttgcggttcaggatgtccaggacagtcttgatatggacaggaaattttatatattaggtctggcgcctaatgcagccaggctggcagtgcgttttttctatcaggacagctttggaaatatattaaagcatataaaagaacattatgacagaatggaaatagtgcgtccggcagccgacgccgtggagtatctgggaatgtggcgtatgctgcaggagaccgtcaataaaaagtccagggataaaaagccggttcccagtatgtccggggcagtgtacagggcaatcatatccggcagccggtatccggcgtccctgtatcaggccgtgcttggcaggatacgggcagagcaggacgacggggacagccggatttataagataaccaggggaagggcagccattatcaaagcatatctgctgaaaaatggaaatgaaaaggaggaaataaccatggcgctgaatgaagacagtaataataccgcgtacatcctgggaagggagtttgcggttctggaggcaatccaggaggacgtcaatccggggattaacgccaccataaaggataagtattttaactctgcatgcgcgacgccggcggcaatctttcccattttatttaagctgaaaaacagccacataaagaagatgaataatggggcgaaggagatatattatgaaaaaatactgggtgatttacagggaaggctgactgtcgcagagggacagaaggcagcctgcccaaggcggcttaccctggaggagcagggaatgttcatattgggatattaccatcagactcagaagagatatgagaagaagataaaggaggaggcttaa

SEQ ID NO: 30 - DNA, 891 bp, Clostridium clostridioforme
  source 1..891: mol_type = genomic DNA
atggcagaggtgattaagaacaggtatgaatttgtggtgttatttgatgtggagaacgggaatccaaacggcgacccggatgcaggcaatctgcccaggattgatccggagagcggatacggtatcgtgacggatgtgtgcttaaaacgtaagatacgcaattatgtggagacactgaaggaagacgagcccgggtataaaatatatatccgggaggatgtacccttaaacagaagtgacaataccgcgtacattgagcttaagacagatgaaaagggaatcaaggaactcaaaaagaaagaccctcaggtagaccagaaaatacgggattttatgtgccgtaactttttcgatatccgagcctttggcgcggtcatgacaacctttgtaaaggcagcgttaaactgcggccaggtgcgtgggcctgtacagctgggatttgccagaagcattgaccccattgtgagtcaggaagtgaccattacccgtgtggctatcaccacagaaaaggacgcggaaaataagagtacggaaatgggaagaaagaatatcgttccctacgccctgtaccgcgcggagggttacatatcagctaatctggccagaaaatccacgggcttttcagaggaggatttggaacttttgtgggatgccatcatcaatatgtttgaaatcgaccattccgcagccaggggaaaaatggcggtccgccggctcatcgtgtttaaacatagtaaggaattgggagattgccctgcatacaaactctttgatgccgtggaagtaaaaaagaatgaggaagtggagtatcccagaaaatacggggattacacggttgccgtccatagggaccagataccggaaacggttgaagtttcggagaagatataa

SEQ ID NO: 31 - DNA, 663 bp, Clostridium clostridioforme
  source 1..663: mol_type = genomic DNA
atggaatacagggaggatgatttcctcatggtgtccggtatccagcatttcaccttttgcaggcgccagtgggcccttatacatattgaacagcagtggaaagagaatgagcataccatagaaggagagctgctccacaaaaaggcccatgacccatatttggcagaaaggcggggcgatgtgatgataagcagggcattgcctgtgtattccaggagtatgggtgtcagcggggaatgcgatattgtagagtttcacagggcggaggatggaatcggactccatgggcacaggggactgttccgggtatttccggtggaatataaaagggggagtccaaaggaatcagaagcagatatactgcagctggtggcacaggccatgtgtctggaggaaatgctgtcagccaggattgaagcgggggcgctgtattacggagagattcaccgcagaaatgtggtggaaattacagatgaactgcgaaaacgggtaagggagatatttcaggagatgcatgaactttatgacaagggatatacaccaaaggtcagatggtcaaagagctgtaatgcctgctccatgaaagatatatgtatgccgaaactgggaaaagcatcacctgtcagggattatatccgtgggaagattgaggaggatgtatga

SEQ ID NO: 32 - DNA, 1032 bp, Clostridium clostridioforme
  source 1..1032: mol_type = genomic DNA
atgaggaagttattaaacaccctgtatgttacttctcctgatggttatctgtcgctggacggggaaaatgtggtaatctatgacaacgatacggaactgggacggataccgctccataatctggagggaatagtgtcctttggatacaggggaatgagcccggccctcatggggggatgcgcggagcgggacatatcactttgcgtcctttcgcctcagggaagatttctggcccgtgtgaccggacgtgtgcggggaaacgtacttctgcgcaggaaacagtaccaggtttccatgaatcaggacgcaagtctgatgatagccaaaaactgtatccttggaaaagtatacaatgccagatgggttctggagagagctgtgagagaccatggacttcagattgacacagagaaggtgaaggatgcggccggattcctgaaacaatccctcaggcgcatagaggaaagccgggacatggctcagctgcgggggtatgaaggggaggatgccagtatttactttggggttttcgaccagctcatattacagcagaaaaaggatttctatttcaagggaaggaacaggcggccgcctttggataatgtgaatgccatgctgtcatttgtgtacacgctgctggccaatacagttgcgtcagcattggaaacagttggcctggacccttacgtgggattcatgcatacggaccggccaggccgcctttccctggcattggatttaatggaggagctgcggccagttctggcagaccgttttgtgcttaccctgattaaccggaagatggtaaacaaaaaggatttttcccggaaagaggacggcgctgtcattatgggggatgaggcacggaagctgttgctgtcagaatggcagaacaagaaaaaggaggtgattacccatccatatctgaatgagaaggtggagtggggaatgattccatttgttcaggccatgcttctgtccagatatctgcgaggcgatatagatgaatacccgccgtttttctggaaatag

SEQ ID NO: 33 - DNA, 291 bp, Clostridium clostridioforme
  source 1..291: mol_type = genomic DNA
atgctggtactgattacatatgatgtgaatacggaaaccgccgcaggcagggccaggttgagaaaggttgcaaagcaatgtgtcaattatggcacccgcgtccagaactccgtatttgaatgtattttggataatgcccagctgataaagctaaaggcgatactgacagatataattgatgaacagacggacagccttaggttttacaaccttgggaataaacacactacaaaagtgaaccatgttgggatgaataaggggattaatgtggaagaaccgctgatattctga

SEQ ID NO: 34 - DNA, 32 bp, Clostridium clostridioforme
  source 1..32: mol_type = genomic DNA
gtctccaccctcgtggtggagtggattgaaat

SEQ ID NO: 35 - DNA, 33 bp, Clostridium clostridioforme
  source 1..33: mol_type = genomic DNA
gtcgaggcccgcgagggccttgtggattgaaat

SEQ ID NO: 36 - protein, 725 aa, Clostridium bolteae
MQYYAHISEDKTRKQTVKDHLTGTARRSELFAEAFRCGAWGYGCGVLHDIGKYSQGFRKRLEGGSITDHATAGAQEMMRLYAGSNPLAAYCAAYCISGHHSGLLDGGRTGDTAGEATLQGRLKKQLEDYSPYKNEVEMPDFPGLPLRQIGKGGFGLSFFIRMLFSCLVDGDYLDTEQFMLGQDAGRGDYDCMDVLLRRLESHVESWLSNDDLTSVNGRRTAILKACLQAGRRRQGLYQLTVPTGGGKTVSSLGFALRHAAEHGLERIIYVIPYTSIIEQNAQVFKEILGKKNVLENHCNVTYEDKESGKELKMEQLAAENWDKPVVVTTNVRFFESLYACRSSECRKLHNIANSVIIFDEAQMLPVKYLMPCIRAISELICNYHCTAVICTATQPSLQPFFPAKMQELKAEELCPDVKGQYDFFKRTKIQLAGRISQEHLVKELEGRQQVLCILNSRKRVQRVYDSIDSRGTYHLSTLMYPKHRKEILKGIRSRLSSGEPCRLISTSLVEAGVDFDFPAVYRELAGIDSVIQAAGRCNREGKRDPEECMTQVFTLEEEEDIHIPRELKLPISVAGQIAQKYEDISSPEAIGEYFTRLYRYKGEGLDAKDVVEQFEQGSRSFMFPFASAASGFRLIESNTRTILIDTEPEAAQIAMQIRQGGHSRELIRQAGQYCVNVYEHDFEALSGAGRLEQIDREFYVLRNKEQYTRERGLVIDVSRGDALMF

SEQ ID NO: 37 - protein, 219 aa, Clostridium bolteae
MSRGVRVRLWGDYALFSRPEMKVERCSYDVMTPSAARGMLEAIYWHPGMRWVIDKIYVRKPIQFTSIRRNEVKSKVLAGNALTAVNGGGKPLYISSKEEIVQRASILLRDVEYVVEAHFEMTPKAVPGDNEGKFKDIIMRRLRRGECYHQPYFGCREFPARFALYEEEEVDTAYDGTERDLGYMLYDLDYSDTEDIKPMFFRAVMKDGVLDVRNCEVVR

SEQ ID NO: 38 - protein, 591 aa, Clostridium bolteae
MILQALVEYYEALERKEKITSPGWCRARVAYGLDISEQGELMGVIPLKKEQQNGKKTVWVPQDLTVPQMVSRSSGVSANFLCDHSGYMLGIDNKGKPERSRECFECAKKKHKDILGNISSPAARAVCRFFDCWAPDRAAENPYLVPELEEIISGSNLVFMIDGEYVHEDPDIRQCWEEYSCQSGTGPEGVCLVTGRREEIARIHSTIKGVQGAQSSGAALVSFNAPAFESYGKEQSFNAPVGTYAAYAYTTALNYLLSDRSHGTTIGDTAVVYWSEEGDERYQNIFACVSEPTMENQEIVDGVFKNLAEGKAVVAQDVKDSLDMEKRFYILGLAPNAARLAVRFFYQDSFGNILKHIKEHYDRMEIVRPAADAVEYLGIWRMLQETVNKKSRDKKPVPSMSGAVYRSIISGSRYPASLYQAVLGRIRAEQDDGDSRIYKITRGRAAIIKAYLLKNGNIREEITMALNEDSNNTAYILGREFAVLEAIQEDANPGINATIKDKYFNSACATPAAIFPILFKLKNSHIKKMNNGAKETYYEKMLCDLQGRLTVAEGQRAACPRRLTLEEQGMFILGYYHQTQKRYEKKIKEEA

SEQ ID NO: 39 - protein, 296 aa, Clostridium bolteae
MAEVIKNRYEFVVLFDVENGNPNGDPDAGNLPRIDPESGYGIVTDVCLKRKIRNYVETLKEDEPGYKIYIREDVPLNRSDNTAYIELKTDEKGIKELKRKDPQVDQKIRDFMCRNFFDIRTFGAVMTTFVKAALNCGQVRGPVQLGFARSIDPIVSQEVTITRVAITTEKDAENKSTEMGRKNIVPYALYRAEGYISANLARKSTGFSEEDLELLWDAIINMFEIDHSAARGKMAVRRLIVFKHSKELGDCPAYKLFDAVEVKRNEDVEYPRKYGDYTVAVHRDQIPETVEVSEKI

SEQ ID NO: 40 - protein, 220 aa, Clostridium bolteae
MEYREDDFLMLSGIQHFIFCRRQWALIHIEQQWKENEHTIVGELLHKKAHDPYLAEKRGDVMISRALPVYSRSMGVSGECDIVEFHRAEDGIGLHGHRGLFRVFPVEYKKGSPKESEADILQLTAQAMCLEEMLSARIEAGALYYGEIHRRNMVEITDELRKRVRDIFQEMHELYDKGYTPRVRWSKSCNACSMKDICLPKLGKASSVRDYIRGKIEEDV

SEQ ID NO: 41 - protein, 343 aa, Clostridium bolteae
MRKLLNTLYVTSPDSYLSLDGENVVIYDNDTELGRIPFHNLEAIVSFGYRGMSPALMGGCAERDISLCVLSPQGRFLARVTGRVRGNVLLRRKQYQVSMNQDASLTIARNCILGKVYNARWVLERAVRDHGLQIDTEKVKEAAGFLKQSLRHIEESRDMAQLRGYEGEDAGIYFGVFDQLILQQKKDFYFKGRNRRPPLDNVNAMLSFVYTLLANTVASALETVGLDPYVGFMHTDRPGRLSLALDLMEELRPVLADRFVLTLINRKMVNKKDFSRKEDGAVIMRDEARKLLLSEWQNKKKEMITHPYLNEKVEWGMIPFVQAMLLSRYLRGDIDEYPPFFWK

SEQ ID NO: 42 - protein, 96 aa, Clostridium bolteae
MLILITYDVNTETTAGRTRLRKVAKQCVNYGTRVQNSVFECILDNTQMIKLKAILTDIIDEQTDSLRFYNLGNKHTTKVDHVGVSKGIKVEEPLIF

SEQ ID NO: 43 - DNA, 2178 bp, Clostridium bolteae
  source 1..2178: mol_type = genomic DNA
atgcaatactatgcgcacatcagtgaggacaagactcgaaaacagaccgtaaaggaccatctcacagggaccgccaggcgttctgaactgtttgcagaagcgttccggtgcggagcatggggatacggatgcggagttctgcacgacatcggaaaatattcccagggatttagaaaacgtctggagggcggttccattacggaccacgccacggcgggagcccaggagatgatgcggctgtatgccgggtccaatcctctggctgcttactgcgccgcttattgcatcagcggccaccattcaggccttctggacgggggaaggaccggagacacggcaggggaggccacgcttcagggaaggcttaaaaagcagttagaggattacagtccgtataaaaatgaagttgagatgccggattttcccgggctgccgctgagacagataggcaagggcggctttggcctgtcattttttattaggatgctgttttcatgccttgtggacggagattatctggatacggaacaattcatgctgggacaggatgcgggaaggggagactatgattgtatggatgtccttctcaggcgtctggaaagccatgtggaatcctggctttccaatgatgatttgacatctgtaaatggaagaaggacggctatactgaaggcttgtttgcaggcaggacgcaggaggcagggactgtaccagctcacggtacctaccggcggaggcaagaccgtgtcctcccttgggtttgccctgcgccacgcggcggaacatggtcttgaacggattatttatgtgatcccgtatacgagtatcatcgaacagaacgcccaggtatttaaggaaatcctgggaaagaaaaatgtcctggaaaaccattgtaatgtaacatatgaggataaggagagcggcaaggaattaaaaatggagcagctggccgcagaaaactgggacaagccggtggtggtcactacaaatgtccggttttttgaatctctctatgcctgcagaagctcggaatgccgtaaactgcataatattgccaacagcgtcatcatatttgatgaggcgcaaatgcttccggtgaaatatctgatgccgtgtattcgggctatcagtgagcttatatgcaattatcactgcacagcagttatctgtacagctacccagccgtccctgcagcccttttttcctgcaaagatgcaggaactgaaggcagaggaactatgtccggatgtaaaaggacagtatgattttttcaaaaggacaaagatacagctggcagggcggatatcccaggaacatctggtgaaggagctggaaggccggcagcaggtcctctgtatcctgaacagcagaaaacgggtgcagcgcgtctacgattccattgatagccgggggacgtaccatttatctaccttgatgtatccaaaacacaggaaggagattttgaaggggattcgcagccggctgtcgtcgggagaaccatgtcggcttatatccaccagcctggtggaagcaggcgtggattttgattttccagccgtatacagggaactggccggaattgacagtgtgatccaggctgccggaaggtgcaaccgggagggcaagagggatccggaagaatgtatgacccaggtatttacgttggaggaagaggaggatatccatataccgcgggaattgaaactgcccatttcagtagccggacagattgcgcagaagtatgaagatatatcgtcgccggaggccataggagaatatttcaccaggctgtaccggtataaaggggagggattggacgcaaaggatgtggtggagcagttcgagcagggaagccgttcatttatgtttccatttgcatcagcagcttccgggttccgcctgattgagagcaataccaggaccattttaattgatacagagccggaggcagcgcagattgccatgcaaatccggcagggcgggcatagcagggagcttatcaggcaggccggacagtactgtgtaaatgtttatgaacatgattttgaagccctgagcggcgcaggccgcctggaacagatagaccgggaattttatgttctgaggaataaggagcagtatacaagggagagggggctggtgattgacgtaagccgcggggatgcccttatgttttaa

SEQ ID NO: 44 - DNA, 660 bp, Clostridium bolteae
  source 1..660: mol_type = genomic DNA
atgagccgaggagtacgggtccggctgtggggcgattacgccctcttcagccgccctgagatgaaggtggaacggtgcagctatgatgtgatgacgccttccgccgcaaggggaatgctggaggcgatctactggcatccggggatgcggtgggtgattgacaaaatctatgtgagaaaacccatccagtttaccagcatccgaagaaatgaggtaaagagcaaggtattggcaggaaacgcattgactgccgtaaacgggggaggaaaaccgctgtatataagcagcaaagaggaaatcgtacagagggcgtccattcttcttcgggatgtggaatatgtggttgaggcccatttcgagatgacccccaaggcagtcccgggggataacgagggtaagtttaaggacattatcatgcgccgcctgcgcaggggagagtgttatcatcagccgtattttggatgccgggaattccctgcccggtttgccctctatgaagaggaggaagtagacactgcatatgacggcacggagagagatttgggatatatgctgtatgatctggattactcagatactgaggacattaaacccatgtttttcagggctgtcatgaaggatggtgttctggatgtaaggaattgtgaggtggtgcgatga

SEQ ID NO: 45 - DNA, 1776 bp, Clostridium bolteae
  source 1..1776: mol_type = genomic DNA
atgattctccaggcgcttgttgaatattatgaagcccttgagcggaaggaaaagataaccagtccgggctggtgcagggccagggtagcgtacgggctggatatttcagagcagggggagcttatgggcgttatcccccttaagaaagagcagcagaacggcaaaaagacagtctgggtaccccaggaccttacggtaccccagatggtttcccgttcttccggtgtatctgccaatttcctgtgcgaccattccggttatatgctgggaattgataataagggaaaaccggagagatccagggaatgttttgagtgcgctaaaaagaagcataaggacattctggggaatatcagcagtcccgcagccagggcggtgtgccgcttttttgactgctgggcgccggaccgggcagcagaaaacccatacctggtgccggaactggaggagatcatatccggttctaatctggtgtttatgatagacggggaatatgtccatgaggacccggatataaggcagtgctgggaagaatacagctgtcagtcaggtacaggacctgagggcgtctgcctggtgacagggcgcagggaggaaatcgccaggattcacagcaccatcaaaggcgttcagggagcccagtccagcggggctgcgctggtttcctttaacgcgcctgcctttgaatcttacggcaaggaacagagctttaacgcgccggtgggtacatacgcggcctatgcctataccacggcgctcaattacctcctttcggaccggtcccatgggacaaccatcggcgacacggccgtggtttactggtccgaggaaggggacgaacggtaccagaatatatttgcctgtgtgtcggagccgaccatggagaaccaggaaatagtggatggggtattcaagaacctggcggagggaaaagcagttgtggcccaggatgttaaggacagtctggatatggaaaagcggttttatatattggggctggcacccaacgcagccaggctggccgtgcgtttcttctatcaggacagctttggaaacatattaaagcatataaaagaacattatgacagaatggaaatagtccgtccggcagccgatgctgtggagtatctggggatatggcgtatgctccaggagacggtcaataaaaaatccagggataaaaagccggttcccagtatgtccggggcagtgtacaggtcaattatatccggcagccggtatccggcgtccctgtatcaggccgtgcttggcaggatacgggcggagcaggacgacggggacagccgtatttataagataaccaggggaagggctgccatcatcaaggcatatctgctaaaaaatggaaatataagggaggaaataaccatggcgcttaatgaagacagcaataataccgcgtacatcctgggaagggaatttgcggttctggaggcaatccaggaggatgccaatccggggatcaacgccaccataaaggataaatattttaattccgcatgcgccacaccggcggcaattttccccattttatttaagctgaaaaacagccacataaagaagatgaacaatggggcaaaagagacatattacgaaaaaatgctgtgtgatttacagggaaggctgacggtggcagagggacagagggcagcctgcccaagaaggcttaccctggaggaacagggaatgttcatattggggtattatcatcagactcagaagagatatgagaagaagataaaggaggaggcttaa

SEQ ID NO: 46 - DNA, 891 bp, Clostridium bolteae
  source 1..891: mol_type = genomic DNA
atggcagaggtgattaagaacaggtatgaattcgtggtattatttgacgtggagaacgggaatccaaacggcgatccggatgcaggcaatctgccgaggattgacccggagagcggatacggcattgtgacggatgtgtgcttaaaacgtaagatacgcaattatgtggaaaccctgaaggaagacgaacccgggtataaaatatatatacgggaggatgtacccttaaacagaagcgacaataccgcgtacattgaacttaagacagatgaaaaaggaatcaaggaactaaaaaggaaagaccctcaggtagaccagaaaatccgtgattttatgtgccgtaacttttttgatatccggacctttggagcggtcatgacaacctttgtaaaggctgctttaaactgcggccaggtgcggggacctgttcagttaggctttgccaggagtattgatcccattgtaagccaggaagtgaccattacccgtgtggccattaccacagaaaaggacgcggaaaacaagagtacggaaatgggaagaaaaaatatcgttccctacgccctgtaccgcgcagaggggtacatatcagctaatctggccagaaaatccacgggcttttcagaagaggatttggaacttttgtgggatgccatcatcaatatgtttgaaattgaccattccgcagccaggggaaaaatggcggtacgccggctcattgtgtttaaacacagtaaggaactgggagattgtcctgcgtacaaactctttgatgccgtggaagtaaaaaggaatgaggatgtggagtatcccagaaaatacggggattacacggttgccgtccatagggatcagattccggagacagttgaagtatcggagaagatataa

SEQ ID NO: 47 - DNA, 663 bp, Clostridium bolteae
  source 1..663: mol_type = genomic DNA
atggaatacagggaggatgacttcctcatgctgtccggtatccagcattttatcttttgcaggcgccagtgggcccttatacatattgaacagcagtggaaggagaacgagcataccatagtaggagaactgctccacaaaaaagcccatgacccatatttggcagaaaagcggggcgatgtcatgataagcagggcattgcccgtgtattccaggagtatgggtgtcagcggggaatgcgatattgtagagttccacagggcggaggatggaatcggtctacatggtcacagggggctgttccgggtatttccggtggagtataaaaaggggagcccaaaggagtcggaagcagatatactgcagctgacggcccaggccatgtgtctggaggaaatgttgtcggccagaattgaggcaggggcgctgtattacggagagattcaccgcaggaatatggtggagatcacagatgaactgcgaaaacgggtgagagatatatttcaggagatgcatgaactttatgacaagggatacacaccaagggtcagatggtcaaagagctgtaatgcctgctccatgaaagatatatgcctgccgaaactgggaaaagcatcatctgtcagagattatatccgtgggaagattgaggaggatgtatga

SEQ ID NO: 48 - DNA, 1032 bp, Clostridium bolteae
  source 1..1032: mol_type = genomic DNA
atgaggaagttattaaataccctgtatgttacctctcctgacagttacctgtcgctggacggagaaaatgtggtaatctatgacaacgatacggaactggggcggatacctttccataatctggaggcaattgtttcttttgggtacaggggaatgagccctgccctcatgggcggatgcgcggagcgggacatatcactttgcgtcctttcgccccagggaagatttttggcacgcgtgaccggacgtgtgcggggaaatgtactgctgcgcaggaaacagtaccaggtatccatgaatcaggacgcaagtctgaccatagccagaaactgtatccttggaaaagtatacaatgccagatgggttctggagagagcggtgagagaccatggacttcagattgacacagagaaggtgaaggaagcggccggatttctgaaacaatccctcaggcacatagaggaaagccgggacatggcccagctgcgggggtatgaaggtgaggatgcaggcatttattttggagttttcgaccagctcatattacagcagaaaaaggatttctatttcaagggacggaacaggcggccgcctttggataatgtgaatgccatgctgtcatttgtgtacacgctgctggccaataccgttgcgtcagcactggaaacagttggcctggatccttatgtgggatttatgcatacggaccggccaggccgcctttccctggctctggacttaatggaggagctgagaccggttcttgcagaccgttttgtgctgaccctgattaaccgtaagatggtaaacaaaaaagatttttcccggaaagaagacggtgctgtcatcatgagagacgaggcacggaagcttttgttgtcagaatggcagaacaagaaaaaggagatgatcacccatccatatctgaatgagaaggtggaatggggaatgattccttttgttcaggccatgcttctgtccagatatctgagaggtgacattgatgaatacccgccgtttttctggaaatag

SEQ ID NO: 49 - DNA, 291 bp, Clostridium bolteae
  source 1..291: mol_type = genomic DNA
atgctgatactgattacatatgatgtgaatacggaaaccactgcaggcaggaccaggctgagaaaggttgcaaagcaatgcgttaattacggcacccgcgtccagaattctgtatttgaatgtattttggataatacccagatgataaagctaaaagccatactgacagatatcattgatgagcagactgacagccttagattttacaacctgggaaataaacacactacaaaggtggaccatgttggggtgagtaaagggattaaagtggaagagccgctgatattctga

SEQ ID NO: 50 - DNA, 33 bp, Clostridium bolteae
  source 1..33: mol_type = genomic DNA
gtctccgtcctcgcgggcggagtgggttgaaat

SEQ ID NO: 51 - DNA, 33 bp, Clostridium bolteae
  source 1..33: mol_type = genomic DNA
gtctccgtcctcgcgggcggagtggcttttcct

SEQ ID NO: 52 - DNA, 33 bp, Clostridium bolteae
  source 1..33: mol_type = genomic DNA
gtcgaggctcgcgagagccttgtggattgaaat

SEQ ID NO: 53 - DNA, 33 bp, Clostridium bolteae
  source 1..33: mol_type = genomic DNA
gtcgaggctcgcgagagccttgcagaccaaaag

SEQ ID NO: 54 - protein, 725 aa, Clostridium bolteae; identical to SEQ ID NO: 36

SEQ ID NO: 55 - protein, 219 aa, Clostridium bolteae; identical to SEQ ID NO: 37

SEQ ID NO: 56 - protein, 591 aa, Clostridium bolteae; identical to SEQ ID NO: 38

SEQ ID NO: 57 - protein, 296 aa, Clostridium bolteae; identical to SEQ ID NO: 39

SEQ ID NO: 58 - protein, 220 aa, Clostridium bolteae; identical to SEQ ID NO: 40

SEQ ID NO: 59 - protein, 343 aa, Clostridium bolteae; identical to SEQ ID NO: 41

SEQ ID NO: 60 - protein, 96 aa, Clostridium bolteae; identical to SEQ ID NO: 42

SEQ ID NO: 61 - DNA, 2178 bp, Clostridium bolteae; identical to SEQ ID NO: 43
  source 1..2178: mol_type = genomic DNA

SEQ ID NO: 62 - DNA, 660 bp, Clostridium bolteae; identical to SEQ ID NO: 44
  source 1..660: mol_type = genomic DNA

SEQ ID NO: 63 - DNA, 1776 bp, Clostridium bolteae; identical to SEQ ID NO: 45
  source 1..1776: mol_type = genomic DNA

SEQ ID NO: 64 - DNA, 891 bp, Clostridium bolteae; identical to SEQ ID NO: 46
  source 1..891: mol_type = genomic DNA

SEQ ID NO: 65 - DNA, 663 bp, Clostridium bolteae; identical to SEQ ID NO: 47
  source 1..663: mol_type = genomic DNA

SEQ ID NO: 66 - DNA, 1032 bp, Clostridium bolteae; identical to SEQ ID NO: 48
  source 1..1032: mol_type = genomic DNA

SEQ ID NO: 67 - DNA, 291 bp, Clostridium bolteae; identical to SEQ ID NO: 49
  source 1..291: mol_type = genomic DNA

SEQ ID NO: 68 - DNA, 33 bp, Clostridium bolteae; identical to SEQ ID NO: 52
  source 1..33: mol_type = genomic DNA

SEQ ID NO: 69 - DNA, 33 bp, Clostridium bolteae; identical to SEQ ID NO: 53
  source 1..33: mol_type = genomic DNA

SEQ ID NO: 70 - DNA, 33 bp, Clostridium bolteae; identical to SEQ ID NO: 50
  source 1..33: mol_type = genomic DNA

SEQ ID NO: 71 - DNA, 33 bp, Clostridium bolteae; identical to SEQ ID NO: 51
  source 1..33: mol_type = genomic DNA

SEQ ID NO: 72 - protein, 725 aa, Clostridium clostridioforme
MQYYAHISDDKMRRQTVKEHLTGTARRSELFAEAFRCGAWGYGCGILHDIGKYTQGFIKRLEGGSITDHATAGAQEMMGLYARTNPLAAYCAAYCISGHHSGLLDGGRNGDTAGEATLQGRLKKQLEDYSPYKDEVEMPDFPGLPLRQIGKGGFSLSFFIRMLFSCLVDGDYLDTEQFMLGHDAGRGDHDCMDVLLRRLESHVESWLSNDDLISVNGRRTAILKACLQAGRRKQGLYQLTVPTGGGKTISSLGFALRHAVEHGLDRIIYVIPYTSIIEQNAQVFKEILGKKNVLENHCNVTYEDNESARELKLEQLAAENWDKPVVVTTNVQFFESLFASRTSVCRKLHNIANSVIIFDEAQMLPVKYLMPCIRAVSELICNYHCTAVICTATQPSLQPFFPAKIQELEAAELCPVVKGQYDFFKRTRIHLAGRISQEHLVKELEGRHQVLCILNSRKRVQRIYESIEDRGTYHLSTLMYPKHRKELLKEIRGRLSSGEPCRLISTSLVEAGVDFDFPAVYRELAGIDSVIQAAGRCNREGKRDPEECITQVFTLEEEEDIHIPQELKLPISVAGQIVGKYEDISSPEAIGEYFTRLYRYKGEGLDAKDVVEQFEQGSRSFMFPFASVASGFRLIESNTRTILIDTEPEAAQIAMQIRRGEHNRELIRQAGQYCVNVFEHDFEALNGAGRLEQIDQEFYVLRNKEQYTRERGLVIDVSRGDAVMF

SEQ ID NO: 73 - protein, 219 aa, Clostridium clostridioforme
MSRGVRVRMWGDYALFSRPEMKVERCSYDVMTPSAARGMLEAVYWHPGMRWVIDKIYVRKPIQFTSIRRNEVKSKVLAGNVLSAINGGGKPLYISSREEIVQRAAILLRDVEYVVEAHFEMTPKAVPGDNEGKFKDIIMRRLRRGECYHRPYFGCREFPAQFALYEEEEVDTAYAGTERDLGYMLFDFDYSDTEDIKPMFFRAVMKDGVLDVRNCEVVR

SEQ ID NO: 74 - protein, 591 aa, Clostridium clostridioforme
MILQALVEYYEALERKGKITSPGWCRARVAYGLDISEQGELKSVIPLKQERQKGKKTVTVPQSLTVPQMVSRSSGVSANFLCDHSGYMLGIDNKGKPERSRECFECAKKKHKDILEHISSPAAGAVCRFFDRWAPDQAAENPCLIPELEEIISGSNLVFMVDGEYVHEDPDIRQCWEEYSRQSGTGPVGVCLVTGRREEIARIHGTIKGVQGAQSSGAALVSFNAPAFESYGKEQSFNAPVGTYAAYAYTTALNYLLGDRTHRTTIGDTAVVYWSEEGDERYQNIFACVSEPTMENQEIVDGVFKNLAEGKAVAAQDVQDSLDMDRKFYILGLAPNAARLAVRFFYQDSFGNILKHIKEHYDRMEIVRPAADAVEYLGMWRMLQETVNKKSRDKKPVPSMSGAVYRAIISGSRYPASLYQAVLGRIRAEQDDGDSRIYKITRGRAAIIKAYLLKNGNEKEEITMALNEDSNNTAYVLGREFAVLEAIQEDANPGINATIKDKYFNSACATPAAIFPILFKLKNSHMKKMNNGAKEIYYEKILGDLQGRLTVAEGQKAACPRRLTLEEQGMFILGYYHQTQKRYEKKIKEED

SEQ ID NO: 75 - protein, 296 aa, Clostridium clostridioforme
MAEVIKNRYEFVVLFDVENGNPNGDPDAGNLPRIDPESGYGIVTDVCLKRKIRNYVETLKEDEPGYKIYIREDVPLNRSDNTAYIELKTDEKGIKELKKKDPQVDQKIRDFMCRNFFDIRTFGAVMTTFVKAALNCGQVRGPVQLGFARSIDPIVSQEVTITRVAITTEKDAENKSTEMGRKNIVPYALYRAEGYISANLARKSTGFSDEDLELLWDAIINMFEIDHSAARGKMAVRRLIVFKHSKELGDCPAYKLFDAVEVKKNEEVEYPRKYGDYTVAVHRDQIPETVEVSEKI

SEQ ID NO: 76 - protein, 220 aa, Clostridium clostridioforme
MEYREDDFLMVSGIQHFTFCRRQWALIHIEQQWKENEHTIEGELLHKKAHDPYLAERRGDVMISRALPVYSRSMGVSGECDIVEFHRAEDGIGLHGHRGLFRVFPVEYKKGSPKESEADILQLAAQAMCLEEMLSARIEAGALYYGEIHRRNVVEITDELRKRVRDIFQEMHELYDKGYTPKVRWSKSCNACSMKDICMPKLGKASPVRDYIRGKIEEDV

SEQ ID NO: 77 - protein, 343 aa, Clostridium clostridioforme
MRKLLNTLYVTSPDSYLSLDGENVVIYDNDTELGRIPLHNLEGIVSFGYRGMSPALMGGCAERDISLCVLSPQGRFLARVTGRVRGNVLLRRKQYQVSMNQDASLMIARNCILGKVYNARWVLERAVRDHGLQIDTEKVKDAAGFLKQSLRRIEESRDMAQLRGYEGEDASIYFGVFDQLILQQKNDFYFKGRNRRPPLDNVNAMLSFVYTLLANTVASALETVGLDPYVGFMHTDRPGRLSLALDLMEELRPVLADRFVLTLINRKMVNKKDFSRKEDGAVIMGDEARKLLLSEWQNKKKEMITHPYLNEKVEWGMIPFVQAMLLSRYLRGDIDEYPPFFWK

SEQ ID NO: 78 - protein, 96 aa, Clostridium clostridioforme; identical to SEQ ID NO: 26

SEQ ID NO: 79 - DNA, 2178 bp, Clostridium clostridioforme
  source 1..2178: mol_type = genomic DNA
atgcaatactacgcgcacatcagcgacgacaagatgcgaaggcagacagtgaaggagcatctgacagggacggccaggcgttctgaactgttcgcagaggctttccggtgcggagcatgggggtatggatgcggaattctgcacgacattggaaaatatacccagggattcataaagcgtctggaaggcggttccattacagaccatgccacggcgggggcccaggagatgatggggctgtatgccaggaccaacccgctggctgcttattgcgccgcttactgtatcagcggccaccattcgggacttctggacgggggaaggaacggcgacacggcaggggaggccacgcttcagggaaggcttaagaagcagttagaggattacagcccatacaaggatgaagttgagatgccggattttcccgggctgcctttgagacaaataggcaagggaggattcagcctgtcatttttcatcagaatgctgttttcatgccttgtggacggagattatctggatacggagcaatttatgctgggacacgatgcgggaaggggggaccatgattgtatggacgtccttctcaggcgtctggaaagccatgtggaatcctggctttccaatgatgatttgatatctgtaaatggaagaaggacggctatactgaaggcatgtctgcaggcaggcagaaggaagcagggactgtaccagctcacggtacctaccggcggaggcaagaccatatcctcccttgggtttgccctgcgccacgcagtggaacatggtcttgaccggattatttatgtgatcccgtatacgagtatcatcgaacagaacgcccaggtatttaaggaaatcctgggaaagaaaaatgttctggaaaaccattgtaatgtgacgtatgaggacaatgagagcgcccgggagttaaaattggaacagttggccgccgagaattgggataagccggtggtggtcactacaaatgtccaattttttgaatcactctttgcctccaggacttcggtatgccgcaaactgcataacattgccaacagtgtgatcatatttgatgaggcacaaatgctgccggtgaaatatctgatgccatgtattcgggctgtcagcgagcttatatgtaattatcactgcacggctgttatctgtacggctacccagccgtccctgcagcccttctttcctgcaaagatacaggaactggaggcggcggaactatgtccggttgtaaaaggacaatatgattttttcaaaagaacaaggatacacctggcaggacggatatcccaggagcatctggtgaaggagctggaaggccggcatcaggtcctttgtatcctgaacagcagaaagcgtgtacagcgtatctacgagtccattgaggaccggggaacgtatcatttatccacattgatgtatccaaagcacaggaaggagcttttgaaggagattcgcggccgtctgtcatcaggagaaccgtgccggcttatttccaccagcctggtggaagcaggcgtggattttgactttccggccgtatacagggagctggccggaattgacagcgtgatccaggctgccggaaggtgcaacagggagggaaagagggatccggaagaatgcattactcaggtatttactttggaggaagaggaggatatccatataccacaggaattaaaactgcctatttcagttgcggggcagattgtgggaaagtatgaagatatatcatcgccggaggccattggagaatatttcaccaggctgtaccggtataaaggggagggattggacgcaaaggatgtggtggagcagtttgagcagggaagccgttccttcatgtttccatttgcatcagtggcttccgggttccgcctgattgagagtaataccaggaccattttaattgatacagagccggaagcagcgcagattgccatgcaaatccggcggggagagcataacagggagcttatcaggcaggcgggacagtactgtgtaaatgtttttgaacatgattttgaagcgttaaacggcgctggccgccttgaacagatagaccaggaattttatgttctgaggaataaggagcagtatacaagggagagaggacttgtgattgacgtaagccgcggcgatgccgtcatgttttga

SEQ ID NO: 80 - DNA, 660 bp, Clostridium clostridioforme
  source 1..660: mol_type = genomic DNA
atgagccgaggagtacgggtccggatgtggggggattatgcccttttcagccgccctgagatgaaggtggaacggtgcagctatgatgttatgactccctccgccgcaaggggtatgctggaggccgtctactggcatccggggatgcggtgggtgattgacaaaatctatgtgagaaaacccatccagtttaccagcatccgaagaaatgaggtaaagagcaaggtattggcaggaaatgtattgtctgctataaacgggggaggaaaaccgctgtatataagcagcagggaggaaatcgtacagagagcggccattcttctgcgggatgtggaatatgtggttgaggctcactttgagatgacccccaaggcagtcccaggggataatgagggtaagtttaaggacattattatgcgccggctccgcaggggtgagtgctatcacagaccgtattttggatgccgggaatttcctgcccagtttgccctttatgaagaggaggaggtagacactgcatatgccggaacggagagggatttggggtatatgctgtttgactttgattactcagatacagaggacattaaacccatgtttttcagggccgtcatgaaggatggtgttctggatgtaaggaattgtgaggtggtgcgatga

SEQ ID NO: 81 - DNA, 1776 bp, Clostridium clostridioforme
  source 1..1776: mol_type = genomic DNA
atgattctccaggcgcttgttgaatattatgaagcccttgagcggaagggaaagataaccagtccgggctggtgcagggccagggtagcgtacgggctggatatttcagagcagggggagcttaagagcgtcattccccttaagcaggagcggcagaaaggtaaaaagacagtcacggtaccgcaaagccttacggtaccccagatggtttcccgttcttccggtgtatccgccaatttcctgtgcgaccattccggttatatgctgggaattgataataaggggaaaccggagcggtccagggaatgttttgagtgcgccaaaaagaagcataaggacattttagagcatatcagcagccccgcggccggggcggtgtgccggttttttgaccgctgggcgccggatcaggcagcggaaaacccatgcctgataccggaactggaggagattatatccggttctaatctggtgttcatggtggatggggaatatgtccatgaggacccggatataaggcagtgctgggaagaatacagccgtcagtcaggcacaggacctgtgggcgtctgcctggtgacagggcgcagggaggaaatagccaggattcacggcaccataaaaggcgtccagggggcccagtccagcggtgctgcgctggtttcctttaatgcacctgcctttgaatcatacggcaaggaacagagctttaacgcaccggtgggaacatacgcggcctatgcctataccacggcgctcaattatctccttggggaccggactcataggacaaccatcggtgacacggcagtggtttactggtccgaggaaggcgacgaacgataccagaatatatttgcgtgcgtatcggagccaacgatggagaaccaggaaatagtggatggggtattcaagaacctggcagagggaaaagcggttgcggctcaggatgtccaggacagtcttgatatggacaggaaattttatatattaggtctggcgcctaatgcagccaggctggcagtgcgttttttctatcaggacagctttggaaatatattaaagcatataaaagaacattatgacagaatggaaatagtgcgtccggcagccgacgccgtggagtatctgggaatgtggcgtatgctgcaggagaccgtcaataaaaagtccagggataaaaagccggttcccagtatgtccggggcagtgtacagggcaatcatatccggcagccggtatccggcgtccctgtatcaggccgtgcttggcaggatacgggcagagcaggacgacggggacagccggatttataagataaccaggggaagggcagccattatcaaagcatatctgctgaaaaatggaaatgaaaaggaggaaataaccatggcgctgaatgaagacagtaataataccgcgtacgtcctgggaagggagtttgcggttctggaggcaatccaggaggacgccaatccggggattaacgccaccataaaggataagtattttaactctgcatgcgcgacgccggcggcaatctttcccattttatttaagctgaaaaacagccacatgaagaagatgaacaatggggcgaaggagatatattatgaaaaaatactgggtgatttacagggaaggctgactgtcgcagaggggcagaaggcagcctgcccaaggcggcttaccctggaggagcagggaatgttcatattgggatattaccatcagactcagaagagatatgagaagaagataaaggaggaggattaa

SEQ ID NO: 82 - DNA, 891 bp, Clostridium clostridioforme
  source 1..891: mol_type = genomic DNA
atggcagaggtgattaagaacaggtatgaatttgtggtgttatttgatgtggagaacggaaatccaaacggcgacccggatgcaggcaatctgccgaggattgatccggagagcggatacggtatcgtgacggatgtgtgcttaaaacgtaagatacgcaattatgtggagacactgaaggaagacgagcccgggtataaaatatatatccgggaggatgtacctttaaacagaagtgacaataccgcgtacattgagcttaagacagatgaaaagggaatcaaggaactcaaaaagaaagaccctcaggtagaccagaaaatccgggattttatgtgccgtaactttttcgatatccgaacctttggcgcggtcatgacaacctttgtaaaggctgcgttaaactgcggccaggtgcgtgggcctgtacagctgggatttgccagaagcattgaccccattgtgagtcaggaagtgaccattacccgtgtggctatcaccacagaaaaggacgcggaaaataagagtacggaaatgggaagaaagaatatcgttccctacgccctgtaccgcgcggagggttacatatcagctaatctggccagaaaatccacgggcttttcagatgaggatctggaacttttgtgggatgccatcatcaatatgtttgaaatcgaccattccgcagccaggggaaaaatggcggtccgccggctcatcgtgtttaaacatagtaaggaactgggagattgccctgcatacaaactctttgatgccgtggaagtaaaaaagaatgaggaagtggagtatcccagaaaatacggggattacacggttgccgtccatagggaccagattccggaaacggttgaagtttcggagaagatataa

SEQ ID NO: 83 - DNA, 663 bp, Clostridium clostridioforme
  source 1..663: mol_type = genomic DNA
atggaatacagggaggatgacttcctcatggtgtccggtatccagcatttcaccttttgcaggcgccagtgggcccttatacatattgaacagcagtggaaagagaatgagcataccatagaaggagagctgctccacaaaaaggcccatgatccatatttggcagaaaggcggggcgatgtgatgataagcagggcattgcctgtgtattccaggagtatgggtgtcagcggggaatgcgatattgtagagtttcacagggcggaggatggaatcggactccatgggcacaggggactgttccgggtatttccggtggaatataaaaaggggagcccaaaggaatcagaagcagatatactgcagctggcggcccaggccatgtgcctggaggaaatgctgtcagccaggattgaagcgggggcgctgtattacggagagattcaccgcaggaatgtggtggagattacagatgaactgcgaaaacgggtaagggatatatttcaggagatgcatgaactttatgacaagggatatacaccaaaggtcagatggtcaaagagctgtaatgcctgctccatgaaagatatatgtatgccgaaactgggaaaagcatcacctgtcagggattatatccgtgggaagattgaggaggatgtatga

SEQ ID NO: 84 - DNA, 1032 bp, Clostridium clostridioforme
  source 1..1032: mol_type = genomic DNA
atgaggaagttattaaacaccctgtatgttacttctcctgacagttatctgtcgctggacggggaaaatgtggtaatctatgacaacgatacggaactgggacggataccgctccataatctggagggaatagtgtcctttggatacaggggaatgagcccggccctcatggggggatgcgcggagcgggacatatcactttgcgtcctttcgcctcagggaagatttctggcccgtgtgaccggacgtgtgcggggaaacgtacttctgcgcaggaaacagtaccaggtttccatgaatcaggacgcaagtctgatgatagccagaaactgtatccttggaaaagtatacaatgccagatgggttctggagagagctgtgagagaccatggacttcagattgacacagagaaggtgaaggatgcggccggattcctgaaacaatccctcaggcgcatagaggaaagccgggacatggctcagctgcgggggtatgaaggggaggatgccagtatttactttggggttttcgaccagctcatattacagcagaaaaatgatttctatttcaagggaaggaacaggcggccgcctttggataatgtgaatgccatgctgtcatttgtgtacacgctgctggccaatacagttgcgtcagcattggaaacagttggcctggacccttacgtgggattcatgcatacggaccggccgggccgcctttccctggcattggatttaatggaggagctgcggccggttctggcagaccgttttgtgcttaccctgattaaccggaagatggtaaacaaaaaggatttttcccggaaagaagacggcgctgtcattatgggggatgaggcacggaagctgttgctgtcagaatggcagaacaagaaaaaggagatgattacccatccatatctgaatgagaaggtggagtggggaatgattccatttgttcaggccatgcttctgtccagatatctgcgaggcgatatagatgaatacccgccgtttttctggaaatag

SEQ ID NO: 85 - DNA, 291 bp, Clostridium clostridioforme; identical to SEQ ID NO: 33
  source 1..291: mol_type = genomic DNA

SEQ ID NO: 86 - DNA, 33 bp, Clostridium clostridioforme; identical to SEQ ID NO: 35
  source 1..33: mol_type = genomic DNA

SEQ ID NO: 87 - DNA, 32 bp, Clostridium clostridioforme
  source 1..32: mol_type = genomic DNA
gtctccaccctcgcggtggagtggattgaaat

SEQ ID NO: 88 - DNA, 32 bp, Clostridium clostridioforme
  source 1..32: mol_type = genomic DNA
atctccaccctcgcggtggagtggattgaaat

SEQ ID NO: 89 - protein, 725 aa, Clostridium clostridioforme
MQYYAHISDDKMRRQTVKEHLTGTARRSELFAEAFRCGAWGYGCGILHDIGKYSQGFIKRLEGGSITDHATAGAQEMMGLYARTNPLAAYCAAYCISGHHSGLLDGGRNGDTAGEATLQGRLKKQLEDYSPYKDEVEMPDFPGLPFRQIGKGGFSLSFFIRMLFSCLVDGDYLDTEQFMLGHDAGRGDHDCMDVLLRRLESHVESWLSNDDLISVNGRRTAILKACLQAGRRKQGLYQLTVPTGGGKTVSSLGFALRHAVEHGLDRIIYVIPYTSIIEQNAQVFKEILGKKNVLENHCNVTYEDNESARELKLEQLAAENWDKPVVVTTNVQFFESLFASRTSACRKLHNIANSVIIFDEAQMLPVKYLMPCIRAVSELICNYHCTAVICTATQPSLQPFFPAKIQELEAAELCPDVKGQYDFFKRTRIHLAGRISQENLVKELEGRHQVLCILNSRKRVQRIYESIEDRGTYHLSTLMYPKHRKELLKEIRGRLSSGEPCRLISTSLVEAGVDFDFPAVYRELAGIDSVIQAAGRCNREGKRDPEECITQVFTLEEEEDIHIPQELKLPISVAGQIVGKYEDISSPKAIGEYFTRLYRYKGEGLDAKDVVEQFEQGSRSFMFPFASVASGFRLIENNTRTILIDTEPEAAQIAMQIRRGEHNRELIRQAGQYCVNVFEHDFEALNGAGRLEQIDQEFYVLRNKEQYTRERGLVIDVSRGDAVMF

SEQ ID NO: 90 - protein, 219 aa, Clostridium clostridioforme; identical to SEQ ID NO: 73

SEQ ID NO: 91 - protein, 591 aa, Clostridium clostridioforme
MILQALVEYYEALERKGKITSPGWCRAKVAYGLDISEQGELKRVIPLKQERQKGKKTVTVPQNLTVPQMVSRSSGVSANFLCDHSGYMLGIDNKGKPERSRECFECAKKKHKDILEHISSPAAGAVCRFFDRWAPDQAAENPCLIPELEDIISGSNLVFMVDGEYVHEDPDIRQCWEEYSRQSGTGPVGVCLVTGRREEIARIHGTIKGVQGAQSSGAALVSFNAPAFESYGKEQSFNAPVGTYAAYAYTTALNYLLGDRTHGTTIGDTAVVYWSEEGDERYQNIFACVSEPTMENQEIVDGVFKNLAEGKAVAAQDVQDSLDMDRKFYILGLAPNAARLAVRFFYQDSFGNILKHIKEHYDRMEIVRPAADAVEYLGMWRMLQETVNKKSRDKKPVSSMSGAVYRAIISGSRYPASLYQAVLGRIRAEQDDGDSRIYKITRGRAAIIKAYLLKNGNEKEEITMALNEDSNNTAYILGREFAVLEAIQEDANPGINATIKDKYFNSACATPAAIFPILFKLKNSHMKKMNNGAKEIYYEKILGDLQGRLTVAEGQKAACPRRLTLEEQGMFILGYYHQTQKRYEKKIKEED

SEQ ID NO: 92 - protein, 296 aa, Clostridium clostridioforme; identical to SEQ ID NO: 23

SEQ ID NO: 93 - protein, 220 aa, Clostridium clostridioforme; identical to SEQ ID NO: 76

SEQ ID NO: 94 - protein, 343 aa, Clostridium clostridioforme
MRKLLNTLYVTSPDSYLSLDGENVVIYDNDTELGRIPLHNLEGIVSFGYRGMSPALMGGCAERDISLCVLSPQGRFLARVTGRVRGNVLLRRKQYQVSMNQDASLMIARNCILGKVYNARWVLERAVRDHGLQIDTEKVKDAAGFLKQSLRRIEESRDMAQLRGYEGEDASIYFGVFDQLILQQKKDFYFKGRNRRPPLDNVNAMLSFVYTLLANTVASALETVGLDPYVGFMHTDRPGRLSLALDLMEELRPVLADRFVLTLINRKMVNKKDFSRKEDGAVIMGDEARKLLLSEWQNKKKEMITHPYLNEKVEWGMIPFVQAMLLSRYLRGDIDEYPPFFWK

SEQ ID NO: 95 - protein, 96 aa, Clostridium clostridioforme; identical to SEQ ID NO: 26

SEQ ID NO: 96 - DNA, 2178 bp, Clostridium clostridioforme
  source 1..2178: mol_type = genomic DNA
atgcaatactacgcgcacatcagcgacgacaagatgcgaaggcagacagtgaaggagcatctgacagggacggccaggcgttctgaactgttcgcagaggctttccggtgcggagcatgggggtatggatgcggaattctgcacgatattggaaaatattcccagggattcatcaagcgtctggaaggcggttccattacagaccatgccacggcgggggcccaggagatgatggggctgtatgccaggaccaacccgctggctgcttattgcgccgcttactgtatcagcggccatcattcgggacttctggacgggggaaggaacggcgacacggcaggggaggccacgcttcagggaaggcttaagaagcaattagaggattacagtccatacaaggatgaagttgagatgccggattttcccgggctgccgttcagacagataggcaagggaggattcagcctgtcatttttcatcagaatgctgttttcatgccttgtggacggagattatctggatacggagcaatttatgctgggacacgatgcgggaaggggagaccatgattgtatggacgtccttctcaggcgtctggaaagccatgtggaatcctggctttccaatgatgatttgatatctgtaaatggaagaaggacggctatactgaaggcatgtctgcaggcaggcagaaggaagcaggggctgtaccagctcacggtacctaccggcggaggcaagaccgtatcctcccttgggtttgccctgcgccacgcagtggaacatggtcttgaccggattatttatgtgatcccgtatacgagtatcatcgaacagaacgcccaggtatttaaggaaatcctgggaaagaaaaatgttctggaaaaccattgtaatgtgacgtatgaggacaatgagagcgcccgggagttaaaattggaacagttggccgccgagaattgggataagccggtggtggtcactacaaatgtccaattttttgaatcactctttgcctccaggacttcggcatgccgcaaactgcataacattgccaacagtgtgatcatatttgatgaggcacaaatgctgccggtgaaatatctgatgccatgtattcgggctgtcagcgagcttatatgtaattatcactgcacggctgttatctgtacggctacccagccgtccctgcagcccttctttcctgcaaagatacaggaactggaggcggcggaactatgtccggatgtaaaaggacaatatgattttttcaaaagaacaaggatacacctggcaggacggatatcccaggaaaatctggtgaaggagctggaaggccggcatcaggtcctttgtatcctgaacagcagaaagcgtgtacagcgtatctacgagtccattgaggaccggggaacgtatcatttatccacattgatgtatccaaagcacaggaaggagcttttgaaggagattcgcggccgtctgtcatcaggagaaccgtgccggcttatttccaccagcctggtggaagcaggcgtggattttgattttccggccgtatacagggagctggccggaattgacagcgtgatccaggctgccggaaggtgcaacagggagggaaagagggatccggaagaatgcattacccaggtatttactttggaggaagaggaggatatccatataccacaggaattaaaactgcctatttcagttgcggggcagattgtgggaaagtatgaagatatatcatcgccgaaggccattggagaatatttcaccaggctgtaccggtataaaggggagggattggacgcaaaggatgtggtggagcagtttgagcagggaagccgttcctttatgtttccatttgcatcagtggcttcagggttccgcctgattgagaacaataccaggaccattttaattgatacagagccggaagcagcgcagattgccatgcaaatccggcggggagagcataacagggagcttatcaggcaggcgggacagtactgtgtaaatgtttttgaacatgattttgaagcgttaaacggcgctggccgccttgaacagatagaccaggaattttatgttctgaggaataaggagcagtatacaagggagagaggacttgtgattgacgtaagccgcggcgatgccgtcatgttttga

SEQ ID NO: 97 - DNA, 660 bp, Clostridium clostridioforme
  source 1..660: mol_type = genomic DNA
atgagccgaggagtacgggtccggatgtggggggattatgcccttttcagccgccctgagatgaaggtggaacggtgcagctatgatgtgatgactccctccgccgcaaggggtatgctggaggccgtctactggcatccggggatgcggtgggtgattgacaaaatctatgtgagaaaacccatccagtttaccagcatccgaagaaatgaggtaaagagcaaggtattggcaggaaatgtattgtctgctataaacgggggaggaaaaccgctgtatataagcagcagggaggaaatcgtacagagagcggccattcttctgcgggatgtggaatatgtggttgaggctcactttgagatgacccccaaggcagtcccaggggataatgagggtaagtttaaggacattattatgcgccggctccgcaggggtgagtgctatcacagaccgtattttggatgccgggaatttcctgcccagtttgccctttatgaagaggaggaggtagacactgcatatgccggaacggagagggatttggggtatatgctgtttgactttgattactcagatacagaggacattaaacccatgtttttcagggccgtcatgaaggatggtgttctggatgtaaggaattgtgaggtggtgcgatga

SEQ ID NO: 98 - DNA, 1776 bp, Clostridium clostridioforme
  source 1..1776: mol_type = genomic DNA
atgattctccaggcgcttgttgaatattatgaagcccttgaacggaagggaaagataaccagtccgggctggtgcagggccaaggtagcgtacgggctggatatttcagagcagggggagcttaagcgcgtcattccccttaagcaggagcggcagaaaggtaaaaagacagtcacggtaccgcagaaccttacggtaccccagatggtttcccgttcttccggtgtatccgccaatttcctgtgcgaccattccggttatatgctgggaattgataataaggggaaaccggagcggtccagggaatgttttgagtgcgccaaaaagaagcataaggacattttagagcatatcagcagccccgcggccggggcggtgtgccggttttttgaccgctgggcgccggatcaggcagcggaaaacccatgcctgataccggaactggaggatattatatctggttctaatctggtgttcatggtggatggggaatatgtccatgaggacccggatataaggcagtgctgggaagaatacagccgtcagtcaggcacaggacctgtgggcgtctgcctggtgacagggcgcagggaggaaatagccaggattcacggtaccataaaaggcgtccagggggcccagtccagcggtgctgcgctggtttcctttaatgcacctgcctttgaatcatacggcaaggaacagagctttaacgcaccggtgggaacatacgcggcctatgcctataccacggcgctcaattatctccttggggaccggactcatgggacaaccatcggtgacacggcagtggtttactggtccgaggaaggcgacgaacgataccagaatatatttgcgtgcgtatcggagccaacgatggagaaccaggaaatagtggatggggtattcaagaacctggcagagggaaaagcggttgcggctcaggatgtccaggacagtcttgatatggacaggaaattttatatattaggtctggcgcccaatgcagccaggctggcagtgcgttttttctatcaggacagctttggaaatatattaaagcatataaaagaacattatgacagaatggaaatagtgcgtccggcagccgacgccgtggagtatctgggaatgtggcgtatgctgcaggagaccgtcaataaaaagtccagggataaaaagccggtttccagtatgtccggagcagtatacagggcaattatatccggcagccggtatccggcgtccctgtatcaggccgtgcttggcaggatacgggcagagcaggacgacggggacagccggatttataagataaccaggggaagggcagccattatcaaagcatatctgctgaaaaatggaaatgaaaaggaggaaataaccatggcgctgaatgaagacagtaataataccgcgtacatcctgggaagggagtttgcggttctggaggcaatccaggaggacgccaatccggggattaacgccaccataaaggataagtattttaactctgcatgcgcgacgccggcggcaatctttcccattttatttaagctgaaaaacagccacatgaagaagatgaacaatggggcgaaggagatatattatgaaaaaatactgggtgatttacagggaaggctgactgttgcagagggacagaaggcagcctgcccaaggcggcttaccctggaggagcagggaatgttcatattgggatattaccatcagactcagaagagatatgagaagaagataaaggaggaggattaa

SEQ ID NO: 99 - DNA, 891 bp, Clostridium clostridioforme
  source 1..891: mol_type = genomic DNA
atggcagaggtgattaagaacaggtatgaatttgtggtgttatttgatgtggagaacgggaatccaaacggcgacccggatgcaggcaatctgcccaggattgacccggagagcggatacggcattgtgacggatgtatgtttaaaacgtaagatacgcaattatgtggagaccctgaaggaagacgagcccgggtataaaatatatatccgggaggatgtacccttaaacagaagtgacaataccgcgtacattgagcttaagacagatgaaaagggaatcaaggaactcaaaaagaaagaccctcaggtagaccagaaaatacgggattttatgtgccgtaactttttcgatatccgagcctttggcgcggtcatgacaacctttgtaaaggctgcgttaaactgcggccaggtgcgtgggcctgtacagctgggatttgccagaagcattgaccccattgtgagtcaggaagtgaccattacccgtgtggccatcaccacagaaaaggacgcggaaaataagagtacggaaatgggaagaaagaatatcgttccctacgccctgtatcgcgcggagggctacatatcagctaatctggccagaaaatccacgggcttttcagaggaggatctggaacttttgtgggatgctatcatcaatatgtttgaaatcgaccattccgcagccaggggaaaaatggcggtccgccggctcatcgtgtttaaacatagtaaggaactgggagattgccctgcatacaaactctttgatgccgtggaagtaaaaaagaatgaggaagtggagtatcccagaaaatacggggattacacggttgccgtccatagggaccagattccggaaacggttgaagtttcggagaagatataa

SEQ ID NO: 100 - DNA, 663 bp, Clostridium clostridioforme
  source 1..663: mol_type = genomic DNA
atggaatacagggaggatgatttcctcatggtgtccggtatccagcatttcaccttttgcaggcgccagtgggcccttatacatattgaacagcaatggaaagagaatgagcataccatagaaggagagctgctccacaaaaaggcccatgacccatatttggcagaaaggcggggcgatgtgatgataagcagggcattgcctgtgtattccaggagcatgggtgtcagcggggaatgcgatattgtagagtttcacagggcggaggatggaatcggactccatgggcacaggggactgttccgggtatttccggtggaatataaaaaggggagcccaaaggaatcagaagcagatatactgcagctggcggcccaggccatgtgtctggaggaaatgctgtcagccaggattgaagcgggggcgctgtattacggagagattcaccgcagaaatgtggtggaaattacagatgaactgcgaaaacgggtaagggatatatttcaggagatgcatgaactttatgacaagggatatacaccaaaggtcagatggtcaaagagctgtaatgcctgctccatgaaagatatatgtatgccgaaactgggaaaagcatcacctgtcagggattatatccgtgggaagattgaggaggatgtatga

SEQ ID NO: 101 - DNA, 1032 bp, Clostridium clostridioforme
  source 1..1032: mol_type = genomic DNA
atgaggaagttattaaacaccctgtatgttacttctcctgacagttatctgtcgctggacggggagaatgtggtaatctatgacaacgatacggaactgggacggataccgctccataatctggagggaatagtgtcctttggatacaggggaatgagcccggccctcatggggggatgcgcggagcgggacatatcactttgcgtcctttcgcctcagggaagatttctggcccgtgtgaccggacgtgtgcggggaaacgtacttctgcgcaggaaacagtaccaggtttccatgaatcaggacgcaagtctgatgatagccagaaactgtatccttggaaaagtatacaatgccagatgggttctggagagagctgtgagagaccatggacttcagattgacacagagaaggtgaaggatgcggccggattcctgaaacaatccctcaggcgcatagaggaaagccgggacatggctcagctgcgggggtatgaaggggaggatgccagtatttactttggggttttcgaccagctcatattacagcagaaaaaggatttctatttcaagggaaggaacaggcggccgcctttggataatgtgaatgccatgctgtcatttgtgtacacgctgctggccaatacagttgcgtcagcattggaaacagttggcctggacccttacgtgggattcatgcatacggaccggccgggccgcctttccctggcattggatttaatggaggagctgcggccggttctggcagaccgttttgtgcttaccctgattaaccggaagatggtaaacaaaaaggatttttcccggaaagaggacggcgctgtcattatgggggatgaggcacggaagctgttgctgtcagaatggcagaacaagaaaaaggagatgattacccatccatatctgaatgagaaggtggagtggggaatgattccatttgttcaggccatgcttctgtccagatatctgcgaggcgatatagatgaatacccgccgtttttctggaaatag

SEQ ID NO: 102 - DNA, 291 bp, Clostridium clostridioforme; identical to SEQ ID NO: 33
  source 1..291: mol_type = genomic DNA

SEQ ID NO: 103 - DNA, 32 bp, Clostridium clostridioforme; identical to SEQ ID NO: 87
  source 1..32: mol_type = genomic DNA

SEQ ID NO: 104 - DNA, 33 bp, Clostridium clostridioforme; identical to SEQ ID NO: 35
  source 1..33: mol_type = genomic DNA

SEQ ID NO: 105 - DNA, 33 bp, Clostridium clostridioforme
  source 1..33: mol_type = genomic DNA
gtcgaggcccgcgagagccttgtggattgaaat

SEQ ID NO: 106 - protein, 725 aa, Clostridium clostridioforme
MQYYAHISDDKARRQTVKEHLTGTARRSELFAEAFRCGAWGYGCGILHDIGKYSQGFRKRLEGGSITDHATAGAQEMMGLYARTNPLAAYCAAYCISGHHSGLLDGGRNGDTAGEATLQGRLKKQLEDYSPYKDEVEMPDFPGLPFRQIGKGGFSLSFFIRMLFSCLVDGDYLDTEQFMLGHDAVRGDHDCMDVLLRRLESHVESWLSNDDLISVNGRRTAILKACLQAGRRKQGLYQLTVPTGGGKTVSSLGFALRHAVEHGLDRIIYVIPYTSIIEQNAQVFKEILGKKNVLENHCNVTYEDNESARELKLEQLAAENWDKPVVVTTNVQFFESLFASRTSACRKLHNIANSVIIFDEAQMLPVKYLMPCIRAVSELICNYHCTAVICTATQPSLQPFFPAKIQELEAAELCPDVKGQYDFFKRTRIHLAGRISQEHLVKELEGRHQVLCILNSRKRVQRIYESIEDRGTYHLSTLMYPKHRKELLKEIRGRLSSGEPCRLISTSLVEAGVDFDFPAVYRELAGIDSVIQAAGRCNREGKRDPEECITQVFTLEEEEDIHIPQELKLPISVAGQIVEKYEDISSPEAIGEYFTRLYRYKGEGLDAKDVVEQFEQGSHSFMFPFASVASEFRLIESNTRTILIDTEPQAAQIAMQIRRGEHNRELIRQAGQYCVNVFEHDFEALNGAGRLEQIDQEFYVLRNKEQYTRERGLVIDVSRGDAVMF

SEQ ID NO: 107 - protein, 219 aa, Clostridium clostridioforme; identical to SEQ ID NO: 21

SEQ ID NO: 108 - protein, 591 aa, Clostridium clostridioforme
MILQALVEYYEALERKGKITSPGWCRAKVAYGLDISEQGELKSVIPLKQERQKGKKTVTVPQNLTVPQMVSRSSGVSANFLCDHSGYMLGIDNKGKPERSRECFECAKKKHKDILEHISSPAARAVCRFFDRWAPDQAAENPCLIPELEEIISGSNLVFMMDGEYVHEDPDIRQCWEEYSRQSGTGHVGVCLVTGRREEIARIHGTIKGVQGAQSSGAALVSFNAPAFESYGKEQSFNAPVGTYAAYAYTTALNYLLGDRTHGTTIGDTAVVYWSEEGDERYQNIFACVSEPTMENQEIVDGVFKNLAEGKAVAAQDVQDSLDMDRKFYILGLAPNAARLAVRFFYQDSFGNILKHIKEHYDRMEIVRPAADAVEYLGMWRMLQETVNKKSRDKKPVPSMSGAVYRAIISGSRYPASLYQAVLGRIRAEQDDGDSRIYKITRGRAAIIKAYLLKNGNEKEEITMALNEDSNNTAYILGREFAVLEAIQEDANPGINATIKDKYFNSACATPAAIFPILFKLKNSHIKKMNNGAKEIYYEKILGDLQGRLTVAEGQKAACPRRLTLEEQGMFILGYYHQTQKRYEKKIKEEA

SEQ ID NO: 109 - protein, 296 aa, Clostridium clostridioforme
MAEVIKNRYEFVVLFDVENGNPNGDPDAGNLPRIDPESGYGIVTDVCLKRKIRNYVETLKEDEPGYKIYIREDVPLNRSDNTAYIELKTDEKGIKELKKKDPQVDQKIRDFMCRNFFDIRAFGAVMTTFVKAALNCGQVRGPVQLGFARSIDPIVSQEVTITRVAITTEKDAENKSTEMGRKNIVPYALYRAEGYISANLARKSTGFSEEDLELLWDAIINMFEIDHSAARGKMAVRRLIVFKHSKELGDCPAYKLFDAVEVKKNEEVEYPRKYGDYTVAVHRDQIPVTVEVSEKI

SEQ ID NO: 110 - protein, 220 aa, Clostridium clostridioforme
MEYREDDFLMVSDIQHFTFCRRQWALIHIEQQWKENEHTIEGELLHKKAHDQYLAERRGDVMISRALPVYSRSMGVSGECDIVEFHRAEDGIGLHGHRGLFRVFPVEYKKGSPKESEADILQLVAQAMCLEEMLSARIEAGALYYGEIHRRNVVEITDELRKRVREIFQEMHELYDKGYTPKVRWSKSCNACSMKDICMPKLGKASPVRDYIRGKIEEDV

SEQ ID NO: 111 - protein, 343 aa, Clostridium clostridioforme; identical to SEQ ID NO: 25

SEQ ID NO: 112 - protein, 96 aa, Clostridium clostridioforme; identical to SEQ ID NO: 26

SEQ ID NO: 113 - DNA, 2178 bp, Clostridium clostridioforme
  source 1..2178: mol_type = genomic DNA
atgcaatactacgcgcacatcagcgacgacaaggcgcgaaggcagacagtgaaggagcatctgacagggacggccaggcgttctgaactgttcgcagaggctttccggtgcggagcatgggggtatggatgcggaattctgcacgacattgggaaatattcccagggattcagaaagcgtctggaaggcggttccattacagaccatgccacggcgggggcccaggagatgatggggctgtatgccaggaccaacccgctggctgcttattgcgccgcttactgtatcagcggccaccattcgggacttctggacggaggaaggaacggcgacacggcaggggaggccacgcttcagggaaggcttaagaagcaattagaggattacagtccatacaaggatgaagttgagatgccggattttcccggactgccgttcagacagataggcaagggaggattcagcctgtcatttttcatcagaatgctgttttcatgccttgtggacggagattatctggatacggagcaatttatgctgggacacgatgcggtaaggggagaccatgattgtatggacgttcttctcaggcgtctggaaagccatgtggaatcctggctttccaatgatgatttgatatctgtaaatggaagaaggacggctatactgaaggcatgtctgcaggcaggcagaaggaagcaggggctgtaccagctcacggtacctaccggcggaggcaagaccgtatcctcccttgggtttgccctgcgccacgcagtggaacatggtcttgaccggattatttatgtgatcccgtatacgagtatcatcgaacagaacgcccaggtatttaaggaaatcctgggaaagaaaaatgttctggaaaaccattgtaatgtgacgtatgaggacaatgagagcgcccgggagttaaaattggaacagttggccgccgagaattgggataagccggtggtggtcactacaaatgtccaattttttgaatcactctttgcctccaggacttcggcatgccgcaaactgcataacattgccaacagtgtgatcatatttgatgaggcacaaatgctgccggtgaaatatctgatgccatgtattcgggctgtcagcgagcttatatgtaattatcactgtacggctgttatctgtacggctacccagccgtccctgcagcccttctttcctgcaaagatacaggaactggaggcggcggaactatgtccggatgtaaaaggacaatatgattttttcaaaagaacaaggatacacctggcaggacggatatcccaggagcatctggtgaaggagctggaaggccggcatcaggtcctttgtatcctgaacagcagaaagcgtgtacagcgtatctacgagtccattgaggaccggggaacgtatcatttatccacattgatgtatccaaagcacaggaaggagcttttgaaggagattcgcggccgtctgtcatcaggagaaccgtgccggcttatttccaccagcctggtggaagcaggcgtggattttgattttccggccgtatacagggagctggccggaattgacagcgtgatccaggctgccggaaggtgcaacagggagggaaagagggatccggaagaatgcattacccaggtatttactttggaggaagaggaggatatccatataccacaggaattaaaactgcctatttcagttgcggggcagattgtggaaaagtatgaagatatatcatcgccggaggccattggagaatatttcaccaggctgtaccggtataaaggggagggattggacgcaaaggatgtggtggagcagtttgagcagggaagccattccttcatgtttccatttgcatcggtggcttccgagttccgcctgattgagagcaataccaggaccattttaattgatacagagccgcaagcagcgcagattgccatgcaaatccggcggggagagcataacagggagcttatcaggcaggcgggacagtactgtgtaaatgtttttgaacatgattttgaagcgttaaacggcgctggccgccttgaacagatagaccaggaattttatgttctgaggaataaggagcagtatacaagggagagaggacttgtgattgacgtaagccgcggcgatgccgtcatgttttga

SEQ ID NO: 114 - DNA, 660 bp, Clostridium clostridioforme; identical to SEQ ID NO: 28
  source 1..660: mol_type = genomic DNA

SEQ ID NO: 115 - DNA, 1776 bp, Clostridium clostridioforme
  source 1..1776: mol_type = genomic DNA
atgattctccaggcgcttgttgaatattatgaagcccttgaacggaagggaaagataaccagtccgggctggtgcagggcaaaggtagcgtacgggctggatatttcagagcagggggagcttaagagcgtcattccccttaagcaggagcggcagaagggtaaaaagacagtcacggtaccgcagaaccttacggtaccccagatggtttcccgttcttccggtgtatccgccaatttcctgtgcgaccattccggttatatgctgggaattgataataaggggaaaccggagcggtccagggaatgttttgagtgcgccaaaaagaagcataaggacattttagagcatatcagcagccccgcggccagggcggtgtgccggttttttgaccgctgggcgccggatcaggcagcggaaaacccatgcctgataccggaactggaggagattatatctggttctaatctggtgttcatgatggatggggaatatgtccatgaggacccggatataaggcagtgctgggaagaatacagccgtcagtcaggcacaggacatgtgggcgtctgcctggtgacagggcgcagggaggaaatagccaggattcacggcaccataaaaggagtccagggggcccagtccagcggtgctgcgctggtttcctttaatgcacctgcctttgaatcatacggcaaggaacagagctttaacgcaccggtgggaacatatgcggcctatgcctataccacggcgctcaattatctccttggggaccggactcatgggacaaccatcggtgacacggcagtggtttactggtccgaggaaggcgacgaacgataccagaatatatttgcgtgcgtatcggagccaacgatggagaaccaggaaatagtggatggggtattcaagaacctggcagagggaaaagcggttgcggctcaggatgtccaggacagtcttgatatggacaggaaattttatatattaggtctggcgcctaatgcagccaggctggcagtgcgttttttctatcaggacagctttggaaatatattaaagcatataaaagaacattatgacagaatggaaatagtgcgtccggcagccgacgccgtggagtatctgggaatgtggcgtatgctgcaggagaccgtcaataaaaagtccagggataaaaagccggttcccagtatgtccggggcagtgtacagggcaatcatatccggcagccggtatccggcgtccctgtatcaggccgtgcttggcaggatacgggcagagcaggacgacggggacagccggatttataagataaccaggggaagggcagccattatcaaagcatatctgctgaaaaatggaaatgaaaaggaggaaataaccatggcgctgaatgaagacagtaataataccgcgtacatcctgggaagggagtttgcggttctggaggcaatccaggaggacgccaatccggggattaacgccaccataaaggataagtattttaactctgcatgcgcgacgccggcggcaatctttcccattttatttaagctgaaaaacagccacataaagaagatgaataatggggcgaaggagatatattatgaaaaaatactgggtgatttacagggaaggctgactgtcgcagagggacagaaggcagcctgcccaaggcggcttaccctggaggagcagggaatgttcatattgggatattaccatcagactcagaagagatatgagaagaagataaaggaggaggcttaa

SEQ ID NO: 116 - DNA, 891 bp, Clostridium clostridioforme
  source 1..891: mol_type = genomic DNA
atggcagaggtgattaagaacaggtatgaatttgtggtgttatttgatgtggagaacgggaatccaaacggcgacccggatgcaggcaatctgcccaggattgatccggagagcggatacggtatcgtgacggatgtgtgcttaaaacgtaagatacgcaattatgtggagacactgaaggaagacgagcccgggtataaaatatatatccgggaggatgtacccttaaacagaagtgacaataccgcgtacattgagcttaagacagatgaaaagggaatcaaggaactcaaaaagaaagaccctcaggtagaccagaaaatacgggattttatgtgccgtaactttttcgatatccgagcctttggcgcggtcatgacaacctttgtaaaggcagcgttaaactgcggccaggtgcgtgggcctgtacagctgggatttgccagaagcattgaccccattgtgagtcaggaagtgaccattacccgtgtggctatcaccacagaaaaggacgcggaaaataagagtacggaaatgggaagaaagaatatcgttccctacgccctgtaccgcgcggagggttacatatcagctaatctggccagaaaatccacgggcttttcagaggaggatttggaacttttgtgggatgccatcatcaatatgtttgaaatcgaccattccgcagccaggggaaaaatggcggtccgccggctcatcgtgtttaaacatagtaaggaattgggagattgccctgcatacaaactctttgatgccgtggaagtaaaaaagaatgaggaagtggagtatcccagaaaatacggggattacacggttgccgtccatagggaccagataccggtaacggttgaagtttcggagaagatataa

SEQ ID NO: 117 - DNA, 663 bp, Clostridium clostridioforme
  source 1..663: mol_type = genomic DNA
atggaatacagggaggatgatttcctcatggtgtccgatatccagcatttcaccttttgcaggcgccagtgggcccttatacatattgaacagcagtggaaagagaatgagcataccatagaaggagagctgctccacaaaaaggcccatgaccaatatttggcagaaaggcggggcgatgtgatgataagcagggcattgcctgtgtattccaggagtatgggtgtcagcggggaatgcgatattgtagagtttcacagggcggaggatggaatcggactccatgggcacaggggactgttccgggtatttccggtggaatataaaaaggggagtccaaaggaatcagaagcagatatactgcagctggtggcacaggccatgtgtctggaggaaatgctgtcagccaggattgaagcgggggcgctgtattacggagagattcaccgcagaaatgtggtggaaattacagatgaactgcgaaaacgggtaagggagatatttcaggagatgcatgaactttatgacaagggatatacaccaaaggtcagatggtcaaagagctgtaatgcctgctccatgaaagatatatgtatgccgaaactgggaaaagcatcacctgtcagggattatatccgtgggaagattgaggaggatgtatga

SEQ ID NO: 118 - DNA, 1032 bp, Clostridium clostridioforme; identical to SEQ ID NO: 32
  source 1..1032: mol_type = genomic DNA

SEQ ID NO: 119 - DNA, 291 bp, Clostridium clostridioforme; identical to SEQ ID NO: 33
  source 1..291: mol_type = genomic DNA

SEQ ID NO: 120 - DNA, 32 bp, Clostridium clostridioforme; identical to SEQ ID NO: 34
  source 1..32: mol_type = genomic DNA

SEQ ID NO: 121 - DNA, 33 bp, Clostridium clostridioforme; identical to SEQ ID NO: 35
  source 1..33: mol_type = genomic DNA

SEQ ID NO: 122 - protein, 230 aa, Erysipelatoclostridium ramosum
MDVYEIKIKLYLLKDIKIEETQTYLAYFIDSVMVKDNMFLGIHETNQYKFYTFDSLYPLAKNGVYQKDNSYMFRIRTLDYQLAQYLYDTLAKNRTKEFQGLTAEVKIIKPKLIKKIYTLTPVILKTEQGYWKNSIKTEDFEKRLKTNLIKKYKDITGEEINEDFQLYYQINFKNKVPVSRKYKGIKLLGDMIELEIAENDNAQKLAFLAIGSGLLEMNARGFGFVNYIYY

SEQ ID NO: 123 - protein, 582 aa, Erysipelatoclostridium ramosum
MKGGYIMLDECLRIFKQELKDNTNLVLNTIILADGDYVLVHDDGTYDVEKIKYSKKDHCFIEKPEDDLYEKLCFYDYQSQVIDTQKTFDLPHKLILSNNYLSYFVKKEKLNNKKKINDATEKYFLALSNPRAKYNGKDLEMYNFVTQNISEVDKRRLLQNKQWLKQYIYNLDGVDYNEKDYLKIFFEADYHQYINEGNRYLLTKIFNCNDYNTYENETVYGVPNYNMQLNSNKPFLKNMTRMHKVPFKLSLEEALIQKQFFDYLLNYANKGKTNIYISSNENDTKRIYFLDNTKKIKKGFNGLYLKIKKDKELKIEYIDTVTDYKEYLSPMFNFRNVIGAFDDEYYQEYNKRNEVEKLLNDVLFSKFLINNYFTDPDSIISIKGENIDVYKKNLITCREAIFAWIHVGAVGNIKNILPKAALETTIHSIKMGHIKLAQRQFNLYFAINEYFNKQENGMKDLRESIRTKINSQQQMKIENDLEYSFAVGQAIAYLQSKSKAKNKTQDFINQFITIKRDDSMKSKLKNLYLHYNYALSTGKTRFNMLFGMIMEYQSDQKINQNMLIAGYIGENLIYEKGENENG

SEQ ID NO: 124 - protein, 305 aa, Erysipelatoclostridium ramosum
MDKRVYGVIGVSSKMANWNADFSGYPRSLSDGTIFGSDKALKYPMRKMWDNEGKKVLYMKSMNFSKKNDTITLVPNSLKERYEKLFAVDDLKKCKDNKEVLTNLMTAVDVKNFGATFAEAKNNISITGAVQFGQGINKYGDSDLETMQILSPFRDTKDAEAKNSTIGTKKIINEAHYFYPFSINPSAYKELVALGVTDGYTEEDYLNFKRTALVAATSFSSNAKEGCQNEFALFVETKLDTYLPNLSEYISFEKADINKIKIECNMLNELEDILNIEIYYNPETTVLESNLEKAKIYNLITKKEV

SEQ ID NO: 125 - protein, 232 aa, Erysipelatoclostridium ramosum
MQALKFTLSGKTAFFKRPEVNTYYFTFGNIHRVALLGIFGAILGYQGYNNTIELPEFYQKLKDIKLSILPQNEKGYFSKKIQYYNNSVGYASKQQGGNLIIKEQWLENVCWDIVVLLDNEEANKLAKAIINKKCIFIPYLGKNDHPANITDPVIVDLQKRDIDKETLDCLFPSGNFKKAEDVYVNFYKYQESLPIGLNEKTGLYEYMTFIHTNFYVDKGAVYSFNDKNIVFY

SEQ ID NO: 126 - protein, 835 aa, Erysipelatoclostridium ramosum
MDIDKYLVSLEKIIINIDQCYGHRDDDHQETINEHIQLCTKYLKEIFKLKKLDSILKSFNISLGEGLSNEGKEMFNELFFNTITFHDTGKINPFFQKNKMNNPVMNYLNPPKNLKSDHSKLSAYIYLGTYLNKLNRLSRNDIGLIKPIVYINAFVISRHHAKIDDFRTYFMDKLDSDDILEKRIIEWMNDEKFISQFTEKLSLDSLNTDEYNYIFEKLEKYEMNIQIDLYAYVRFLYSLLVFCDYYATSEFYGDNKNNVMLKDADFGEIIELYDNSELVTKIRKNDIGKEKINALRTKVFLEAERNLLENIDKNIFYLEAPTGSGKSNTALNLSLRLIENDDSLNKIIYVYPFNTLVEQNLASLNKIFGNSKAMNNIAVVNSTTPLKKDEDENFSDEYQKALLDRQFLTYPIILTTHVGLFDTFFGNNRESAFGLCQYANSVIVLDEIQNYRIEIWNEMIIFLKEFAQILNLKIIIMSATLPDLELLTDDRSNSVSLITNPQEYFLNPIFKDRVKTDYSLINVENTEEALLNHVLEMNKLKKKIMIEFIVRKSAEKFYRQLKEIELDCEVLFISGFDSVLEREKIISTVKTSKHLILVATQVIEAGVDIDMDIGYKDISKLDSEEQFMGRINRSCKEDGKGIVYFFNLNKATNIYKGDERVAQKNLTLFNDDMKKVLLKKNFNYYYQKLLKTLANRAKKPEEKNVPNFFRESVACLDYQAVSKRMELILDTRDRVTIFLGRTVINIDGEEFDGKDIWNQYVDLLKNNEMDYAKKVYELSVVKSMMNYFMYQVIKKNQFDFKEQVGDIYYIENGDDYILDGKLNTALFETEDELFI

SEQ ID NO: 127 - protein, 164 aa, Erysipelatoclostridium ramosum
MEVNGTLINYYFHCQRQCYLHGNRLNLEDESENVKIGKALHEEKYKDDKNSEIAIDNIRLDKLTAKYLTETKKSDADLVAAKWQLLYYLKILKSKGIIRKGRIEVIEKNKQNKSFIEVELTEIEEKELDKIVIKIKELLENDQIPPVLNENKCKKCAYYAYCYI

SEQ ID NO: 128 - protein, 332 aa, Erysipelatoclostridium ramosum
MKSTKYITSMGDLKRKDDSLCFRKSGKNIYLPVENIKEIYCLNEISLNTKLLDFISSKNIIIHFFNYYQGYSGTFYPKDKYTSGKLLIKQVETFNNYRIDIAKAIVNGIAINIDETLYHYYKHGKNEVKETIDWLRKEVPKRLKKAEDIKMIMAVEGEIWQRFYSMFIYILPEDFIMNKRVKRPPDNPINALISFGNTLLYTKTISAIYQTHLNQSISFLHEPTEQRFSLSLDLSEVFKPIIVFKTIFELVNTKRLTVEKHFDKKTNYCLLNDKGRDIFIEAFENRMETKFLHTKLKRKITYKTAIKYDGYKLIKTIFENKLFIPFSIKDKY

SEQ ID NO: 129 - protein, 95 aa, Erysipelatoclostridium ramosum
MKIKNHNYVIVCYDIGEKRVNKIFKICKKYLPHYQYSIFKGPITPSKLILLKKELKKAINKEEDCVSIIKLQSEDSFDEEILGSQKEGNEDSLII

SEQ ID NO: 130 - DNA, 693 bp, Erysipelatoclostridium ramosum
  source 1..693: mol_type = genomic DNA
atggatgtatatgaaataaagataaaattatatttattgaaagatataaaaattgaagaaacacagacttatttagcatactttattgatagtgtaatggttaaagataatatgtttttaggtatacatgagactaatcagtataagttttatacttttgatagtttgtatcctttagctaaaaatggggtttatcaaaaagataattcgtatatgtttagaataagaacgttagattatcaattagctcaatatttatatgatactttagctaaaaatcggactaaagaatttcaaggtctgaccgctgaggttaaaataattaaaccaaaattaatcaaaaaaatatatacacttacaccagtgattttaaaaacagagcagggatactggaaaaacagcattaaaacagaggattttgaaaaacgattaaaaactaatttaattaaaaaatacaaagatataactggtgaagaaattaatgaagattttcaattatattatcaaattaattttaaaaataaggttccggtatcgagaaaatataaaggaattaaattattaggggatatgattgaattagaaattgcagaaaatgataatgctcaaaaattagcttttttagcaatcggatcaggtttgttagaaatgaacgctagaggatttggatttgttaattacatatattattga

SEQ ID NO: 131 - DNA, 1749 bp, Erysipelatoclostridium ramosum
  source 1..1749: mol_type = genomic DNA
ttgaaaggaggttatataatgcttgatgaatgtttacgaatttttaagcaagagcttaaagataatacaaatttagttttaaatacaataattttggctgacggggattatgttttagtacatgatgatggtacttatgatgtagaaaaaattaaatattcaaaaaaagatcattgttttattgaaaaacctgaagatgatctatatgaaaaattatgtttttatgattatcaaagtcaagtaatagatacccaaaaaacttttgatcttcctcataaattaattctttcaaataactatttatcatattttgtaaaaaaagaaaaattgaataataaaaagaaaattaatgatgcaactgaaaaatattttttagcattaagtaatcctagagctaagtataatggtaaagatttagagatgtataattttgtgactcaaaatatcagcgaagttgataaaagaagattattgcaaaataagcagtggttaaaacaatatatttataatttggatggtgttgattataatgaaaaagattatttaaagattttttttgaagcagattatcatcaatatattaatgaaggaaaccgttatcttttgacaaaaatatttaactgtaacgattataacacttatgaaaatgaaacagtgtatggagtaccaaattataatatgcaattaaattctaacaaaccatttttaaaaaatatgacaagaatgcataaagttccatttaaactttccttggaagaagcattgatacaaaaacagttttttgattatttattaaattatgctaataaaggtaaaacaaatatctatattagtagtaatgagaatgatactaaaagaatttattttttagataacaccaaaaaaataaaaaagggatttaatgggctttatcttaaaattaaaaaagacaaagaattaaaaatagagtatatagatacagttactgattataaagaatatttaagtcctatgtttaattttcgaaatgtgattggtgcttttgatgatgaatactatcaagagtataataaaagaaatgaggtagaaaaattacttaatgatgtcttgttttccaagtttttaattaataattattttacggatccagatagtataataagtattaagggtgaaaatattgatgtttataaaaagaatcttattacgtgtagagaagcaatttttgcttggatacatgtaggggcagtgggaaatattaaaaatattttgccaaaagcagcgttagaaacaacgattcactctataaaaatgggacatattaaattggctcaaagacaatttaatctttattttgctataaatgaatattttaataaacaggagaatggtatgaaagatctaagagaaagtattagaacaaaaataaattcccaacagcaaatgaaaatagaaaatgatttagaatatagttttgcagttggacaagcaatagcttatcttcagtcaaagagcaaagctaagaataaaactcaagattttattaatcaatttataactattaagagagatgattctatgaaatcaaaattgaaaaatttatacttacattataattatgctttatctacaggtaagacacgtttcaatatgttgtttgggatgattatggaatatcagagtgatcaaaaaataaatcaaaacatgttgattgctggttatattggagaaaacttaatttatgaaaagggagaaaacgaaaatggataa

SEQ ID NO: 132 - DNA, 918 bp, Erysipelatoclostridium ramosum
  source 1..918: mol_type = genomic DNA
atggataaaagagtatatggagtgattggagtatcatcaaaaatggcaaattggaatgctgatttttctggctatcctagaagtctatctgatggaacgatttttggtagtgacaaagctttaaaatatccaatgagaaaaatgtgggacaatgaaggtaaaaaggtattatatatgaaatctatgaatttttcaaaaaaaaatgatactataacattagtaccaaattcattaaaagagagatatgaaaaattatttgctgttgatgatttaaaaaaatgtaaagataataaggaagtattaactaatttaatgactgctgttgatgttaaaaactttggagcaacatttgcagaagctaaaaataatatttcaattactggtgctgttcagtttgggcagggaattaataaatatggtgatagtgatcttgaaacaatgcaaatactatcaccatttagagatacaaaagatgcagaagcaaaaaattcaactatagggactaaaaaaataatcaatgaagcacattatttctatcctttttcaattaatcctagtgcttataaagaacttgtggctttaggagtcacagatggctacactgaagaagattatttaaattttaaaagaacggctttagtagcagcaacttctttttcatcaaacgctaaagaaggatgtcaaaatgaatttgctttatttgtggaaactaaattggatacatatttaccaaatttaagcgagtatattagttttgaaaaagcagatattaataaaattaaaattgaatgtaacatgttaaatgaattagaagatattttaaatattgaaatttattataatcctgaaacaacagttttagaatctaatttagaaaaagctaaaatctataatttgattactaaaaaagaggtgtag

SEQ ID NO: 133 - DNA, 699 bp, Erysipelatoclostridium ramosum
  source 1..699: mol_type = genomic DNA
atgcaagcacttaagtttactttaagtggtaaaactgctttttttaagcgaccggaagtaaatacgtattattttacttttggtaatattcatagagttgcgctattaggaatatttggggcaattttaggttatcagggctataataatacaattgaactaccagagttttatcaaaaattaaaagatatcaaattatctattttgcctcaaaatgaaaagggttatttttcaaagaaaattcaatattataacaattcagttggttatgcatccaaacaacaaggaggtaatttaattataaaagaacaatggctggaaaatgtttgttgggatattgttgttttgttggataatgaggaggcgaataaattagctaaagctataataaacaaaaaatgtatttttataccctatttaggtaaaaatgatcatccagctaatatcactgatcctgtaattgttgatttacaaaaacgtgatattgataaagaaacgcttgactgcttatttccttcaggaaatttcaaaaaagcagaggatgtatatgttaatttttataaatatcaagaaagtttaccaattggattaaatgaaaaaacgggattgtatgagtatatgacatttattcatacgaacttttatgtggataaaggtgcagtatatagttttaatgataaaaacattgtattttattaa

SEQ ID NO: 134 - DNA, 2508 bp, Erysipelatoclostridium ramosum
  source 1..2508: mol_type = genomic DNA
atggatatcgataaatatctagtttcgctagaaaaaataataattaatattgatcagtgctatggacatagagatgatgatcatcaagaaacaattaatgaacatattcaattatgcacgaaatatttaaaagagatatttaaattaaagaaattggattcaattttaaaatcttttaacatttctttaggagaggggttatcaaatgaaggaaaagaaatgtttaatgagttattttttaacacaattacctttcatgataccggaaaaataaatccgttttttcaaaaaaataaaatgaataatccagtaatgaattatttaaatccgccaaaaaatttaaaatctgatcattcgaaattatcggcatacatctatttaggaacttatttaaataaattgaataggttatcaaggaatgatattggattaataaaaccgattgtttatataaatgcatttgttatttctcgacatcatgctaagattgatgattttagaacatattttatggataaacttgacagtgatgatattttggaaaaaagaattattgaatggatgaacgatgagaaatttataagtcagtttactgagaaattaagtctcgattcattgaatacagatgagtataattatatatttgaaaagctagagaaatatgaaatgaatatacaaatagatctatatgcttatgttagattcttatattcacttctggttttttgtgattattatgcaacaagtgaattttatggtgataataaaaacaatgtaatgcttaaagatgctgattttggtgaaatcattgaactatatgataatagtgaattagttactaagattagaaaaaatgatattggtaaagaaaaaatcaatgcattaagaacaaaagtattcttagaagcggaaagaaatttactagaaaatattgataaaaatattttttatttagaagctccaaccgggagtggtaaaagtaacacagcattaaatcttagtttacgtttaattgaaaatgatgatagtttaaataagataatatatgtatatccttttaatacattagtagaacaaaatctagcatcgttaaataaaatttttggtaatagtaaggctatgaataatattgctgtcgttaattctacaactccattaaaaaaggatgaagatgaaaacttttcggatgaatatcaaaaagcattattagaccgtcaatttctaacatatccaattattctaacgacccacgtaggtctgtttgatactttctttggaaataaccgagaatcagcgtttggtctatgtcaatatgcaaattcggtaatagttttagatgaaatccaaaattatcgaattgaaatatggaatgagatgatcatcttcttaaaagagtttgctcaaattcttaatctaaaaataattatcatgtcagcaacattaccagatttagaattacttacagatgatcgtagtaattcagtttcattaataacaaatcctcaagaatattttttaaatccaatttttaaagatcgagtaaaaacagattatagtctaataaatgtagaaaatactgaagaagcgttattaaatcatgttttagagatgaataagctaaagaaaaaaataatgattgagtttattgttagaaaaagtgcagaaaagttctatcgtcaattaaaagagatagaattagattgtgaagttttatttattagtggttttgatagtgtattagaaagagaaaaaatcattagtactgttaaaacttcaaaacatcttattttagtagcaacccaggtaattgaagccggtgtcgatattgatatggatatcggttataaagatatatctaaacttgatagtgaagaacagtttatgggaagaataaatcgctcatgtaaagaagatggaaaaggaatagtatatttctttaatttaaataaggcaaccaatatttataagggggatgaacgtgttgcacagaaaaatttaacattgtttaatgatgatatgaaaaaagtattgttaaaaaagaactttaattattattaccaaaaacttcttaagactctagcaaatagagctaaaaagcctgaagaaaaaaatgttcccaatttttttagagaaagtgttgcgtgtttagattatcaggcagtaagtaaacgaatggaattgatattagatactagagatcgagtaacgatatttttaggaagaacggtaattaatattgatggggaagaatttgatggtaaagatatttggaatcagtatgtagatttattgaaaaataatgaaatggattatgctaaaaaagtatatgagttgtcagtggttaaaagtatgatgaattattttatgtatcaagttattaaaaaaaatcaatttgattttaaagagcaggttggagatatttattatattgaaaatggtgatgattatattttagatggtaaattaaatacagccctttttgaaactgaggatgagttatttatatag

SEQ ID NO: 135 - DNA, 495 bp, Erysipelatoclostridium ramosum
  source 1..495: mol_type = genomic DNA
atggaagtaaatggaacattaataaactattattttcattgtcaaaggcaatgttatcttcatggtaatcgacttaatttagaggatgaaagtgaaaatgtgaagattggtaaagcattgcatgaagaaaagtataaagatgataagaattcagagattgcaattgataatattaggttagataaattaactgcaaaatatttaactgaaacaaaaaagtctgatgctgatttagttgcagcaaagtggcaactattatattatcttaaaattttaaagtcaaaaggaataatacgaaaaggacgaatagaagtaattgaaaagaataaacaaaataaatcttttattgaagtggagctaacagaaattgaagaaaaagaattagataaaattgttattaaaattaaagaattattagaaaatgatcaaataccaccagttttaaatgaaaataaatgtaaaaaatgtgcatattatgcatattgttatatatag

SEQ ID NO: 136 - DNA, 999 bp, Erysipelatoclostridium ramosum
  source 1..999: mol_type = genomic DNA
atgaaaagtactaaatacataactagtatgggagatttaaaacgtaaagatgattcgctttgttttagaaaaagtggtaagaatatatatttaccagtagaaaatataaaagagatatattgtttaaatgaaatttctcttaatactaaattattagattttatctcatctaaaaatataattattcatttttttaactattatcagggttatagtggtaccttttatccaaaagataaatatactagtggtaaattgttgattaaacaggttgaaacatttaataattatcgtattgatattgcaaaagcaatagttaatggtattgcaattaacattgatgaaactttatatcactattataagcatggtaaaaatgaggttaaagaaacaatagactggttaagaaaagaggtgccaaaacgtttaaaaaaagcagaagatattaaaatgattatggcggttgaaggagaaatatggcaacgtttttatagtatgtttatatatatattacctgaagattttataatgaacaaaagagtaaagagaccacctgataatccaattaatgcactaatttcttttggtaacactttactttatacaaaaacaatttcagcgatatatcaaactcacttaaatcaaagtatttcttttttacatgaacctacagaacaaagattttctttgtctttagatttaagtgaagtatttaaaccgatcattgtttttaaaacaatctttgaattagtcaatactaaaagattaacggttgagaaacattttgataagaaaactaattattgtcttttaaatgataaaggtagggatatatttattgaagcttttgaaaatagaatggaaactaaatttttacatactaaattaaagagaaagataacatataaaacagctattaaatatgatggttataaattaattaaaacaatttttgaaaataaattatttataccattttctataaaggataaatattaa

SEQ ID NO: 137 - DNA, 288 bp, Erysipelatoclostridium ramosum
  source 1..288: mol_type = genomic DNA
atgaaaataaaaaatcataactatgtaatcgtctgttacgatattggggaaaaaagagtaaataaaatatttaagatatgtaaaaaatatcttcctcattatcaatattctatttttaagggtcctattaccccttctaaattaattttattaaaaaaagaactaaaaaaagctataaacaaagaagaagattgtgtaagtattattaaattacagagtgaggattcatttgatgaagaaattttagggagtcaaaaagagggaaatgaagatagtttaattatataa

SEQ ID NO: 138 - DNA, 30 bp, Erysipelatoclostridium ramosum
  source 1..30: mol_type = genomic DNA
gtttaataacaacataagatgtattgaaat

SEQ ID NO: 139 - DNA, 30 bp, Erysipelatoclostridium ramosum
  source 1..30: mol_type = genomic DNA
gtttaataacaacgagatattaaagtgaaa

SEQ ID NO: 140 - protein, 218 aa, Clostridium sp.
MKEVQICQVIYKDIKVNRSTAPMIRGYFANKYNEIEQMHNHKDDKFIYRYPHIQYKVLNNNPVLLGIEDGAKIIQEMTFFLDDVIKIGQKEFESNQREISFKKVKLGVSDKIIKYKFITPWLALNQNNIKKYTAADLIEKEEILKRVLIGNILSFSKGMDYTVDETIKVKLDLNQIDVKFKQKEMLAFIGEFFINFEIPDNFAIGKGISRGFGTVKKI

SEQ ID NO: 141 - protein, 337 aa, Clostridium sp.
MEIDKSYLDEVWVNIIEEVQKDANQSYFIIITKNNKKPRELFTQKYEQKIKETYLISDTKLNKSLKQSKCHLCGQRKKTYDTAIYKCFTNDKEIYKNTDKYNFSICEDCLLDVLNGRNYINDILKISWIGSEVMFLPHEFNVYIKEIYEEIDNDKHITSLLAQIKEAEEEVLDEISKTNCMTDIIFFSDPKSSSEWKITYAIRDVMPSRMSIISRNISKYKNKKIDAYFCLAKIMNYLCYSDGKFDSKNKDRMRLLDIIFHGSKYSRELFFNKVMSKYKTKYFESLNNKKATNRKYIIKDIHEIYNFMCNCGCLENSWMMINEEGEIMKYNNISEFF

SEQ ID NO: 142 - protein, 292 aa, Clostridium sp.
MSNTKSREFLLIWDSTMSNPNGDMLNENRPRQDEITGQLEASDVRVKRFAREEWINKGENVLVQTVEEKGKIQTCQKRVETIQKSAKIKDDDIEKYLTDNYIDVKLFGAVITKPKRDILGPLQVAWSKSVHEAQIKFMQGNAAYASSEGKSQASIWTKYISPYALFKTYAVYNNLVAKKQNINVEDKDIESFIEALIDGMKHYRSTSKNQMPRLLVEVIYNNSKLDGELDYIDIIKDVEDEELRNIDQVQIDLSKLSRYYDLKKDDIEKIVIYTHPSVKLLNIDDRFELKTI

SEQ ID NO: 143 - protein, 224 aa, Clostridium sp.
MKCLVFKVRGDYARFRKSYTTTSALTYLLIHPVAVRGLIGAMLGIDREKLYEKTKDIEVAIQVVNQVRKDMQSFNLVNMKPNDKIFRFPSNVEFLRNVEYRIFIKAEDKTLYEIDNCIKQGEYIFTPYLGSSEHIAKIEYEGIYNCEKLNSGNYKVISAVSLEENKIYFGNGNIILMTDNIPTRRDIDREYIEYKKVIFATNENTINVETENCYKVGGYNVIFI

SEQ ID NO: 144 - protein, 757 aa, Clostridium sp.
MSYSYKLKSHPTQSLYDHLTGVRDIALKTHKHHTIKPEIDDFIEIVCMCHDFGKGTTYFQRYIENDFRGIEKDHGPISAMFTYWMLPDKWKHLGFLIVKKHHGDINNASDECRIDEVSWDFKKQIKDILDNTIDELNQIYDKYLEDKNIEAFLNWLDDESNLKSIKKEFRKKKYNIEDLLLCEYVYSLLLTGDKSQLIRNDAYIPDKQYPLSFIENYKADLVKDALIKNPKLKESDVFNLRNEIYDDMINKLDTIDFDKENIFSINVPTGTGKTILAYSAAFYICNKIIKNNLNIRPHIIYSLPFTSVIDQNYEVLKEIVENNINKEISSEDLMKFHSVVPIEYENFEGYDARFCFENWQSKIISTTFVQLLNTIFKIGKNSIVNRFHRLANSVIILDEVQAIDEKYYKIISEFISIMVRQYNCYIILVTATMPMLLDTIDLIEDKEKYFRKLNRIEIINNSESEITLDEFEDIVLEDICENKDKSFLIVLNTVKSSKNIYKYLKENTDRDIMYLSTEIYPKLRLEKINKIKNSDKKYVVVSTQLIEAGVDIDMDIVYRDFSTLDSINQTAGRANRNGVGGKGIVKLYKIVDNDRRICNYIYPRYLLNATEEVLDGKDIIEEKDIYDCNKEYFLKVKNRLSNDTSDELLDLIPKLQFKKFRDKFELIENDEFRRVDIIVNADNITSSIIQQLENDNEIDNIDLKNKFRILRQYIVSVPRKDMSEINDIGLKKYNITYINKEDYSEEEGIIRQKQVIW

SEQ ID NO: 145 - protein, 203 aa, Clostridium sp.
MKSSQSFFNEFDYYITPSELIEFNYCKRFTYYMKCLNVPQFEENRFKVQKGKEIHSKKENENKQYLRKKLGVIDKKINVDLYSEKYKIKGIVDEVLTLSDNTMAPLDYKFAKFDDVLYKTYKNQMLMYSIMIEEMYNCKVNKCFLVYTRSSNLIKEFNVEEKELKKLKKDIRDYFKVMEGYFPKATSSKARCIDCCYRNICIK

SEQ ID NO: 146 - protein, 328 aa, Clostridium sp.
MKLVVNSEGVYLTKVGECFCLKKEGQKQEIAAKKVEQIIITTSTALSTDAIELAVENNIDIVFLKKYGKPFGRVWHSKVGSISTIRKRQLNLDSNKMGTILVKEFLTQKMTNQVEFLNELSMNRRDNRKIEIKEDVEKIKGLIEKIKSISEESKINDIRGSLQGYEGTASRIYFSRLSDLMPEKYKFNGRSKNPAKDEFNSMLNYGYGIMYSNVERACILAGLDPYIGIMHVDNYNRQAFVFDLIEMYRIYIDKIVFKLFTTKKVKKEHFDKVEGGLYLNKAGKEILISKYNEEIEKKIQYKGRKIELQNIIQYDCHNIANKILKEGD

SEQ ID NO: 147 - protein, 96 aa, Clostridium sp.
MLVWVVYDISSNKVRRNAIKACKNVGLYRVQKSVFLGEIEENDFDELKLKMEDIIDLTSDSVYIFPMTKKGLNQAGLIGQVFDKELVTDEIISKFF

SEQ ID NO: 148 - DNA, 657 bp, Clostridium sp.
  source 1..657: mol_type = genomic DNA
atgaaagaagttcaaatatgtcaggtaatatataaagatataaaagtaaatagatctacagcacctatgataagaggatattttgcaaataaatacaatgaaatagaacaaatgcataatcataaagatgataaatttatatatagataccctcatattcaatataaagtactaaataataatcccgtattgttgggaatagaagatggtgcaaaaataattcaagaaatgacattttttttagatgatgtaataaaaatcggtcaaaaagaatttgaatctaaccaaagagaaatatcttttaaaaaagttaaattaggagttagtgataaaataataaaatataaatttataactccatggttagctttaaatcaaaataatattaaaaaatatacagcagcagatctaatagaaaaagaagaaatactaaaaagagtacttataggaaatattttgtctttttcaaaaggaatggattatacagttgacgaaactataaaagtaaagttagacttaaatcaaatagatgttaaatttaaacaaaaagaaatgttagcttttataggagaattttttataaactttgaaataccagataattttgcgataggtaaaggaatatcgagaggatttggaactgtaaaaaagatttaa

SEQ ID NO: 149 - DNA, 1014 bp, Clostridium sp.
  source 1..1014: mol_type = genomic DNA
atggaaatagacaaaagttatctagatgaagtttgggtaaatataatcgaggaagttcaaaaagatgcaaatcaatcgtattttattataatcacaaaaaataacaaaaaacctagagaattattcacacaaaaatatgaacaaaaaataaaagagacatatctaatatcagacactaaattaaataaatccctaaaacaaagtaagtgccacttatgtggtcaaagaaaaaaaacttatgacacagcgatatataaatgttttacaaatgataaagaaatttataaaaatacagataaatataacttctctatatgtgaagattgcttattagatgtattaaacggaagaaactatatcaatgatattctaaaaataagctggattggttctgaagtgatgtttttgccacatgaatttaatgtatatataaaagaaatatatgaagaaatagataatgataaacatataacatctttactcgctcaaataaaagaagctgaggaagaagtacttgatgaaatttcaaagacaaattgtatgacagatataatatttttctcagatcctaaaagtagttctgagtggaagattacttatgcaataagagatgtaatgccttctagaatgagtataatttcaagaaatataagcaaatataaaaataagaaaatagatgcatatttttgtttagctaaaattatgaattatctatgttatagcgatggaaagtttgattctaagaataaagatagaatgagattgttagatattatttttcatggaagtaaatatagtagagaactatttttcaacaaagttatgtcgaaatacaaaacaaaatactttgaaagcttaaacaataaaaaggctacaaacagaaaatacataataaaagatatacacgaaatatataactttatgtgtaattgtgggtgtttagaaaattcatggatgatgataaatgaggagggtgaaattatgaaatacaataatataagtgagtttttttga

SEQ ID NO: 150 - DNA, 879 bp, Clostridium sp.
  source 1..879: mol_type = genomic DNA
atgtcaaacacaaaatcaagagaatttttattaatatgggattcaacaatgtcaaatccaaatggagatatgttaaatgaaaacagaccacgtcaagatgagattacaggtcaattagaagcatctgatgtcagagtaaaacgttttgcaagagaagaatggataaataaaggtgaaaatgtattagttcaaacagttgaagaaaaaggtaaaatacaaacttgtcaaaaaagagttgaaactattcaaaaatctgcaaaaataaaagatgacgacattgaaaaatatttaactgataattatatagatgttaaattatttggggcagttataacaaagccaaaaagagatatattaggtcctcttcaagttgcatggagcaaatctgtacatgaagctcaaataaagtttatgcaaggtaatgcagcatatgcgagttcagaaggaaaatcacaagcatctatttggacaaagtatatatctccatatgcactatttaaaacatatgctgtttataataacttagtagcaaaaaaacaaaatataaatgtagaagataaagatatagaatcatttatagaagcacttatagatggtatgaaacattatagaagtacatctaaaaatcaaatgcctagattattagtagaagtaatttataataatagcaaattagatggagaactagattatattgatataataaaagatgttgaagatgaagaacttagaaatatagaccaagttcaaatagatttatcaaaattatcaagatattatgatttgaagaaagatgatatagaaaaaatagttatttatacacatccaagtgtaaaactattaaatatagacgatagatttgaacttaaaactatatag

SEQ ID NO: 151 - DNA, 675 bp, Clostridium sp.
  source 1..675: mol_type = genomic DNA
atgaaatgtttagtttttaaagttagaggtgattatgcaagatttagaaaatcatatacaacaacctcagctctaacttatttattaattcatccagtagctgttagagggttaataggtgctatgcttgggattgatagagaaaaactctatgaaaaaacaaaagatattgaagtcgctattcaagttgtaaatcaagttagaaaagatatgcaatcttttaatttagtaaatatgaaagctaatgataaaattttcagatttccatcaaatgtagagtttttgagaaatgtagaatatagaatatttataaaagcagaggataaaactttatatgaaattgataattgcataaaacaaggggaatatatatttactccatacttaggttcatctgaacatatagccaaaatagagtatgaaggtatttataactgtgaaaaattaaatgcaggaaattataaagtaattagtgcagtatccttagaagaaaataaaatatattttggagatagtgatataatacttatgactgataatattccaacaagaagggatatagatagagaatatatagaatataaaaaagtgatatttgctacaaatgaaaatactataaatgtaggtacagaaaattgctataaagtaggtggatataatgtcatattcatataa

SEQ ID NO: 152 - DNA, 2274 bp, Clostridium sp.
  source 1..2274: mol_type = genomic DNA
atgtcatattcatataaattaaaatcacatcctacccaaagcttatatgatcatcttacaggggttagagatatagcattaaaaacacataaacaccatactataaaacctgaaatagatgattttatagaaattgtatgtatgtgccatgactttggaaagggaactacatattttcagagatatattgaaaatgattttagaggaatagaaaaggatcatggacctatttcagctatgtttacatattggatgcttccagataagtggaagcatctagggtttcttatagtgaaaaaacaccacggagatataaataatgcaagtgatgagtgcagaattgatgaggtatcttgggattttaaaaaacagataaaagatatattggataatactatagatgaattaaatcaaatatatgataaatacttagaagataaaaatatagaagcatttttaaattggttggatgatgagtctaatttaaaaagtataaaaaaagaatttagaaagaaaaaatataatatagaagatttacttttatgtgaatatgtatattctcttcttttaacaggagataaaagtcaacttatacgaaatgatgcctatataccagataaacaatatccactttcttttattgaaaattataaggcagatttagttaaagatgcgcttattaaaaatccaaagttaaaagaatcagatgtgttcaatttaagaaatgaaatatatgatgatatgataaataaattagatactatagactttgataaagaaaatattttttctataaatgttccaacaggaacaggcaaaactattttggcttattctgcagctttttatatttgtaataagataataaaaaataatttaaatattagacctcatattatatattcattaccatttacatctgtaatagatcaaaattatgaggtgttaaaagagatagttgaaaacaatataaataaagaaatatcatctgaagacttgatgaagtttcatagtgttgtgcctatagagtatgaaaactttgaaggatatgatgcacgtttttgttttgaaaattggcaaagtaaaataatatcaacgacatttgtacagcttttaaatactatatttaaaattggaaaaaactcaatagtaaatagatttcatagacttgcaaattcagttataattttggacgaggttcaagcaattgacgaaaaatattataaaataatttcagagtttataagtataatggtcagacagtacaattgttatattatattagttactgctacaatgccgatgcttttagatactatagatcttattgaagataaggaaaaatactttagaaaactaaatagaattgagataattaataattcagaaagtgaaataactttagatgaatttgaagatattgttttagaggatatttgtgaaaataaagataaaagctttcttattgttttaaatactgtaaaaagttctaaaaatatttataagtacttaaaagaaaatacagatagagatattatgtatctttctacagagatttatccaaagttaagactagaaaaaataaataaaataaaaaatagcgataaaaaatacgtagttgtatcaactcaacttatagaagctggcgtagatatagacatggatatagtatatagagatttttcaacattagattcaataaaccaaacagcaggaagggcaaatagaaatggtgttggaggaaaaggaatagtaaaactttataaaatagtagataatgatagaagaatatgcaattatatttatccaagatatctactaaatgcaacagaagaagttttagatggaaaagatattatcgaagaaaaagatatttatgactgtaataaagagtattttttaaaagtaaaaaatagactatcaaatgatacatcagatgaacttttagacttgattccaaaattacaatttaaaaagtttagagacaagtttgaacttatagaaaatgatgaatttagacgagtggacataatagtaaatgctgataatattacgagttctataatacaacaattagaaaatgataatgaaatagataatattgatttaaaaaataagtttaggatattaagacaatatatagtatctgttcctagaaaagatatgtctgaaattaatgatatagggttgaaaaaatataatataacatatataaataaagaagattattcagaagaagaaggtattattagacaaaaacaagtaatctggtag

SEQ ID NO: 153 - DNA, 612 bp, Clostridium sp.
  source 1..612: mol_type = genomic DNA
atgaaatcatctcaaagtttttttgatgaattcgattactatataacaccttctgagcttatagaatttaattattgtaaaagatttacttattatatgaaatgcttgaacgtaccacaatttgaagaaaatagatttaaagtacaaaaaggaaaagaaatacattctaaaaaagaaaatgaaaataaacagtatcttcgtaaaaaattaggtgttatagataaaaaaataaatgttgatttatattctgaaaaatataaaattaagggtatagtagatgaagttttaacattaagtgataatactatggcacctttggattataaatttgctaaatttgatgatgtactatataaaacatataaaaatcaaatgttgatgtactctattatgattgaagaaatgtataactgcaaagtaaataaatgctttttagtttatacaagaagctcaaatttaataaaagaatttaatgtagaagaaaaagaattaaaaaagctaaaaaaagatataagagattattttaaagtgatggaaggatattttccaaaagcgacatcatcaaaagctagatgtatagattgttgctatagaaatatatgcataaaataa

SEQ ID NO: 154 - DNA, 987 bp, Clostridium sp.
  source 1..987: mol_type = genomic DNA
atgaagctagttgttaattcggaaggagtatatttgactaaagtaggcgaatgtttttgtttgaaaaaagaggggcagaaacaagaaattgctgctaagaaggttgaacaaataattataacgacatctacagctttgtctactgatgctattgagttagctgttgagaataatattgatatagtgtttttaaaaaagtatggaaaaccttttggaagagtatggcattctaaagttggtagcataagtactattagaaaaagacagttaaatttagattctaataaaatgggtacaattttagtaaaagagtttttgactcaaaagatgactaatcaggttgaatttttaaatgaattatctatgaatagaagagataatagaaaaattgaaataaaagaagatgtagaaaaaataaaggggttaatagaaaaaattaaatctatatcagaagaatcaaaaataaatgatataagaggaagtttacaaggatatgaagggactgcaagcagaatatatttctctagattatcagatttaatgccagaaaaatataagtttaatggacgtagcaaaaatccagctaaagatgaattcaactgtatgttaaattacggatatgggataatgtattctaatgtagagagagcatgtatattagcggggttagatccttatataggaataatgcatgttgataattataatagacaagcttttgtatttgacttaattgaaatgtataggatttatattgataaaattgtatttaaactttttacaactaagaaagttaaaaaagaacattttgataaggttgaaggaggattatatctaaataaagctggaaaagaaatattaatttccaagtataacgaagaaatagaaaagaaaattcaatataaaggtagaaagatagaattacaaaatataattcaatatgactgtcataatatagccaataaaattttaaaggagggagattga

SEQ ID NO: 155 - DNA, 291 bp, Clostridium sp.
  source 1..291: mol_type = genomic DNA
ttgttagtctgggtagtatacgatatttcgagtaataaggttagacgaaatgctattaaagcgtgtaaaaatgttggactatacagagtacaaaaaagcgtgtttttaggtgaaatagaagaaaatgatttcgatgaattgaagctaaaaatggaagatataatagatttgactagtgattcagtatatatattcccaatgactaaaaaaggactgaatcaagcaggattaattggacaagtttttgataaggagttggttactgatgaaatcatctcaaagtttttttga

SEQ ID NO: 156 - DNA, 32 bp, Clostridium sp.
  source 1..32: mol_type = genomic DNA
gtttcaatccacgcacccgtgcagggtgcgac

SEQ ID NO: 157 - DNA, 30 bp, Clostridium sp.
  source 1..30: mol_type = genomic DNA
gtttcaattccaacatggtacgattaaagc

SEQ ID NO: 158 - DNA, 30 bp, Clostridium sp.
  source 1..30: mol_type = genomic DNA
gtttcaattccaatatggcacgattaaagc

SEQ ID NO: 159 - DNA, 30 bp, Clostridium sp.
  source 1..30: mol_type = genomic DNA
tgctttattacaatgtggtaagagtaaagc

SEQ ID NO: 160 - DNA, 29 bp, Clostridium sp.
  source 1..29: mol_type = genomic DNA
tttcaattccaacttggtacgattaaaac

SEQ ID NO: 161 - DNA, 30 bp, Clostridium sp.
  source 1..30: mol_type = genomic DNA
aacgcaattccaacttggtacgattaaaac

SEQ ID NO: 162 - DNA, 30 bp, Clostridium sp.
  source 1..30: mol_type = genomic DNA
gtttcaattccaacttggtacgattaaaac

SEQ ID NO: 163 - DNA, 30 bp, Clostridium sp.
  source 1..30: mol_type = genomic DNA
gttgcaattccaatatggtaagattaaagc

SEQ ID NO: 164 - DNA, 27 bp, Clostridium sp.
  source 1..27: mol_type = genomic DNA
tcaattccaatatggtacgattaaaac

SEQ ID NO: 165 - DNA, 403 bp, Clostridium scindens
  source 1..403: mol_type = genomic DNA
ttaaatcacaagttattttgacgcaggggcagtatgcctctgcgtcttttgaatattgttagaaagattttcttactatagccaacaagaattaagggtggtggaatagtatgtctaaaggagttcgagacggcagtccttcttatcattaaaactttatgaaaaagctaattgagaaattagaaatataaaaaaagaataaatttccttgaaaattagatatcgattcagtagtgcgaatgtgaagcgtacataaaaatcccaggatattcgcaccaaaaaagttgcacaaaaatagtgtattgtattacaatatagattagatgagagaatgtatctaaaggtaactgtaatatagcacaaaaattttgcagttatatattaaaaattggataagtttgct

SEQ ID NO: 166 - DNA, 180 bp, Clostridium scindens
  source 1..180: mol_type = genomic DNA
agtggtttagtgcgaatgtgaagtgcacatgaaaatgcagggagattcgcacctggatattgatgatatgagggctagggaagttataatttgttgtgtaggtgttggaaaaagttcttttgtttggcgttttgcataagagattggctgtatattaggagttttatgtgcaaaattgct

SEQ ID NO: 167 - DNA, 185 bp, Clostridium scindens
  source 1..185: mol_type = genomic DNA
caattgaaatttggtgcgaatgtcaagcaaacataaaattccagggggattcgcaccaaaaaaagatacaaaatggaatggtaatgctaagaatatgtaataaaaactggtgataaatatatgaaataatagttaatttgttggaaattaacaaaaagtcctgtataatattatatgatattgca

SEQ ID NO: 168 - DNA, 213 bp, Clostridium clostridioforme
  source 1..213: mol_type = genomic DNA
agtgtgatttctgtgaggcggaatcccatcaaaccaggtgcgaatgccaagctaacatgaaatgtccgggagattcgcacctgaaaaacagaggaaaagccgtgaaaataggctgggtgaatggatggaggaggttgaaataagggaggggggggggggacataatgcataaaaagttagggggatgtggagggattgttggatagttttgct

SEQ ID NO: 169 - DNA, 210 bp, Clostridium clostridioforme
  source 1..210: mol_type = genomic DNA
cctggccgcgggcggtaaagcggtataaagtattttggtgcgaacctgaagcaaacatggatttacggggagattcgcaccggaaaatttgcacaatgagaacggatttgcgagtgtgcagggatggttggccgtgatttgagggggtgggatatgatttgtttgtggaaatgtgttgaagaatagtagtggttattgtgtagtattgct

SEQ ID NO: 170 - DNA, 210 bp, Clostridium bolteae
  source 1..210: mol_type = genomic DNA
cctggctgcgggcggtaaggaggtttcaaatagtttggtgcgaaccagaagcaaacatgaatttacggggggattcgcaccggaaaaactgcacagtgagaacggttttggatgtgtgcagggcaggttggcggtgatttgagggagtatgatgtgatttgattgtggaaatgtgatgaagaattgttggtttttttgtgtagtattgct

SEQ ID NO: 171 - DNA, 207 bp, Clostridium bolteae
  source 1..207: mol_type = genomic DNA
agttggtttctgtgagttgggagcccttcaaaccaggtgcgaatgccaagctgacatgaaatgccagggagattcgcacctgaaaaacagaggaaaaggccgtgaaagcgggctgggtgaatggactgaattggttgaactggggagagaagagcataatgcataaaatgttaggggggatgtggagtaggtttggataattttgct

SEQ ID NO: 172 - DNA, 117 bp, Clostridium scindens
  source 1..117: mol_type = genomic DNA
ttaaatggtaaaagggcttgaaaaacaaatagatttgaaatataatcagagatagccgagcaaacgtaaaaaattataaatgccgaaaaggcgctatatttaaggaggatattcaaa

SEQ ID NO: 173 - DNA, 118 bp, Clostridium clostridioforme
  source 1..118: mol_type = genomic DNA
gatagattcttgataaaaggcttgtaaactaaaggaaaatagaatataatggttgatagcctgacattaactataatagcccactataaagggcgcaaattaaggaggacgttataaa

SEQ ID NO: 174 - DNA, 118 bp, Clostridium bolteae
  source 1..118: mol_type = genomic DNA
gatagattcttgataaaaggcttgtaaactaaaggaaaatagaatataatggttgatagcctgacattaactataatagcccattatacagggcgcaaattaaggaggacgttataaa

SEQ ID NO: 175 - DNA, 119 bp, Clostridium clostridioforme
  source 1..119: mol_type = genomic DNA
atcgtgatataagtatatagcagaaacagagagtgataacagagcctaggtatcgaaacaggatgcctggctcttttgctttaccaggcatatgaacgttgtggattaaaggagaaaag

SEQ ID NO: 176 - DNA, 119 bp, Clostridium bolteae
  source 1..119: mol_type = genomic DNA
atcgtgatataagtatatagcagaatcagagagtgataacagagcttgggcatcgaaacaggatgccgggctcttttgttttgtcaggcatatgagcgctgcggattaaaggagaaaag

SEQ ID NO: 177 - DNA, 310 bp, Clostridium sp.
  source 1..310: mol_type = genomic DNA
aattaatttttatataggaggtgagtttatgagttgtatcacaatgagttttaaatgcctgttctgttccaaacgttcaggctgtccaattgcattttaattgaaggaggttattatatgggtattgtaaaattaaatagtttttataggcattgtgcaatatgtggtttgtgcagtgtctttttttatgtataaattgattaatttaaaatgaggaatacagattaaaaaaagacaaagaatttagttgaatttgatggctttttttttgagggtaatgtctaaaaaaatattataaaaagttggtact

SEQ ID NO: 178 - DNA, 269 bp, Clostridium sp.
  source 1..269: mol_type = genomic DNA
tttttcaatattgtagactttttggtgtatttagtataaaatctaataaagtaattaaatatcttatctaatattgaaatattctaacctttaaatttaaagtttttgcggtattttagcttattttttaatccaactgccacgcaccgctgataccaacggtttgaggtacttttaaggacgtaaagtttttcttatatgtaagaaaaaagttgtaccccgcaacaggggtctgaaagctagtatttgcaagggctgaaaatttaggg

SEQ ID NO: 179 - DNA, 303 bp, Clostridium sp.
  source 1..303: mol_type = genomic DNA
ataaaattaaacttccccgaaattttatttttggggagttatttttatttttactgtttatgacacttctttcaactacataaaaaatctttatctttagagttatttatgtaaatcactccttattatacatataaatttatacaaataataacataaattgtaaaattattattaatgttcatatcttaaaaatctattttttatctttaaaacatcttacatattttagcaaatttcattatgtaaaacaccctatttaataaaacaatttaaacagagtagaaaaatagtaaaactttt

SEQ ID NO: 180 - DNA, 186 bp, Clostridium sp.
  source 1..186: mol_type = genomic DNA
ggaggctgcagtgcatatggatatttctgtggagtttgtagagacttgcttgtatgagataattgtaagaaaaaaagagagttagattttttaactctcttttgaaatttcaacttcaaaataccaaagaaaaactttttgaatctcttttcatattaagtagtttagtttctatactattttatt

SEQ ID NO: 181 - DNA, 205 bp, Clostridium sp.
  source 1..205: mol_type = genomic DNA
aaaagatttttaatagttttgttcctacaaaattattggaaattttgaactttgacaattgaatgtgttggaattacttggtttgagatagatgttaataagctaaaaaatgcattttttttaatgggaaatcattgttttgaggttagatgaaaataaagcaacacattgaaaatcagtaattccaatggttttaggttctacg

SEQ ID NO: 182 - DNA, 147 bp, Clostridium sp.
  source 1..147: mol_type = genomic DNA
ataatcacctcctgcaaagaatattaataattacatatattaataaaaccttatatttagataatagtcctattgttattatatgtcaataaatgtttttaaattttaatccttgttttattggaattttataattacatcgattaa

SEQ ID NO: 183 - DNA, 174 bp, Erysipelatoclostridium ramosum
  source 1..174: mol_type = genomic DNA
tttaccaggctcaatttctttttaatgtcctaaaactgttgggagagtaagcgtttaagagaattttactagcaattataaaaaagtaaaatcatttggtaaaaaagtagaaaagtattgatagatataggttttgaggtataatagaaagtgtaaaatggcttatttactagg

SEQ ID NO: 184 - DNA, 260 bp, Clostridium sp.
  source 1..260: mol_type = genomic DNA
tccagataaattaaacttatataattaaatccaggctggttatataattatataatcatcctgtaatataaaaattaaaaataatattataaaaagtaaacaaaaagacaacaaaaaacaactaatcgagaaaaataataacaaaattaaaattaaaattaaaatacatagaaatttatgaacttacccatcaatttgtttgtgatattttatgttttttacaaactatgacttgattagttcaatggaaggagaataac

SEQ ID NO: 185 - DNA, 84 bp, Erysipelatoclostridium ramosum
  source 1..84: mol_type = genomic DNA
tatccaatcaaaatcttattgattttttcaactaaataaattagaatgtatatgtacaaaaatataaactaggaggaaccttaa

SEQ ID NO: 186 - DNA, 42 bp, Clostridium sp.
  source 1..42: mol_type = genomic DNA
aaaagatcccggattctgtatgatgcagagtccgggattttt

SEQ ID NO: 187 - DNA, 31 bp, Clostridium sp.
  source 1..31: mol_type = genomic DNA
ggaacccctggccaatatggtcaggggttct

SEQ ID NO: 188 - DNA, 45 bp, Clostridium sp.
  source 1..45: mol_type = genomic DNA
atgaattgcagaaatgcatttcagatatttttgaaccttgaaaac

SEQ ID NO: 189 - DNA, 31 bp, Clostridium sp.
  source 1..31: mol_type = genomic DNA
cccctatttttgtgcaatatgtagaaaaata

SEQ ID NO: 190 - DNA, 39 bp, synthetic construct
  misc_feature 1..39: note = rho independent terminator
  source 1..39: mol_type = other DNA
aaaaaaaaaccccgcccctgacagggcggggtttttttt

SEQ ID NO: 191 - DNA, 39 bp, Lactobacillus crispatus
  source 1..39: mol_type = genomic DNA
caaaaaaagcatgagaattaattttctcatgcttttttg

SEQ ID NO: 192 - DNA, 38 bp, Lactobacillus crispatus
  source 1..38: mol_type = genomic DNA
aaaaaagatgcacttcttcacaggagcgcatctttttt

SEQ ID NO: 193 - DNA, 32 bp, Lactobacillus crispatus
  source 1..32: mol_type = genomic DNA
caaaaagagcggctataggccgctttttttgc

SEQ ID NO: 194 - DNA, 37 bp, Lactobacillus crispatus
  source 1..37: mol_type = genomic DNA
gtaaaaatggcttgcgtgttgcaagccatttttttac

SEQ ID NO: 195 - DNA, 54 bp, synthetic construct
  source 1..54: mol_type = other DNA
atgctaatggagtttactacaagttaatccggcaaagctaaatggccggcccgt

SEQ ID NO: 196 - DNA, 10 bp, Clostridium sp.
  source 1..10: mol_type = other DNA
ttacaattta

SEQ ID NO: 197 - DNA, 10 bp, Clostridium sp.
  source 1..10: mol_type = other DNA
gtttttataa

SEQ ID NO: 198 - DNA, 10 bp, Clostridium sp.
  source 1..10: mol_type = other DNA
atcccattta

SEQ ID NO: 199 - DNA, 10 bp, Clostridium sp.
  source 1..10: mol_type = other DNA
cattttcttt

SEQ ID NO: 200 - DNA, 10 bp, Clostridium sp.
  source 1..10: mol_type = other DNA
accattttta

SEQ ID NO: 201 - DNA, 10 bp, Clostridium sp.
  source 1..10: mol_type = other DNA
aatttagcta

SEQ ID NO: 202 - DNA, 10 bp, Clostridium sp.
  source 1..10: mol_type = other DNA
ttctttttta

SEQ ID NO: 203 - DNA, 10 bp, Clostridium sp.
  source 1..10: mol_type = other DNA
ataatctgta

SEQ ID NO: 204 - DNA, 10 bp, Clostridium sp.
  source 1..10: mol_type = other DNA
tgaaagttta

SEQ ID NO: 205 - DNA, 10 bp, Clostridium sp.
  source 1..10: mol_type = other DNA
accgagtcgg

SEQ ID NO: 206 - DNA, 10 bp, Clostridium sp.
  source 1..10: mol_type = other DNA
actttattta

SEQ ID NO: 207 - DNA, 10 bp, Clostridium sp.
  source 1..10: mol_type = other DNA
tatatattct

SEQ ID NO: 208 - DNA, 10 bp, Clostridium sp.
  source 1..10: mol_type = other DNA
gtattattta

SEQ ID NO: 209 - DNA, 10 bp, Clostridium sp.
  source 1..10: mol_type = other DNA
cccagaaggt

SEQ ID NO: 210 - DNA, 10 bp, Clostridium sp.
  source 1..10: mol_type = other DNA
gtaacattta

SEQ ID NO: 211 - DNA, 10 bp, Clostridium sp.
  source 1..10: mol_type = other DNA
ttataattaa

SEQ ID NO: 212 - DNA, 10 bp, Clostridium sp.
  source 1..10: mol_type = other DNA
tcttctttta

SEQ ID NO: 213 - DNA, 10 bp, Clostridium sp.
  source 1..10: mol_type = other DNA
tttacagcca

SEQ ID NO: 214 - DNA, 10 bp, Clostridium sp.
  source 1..10: mol_type = other DNA
tttagtttta

SEQ ID NO: 215 - DNA, 10 bp, Clostridium sp.
  source 1..10: mol_type = other DNA
cagattgttc

SEQ ID NO: 216 - DNA, 10 bp, Clostridium sp.
  source 1..10: mol_type = other DNA
cgattattta

SEQ ID NO: 217 - DNA, 10 bp, Clostridium sp.
  source 1..10: mol_type = other DNA
taactgagat

SEQ ID NO: 218 - DNA, 10 bp, Clostridium sp.
  source 1..10: mol_type = other DNA
agtcatttta

SEQ ID NO: 219 - DNA, 10 bp, Clostridium sp.
  source 1..10: mol_type = other DNA
ttacagctat

SEQ ID NO: 220 - DNA, 10 bp, Clostridium sp.
  source 1..10: mol_type = other DNA
attttcttta

SEQ ID NO: 221 - DNA, 10 bp, Clostridium sp.
  source 1..10: mol_type = other DNA
ccaatatcta

SEQ ID NO: 222 - DNA, 10 bp, Clostridium sp.
  source 1..10: mol_type = other DNA
tctgctttta

SEQ ID NO: 223 - DNA, 10 bp, Clostridium sp.
  source 1..10: mol_type = other DNA
ttatcagtta

SEQ ID NO: 224 - DNA, 10 bp, Clostridium sp.
  source 1..10: mol_type = other DNA
gagcttttta

SEQ ID NO: 225 - DNA, 10 bp, Clostridium sp.
  source 1..10: mol_type = other DNA
actctctatt

SEQ ID NO: 226 - DNA, 10 bp, Clostridium sp.
  source 1..10: mol_type = other DNA
cactccttta

SEQ ID NO: 227 - DNA, 10 bp, Clostridium sp.
  source 1..10: mol_type = other DNA
ttttgcaaca

SEQ ID NO: 228 - DNA, 10 bp, Clostridium sp.
  source 1..10: mol_type = other DNA
ctggtgttta

SEQ ID NO: 229 - DNA, 10 bp, Clostridium sp.
  source 1..10: mol_type = other DNA
aactttcact

SEQ ID NO: 230 - DNA, 10 bp, Clostridium sp.
  source 1..10: mol_type = other DNA
catctgtaag

SEQ ID NO: 231 - DNA, 10 bp, Clostridium sp.
  source 1..10: mol_type = other DNA
aaagttttgc

SEQ ID NO: 232 - DNA, 10 bp, Clostridium sp.
  source 1..10: mol_type = other DNA
tttattttta

SEQ ID NO: 233 - DNA, 10 bp, Clostridium sp.
  source 1..10: mol_type = other DNA
aagcgttttg

SEQ ID NO: 234 - DNA, 10 bp, Clostridium sp.
  source 1..10: mol_type = other DNA
aattcattta

SEQ ID NO: 235 - DNA, 10 bp, Clostridium sp.
  source 1..10: mol_type = other DNA
ctcctacatc

SEQ ID NO: 236 - DNA, 10 bp, Clostridium sp.
  source 1..10: mol_type = other DNA
aaggtcttta

SEQ ID NO: 237 - DNA, 10 bp, Clostridium sp.
  source 1..10: mol_type = other DNA
ttgaattttt

SEQ ID NO: 238 - DNA, 10 bp, Clostridium sp.
  source 1..10: mol_type = other DNA
tagattttta

SEQ ID NO: 239 - DNA, 10 bp, Clostridium sp.
  source 1..10: mol_type = other DNA
tattgttttt

SEQ ID NO: 240 - DNA, 10 bp, Clostridium sp.
  source 1..10: mol_type = other DNA
tttgcattta

SEQ ID NO: 241 - DNA, 10 bp, Clostridium sp.
  source 1..10: mol_type = other DNA
ccactgaatt

SEQ ID NO: 242 - DNA, 10 bp, Clostridium sp.
  source 1..10: mol_type = other DNA
aaatgcttta

SEQ ID NO: 243 - DNA, 10 bp, Clostridium sp.
  source 1..10: mol_type = other DNA
atccttttga

SEQ ID NO: 244 - DNA, 10 bp, Clostridium sp.
  source 1..10: mol_type = other DNA
agattgttta

SEQ ID NO: 245 - DNA, 10 bp, Clostridium sp.
  source 1..10: mol_type = other DNA
aagaatgcaa

SEQ ID NO: 246 - DNA, 10 bp, Clostridium sp.
  source 1..10: mol_type = other DNA
agttttttta

SEQ ID NO: 247 - DNA, 10 bp, Clostridium sp.
  source 1..10: mol_type = other DNA
ttatagcaaa

SEQ ID NO: 248 - DNA, 10 bp, Clostridium sp.
  source 1..10: mol_type = other DNA
atgattttta

SEQ ID NO: 249 - DNA, 10 bp, Clostridium sp.
  source 1..10: mol_type = other DNA
atacaagaat

SEQ ID NO: 250 - DNA, 10 bp, Clostridium sp.
  source 1..10: mol_type = other DNA
gaggtgttta

SEQ ID NO: 251 - DNA, 10 bp, Clostridium sp.
  source 1..10: mol_type = other DNA
aatgcattag

SEQ ID NO: 252 - DNA, 10 bp, Erysipelatoclostridium ramosum
  source 1..10: mol_type = other DNA
attataatca

SEQ ID NO: 253 - DNA, 10 bp, Erysipelatoclostridium ramosum
  source 1..10: mol_type = other DNA
tggaggcgtt

SEQ ID NO: 254 - DNA, 10 bp, Erysipelatoclostridium ramosum
  source 1..10: mol_type = other DNA
accttcatcg

SEQ ID NO: 255 - DNA, 10 bp, Erysipelatoclostridium ramosum
  source 1..10: mol_type = other DNA
tgaattttca

SEQ ID NO: 256 - DNA, 10 bp, Erysipelatoclostridium ramosum
  source 1..10: mol_type = other DNA
agcattatca

SEQ ID NO: 257 - DNA, 10 bp, Erysipelatoclostridium ramosum
  source 1..10: mol_type = other DNA
gtaaatcgtt

SEQ ID NO: 258 - DNA, 10 bp, Erysipelatoclostridium ramosum
  source 1..10: mol_type = other DNA
ttcaagatca

SEQ ID NO: 259 - DNA, 10 bp, Erysipelatoclostridium ramosum
  source 1..10: mol_type = other DNA
cttaaatctc

SEQ ID NO: 260 - DNA, 10 bp, Clostridium bolteae
  source 1..10: mol_type = other DNA
cgagcctgtc

SEQ ID NO: 261 - DNA, 10 bp, Clostridium bolteae
  source 1..10: mol_type = other DNA
aggggcattg

SEQ ID NO: 262 - DNA, 10 bp, Clostridium bolteae
  source 1..10: mol_type = other DNA
acagttattc

SEQ ID NO: 263 - DNA, 10 bp, Clostridium bolteae
  source 1..10: mol_type = other DNA
attctttgtg

SEQ ID NO: 264 - DNA, 10 bp, Clostridium bolteae
  source 1..10: mol_type = other DNA
attcagtttc

SEQ ID NO: 265 - DNA, 10 bp, Clostridium bolteae
  source 1..10: mol_type = other DNA
cctgcatcat

SEQ ID NO: 266 - DNA, 10 bp, Clostridium bolteae
  source 1..10: mol_type = other DNA
tgccgggttc

SEQ ID NO: 267 - DNA, 10 bp, Clostridium bolteae
  source 1..10: mol_type = other DNA
attgatattt

SEQ ID NO: 268 - DNA, 10 bp, Clostridium bolteae
  source 1..10: mol_type = other DNA
ctgcaagctc

SEQ ID NO: 269 - DNA, 10 bp, Clostridium bolteae
  source 1..10: mol_type = other DNA
tcacaacgac

SEQ ID NO: 270 - DNA, 10 bp, Clostridium bolteae
  source 1..10: mol_type = other DNA
cagttctttc

SEQ ID NO: 271 - DNA, 10 bp, Clostridium bolteae
  source 1..10: mol_type = other DNA
cgctcctgcg

SEQ ID NO: 272 - DNA, 10 bp, Clostridium bolteae
  source 1..10: mol_type = other DNA
gccaaccctg

SEQ ID NO: 273 - DNA, 10 bp, Clostridium bolteae
  source 1..10: mol_type = other DNA
caattgaccg

SEQ ID NO: 274 - DNA, 10 bp, Clostridium bolteae
  source 1..10: mol_type = other DNA
tcttcatttc

SEQ ID NO: 275 - DNA, 10 bp, Clostridium bolteae
  source 1..10: mol_type = other DNA
cctcaatcct

SEQ ID NO: 276 - DNA, 10 bp, Clostridium bolteae
  source 1..10: mol_type = other DNA
cgatggcttc

SEQ ID NO: 277 - DNA, 10 bp, Clostridium bolteae
  source 1..10: mol_type = other DNA
ccccctgctt

SEQ ID NO: 278 - DNA, 10 bp, Clostridium bolteae
  source 1..10: mol_type = other DNA
cccctgcctc

SEQ ID NO: 279 - DNA, 10 bp, Clostridium bolteae
  source 1..10: mol_type = other DNA
ttgctggccc

SEQ ID NO: 280 - DNA, 10 bp, Clostridium bolteae
  source 1..10: mol_type = other DNA
acggtccctc

SEQ ID NO: 281 - DNA, 10 bp, Clostridium bolteae
  source 1..10: mol_type = other DNA
tttacggatt

SEQ ID NO: 282 - DNA, 10 bp, Clostridium bolteae
  source 1..10: mol_type = other DNA
ggataagttc

SEQ ID NO: 283 - DNA, 10 bp, Clostridium bolteae
  source 1..10: mol_type = other DNA
tcaaacccgc

SEQ ID NO: 284 - DNA, 10 bp, Clostridium bolteae
  source 1..10: mol_type = other DNA
ggctttgctt

SEQ ID NO: 285 - DNA, 10 bp, Clostridium bolteae
  source 1..10: mol_type = other DNA
gttaagtttg

SEQ ID NO: 286 - DNA, 10 bp, Clostridium bolteae
  source 1..10: mol_type = other DNA
atcatcactc

SEQ ID NO: 287 - DNA, 10 bp, Clostridium bolteae
  source 1..10: mol_type = other DNA
tgggtgttat

SEQ ID NO: 288 - DNA, 10 bp, Clostridium bolteae
  source 1..10: mol_type = other DNA
cgccgagttc

SEQ ID NO: 289 - DNA, 10 bp, Clostridium bolteae
  source 1..10: mol_type = other DNA
ctatatttaa

SEQ ID NO: 290 - DNA, 10 bp, Clostridium bolteae
  source 1..10: mol_type = other DNA
cgtgcttttc

SEQ ID NO: 291 - DNA, 10 bp, Clostridium bolteae
  source 1..10: mol_type = other DNA
atcaaactta

SEQ ID NO: 292 - DNA, 10 bp, Clostridium bolteae
  source 1..10: mol_type = other DNA
tgatgcactt

SEQ ID NO: 293 - DNA, 10 bp, Clostridium bolteae
  source 1..10: mol_type = other DNA
tttttacagc

SEQ ID NO: 294 - DNA, 10 bp, Clostridium bolteae
  source 1..10: mol_type = other DNA
atttggcccc

SEQ ID NO: 295 - DNA, 10 bp, Clostridium bolteae
  source 1..10: mol_type = other DNA
gacctggatt

SEQ ID NO: 296 - DNA, 10 bp, Clostridium clostridioforme
  source 1..10: mol_type = other DNA
aaggtgcttc

SEQ ID NO: 297 - DNA, 10 bp, Clostridium clostridioforme
  source 1..10: mol_type = other DNA
cggtatttgt

SEQ ID NO: 298 - DNA, 10 bp, Clostridium clostridioforme
  source 1..10: mol_type = other DNA
gtaaatgttc

SEQ ID NO: 299 - DNA, 10 bp, Clostridium clostridioforme
  source 1..10: mol_type = other DNA
cagtgagagg

SEQ ID NO: 300 - DNA, 10 bp, Clostridium clostridioforme
  source 1..10: mol_type = other DNA
ttatggattc

SEQ ID NO: 301 - DNA, 10 bp, Clostridium clostridioforme
  source 1..10: mol_type = other DNA
ggaggaacaa

SEQ ID NO: 302 - DNA, 10 bp, Clostridium clostridioforme
  source 1..10: mol_type = other DNA
cacgctcttc

SEQ ID NO: 303 - DNA, 10 bp, Clostridium clostridioforme
  source 1..10: mol_type = other DNA
gcgtgcaatg

SEQ ID NO: 304 - DNA, 10 bp, Clostridium clostridioforme
  source 1..10: mol_type = other DNA
tgggtatttc

SEQ ID NO: 305 - DNA, 10 bp, Clostridium clostridioforme
  source 1..10: mol_type = other DNA
tcagtttagc

SEQ ID NO: 306 - DNA, 10 bp, Clostridium clostridioforme
  source 1..10: mol_type = other DNA
gctgtctttc

SEQ ID NO: 307 - DNA, 10 bp, Clostridium clostridioforme
  source 1..10: mol_type = other DNA
actacgtcta

SEQ ID NO: 308 - DNA, 10 bp, Clostridium clostridioforme
  source 1..10: mol_type = other DNA
tatgtcattc

SEQ ID NO: 309 - DNA, 10 bp, Clostridium clostridioforme
  source 1..10: mol_type = other DNA
gtgataactg

SEQ ID NO: 310 - DNA, 10 bp, Clostridium clostridioforme
  source 1..10: mol_type = other DNA
taacaacact

SEQ ID NO: 311 - DNA, 10 bp, Clostridium clostridioforme
  source 1..10: mol_type = other DNA
gaaacaggtt

SEQ ID NO: 312 - DNA, 10 bp, Clostridium clostridioforme
  source 1..10: mol_type = other DNA
taaaaaccta

SEQ ID NO: 313 - DNA, 10 bp, Clostridium clostridioforme
  source 1..10: mol_type = other DNA
gaagagggca

SEQ ID NO: 314 - DNA, 10 bp, Clostridium clostridioforme
  source 1..10: mol_type = other DNA
cggaagctct

SEQ ID NO: 315 - DNA, 10 bp, Clostridium clostridioforme
  source 1..10: mol_type = other DNA
agccattgtt

SEQ ID NO: 316 - DNA, 10 bp, Clostridium clostridioforme
  source 1..10: mol_type = other DNA
tcttccattc

SEQ ID NO: 317 - DNA, 10 bp, Clostridium clostridioforme
  source 1..10: mol_type = other DNA
tcctgtttat

SEQ ID NO: 318 - DNA, 10 bp, Clostridium clostridioforme
  source 1..10: mol_type = other DNA
cttttttggg

SEQ ID NO: 319 - DNA, 10 bp, Clostridium clostridioforme
  source 1..10: mol_type = other DNA
ggagatatac

SEQ ID NO: 320 - DNA, 10 bp, Clostridium clostridioforme
  source 1..10: mol_type = other DNA
ctatcttttc

SEQ ID NO: 321 - DNA, 10 bp, Clostridium clostridioforme
  source 1..10: mol_type = other DNA
cgcacacgca

SEQ ID NO: 322 - DNA, 10 bp, Clostridium clostridioforme
  source 1..10: mol_type = other DNA
tggatacttc

SEQ ID NO: 323 - DNA, 10 bp, Clostridium clostridioforme
  source 1..10: mol_type = other DNA
atggtagagc

SEQ ID NO: 324 - DNA, 10 bp, Clostridium clostridioforme
  source 1..10: mol_type = other DNA
ctgattcttc

SEQ ID NO: 325 - DNA, 10 bp, Clostridium clostridioforme
  source 1..10: mol_type = other DNA
ccggattgga

SEQ ID NO: 326 - DNA, 10 bp, Clostridium clostridioforme
  source 1..10: mol_type = other DNA
attgattttc

SEQ ID NO: 327 - DNA, 10 bp, Clostridium clostridioforme
  source 1..10: mol_type = other DNA
tatcttgcaa

SEQ ID NO: 328 - DNA, 10 bp, Clostridium clostridioforme
  source 1..10: mol_type = other DNA
cggaaggttc

SEQ ID NO: 329 - DNA, 10 bp, Clostridium clostridioforme
  source 1..10: mol_type = other DNA
ttcccggctc

SEQ ID NO: 330 - DNA, 10 bp, Clostridium clostridioforme
  source 1..10: mol_type = other DNA
cttttccttc

SEQ ID NO: 331 - DNA, 10 bp, Clostridium clostridioforme
  source 1..10: mol_type = other DNA
gacgtaacct

SEQ ID NO: 332 - DNA, 10 bp, Clostridium clostridioforme
  source 1..10: mol_type = other DNA
ggatgagttt

SEQ ID NO: 333 - DNA, 10 bp, Clostridium clostridioforme
  source 1..10: mol_type = other DNA
gagatggaca

SEQ ID NO: 334 - DNA, 10 bp, Clostridium clostridioforme
  source 1..10: mol_type = other DNA
tgactccttc

SEQ ID NO: 335 - DNA, 10 bp, Clostridium clostridioforme
  source 1..10: mol_type = other DNA
tccacggtat

SEQ ID NO: 336 - DNA, 10 bp, Clostridium clostridioforme
  source 1..10: mol_type = other DNA
gtaaaaccaa

SEQ ID NO: 337 - DNA, 10 bp, Clostridium clostridioforme
  source 1..10: mol_type = other DNA
gaacgtgttc

SEQ ID NO: 338 - DNA, 10 bp, Clostridium clostridioforme
  source 1..10: mol_type = other DNA
gggcggtttc

SEQ ID NO: 339 - DNA, 10 bp, Clostridium clostridioforme
  source 1..10: mol_type = other DNA
tgacaacagg

SEQ ID NO: 340 - DNA, 10 bp, Clostridium clostridioforme
  source 1..10: mol_type = other DNA
cccatgtttc

SEQ ID NO: 341 - DNA, 10 bp, Clostridium clostridioforme
  source 1..10: mol_type = other DNA
ggcggttgtc

SEQ ID NO: 342 - DNA, 10 bp, Clostridium clostridioforme
  source 1..10: mol_type = other DNA
caaaactttc

SEQ ID NO: 343 - DNA, 10 bp, Clostridium clostridioforme
  source 1..10: mol_type = other DNA
acatcagtga

SEQ ID NO: 344 - DNA, 10 bp, Clostridium clostridioforme
  source 1..10: mol_type = other DNA
gtacgtgttc

SEQ ID NO: 345 - DNA, 10 bp, Clostridium clostridioforme
  source 1..10: mol_type = other DNA
cggtcagggc

SEQ ID NO: 346 - DNA, 10 bp, Clostridium clostridioforme
  source 1..10: mol_type = other DNA
caactatttc

SEQ ID NO: 347 - DNA, 10 bp, Clostridium clostridioforme
  source 1..10: mol_type = other DNA
tactgtcatt

SEQ ID NO: 348 - DNA, 10 bp, Clostridium clostridioforme
  source 1..10: mol_type = other DNA
gctgctatct

SEQ ID NO: 349 - DNA, 10 bp, Clostridium clostridioforme
  source 1..10: mol_type = other DNA
ctttgtcaac

SEQ ID NO: 350 - DNA, 10 bp, Clostridium clostridioforme
  source 1..10: mol_type = other DNA
cccccttttc

SEQ ID NO: 351 - DNA, 10 bp, Clostridium clostridioforme
  source 1..10: mol_type = other DNA
ccttttatat

SEQ ID NO: 352 - DNA, 10 bp, Clostridium clostridioforme
  source 1..10: mol_type = other DNA
gaaacataca

SEQ ID NO: 353 - DNA, 10 bp, Clostridium clostridioforme
  source 1..10: mol_type = other DNA
aagtgtacgc

SEQ ID NO: 354 - DNA, 10 bp, Clostridium scindens
  source 1..10: mol_type = other DNA
tcattgcttt

SEQ ID NO: 355 - DNA, 10 bp, Clostridium scindens
  source 1..10: mol_type = other DNA
tttcgcaatt

SEQ ID NO: 356 - DNA, 10 bp, Clostridium scindens
  source 1..10: mol_type = other DNA
ttcttcattt

SEQ ID NO: 357 - DNA, 10 bp, Clostridium scindens
  source 1..10: mol_type = other DNA
atgtgccctg

SEQ ID NO: 358 - DNA, 10 bp, Clostridium scindens
  source 1..10: mol_type = other DNA
atagaatttt

SEQ ID NO: 359 - DNA, 10 bp, Clostridium scindens
  source 1..10: mol_type = other DNA
gtctataatc

SEQ ID NO: 360 - DNA, 10 bp, Clostridium scindens
  source 1..10: mol_type = other DNA
tacttttttc

SEQ ID NO: 361 - DNA, 10 bp, Clostridium scindens
  source 1..10: mol_type = other DNA
gggaaaccat

SEQ ID NO: 362 - DNA, 10 bp, Clostridium scindens
  source 1..10: mol_type = other DNA
tttgaacgct

SEQ ID NO: 363 - DNA, 10 bp, Clostridium scindens
  source 1..10: mol_type = other DNA
ggcgggatgc

SEQ ID NO: 364 - DNA, 10 bp, Clostridium scindens
  source 1..10: mol_type = other DNA
ttcccggttc

SEQ ID NO: 365 - DNA, 10 bp, Clostridium scindens
  source 1..10: mol_type = other DNA
aagttcttcc

SEQ ID NO: 366 - DNA, 10 bp, Clostridium scindens
  source 1..10: mol_type = other DNA
aagatgttcc

SEQ ID NO: 367 - DNA, 10 bp, Clostridium scindens
  source 1..10: mol_type = other DNA
aggccaacta

SEQ ID NO: 368 - DNA, 10 bp, Clostridium scindens
  source 1..10: mol_type = other DNA
tatacagttt

SEQ ID NO: 369 - DNA, 10 bp, Clostridium scindens
  source 1..10: mol_type = other DNA
gtccacatct

SEQ ID NO: 370 - DNA, 10 bp, Clostridium scindens
  source 1..10: mol_type = other DNA
ttctgggttt

SEQ ID NO: 371 - DNA, 10 bp, Clostridium scindens
  source 1..10: mol_type = other DNA
ctgggtttgg

SEQ ID NO: 372 - DNA, 10 bp, Clostridium scindens
  source 1..10: mol_type = other DNA
ggatccgttc

SEQ ID NO: 373 - DNA, 10 bp, Clostridium scindens
  source 1..10: mol_type = other DNA
ggaaggatcg

SEQ ID NO: 374 - DNA, 10 bp, Clostridium scindens
  source 1..10: mol_type = other DNA
gatatattcc

SEQ ID NO: 375 - DNA, 10 bp, Clostridium scindens
  source 1..10: mol_type = other DNA
tgagtttacg

SEQ ID NO: 376 - DNA, 10 bp, Clostridium scindens
  source 1..10: mol_type = other DNA
ggatggtctc

SEQ ID NO: 377 - DNA, 10 bp, Clostridium scindens
  source 1..10: mol_type = other DNA
aacaacctgc

SEQ ID NO: 378 - DNA, 10 bp, Clostridium scindens
  source 1..10: mol_type = other DNA
tcagcacgat

SEQ ID NO: 379 - DNA, 10 bp, Clostridium scindens
  source 1..10: mol_type = other DNA
gaaggcgtgt

SEQ ID NO: 380 - DNA, 10 bp, Clostridium scindens
  source 1..10: mol_type = other DNA
taattgttca

SEQ ID NO: 381 - DNA, 10 bp, Clostridium scindens
  source 1..10: mol_type = other DNA
ctggcctgtc

SEQ ID NO: 382 - DNA, 10 bp, Clostridium scindens
  source 1..10: mol_type = other DNA
tggatcattt

SEQ ID NO: 383 - DNA, 10 bp, Clostridium scindens
  source 1..10: mol_type = other DNA
agaactcacg

SEQ ID NO: 384 - DNA, 10 bp, Clostridium scindens
  source 1..10: mol_type = other DNA
aaataacttc

SEQ ID NO: 385 - DNA, 10 bp, Clostridium scindens
  source 1..10: mol_type = other DNA
tgcgaccacg

SEQ ID NO: 386 - DNA, 10 bp, Clostridium scindens
  source 1..10: mol_type = other DNA
atcttcatca

SEQ ID NO: 387 - DNA, 10 bp, Clostridium scindens
  source 1..10: mol_type = other DNA
gaactgtcag

SEQ ID NO: 388 - DNA, 10 bp, Clostridium scindens
  source 1..10: mol_type = other DNA
cataatcttt

SEQ ID NO: 389 - DNA, 10 bp, Clostridium scindens
  source 1..10: mol_type = other DNA
attgccagcc

SEQ ID NO: 390 - DNA, 10 bp, Clostridium scindens
  source 1..10: mol_type = other DNA
caatgaattt

SEQ ID NO: 391 - DNA, 10 bp, Clostridium scindens
  source 1..10: mol_type = other DNA
gtcgatgccg

SEQ ID NO: 392 - DNA, 10 bp, Clostridium scindens
  source 1..10: mol_type = other DNA
tctccagttc

SEQ ID NO: 393 - DNA, 10 bp, Clostridium scindens
  source 1..10: mol_type = other DNA
acttctggag

SEQ ID NO: 394 - DNA, 10 bp, Clostridium scindens
  source 1..10: mol_type = other DNA
tgcgctcttt

SEQ ID NO: 395 - DNA, 10 bp, Clostridium scindens
  source 1..10: mol_type = other DNA
ggtcaaacgg

SEQ ID NO: 396 - DNA, 10 bp, Clostridium scindens
  source 1..10: mol_type = other DNA
acctgtcttt

SEQ ID NO: 397 - DNA, 10 bp, Clostridium scindens
  source 1..10: mol_type = other DNA
aacagtacct

SEQ ID NO: 398 - DNA, 10 bp, Clostridium scindens
  source 1..10: mol_type = other DNA
gtaaccatgt

SEQ ID NO: 399 - DNA, 10 bp, Clostridium scindens
  source 1..10: mol_type = other DNA
aaatgccgca

SEQ ID NO: 400 - DNA, 10 bp, Clostridium scindens
  source 1..10: mol_type = other DNA
tgtatctttt

SEQ ID NO: 401 - DNA, 10 bp, Clostridium scindens
  source 1..10: mol_type = other DNA
ttttgtcgcg

SEQ ID NO: 402 - DNA, 10 bp, Clostridium scindens
  source 1..10: mol_type = other DNA
cggggtattc

SEQ ID NO: 403 - DNA, 10 bp, Clostridium scindens
  source 1..10: mol_type = other DNA
acggattaaa

SEQ ID NO: 404 - DNA, 10 bp, Clostridium scindens
  source 1..10: mol_type = other DNA
ggttaccctt

SEQ ID NO: 405 - DNA, 10 bp, Clostridium scindens
  source 1..10: mol_type = other DNA
ggcaaatatg

SEQ ID NO: 406 - DNA, 10 bp, Clostridium scindens
  source 1..10: mol_type = other DNA
atgcggattt

SEQ ID NO: 407 - DNA, 10 bp, Clostridium scindens
  source 1..10: mol_type = other DNA
aacgctcact

SEQ ID NO: 408 - DNA, 10 bp, Clostridium scindens
  source 1..10: mol_type = other DNA
aattttcttt

SEQ ID NO: 409 - DNA, 10 bp, Clostridium scindens
  source 1..10: mol_type = other DNA
ctttgctgag

SEQ ID NO: 410 - DNA, 83 bp, synthetic construct
  source 1..83: mol_type = other DNA
taagatgtattgaaatatgaccacaaagacacgctcggattagcagtatacgtttaataacaacataagatgtattgaaatta

SEQ ID NO: 411 - DNA, 65 bp, synthetic construct
  source 1..65: mol_type = other DNA
attgaaatatgaccacaaagacacgctcggattagcagtatacgtttaataacaacataagatgt

SEQ ID NO: 412 - RNA, 65 nt, synthetic construct
  source 1..65: mol_type = other RNA
attgaaatatgaccacaaagacacgctcggattagcagtatacgtttaataacaacataagatgt

SEQ ID NO: 413 - DNA, 123 bp, synthetic construct
  source 1..123: mol_type = other DNA
gctgaagggcatcgacttcaaggaggacggcaacatcctggggcacaagctggagtacaactacaacagccacaacgtctatatcatggccgacaagcagaagaacggcatcaaggtgaactt

SEQ ID NO: 414 - DNA, 96 bp, synthetic construct
  source 1..96: mol_type = other DNA
gtttaataacaacataagatgtattgaaatacttcaaggaggacggcaacatcctggggcacaagcgtttaataacaacataagatgtattgaaat

SEQ ID NO: 415 - DNA, 96 bp, synthetic construct
  source 1..96: mol_type = other DNA
gtttaataacaacataagatgtattgaaataggccgacaagcagaagaacggcatcaaggtgaactgtttaataacaacataagatgtattgaaat